(19) United States
(12) Patent Application Publication
(10) Pub. No.: US 2009/0263516 A1
(43) Pub. Date: Oct. 22, 2009

(54) Plant Extract Composition and Their Use to Modulate Cellular Activity

(75) Inventor: Benoit CYR, St. Augustin de Desmaures (CA)

Correspondence Address: SHEPPARD, MULLIN, RICHTER & HAMPTON LLP, 990 Marsh Road, Menlo Park, CA, 94025, US

(73) Assignee: Biopharmacopae Design International Inc., Saint-Foy (CA)

(21) Appl. No.: 12/263,114

(22) Filed: Oct. 31, 2008

Related U.S. Application Data
(63) Continuation of application No. 10/526,387, filed on Oct. 6, 2005, now abandoned.

(30) Foreign Application Priority Data
Aug. 30, 2002 (CA) 2400 936

(51) Int. Cl. A61K 36/8962; A61K 36/00; A61P 35/00; C12N 5/06; A61K 36/31; A61K 36/899

(52) U.S. Cl. 424/754; 424/725; 435/375; 424/774; 424/779; 424/755; 424/750; 424/777

(57) ABSTRACT

Extracts from plant material, or semi-purified/purified molecules or compounds prepared from the extracts that demonstrate the ability to modulate one or more cellular activities are provided. The extracts are capable of slowing down, inhibiting or preventing cell migration, for example, the migration of endothelial cells or neoplastic cells and thus, the use of the extracts to slow down, inhibit or prevent abnormal cell migration in an animal is also provided. Methods of selecting and preparing the plant extracts and methods of screening the extracts to determine their ability to modulate one or more cellular activity are described. The purification or semi-purification of one or more molecules from the described extracts is also contemplated as well as the use of these molecules, alone or in combination with an extract, to slow down, inhibit or prevent abnormal cell migration in an animal.

### CROSS-REFERENCE TO RELATED APPLICATION

This application is a continuation of U.S. patent application Ser. No. 10/526,387 filed on Oct. 6, 2005, which claims the benefit under 35 U.S.C. § 371 to PCT Application No. PCT/CA2003/001284 filed on Sep. 2, 2003, which claims priority to Canadian Application No. 2,400,936, filed on Aug. 30, 2002, all of which are incorporated herein in their entireties.

### FIELD OF INVENTION

The invention pertains to the field of modulators of cellular activity, specifically within the field of inhibitors of extracellular proteases.

### BACKGROUND OF THE INVENTION

The cells of tissues are generally in contact with a network of large extracellular macromolecules that occupies the spaces in a tissue between the component cells and also occupies the space between adjacent tissues. This extracellular matrix functions as a scaffolding on which the cells and tissue are supported and is involved actively in regulating interaction of the cells that contact it. The principal macromolecules of the extracellular matrix include the collagens (the most abundant proteins in the body) and glycosaminoglycans (complex polysaccharides which are usually bonded also to protein and then termed proteoglycans). The macromolecules that comprise the extracellular matrix are produced typically by the cells in contact therewith, for example, epithelial cells in contact with a basement membrane and fibroblasts embedded in connective tissue.

The glycosaminoglycan (proteoglycan) molecules form a highly hydrated matrix (a gel) in which elastic or fibrous proteins (such as collagen fibres) are embedded. The aqueous nature of the gel permits diffusion of metabolically required substances between the cells of a tissue and between tissues. Additional proteins that may be found in extracellular matrix include elastin, fibronectin and laminin.

The term “connective tissue” refers to extracellular matrix plus specialised cells such as, for example, fibroblasts, chondrocytes, osteoblasts, macrophages and mast cells found therein. The term “interstitial tissue” is best reserved for an extracellular matrix that stabilises a tissue internally, filling the gaps between the cells thereof. There are also specialised forms of extracellular matrix (connective tissue) that have additional functional roles-cornea, cartilage and tendon, and when calcified, the bones and teeth.

A structural form of extracellular matrix is the basal lamina (basement membrane). Basal laminae are thin zones of extracellular matrix that are found under epithelium or surrounding, for example, muscle cells or the cells that electrically insulate nerve fibres. Generally speaking, basal laminae separate cell layers from underlying zones of connective tissue or serve as a boundary between two cell layers wherein a basal lamina can serve as a pathway for invading cells associated with pathologic processes, or for structural organisation associated with tissue repair (i.e. as a blueprint from which to regenerate original tissue architecture and morphology).

The regulated turnover of extracellular matrix macromolecules is critical to a variety of important biological processes. Localised degradation of matrix components is required when cells migrate through a basal lamina, as when white blood cells migrate across the vascular basal lamina into tissues in response to infection or injury, or when cancer cells migrate from their site of origin to distant organs via the bloodstream or lymphatic vessels, during metastasis. In normal tissues, the activity of extracellular proteases is tightly regulated and the breakdown/production of connective tissue is in dynamic equilibrium, such that there is a slow and continual turnover due to degradation and resynthesis in the extracellular matrix of adult animals.

In each of these cases, matrix components are degraded by extracellular proteolytic enzymes that are secreted locally by cells. These proteases belong to one of four general classes: many are metalloproteinases, which depend on bound Ca²⁺ or Zn²⁺ for activity, while the others are serine, aspartic and cysteine proteases, which have a highly reactive serine, aspartate or cysteine residue in their respective active site (Vincenti et al., (1994) Arthritis and Rheumatism, 37: 1115-1126). Together, metalloproteinases, serine, aspartate and cysteine proteases cooperate to degrade matrix proteins such as collagen, laminin, and fibronectin.

Several mechanisms operate to ensure that the degradation of matrix components is tightly controlled. First, many proteases are secreted as inactive precursors that can be activated locally. Second, the action of proteases is confined to specific areas by various secreted protease inhibitors, such as the tissue inhibitors of metalloproteases and the serine protease inhibitors known as serpins. These inhibitors are specific for particular proteases and bind tightly to the activated enzyme to block its activity. Third, many cells have receptors on their surface that bind proteases, thereby confining the enzyme to where it is needed.

Many pathogenic bacteria produce extracellular metalloproteases, of which many are zinc containing proteases that can be classified into two families, the thermolysin (neutral) proteases and the serralysin (alkaline) proteases.

A number of patents and publications report the inhibition of one or more extracellular proteases by compounds extracted from plants. For example, Sun et al., (1996) Phytotherapy Res., 10: 194-197, reports the inhibition in vitro of stromelysin (MMP-3) and collagenase by betulinic acid extracted from Doliocarpus verruculosis. Sazuka et al, (1997) Biosci. Biotechnol. Biochem., 61: 1504-1506, reports the inhibition of gelatinases (MMP-2 and MMP-9) and metastasis by compounds isolated from green and black teas. Kumagai et al, JP 08104628 A2, Apr. 1, 1996 (CA 125: 67741) reports the use of flavones and anthocyanines isolated from Scutellaris baicanlensis roots to inhibit collagenase. Gervasi et al., (1996) Biochem. Biophys. Res. Comm., 228: 530-538, reports the regulation of MMP-2 by some plant lectins and other saccharides. Dubois et al., (1998) FEBS Lett., 427: 275-278, reports the increased secretion of deleterious gelatinase-B (MMP-9) by some plant lectins. Nagase et al., (1998) Planta Med., 64: 216-219, reports the weak inhibition of collagenase (MMPs) by delphinidin, a flavonoid isolated from Solanum melongena. Other reports discuss the use of extracts to inhibit extracellular proteases. For example, Asano et al., (1998) Immunopharmacology, 39: 117-126, reports the inhibition of TNF-α production using Tripterygium wilfordii Hook F. extracts. Maheu et al., (1998) Arthritis Rheumatol., 41: 81-91, reports the use of avocado/soy bean non-saponifiable extracts in the treatment of arthritis. Makimura et al., (1993) J. Periodontol., 64: 630-636, also reports the use of green tea extracts to inhibit collagenases in vitro. Obayashi et al., (1998) Nippon Keshonin Gijutsusha Kaishi, 32: 272-279 (CA 130: 92196) reports the inhibition of collagenase-I (MMP-1) from human fibroblast and neutrophil elastase by plant extract from Eucalyptus and Elder.

When a plant is stressed, several biochemical processes are activated and many new chemicals, in addition to those constitutively expressed, are synthesised as a response. These chemicals include enzymes, enzyme inhibitors (especially protease inhibitors), lectins, alkaloids, terpenes, oligosaccharides, and antibiotics. The biosynthesis of these defense chemicals and secondary metabolites is not yet fully understood. The most studied system is the production of protease inhibitors following pest attack or mechanical wounding. On the other hand, several inducible chemicals are the products of complex biochemical pathways, which require several biosynthetic enzymes to be activated.

It has been shown that many chemicals can be used to “stress” plants and to artificially stimulate biosynthesis of several new and constitutive defense chemicals. Also, different types of stress can activate distinct metabolic defense pathways, thereby leading to production of a variety of chemicals. Although the various biosynthetic defense pathways share some similarities, these pathways are characteristic of specific plant species. Therefore, treating many plants with many types of stress can lead to a vast number of collections of diverse chemicals from plant origin.

In addition to pests, fungi, and other pathogenic attacks, stressors include drought, heat, water and mechanical wounding. Furthermore, many chemicals can act as stressors that activate gene expression; these include: hydrogen peroxide, ozone, sodium chloride, jasmonic acid and derivatives, α-linoleic acid, γ-linoleic acid, salicylic acid, abscesic acid, volicitin, small oligopeptides, among others.

The use of abiotic stressors on plants has been the focus of intense studies in plant science. Artificial stresses have been used to stimulate the production of natural plant protease inhibitors for insect digestive proteases, in order to enhance crop protection against certain pests and herbivores. They have proven useful in combination with plants genetically modified to express other protease inhibitor genes. Finally, in the area of molecular farming, stresses have been used to stimulate gene expression in plants genetically modified to include an inducible coding sequence for a protein of nutraceutical and/or medicinal interest (Ryan and Farmer, U.S. Pat. No. 5,935,809).

Likewise, the use of gene activators or elicitors have been described to enhance the production of volatile chemicals in plant cell cultures. These elicitors have been demonstrated to induce the activity of several enzymes such as for example phenylalanine ammonia lyase, therefore leading to an increase in the production of plant volatile components.

### SUMMARY OF THE INVENTION

An object of the invention is to provide plant extract compositions and their use to modulate cellular activity. In accordance with one aspect of the present invention, there is provided a plant extract that inhibits the activity of at least one extracellular protease, said extract having at least one of the following properties: (i) is capable of slowing down or inhibiting migration of endothelial cells, and (ii) is capable of slowing down or inhibiting migration of neoplastic cells.

In accordance with another aspect of the present invention, there is provided a sub-library of plant extracts, said sub-library being prepared by a process comprising:
(a) harvesting plant material from selected plants;
(b) contacting said plant material with a solvent to provide a plurality of potential extracts;
(c) analysing each potential extract for inhibitory activity against at least one extracellular protease;
(d) selecting those potential extracts that are capable of inhibiting the activity of at least one extracellular protease to provide a library of extracts;
(e) analysing the ability of each extract in said library to slow down migration of endothelial or neoplastic cells in vitro, and
(f) selecting those extracts that are capable of slowing down migration of said endothelial or neoplastic cells to provide a sub-library of plant extracts.

In accordance with another aspect of the present invention, there is provided a pharmaceutical composition comprising a plant extract of the invention and a pharmaceutically acceptable diluent, excipient or carrier.

In accordance with another aspect of the present invention, there is provided a use of a plant extract of the invention to slow down, inhibit or prevent angiogenesis in an animal in need thereof.

In accordance with another aspect of the present invention, there is provided a use of a plant extract of the invention to slow down, inhibit or prevent metastasis in an animal in need thereof.

In accordance with another aspect of the present invention, there is provided a use of a plant extract of the invention in the manufacture of a medicament.

In accordance with another aspect of the present invention, there is provided a use of a plant extract to slow down cell migration in an animal in need thereof, wherein said plant extract inhibits the activity of at least one extracellular protease and has at least one of the following properties: (i) is capable of slowing down or inhibiting migration of endothelial cells, and (ii) is capable of slowing down or inhibiting migration of neoplastic cells.

In accordance with another aspect of the present invention, there is provided a process for preparing a sub-library of plant extracts that are capable of slowing down or inhibiting cell migration, said process comprising:
(a) harvesting plant material from selected plants;
(b) contacting said plant material with a solvent to provide a plurality of potential extracts;
(c) analysing each potential extract for inhibitory activity against at least one extracellular protease;
(d) selecting those potential extracts that are capable of inhibiting the activity of at least one extracellular protease provide a library of extracts;
(e) analysing the ability of each extract in said library to slow down migration of endothelial or neoplastic cells in vitro, and
(f) selecting those extracts that are capable of slowing down migration of said endothelial or neoplastic cells to provide a sub-library of plant extracts.

In accordance with another aspect of the present invention, there is provided a process for identifying a plant extract capable of inhibiting cell migration, said process comprising:
(a) harvesting plant material from a selected plants;
(b) contacting said plant material with a solvent to provide a plurality of potential extracts;
(c) analysing each potential extract for inhibitory activity against at least one extracellular protease;
(d) selecting those potential extracts that are capable of inhibiting the activity of at least one extracellular protease provide a library of plant extracts;
(e) analysing the ability of each plant extract in said library to slow down migration of endothelial or neoplastic cells in vitro, and
(f) selecting a plant extract that is capable of slowing down migration of said endothelial or neoplastic cells.

In accordance with another aspect of the present invention, there is provided a plant extract produced by the above process.

### BRIEF DESCRIPTION OF THE FIGURES

FIG. 1 presents an overview of a procedure that can be followed in one embodiment of the invention in order to generate plant extracts, each of which is derived from solid plant material.

FIG. 2 describes in further detail, a procedure that can be followed in one embodiment of the invention in order to generate the extracts of the invention.

FIG. 3 presents an overview of a commercial procedure that can be followed in one embodiment of the invention in order to prepare extracts of the invention.

FIG. 4 (a) untreated control cells; (b) show cells treated with an extract of the present invention having a concentration of 0.5×; (c) shows cells treated with an extract of the present invention having a concentration of 1×.

FIG. 5 (a) shows untreated cells; (b) shows cells plus a positive control; (c) shows cells treated with an extract of the present invention having a concentration of 1×; (d) shows cells treated with an extract of the present invention having a concentration of 2×.

### DETAILED DESCRIPTION OF THE INVENTION

The present invention provides for extracts from plant material, or semi-purified/purified molecules or compounds prepared from the extracts, that are capable of inhibiting one or more extracellular protease and that demonstrate the ability to modulate one or more cellular activities. In one embodiment of the invention the extracts are capable of slowing down, inhibiting or preventing cell migration, for example, the migration of endothelial cells or neoplastic cells. The present invention also provides for the use of the extracts to slow down, inhibit or prevent abnormal cell migration in an animal, and thus can be used, for example, in the alleviation of conditions where there is a need to slow down angiogenesis or neoplastic cell invasion.

The present invention further provides for methods of selecting and preparing the plant extracts and for methods of screening the extracts to determine their ability to modulate one or more cellular activity. The invention additionally provides for the purification or semi-purification of one or more molecules from the extract and for the use of the semi-purified/purified molecules, alone or in combination with an extract, to slow down, inhibit or prevent abnormal cell migration in an animal.

### DEFINITIONS

Unless defined otherwise, all technical and scientific terms used herein have the same meaning as commonly understood by one of ordinary skill in the art to which this invention belongs.

The term “potential plants,” as used herein, is intended to include all species of the Kingdom Plantae, including terrestrial, aquatic or other plants under the Division Chlorophyta, Division Rhodophora, Division Paeophyta, Division Bryophyta and Division Tracheophyta; Subdivision Lycopsida, Subdivision Sphenopsida, Subdivision Pteropsida and Subdivision Spermopsida; Class Gymnospermae, Class Angiospermae, Subclass Dicotyledonidae and Subclass Monocotyledonidae. In general terms, all plants, herbs, and lower plants such as fungi and algae are considered to be potential plants in accordance with the present invention.

The term “plant material,” as used herein, refers to any part or parts of a plant taken either individually or in a group. Examples include, but are not limited to, leaves, flowers, roots, seeds, stems, and other part of a plant, including those plants described herein as potential plants of the invention.

The term “extracellular protease,” as used herein, refers to an enzyme that is capable of degrading proteins (i.e. proteolysis) and which is secreted outside the cell. The cell can be prokaryotic or eukaryotic. Examples of extracellular proteases include, but are not limited to, matrix metalloproteases (MMPs), cathepsins, elastase, plasmin, TPA, uPA, kallikrein, ADAMS family members, neprilysin, gingipain, clostripain, thermolysin, serralysin, and other bacterial and viral proteases.

The term “panel of extracellular proteases,” refers to an array of distinct extracellular proteases that are used to perform routine assays to monitor the presence or absence of inhibitory activity throughout an extraction process of the invention. A panel typically comprises at least two proteases, but may for some purposes comprise as few as one protease. One skilled in the art would appreciate that as high throughput screening techniques develop, one could routinely assay for the presence or absence of inhibitory activity against as many extracellular proteases as the technology permits.

The term “potential pre-extract,” refers to refers to a composition prepared by contacting a solvent with plant material following the procedures described herein, which has not yet been determined to possess inhibitory activity against one or more extracellular protease.

The term “potential extract,” as used herein, refers to a potential pre-extract that has been subjected to one or more separation and/or purification step.

The term “extract of the invention,” as used herein, refers to a composition prepared by contacting a solvent with plant material following the procedures described herein, which demonstrates inhibitory activity against one or more extracellular protease and demonstrates an ability to modulate one or more cellular activity.

The term “protease inhibitor,” as used herein, refers to a molecule or compound that attenuates the proteolytic activity of proteases. A protease inhibitor may or may not be proteinaceous.

The term “stressor,” as used herein, refers to a factor, such as a physical stress, a chemical compound, or a biological agent that is used to elicit production of extracellular protease inhibitors as a result of activation of a defense response in a plant. Elicitors and inducers are also considered to be stressors.

The term “substantially purified” or “substantially pure” or “isolated,” when used in reference to a molecule or molecules having protease inhibitor activity, refers to a form of the molecule(s) that is relatively free of proteins, nucleic acids, lipids, carbohydrates or other materials with which it is naturally associated in a plant. As disclosed herein, a plant extract of the invention is considered to be substantially purified, in that it is removed from the plant tissue from which it is derived. In addition, molecules or compounds having protease inhibitor activity that are present within the extract can be further purified using routine and well-known methods such as those described herein. As such, a substantially pure protease inhibitor of the invention can constitute at least about one or a few percent of a sample, for example, at least about five percent of a sample. In one embodiment, the substantially pure protease inhibitor constitutes at least about twenty percent of a sample. In another embodiment, the protease inhibitor can be further purified to constitute at least about fifty percent of a sample. In a further embodiment, the protease inhibitor can be further purified to constitute at least about eighty percent of a sample. In other embodiments, the protease inhibitor can be further purified to constitute at least about ninety percent or at least about ninety-five percent or more of a sample. A determination that a protease inhibitor of the invention is substantially pure can be made using methods such as those disclosed herein or otherwise known in the art, for example, by performing electrophoresis and identifying the particular molecule as a relatively discrete band.

The term “cell migration,” as used herein, refers to the movement, typically abnormal, of a cell or cells from one locus to another. Examples of cell migration include the movement of cells through the extracellular matrix and/or basal lamina during angiogenesis or cell invasion.

Other chemistry terms herein are used according to conventional usage in the art, as exemplified by The McGraw-Hill Dictionary of Chemical Terms (ed. Parker, S., 1985), McGraw-Hill, San Francisco, incorporated herein by reference).

### Preparation of Plant Extracts

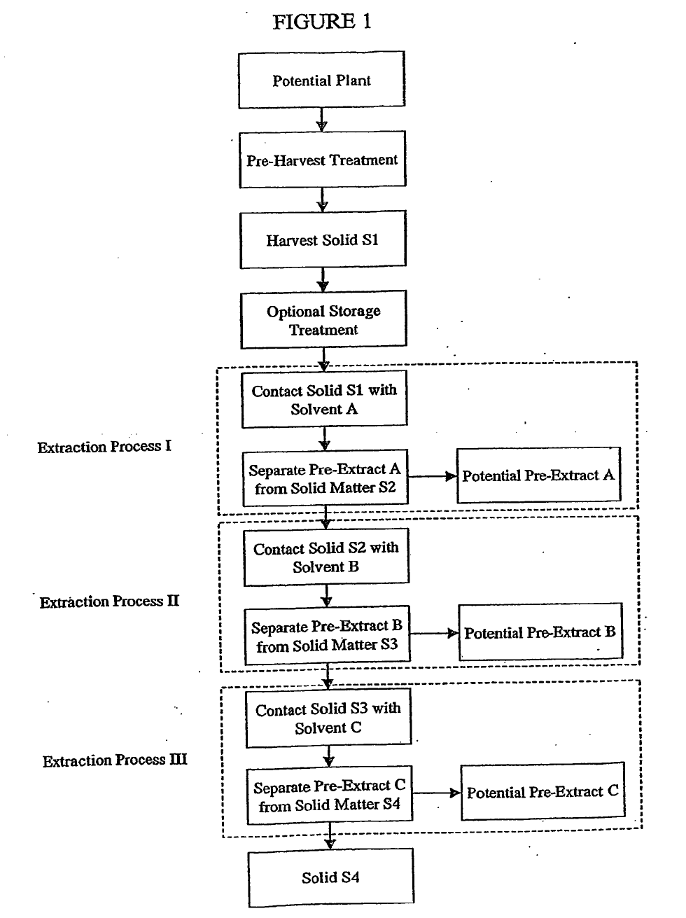

With reference to FIG. 1, one embodiment of the present invention provides a process for producing an extract of the invention that begins with the selection of a plant species. Once the plant species has been chosen, a pre-harvest treatment is selected, for example treatment with water, or treatment with water in addition to a stressor or a combination of stressors. The stress can be applied separately from the water (if the stress is drought, then the water would not be provided for the period in which the plant is to be stressed) or concomitantly. The next step of the process involves choosing whether the treated plant will be treated for storage and stored prior to contacting plant material with the first solvent or whether it will be used directly. The plant material is next treated with the first solvent after which the liquid is separated from the solid material (solid S2), wherein the liquid becomes Fraction F1 or Pre-Extract A. The solid S2 is treated with the second solvent and the liquid is again separated from the solid material (solid S3), wherein the liquid becomes Fraction F2 or Pre-Extract B. Finally, the solid S3 is treated with the third solvent and the liquid from this treatment is separated from the solid material (solid S4).

### Plant Material

Plant material suitable for use in preparing an extract of the invention is derived from a “potential plant.” Potential plants include all species of the Kingdom Plantae, including terrestrial, aquatic or other plants that can be subjected to the methodology described herein in order to generate an extract that can be tested against a panel of extracellular proteases. Those plants which yield an extract demonstrating inhibitory activity against an extracellular protease and an ability to modulate cellular activity are considered to be plants and extracts comprising the subject matter of the invention.

Examples of potential plants include, but are not limited to, those belonging to the following classifications: Superdivision Spermatophyta—Seed plants; Division Coniferophyta—Conifers; Class Pinopsida, Order Pinales; Family Araucariaceae—Araucaria family; Family Cephalotaxaceae—Plum Yew family; Family Cupressaceae—Cypress family; Family Pinaceae—Pine family; Family Podocarpaceae—Podocarpus family; Family Taxodiaceae—Redwood family; Order Taxales, Family Taxaceae—Yew family; Division Cycadophyta—Cycads, Class Cycadopsida, Order Cycadales, Family Cycadaceae—Cycad family; Family Zamiaceae—Sago-palm family; Division Ginkgophyta—Ginkgo, Class Ginkgoopsida, Order Ginkgoales, Family Ginkgoaceae—Ginkgo family; Division Gnetophyta—Mormon tea and other gnetophytes, Class Gnetopsida, Order Ephedrales, Family Ephedraceae—Mormon-tea family; Order Gnetales, Family Gnetaceae—Gnetum family; Division Magnoliophyta—Flowering plants, Class Liliopsida—Monocotyledons, Subclass Alismatidae, Order Alismatales, Family Alismataceae—Water-plantain family, Family Butomaceae—Flowering Rush family, Family Limnocharitaceae—Water-poppy family; Order Hydrocharitales, Family Hydrocharitaceae—Tape-grass family; Order Najadales, Family Aponogetonaceae—Cape-pondweed family, Family Cymodoceaceae—Manatee-grass family, Family Juncaginaceae—Arrow-grass family, Family Najadaceae—Water-nymph family, Family Posidoniaceae—Posidonia family, Family Potamogetonaceae—Pondweed family, Family Ruppiaceae—Ditch-grass family, Family Scheuchzeriaceae—Scheuchzeria family, Family Zannichelliaceae—Horned pondweed family, Family Zosteraceae—Eel-grass family; Subclass Arecidae, Order Arales, Family Acoraceae—Calamus family, Family Araceae—Arum family, Family Lemnaceae—Duckweed family; Order Arecales, Family Arecaceae—Palm family; Order Cyclanthales, Family Cyclanthaceae—Panama Hat family; Order Pandanales, Family Pandanaceae—Screw-pine family; Subclass Commelinidae, Order Commelinales, Family Commelinaceae—Spiderwort family, Family Mayacaceae—Mayaca family, Family Xyridaceae—Yellow-eyed Grass family; Order Cyperales, Family Cyperaceae—Sedge family, Family Poaceae—Grass family; Order Eriocaulales, Family Eriocaulaceae—Pipewort family; Order Juncales, Family Juncaceae—Rush family; Order Restionales, Family Joinvilleaceae—Joinvillea family; Order Typhales, Family Sparganiaceae—Bur-reed family, Family Typhaceae—Cat-tail family; Subclass Liliidae, Order Liliales, Family Agavaceae—Century-plant family, Family Aloeaceae—Aloe family, Family Dioscoreaceae—Yam family, Family Haemodoraceae—Bloodwort family, Family Hanguanaceae—Hanguana family, Family Iridaceae—Iris family, Family Liliaceae—Lily family, Family Philydraceae—Philydraceae family, Family Pontederiaceae—Water-Hyacinth family, Family Smilacaceae—Catbrier family, Family Stemonaceae—Stemona family, Family Taccaceae—Tacca family; Order Orchidales, Family Burmanniaceae—Burmannia family, Family Orchidaceae—Orchid family; Subclass Zingiberidae, Order Bromeliales, Family Bromeliaceae—Bromeliad family; Order Zingiberales, Family Cannaceae—Canna family, Family Costaceae—Costus family, Family Heliconiaceae—Heliconia family, Family Marantaceae—Prayer-Plant family, Family Musaceae—Banana family, Family Zingiberaceae—Ginger family; Class Magnoliopsida—Dicotyledons, Subclass Asteridae, Order Asterales, Family Asteraceae—Aster family; Order Callitrichales, Family Callitrichaceae—Water-starwort family, Family Hippuridaceae—Mare's-tail family; Order Calycerales, Family Calyceraceae—Calycera family; Order Campanulales, Family Campanulaceae—Bellflower family, Family Goodeniaceae—Goodenia family, Family Sphenocleaceae—Spenoclea family; Order Dipsacales, Family Adoxaceae—Moschatel family, Family Caprifoliaceae—Honeysuckle family, Family Dipsacaceae—Teasel family, Family Valerianaceae—Valerian family; Order Gentianales, Family Apocynaceae—Dogbane family, Family Asclepiadaceae—Milkweed family, Family Gentianaceae—Gentian family, Family Loganiaceae—Logania family; Order Lamiales, Family Boraginaceae—Borage family, Family Lamiaceae—Mint family, Family Lennoaceae—Lennoa family, Family Verbenaceae—Verbena family; Order Plantaginales, Family Plantaginaceae—Plantain family; Order Rubiales, Family Rubiaceae—Madder family; Order Scrophulariales, Family Acanthaceae—Acanthus family, Family Bignoniaceae—Trumpet-creeper family, Family Buddlejaceae—Butterfly-bush family, Family Gesneriaceae—Gesneriad family, Family Lentibulariaceae—Bladderwort family, Family Myoporaceae—Myoporum family, Family Oleaceae—Olive family, Family Orobanchaceae—Broom-rape family, Family Pedaliaceae—Sesame family, Family Scrophulariaceae—Figwort family; Order Solanales, Family Convolvulaceae—Morning-glory family, Family Cuscutaceae—Dodder family, Family Fouquieriaceae—Ocotillo family, Family Hydrophyllaceae—Waterleaf family, Family Menyanthaceae—Buckbean family, Family Polemoniaceae—Phlox family, Family Solanaceae—Potato family; Subclass Caryophyllidae, Order Caryophyllales, Family Achatocarpaceae—Achatocarpus family, Family Aizoaceae—Fig-marigold family, Family Amaranthaceae—Amaranth family, Family Basellaceae—Basella family, Family Cactaceae—Cactus family, Family Caryophyllaceae—Pink family, Family Chenopodiaceae—Goosefoot family, Family Molluginaceae—Carpet-weed family, Family Nyctaginaceae—Four o'clock family, Family Phytolaccaceae—Pokeweed family, Family Portulacaceae—Purslane family; Order Plumbaginales, Family Plumbaginaceae—Leadwort family; Order Polygonales, Family Polygonaceae—Buckwheat family; Subclass Dilleniidae, Order Batales, Family Bataceae—Saltwort family; Order Capparales, Family Brassicaceae—Mustard family, Family Capparaceae—Caper family, Family Moringaceae—Horse-radish tree family, Family Resedaceae—Mignonette family; Order Diapensiales, Family Diapensiaceae—Diapensia family; Order Dilleniales, Family Dilleniaceae—Dillenia family, Family Paeoniaceae—Peony family; Order Ebenales, Family Ebenaceae—Ebony family, Family Sapotaceae—Sapodilla family, Family Styracaceae—Storax family, Family Symplocaceae—Sweetleaf family; Order Ericales, Family Clethraceae—Clethra family, Family Cyrillaceae—Cyrilla family, Family Empetraceae—Crowberry family, Family Epacridaceae—Epacris family, Family Ericaceae—Heath family, Family Monotropaceae—Indian Pipe family, Family Pyrolaceae—Shinleaf family; Order Lecythidales, Family Lecythidaceae—Brazil-nut family; Order Malvales, Family Bombacaceae—Kapok-tree family, Family Elaeocarpaceae—Elaeocarpus family, Family Malvaceae—Mallow family, Family Sterculiaceae—Cacao family, Family Tiliaceae—Linden family; Order Nepenthales, Family Droseraceae—Sundew family, Family Nepenthaceae—East Indian Pitcher-plant family, Family Sarraceniaceae—Pitcher-plant family; Order Primulales, Family Myrsinaceae—Myrsine family, Family Primulaceae—Primrose family, Family Theophrastaceae—Theophrasta family; Order Salicales, Family Salicaceae—Willow family; Order Theales, Family Actinidiaceae—Chinese Gooseberry family, Family Caryocaraceae—Souari family, Family Clusiaceae—Mangosteen family, Family Dipterocarpaceae—Meranti family, Family Elatinaceae—Waterwort family, Family Marcgraviaceae—Shingle Plant family, Family Ochnaceae—Ochna family, Family Theaceae—Tea family; Order Violales, Family Begoniaceae—Begonia family, Family Bixaceae—Lipstick-tree family, Family Caricaceae—Papaya family, Family Cistaceae—Rock-rose family, Family Cucurbitaceae—Cucumber family, Family Datiscaceae—Datisca family, Family Flacourtiaceae—Flacourtia family, Family Frankeniaceae—Frankenia family, Family Loasaceae—Loasa family, Family Passifloraceae—Passion-flower family, Family Tamaricaceae—Tamarix family, Family Turneraceae—Turnera family, Family Violaceae—Violet family; Subclass Hamamelidae, Order Casuarinales, Family Casuarinaceae—She-oak family; Order Fagales, Family Betulaceae—Birch family, Family Fagaceae—Beech family; Order Hamamelidales, Family Cercidiphyllaceae—Katsura-tree family, Family Hamamelidaceae—Witch-hazel family, Family Platanaceae—Plane-tree family; Order Juglandales, Family Juglandaceae—Walnut family; Order Leitneriales, Family Leitneriaceae—Corkwood family; Order Myricales, Family Myricaceae—Bayberry family; Order Urticales, Family Cannabaceae—Hemp family, Family Cecropiaceae—Cecropia family, Family Moraceae—Mulberry family, Family Ulmaceae—Elm family, Family Urticaceae—Nettle family; Subclass Magnoliidae, Order Aristolochiales, Family Aristolochiaceae—Birthwort family; Order Illiciales, Family Illiciaceae—Star-anise family, Family Schisandraceae—Schisandra family; Order Laurales, Family Calycanthaceae—Strawberry-shrub family, Family Hernandiaceae—Hemandia family, Family Lauraceae—Laurel family, Family Monimiaceae—Monimia family; Order Magnoliales, Family Annonaceae—Custard-apple family, Family Canellaceae—Canella family, Family Magnoliaceae—Magnolia family, Family Myristicaceae—Nutmeg family, Family Sonneratiaceae—Sonneratia family, Family Winteraceae—Wintera family; Order Nymphaeales, Family Cabombaceae—Water-shield family, Family Ceratophyllaceae—Hornwort family, Family Nelumbonaceae—Lotus-lily family, Family Nymphaeaceae—Water-lily family; Order Papaverales, Family Fumariaceae—Fumitory family, Family Papaveraceae—Poppy family; Order Piperales, Family Chloranthaceae—Chloranthus family, Family Piperaceae—Pepper family, Family Saururaceae—Lizard's-tail family; Order Ranunculales, Family Berberidaceae—Barberry family, Family Lardizabalaceae—Lardizabala family, Family Menispermaceae—Moonseed family, Family Ranunculaceae—Buttercup family, Family Sabiaceae—Sabia family; Subclass Rosidae, Order Apiales, Family Apiaceae—Carrot family, Family Araliaceae—Ginseng family; Order Celastrales, Family Aquifoliaceae—Holly family, Family Celastraceae—Bittersweet family, Family Corynocarpaceae—Karaka family, Family Hippocrateaceae—Hippocratea family, Family Icacinaceae—Icacina family, Family Stackhousiaceae—Stackhousia family; Order Cornales, Family Cornaceae—Dogwood family, Family Garryaceae—Silk Tassel family, Family Nyssaceae—Sour Gum family; Order Euphorbiales, Family Buxaceae—Boxwood family, Family Euphorbiaceae—Spurge family, Family Simmondsiaceae—Jojoba family; Order Fabales, Family Fabaceae—Pea family; Order Geraniales, Family Balsaminaceae—Touch-me-not family, Family Geraniaceae—Geranium family, Family Limnanthaceae—Meadow-Foam family, Family Oxalidaceae—Wood-Sorrel family, Family Tropaeolaceae—Nasturtium family; Order Haloragales, Family Gunneraceae—Gunnera family, Family Haloragaceae—Water Milfoil family; Order Linales Family Erythroxylaceae—Coca family, Family Linaceae—Flax family; Order Myrtales, Family Combretaceae—Indian Almond family, Family Lythraceae—Loosestrife family, Family Melastomataceae—Melastome family, Family Myrtaceae—Myrtle family, Family Onagraceae—Evening Primrose family, Family Punicaceae—Pomegranate family, Family Thymelaeaceae—Mezereum family, Family Trapaceae—Water Chestnut family; Order Podostemales, Family Podostemaceae—River-weed family; Order Polygalales, Family Krameriaceae—Krameria family, Family Malpighiaceae—Barbados Chemy family, Family Polygalaceae—Milkwort family; Order Proteales, Family Proteaceae—Protea family; Order Rafflesiales, Family Rafflesiaceae—Rafflesia family; Order Rhamnales, Family Elaeagnaceae—Oleaster family, Family Rhamnaceae—Buckthorn family, Family Vitaceae—Grape family; Order Rhizophorales, Family Rhizophoraceae—Red Mangrove family; Order Rosales, Family Brunelliaceae—Brunellia family, Family Chrysobalanaceae—Cocoa-plum family, Family Connaraceae—Cannarus family, Family Crassulaceae—Stonecrop family, Family Crossosomataceae—Crossosoma family, Family Cunoniaceae—Cunonia family, Family Grossulariaceae—Currant family, Family Hydrangeaceae—Hydrangea family, Family Pittosporaceae—Pittosporum family Family Rosaceae—Rose family, Family Saxifragaceae—Saxifrage family, Family Surianaceae—Suriana family; Order Santalales, Family Balanophoraceae—Balanophora family, Family Eremolepidaceae—Catkin-mistletoe family, Family Loranthaceae—Showy Mistletoe family, Family Olacaceae—Olax family, Family Santalaceae—Sandalwood family, Family Viscaceae—Christmas Mistletoe family; Order Sapindales, Family Aceraceae—Maple family, Family Anacardiaceae—Sumac family, Family Burseraceae—Frankincense family, Family Hippocastanaceae—Horse-chestnut family, Family Meliaceae—Mahogany family, Family Rutaceae—Rue family, Family Sapindaceae—Soapberry family, Family Simaroubaceae—Quassia family, Family Staphyleaceae—Bladdernut family, Family Zygophyllaceae—Creosote-bush family.

In one embodiment, potential plants comprise: Abelmoschus esculentus; Abies balsamea; Abies lasiocarpa; Achillea millefolium; Achillea tomentosa; Aconitum napellus; Aconitum spp.; Acorus calamus; Actaea racemosa; Actinidia arguta; Actinidia chinensis; Adiantum pedatum; Adiantum tenerum; Aesculus hippocastanum; Aframomum melegueta; Agaricus bisporus; Agastache foeniculum; Ageratum conyzoides; Agrimonia eupatoria; Agropyron cristatum; Agropyron repens; Agrostis alba; Agrostis stolonifera; Alcea rosea; Alchemilla mollis; Alkanna tinctoria; Allium ampeloprasum; Allium cepa; Allium fistulosum; Allium grande; Allium porrum; Allium sativum; Allium schoenoprasum; Allium tuberosum; Allium victorialis; Aloe vera; Alpinia officinarum; Althaea officinalis; Amaranthus caudatus; Amaranthus retroflexus; Amaranthus tricolor; Ambrosia artemisiifolia; Amelanchier alnifolia; Amelanchier canadensis; Amelanchier sanguinea; Amelanchier sanguinea×A. laevis; Amsonia tabernaemontana; Ananas comosus; Anaphalis margaritacea; Anethum graveolens; Angelica archangelica; Angelica dahurica; Angelica sinensis; Anthemis tinctoria; Anthoxanthum odoratum; Anthriscus cerefolium; Anthurium guildingii; Apium graveolens; Apocynum cannabinum; Arachis hypogaea; Aralia cordata; Aralia nudicaulis; Arctium lappa; Arctium minus; Arctostaphylos uva-ursi; Armoracia rusticana; Aronia melanocarpa; Aronia×prunifolia; Arrhenatherum elatius; Artemisia abrotanum; Artemisia absinthium; Artemisia dracunculus; Artemisia ludoviciana; Artemisia vulgaris; Asarum europaeum; Asclepias incamata; Asclepias tuberosa; Asparagus officinalis; Aster spp.; Astilbe×arendsii; Astilboides tabularis; Athyrium asperum; Atriplex hortensis; Atropa belladonna; Avena sativa; Averrhoa carambola; Baptisia tinctoria; Beckmannia eruciformis; Begonia convolvulacea; Begonia eminii; Begonia glabra; Begonia mannii; Begonia polygonoides; Bellis perennis; Berberis vulgaris; Beta vulgaris; Betula alleghaniensis; Betula glandulosa; Boesenbergia rotunda; Boletus edulis; Borago officinalis; Brassica cepticepa; Brassica juncea; Brassica napus; Brassica nigra; Brassica oleracea; Brassica rapa; Bromus inermis; Buddleja davidii; Bupleurum falcatum; Butomus umbellatus; Caladium spp.; Calamagrostis arundiflora; Calamintha nepeta; Calendula officinalis; Camellia sinensis; Campanula rapunculus; Canna indica; Cantharellus cibarius; Capsella bursa-pastoris; Capsicum annuum; Capsicum frutescens; Carex morrowii; Carica papaya; Carthamus tinctorius; Carum carvi; Carya cordiformis; Castanea spp.; Centaurea solstitialis; Cerastium tomentosum; Chaerophyllum bulbosum; Chamaemelum nobile; Chelidonium majus; Chenopodium album; Chenopodium bonus-henricus; Chenopodium quinoa; Chrysanthemum coronarium; Cicer arietinum; Cichorium endivia subsp. endivia; Cichorium intybus; Cinnamomum verum; Cirsium arvense; Cissus discolor; Citrullus colocynthis; Citrullus lanatus; Citrus limettoides; Citrus limon; Citrus reticulata; Citrus sinensis; Citrus×paradisi; Clematis armandii; Clematis chiisanensis; Coccoloba caracasana; Cocos nucifera; Coix lacryma-jobi; Colocasia spp.; Convallaria majalis; Conyza canadensis; Corchorus olitorius; Coriandrum sativum; Cornus canadensis; Cornus mas; Cosmos sulphureus; Cotinus coggygria; Crataegus sanguinea; Crataegus spp.; Crataegus submollis; Crithmum maritimum; Cryptotaenia canadensis; Cucumis anguria; Cucumis melo; Cucumis metuliferus; Cucumis sativus; Cucurbita maxima; Cucurbita moschata; Cucurbita pepo; Cullen corylifolium; Cuminum cyminum; Curcuma longa; Curcuma zedoaria; Cyclonia oblonga; Cymbopogon citratus; Cymbopogon martinii; Cynara cardunculus subsp. cardunculus; Cyperus esculentus; Dactylis glomerata; Datisca cannabina; Datura metel; Datura stramonium; Daucus carota; Digitalis purpurea; Dimocarpus longan; Dioscorea batatas; Diospyros kaki; Dipsacus sativus; Dirca palustris; Dolichos lablab; Dryopteris filix-mas; Echinacea purpurea; Echinochloa frumentacea; Eleusine coracana; Equisetum hyemale; Erigeron speciosus; Eriobotrya japonica; Eruca vesicaria; Erysimum perofskianum; Eschscholzia californica; Fagopyrum esculentum; Fagopyrum tataricum; Festuca rubra; Filipendula rubra; Filipendula ulmaria; Filipendula vulgaris; Foeniculum vulgare; Forsythia×intermedia; Fortunella spp.; Fragaria×ananassa; Frangula alnus; Fucus vesiculosus; Fumaria officinalis; Galinsoga quadriradiata; Galium odoratum; Gaultheria hispidula; Gaultheria procumbens; Genista multibracteata; Gentiana lutea; Gentiana macrophylla; Geum rivale; Ginkgo biloba; Glechoma hederacea; Glyceria maxima; Glycine max; Glycyrrhiza glabra; Gossypium herbaceum; Guizotia abyssinica; Hamamelis virginiana; Hedeoma pulegioides; Hedychium spp.; Helianthus annuus; Helianthus strumosus; Helianthus tuberosus; Helichrysum angustifolium; Helichrysum thianschanicum; Heliotropium arborescens; Helleborus niger; Herba schizonepetae; Hibiscus cannabinus; Hordeum hexastichon; Hordeum vulgare; Hordeum vulgare subsp. vulgare; Houttuynia cordata; Humulus lupulus; Hydrastis canadensis; Hylotelephium spp.; Hymenoxys hoopesii; Hyoscyamus niger; Hypericum henryi; Hypericum perforatum; Hypericum spp.; Hypomyces lactifluorum; Hyssopus officinalis; Iberis amara; Iberis sempervirens; Inula helenium; Ipomoea batatas; Iris versicolor; Isatis tinctoria; Jeffersonia diphylla; Juglans nigra; Juniperus communis; Kochia scoparia; Koeleria glauca; Kolkwitzia amabilis; Krameria lappacea; Lactuca sativa; Lactuca serriola; Laportea canadensis; Laserpitium latifolium; Lathyrus sativus; Lathyrus sylvestris; Laurus nobilis; Lavandula angustifolia; Lavandula latifolia; Ledum groenlandicum; Lens culinaris subsp. culinaris; Lentinus edodes; Leonurus cardiaca; Lepidium sativum; Leucanthemum vulgare; Levisticum officinale; Ligularia dentata; Ligustrum vulgare; Linaria vulgaris; Lindera benzoin; Linum usitatissimum; Litchi chinensis; Lolium multiflorum; Lolium perenne; Lonicera ramosissima; Lonicera syringantha; Lotus comiculatus; Lotus tetragonolobus; Lunaria annua; Lupinus polyphyllus; Luzula sylvatica; Lychnis chalcedonica; Lycopersicon esculentum; Lycopersicon pimpinellifolium; Lysimachia clethroides; Lythrum salicaria; Madia sativa; Magnolia stellata; Malus hupehensis; Malus prunifolia; Malus spp.; Malva moschata; Malva sylvestris; Mangifera indica; Manihot esculenta; Marrubium vulgare; Matricaria recutita; Matricaria spp.; Medicago sativa; Melaleuca alternifolia; Melilotus albus; Melilotus officinalis; Melissa officinalis; Mentha arvensis; Mentha pulegium; Mentha spicata; Mentha suaveolens; Mentha×piperita; Menyanthes trifoliata; Microlepia platyphylla; Miscanthus sacchariflorus; Miscanthus sinensis; Momordica charantia; Monarda didyma; Monarda fistulosa; Monarda spp.; Musa×paradisiaca; Myrica pensylvanica; Nasturtium officinale; Nepeta cataria; Nicotiana rustica; Nicotiana tabacum; Nigella sativa; Ocimum Basilicum; Oenothera biennis; Onobrychis viciifolia; Ophiopogon japonicus; Opuntia spp.; Origanum majorana; Origanum vulgare; Oryza sativa; Oxalis deppei; Oxyria digyna; Paeonia rubra; Paeonia spp.; Panax quinquefolius; Panicum miliaceum; Passiflora caerulea; Passiflora spp.; Pastinaca sativa; Pennisetum alopecuroides; Perilla frutescens; Persea americana; Petasites japonicus; Petroselinum crispum; Peucedanum cervaria; Peucedanum oreaselinum; Pfaffia paniculata; Phacelia tanacetifolia; Phalaris arundinacea; Phalaris canariensis; Phaseolus acutifolius; Phaseolus coccineus; Phaseolus vulgaris; Philadelphus coronarius; Phleum pratense; Phlox paniculata; Phoenix dactylifera; Physalis grisea; Physalis philadelphica; Physalis spp.; Physostegia virginiana; Phytolacca americana; Pimpinella anisum; Pisum sativum; Plantago coronopus; Plantago major; Plectranthus fruticosus; Plectranthus spp.; Pleurotus spp.; Plumbago zeylanica; Poa compressa; Poa pratensis; Podophyllum peltatum; Polygonatum odoratum; Polygonum aviculare; Polygonum chinense; Polygonum pensylvanicum; Polygonum persicaria; Pongamia pinnata; Pontederia cordata; Populus incrassata; Populus tremula; Populus×petrowskyana; Portulaca oleracea; Potentilla anserina; Poterium sanguisorba; Primula veris; Prunella vulgaris; Prunus armeniaca; Prunus cerasus; Prunus persica; Prunus spp.; Prunus tomentosa; Psathyrostachys juncea; Psidium guajava; Psidium spp.; Pteridium aquilinum; Pulmonaria officinalis; Pulmonaria saccharata; Punica granatum; Pyrus communis; Pyrus pyrifolia; Raphanus raphanistrum; Raphanus sativus; Rehmannia glutinosa; Reseda luteola; Reseda odorata; Rheum officinale; Rheum palmatum; Rheum×hybridum; Rhus aromatica; Rhus trilobata; Ribes grossularia; Ribes nigrum; Ribes rubrum; Ribes sylvestre; Ribes uva-crispa; Ribes×nidigrolaria; Ricinus communis; Rosa rugosa; Rosmarinus officinalis; Rubus allegheniensis; Rubus canadensis; Rubus idaeus; Rubus occidentalis; Rubus thibetanus; Rumex acetosa; Rumex acetosella; Rumex crispus; Rumex patientia; Rumex scutatus; Ruta graveolens; Saccharum officinarum; Salix purpurea; Salvia elegans; Salvia officinalis; Salvia sclarea; Salvia sylvestris; Sambucus canadensis; Sambucus ebulus; Sambucus nigra; Sanguisorba minor; Sanguisorba officinalis; Santolina chamaecyparissus; Saponaria officinalis; Satureja hortensis; Satureja montana; Satureja repandra; Scolymus hispanicus; Scorzonera hispanica; Scrophularia nodosa; Scutellaria lateriflora; Secale cereale; Sechium edule; Senecio vulgaris; Serenoa repens; Serratula tinctoria; Sesamum indicum; Setaria italica; Sidalcea spp.; Silene vulgaris; Silybum marianum; Sinapis alba subsp. alba; Sium sisarum; Solanum dulcamara; Solanum melongena; Solanum scabrum; Solanum tuberosum; Solidago canadensis; Solidago spp.; Solidago virgaurea; Solidago×hybrida; Sonchus oleraceus; Sorghum bicolor; Sorghum×drummondii; Spinacia oleracea; Stachys affinis; Stachys byzantina; Stachys macrantha; Stellaria graminea; Stellaria media; Stipa capillata; Symphytum officinale; Tamarindus indica; Tanacetum balsamita; Tanacetum balsamita subsp. balsamita; Tanacetum cinerariifolium; Tanacetum parthenium; Tanacetum vulgare; Taraxacum officinale; Tetradenia riparia; Teucrium chamaedrys; Thalictrum aquilegiifolium; Thlaspi arvense; Thuja occidentalis; Thymus fragantissimus; Thymus herba-barona; Thymus praecox subsp. arcticus; Thymus pseudolanuginosus; Thymus serpyllum; Thymus vulgaris; Thymus×citriodorus; Tiarella cordifolia; Tiarella spp.; Tragopogon porrifolius; Tragopogon spp.; Trichosanthes kirilowii; Trifolium hybridum; Trifolium incarnatum; Trifolium pannonicum; Trifolium pratense; Trifolium repens; Trigonella foenum-graecum; Triticum aestivum; Triticum aestivum subsp. spelta; Triticum turgidum; Trollius×cultorum; Tropaeolum majus; Tsuga canadensis; Tsuga diversifolia; Tsuga mertensiana; Tussilago farfara; Typha latifolia; Ulmus americana; Urtica dioica; Uvularia perfoliata; Vaccinium angustifolium; Vaccinium corymbosum; Vaccinium macrocarpon; Valeriana officinalis; Valerianella locusta; Veratrum viride; Verbascum thapsus; Verbena officinalis; Veronica officinalis; Viburnum opulus; Vicia faba; Vicia sativa; Vicia villosa; Vigna angularis; Vigna mungo; Vigna unguiculata; Vinca minor; Vitis spp.; Weigela coraeensis; Weigela hortensis; Withania somnifera; ×Triticosecale spp.; Xanthium sibiricum; Xanthium strumarium; Yucca filamentosa; Zea mays; Zingiber officinale; Achillea ptarmica; Ajuga reptans; Aster spp; Astilbe chinensis; Bergenia×schmidtii; Brassica chinensis; Butomus umbellatus; Buxus microphylla; Carpinus caroliniana; Centaurea dealbata; Chaenomeles×superba; Clematis alpina; Coreopsis verticillata; Cornus alba; Cornus sericea; Corylus maxima; Crambe cordifolia; Cyperus alternifolius; Dahlia spp.; Euphorbia amygdaloides; Fuchsia spp.; Fuchsia magellanica; Galium aparine; Geranium sanguineum; Geranium phaeum; Geranium pratense; Geranium sanguineum; Geranium×cantabrigiense; Glaux Maritima; Hamamelis mollis; Hedychium coronarium; Helenium spp.; Herba Schizonepetae; Hosta sieboldiana; Hydrangea quercifolia; Ipomoea aquatica; Lamiastrum galeobdolon; Magnolia×loebneri; Malva verticillata; Matteuccia pensylvanica; Microbiata decussata; Montia perfoliata; Ocimum tenuiflorum; Oenothera fruticosa subsp fruticosa; Onoclea sensibilis; paeonia suffruticosa; Penstemon digitalis; Petasites japonicus; Physalis alkekengi; Pinus cembra; Pinus mugo; Potentilla fruticosa; Rhododendron spp.; ribes americanum; Rodgersia spp.; Rodgersia podophylla; Rubus arcticus; Rubus phoenicolasius; Rubus pubescens; Rudbeckia maxima; Sempervivum tectorum; Soleirolia soleirolii; Solidago caesia; Staphylea trifolia; Stephanandra incisa; Stewartia pseudocamellia; Strelitzia reginae; Symphoricarpos orbiculatus; Symphoricarpos albus; Taxus×media; Vernonia gigantea; Veronica austriaca ssp teucrium; Veronica beccabunga and Viburnum plicatum.

In another embodiment, potential plants comprise: Abies cephalonica, Abies firma, Acer campestre, Acer mandshurica, Acer palmaturn “burgundy,” Acer tataricum, Acer truncatum, Acolypha hispida, Aconitum napellus, Actinidi colonicta, Actinidia chinensis, Actinidia colomicta, Adansonia digitata, Adianthum radiatum, Adianthum trapezieformis, Aechmea luddemoniana, Aesculus hippocastanum, Aesculus hypocastanum, Aesculus waertilensis, Aesculus woerlitzenis, Aessopteria crasifolia, Agastache mexuicana, Agatis robusta, Ageratum conizoides, Aglaonema commutatus, Agrimonia eupatora, Ailantus altissima, Alchemilla sp., Alium cernum (wild), Allium fistulosum, Allium nutans, Allium sp., Alum japonica, Amelanchier spicata, Amigdalus nana, Ananas comosus, Anemonajaponica, Antericum ramosum, Anthurium altersianum, Anthurium andreanum, Anthurium elegans, Anthurium hookeri, Anthurium magnificurn, Anthyrium filis-femina, Anthyrium nopponicum, Aralis mandshurica, Archirantus bidentata, Armoracea rusticana, Armoraica ristica, Artemisia dracunculus, Asimina triloba, Asorum canadensis, Asplenium australasicum, Aster-Nova anglicae, Astragulus sinicus, Atropa Belladonna, Austolachia australis, Bactisia australis, Barbaric sp., Berberis thungergi, Berberis vulgaris, Bergenia crassifolia, Betula alba, Betula daurica, Betula nigra, Betula nigra (flower), Betula nigra (leaf), Betula pendula, Betula pendula, Bocconia cordata, Boechimeria boloba, Boxus sempervirens, Brassica juncea, Brassica napa, Bromelia balansae, Brugmansi graveolens (ralf), Brugmansia suaveolens, Brugmansia suaveolens (old), Brugmansia suaveolens (young), Buxus microphilla “japonica,” Buxus microphylla “japonica,” Cachris alpina, Cactus officinalis, Calathea zebrina, Calicatus floridus, Campanula carpatica, Capparis spinosa inemis, Carica papaya, Carlina acaulis, carpinifolia, Carum capsicum, Caryota ureus, Casia hebecarpa, Castanea sativa, Celosia cristata, Celtis occidentalis, Celtis occidentalis, Centauria maculata, Cerasus japonica, Cerasus maghabab, Ceratoramia mexicana, Chaernomelis superba, Chamaechrista fasciculata, Charnaeciparis pisifera, Chelidonium majus, Cistus incanus, Citinis coggriaria, Clematis rectae, Clerodendrum speciossicum, Cobiaeum varilartum, Cocculus laurifolius, Comus mass, Convalaria majalis, Coronolla varia, Coryllus avelana, Corylus avelana, Cotoneaster fangianus, Cotoneaster horisontalis, Cotynus cogygria, Cramble cardifolia, Crataegus praegophyrum, Crategus macrophyllum, Crytomium fortunei, Cupress lusitanica, Cupressus sempervirens, Cupressus sempervirens, Cycas cirinalis, Cyclonia oblonga, Cynnamonum zeylonicum, Darura stramonium, Deutria scabra, Dieffenbachia leopoldii, Dieffenbachia segiunae, Digitalis lutea, Diopiros kaka, Dracaena fragrans, Dracaena sp., Dryopteris filis-max, Echinops sphae, Eleagnus angustifolia, Eleagnus cemutata, Encephalaris horridum, Epilobium augustifolium, Equisetum variegatum, Eriobotria japonica, Erungium campestre, Erythrinia caffra, Erythrinia crista, Erythrinia glabeliferus, Eucaliptus rudis, Eucomia ulurifolia, Euonimus elata, Euonomus europea, Euonomus verrucosa, Fagopyrum suffruticosum, Fagus silvatica, Fautenousus qualiqualia, Feucrium hamedris, Ficus benjamina, Ficus benjaminii, Ficus elastica, Ficus purnila, Ficus religiosa, Ficus sp., Ficus triangularis, Filipendula ulmaria, Filipendula vulgrais, Foenix zeulonica, Forsithsia suspensa, Forsitsia europea, Fraxinus exelsior, Gallium sporium, Gardenia jasminoides, Gaultheria procumbens, Gentiana cruciata, Gentiana littorala, Gentiana macrophilla, Gentiana tibetica, Geranium maculata, Geum fanieri, Geum macrophyllum, Gingko biloba, Gnetum guemon, Gratiola officinalis, Gravilea robusta, Gravilea robusta, Gravilia robusta, Haser trilobum, Helianthus annus, Heraclelum pubescens, Hernerocalis spp., Hhaemanthus katharina, Hissopus zeraucharicus, Hiuga reptans, Hosta fortuna, Hosta fortunaea, Hosta lancefolia, Hosta zibalda, Hydrocotile asiatica, Hydrocotile asiatica, Hyppoach rhamnoides, Ilex agnifolium, Ilex cornuta, Inula hilenium, Ipomea tricolor, Iris alida, Iris pseudocarpus, Jacobinia sp., Jasminum frutocarus, Juca sp., Juglands regia, Juniperus “blue pacific,” Keyleiteria paniculata, Kolkwitzia amabilis, Korria japonica, Lal lab purpurea, Lapia dulcis, Larix dedidua, Laurus nobilis, Laurus nobilis, Lavandula officinalis, Lavandula officinalis, Leontopodium alpinum, Liatris spinata, Liclum barbatum, Ligustum vulgare, Linium hirsutum, Lippa dulcis, Livistona fragrans, Lobelia siphitica, Luglands nigra, Lupinus luteaus, Lycodium japonicum, Magnolia cobus, Magnolia loebheril, Magnolia agrifolia, Matteucia strutioptoris, Mespilus germanica, Metasequoia glyptotrobioldes, Metrosideros excelsa, Microlepia platphylla, Microsorium punctatum, Minispermum dauricum, Mirica certifera, Monstera deliciosa, Monstera pertusa, Morus alba, Murraya exotica, Musa textilis (Leaf), Musa textilis (Stem), Myrthus communis, Myrthus comunis, Nepeta cataria, Nicodemia diversifolia, Nicotiana tabacum, Olea europaea, Olea olcaster, Oreopanax capitata, Origanurn vulgare, Osmanthus spp., Osmunda regalis, Osmundastrum claytonionum, Ostrea carpinifolia, Ostrea connote, Oxobachus nictogenea, Pachyra affinis, Paeonia daurica, Paeonia lactiflora, Paeonia suffructicisa, Parrotia persica, Parthenosicus tricuspidata, Pegamun hamalis, Pelagonium zonale, Pelargoniurn zonale, Pentaphylloides fruticosa, Phebodium aureum, Philodendron amurense, Phylidendron speciosus, Phyllanthus grandifolium, Phyllitis scolopendrium, Phymatosorus scolopendria, Physalis creticola, Picea schrenkiana, Pieras japonica, Pigelia pennata, Pinus bungiana, Pinus pinea, Pinus pumila, Pinus salinifolia, Pinus silvestris, Pinus sirtrobus, Pinus strobus, Piper chaba, Piper nigrum, Pithecelobium unguis, Pittisporum tibica, Plantago major, Plantago minor, Platanus acidentalis, Platicada grandiflora, Podocarpus spinulosus, Podophyllum amodii, Poligonum aviculare, Poligomun latifolia, Polygonium odoratum, Polygonum cuspidatum, Polymonium ceruleum, Polyschium braunii, Portulaca oleacea, Portulaca olleracea, Potentilla alba, Poterium sangiusorba, Princepia sp., Prunella vulgaris, Prunus cerasifera, Prunus serotica, Prunus xocane, Pseudotsuga menzisia, Psidium guajava, Psychotria metbacteriodomasica, Psychotria nigropunctata, Pterigota alata, Puansetia sp., Pulmonaria molissima, Quercus castanufolia, Quercus imbricaria, Quercus nigra, Quercus robur “fastigiata,” Quercus rubra, Quercus trojana, Ratibiunda columnus-Fera, Rauwolfia tetraphylla, Reseda luteola, Rhododendron spp., Rhus toxicodenta, Rimula japonica, Rosa cocanica, Rosa multiflora, Ruschia indurata, Ruta graveolens, Salis babilonics, Salix tamarisifolia, Sambucus niora, Sanchezia nobilis, Schisandra chinensis, Scotch pine, Scutellaria certicola, Scutellarian altissima, Sedum album, Sedum telchium, Senecio platifilla, Senseviera sp., Sering a josiceae, Seruginea suffruticisa, Sesbania exaltata, Sesbania speciosa, Sibirea altaiensis, Siring a vulgaris, Sluffera sp., Sorbocotoneaster sp., Sorbus aucuparia, Sorbus cominicta, Spartina potentiflora, Spathiphyllum cochlearispatum, Spathiphyllum grandiflorum, Stachis lanata, Stepochlaena tenuifolia, Sterulia elata, Stevartia coreana, Strelitzia reglinae, Sulda sanganea, Sundapsis spp., Symphitium officinalis, Syngonium aurutum, Syngoniurn podophyllum, Taccus bacata, Tagetes minuta, Talictrum minus, Talictrum sp., Tamarindus india, Tapeinochilos spectabilis, Taraxacum officinalis, Taxodium dixticum, Taxodium dixticum (Acetic acid), Taxodium dixticum (H₂O), Taxus cuspidata, Taxus hiksii, Taxus media, Tetraclinis articulata hinensis, Thalictum flavum, Thuja occidentalis, Thuja occidentalis, Thymus camosus, Thymus carnosus, Thymus cretaceus, Thymus cytridorus “aureus,” Thymus lemabarona, Thymus portugalense, Thymus praecox, Thymus praecox “arcticus,” Thymus pseudolamginosus, Thymus puleglodes “lemons,” Thymus puliglodes, Thymus serphylum, Thymus serphylum (wild), Thymus speciosa, Thymus thrasicus, Thymus vulgaris, Thymus vulgaris “argenteus,” Thymus vulgaris “oregano,” Thymus wooly, Trambe pontica, Trevesia sungaica, Trifolium pratense, Tsuga canadensis “penola,” Tuja orientalis “eligantissima,” Tula ocidentalis “columbia,” Tulip tree, Turnera ulmifolia, Ulmus pumila, Uschusa sp., Valeriana officinalis, Veratrum nigrum, Verium oleander, Viburnum opulus, Vinca minor, Vincetocsicum officinalis, Vitis labrissa, Xanthosoma sagittifolium (leaf), Xanthosoma sagittifolium (stem), Xeupressocyparis deylandii, Yucca elephantipes, Zelcova and Zingiber officinalis.

Another group of potential plants comprise the plants that are indigenous to arid regions, for example, those located between 35° north latitude and 35° south latitude. In accordance with another embodiment of the present invention, therefore, potential plants comprise: the agave, Agavaceae, family including such members as: Yucca elata, Y. breviflora, Agave deserti, A. chrysantha, Dasylirion wheeleri; the buckwheat, Polygonaceae, family, such as Eriogonum fasciculatum; the crowfoot, Ranunculaceae, family, such as Delphinium scaposum, Anemone tuberosa and D. parishii; the poppy, Papaveraceae, family, including Platystemon californicus, Argemone pleiacantha, Corydalis aurea, Eschschoizia californica and Ar. corymbosa; members of the mustard, Cruciferae, family, such as Dithyrea californica, Streptanthus carinatus and Lesquerella gordoni; members of the legume, Leguminosae, family, such as Acacia greggii, Prosopis velutina, A. constrica, Senna covesii, Cercidium floridum, C. microphyllum, Lotus huminstratus, Krameria parvifolia, Parkinsonia aculeata, Calliendia eriophylla, Lupinus arizonicus, Olyneya tesota, Astragalus lentiginosus, Psorothamunus spinosus and Lupinus sparsiflorus; members of the loasa family, Loasaceae, including Mentzelia involucrata, M. pumila and Mohavea Confertiflora; members of the cactus, Cactaceae, family, such as Carnegiea gigantia, Opuntia leptocaulis, Ferocactus wislizenii, O. bigelovii, O. pheacantha, O. versicolor, O. fulgida, Echinocereus engelmannii, Mammillaria microcarpa, O. basilaris, Stenocereins thurberi, O. violacea, M. tetrancistra, O. ramosissima, O. acanthocarpa, E. pectinatins and O. arbuscula; members of the evening primrose, Onagraceae, family, such as Oenothera deltoides, Camissonia claviformis and Oe. primiveris; members of the milkweed, Asclepiadaceae, family, including Asclepias erosa, A. sublata and Sarcostemma cynanchoides; members of the borage, Boraginaceae, family, such as Cryptantha augustifolia and Amsinckia intermedia; members of the sunflower, Compositae, family, including Baccharis sarothroides, Monoptiilon belloides, Erieron divergens, Zinnia acerosa, Melampodium leucanthan, Chaenactis fremontii, Calycoseris wrightii, Malacothrix californica, Helianthus annus, H. niveus, Geraea canescens, Hymenothrix wislizenii, Encelia farinosa, Psilostrophe cooperi, Baileya multiradiata, Bebbia juncea, Senecio douglasii, Trixis californica, Machaeranthera tephrodes, Xylorhiza tortifolia, Cirsium neomexicanum, Antennaria parviflora and Ch. douglasii; members of the caltrop, Zygophyllaceae, family, including Larrea tridentata and Kallstroemia grandiflora; members of the mallow, Malvaceae, family, including Hibiscus coulteri, H. denudatus and Sphaeralcea ambigua; members of the phlox, Polemoniaceae, family, such as Luanthus aureus; members of the unicorn plant, Martyniaceae, family, such as Proboscidiea altheaefolia; members of the gourd, Cucurbitaceae, family, such as Cucurbita digitata; members of the lily, Lilaceae, family, including Calochortus kennedyi, Dichelostemma pulchellum, Allium macropetalum and Hesperocallis indulata; members of the ocotillo, Fouquieriaceae, family, including Fouquieria splendens; members of the figwort, Scrophulariaceae, family, such as Castilleja sp., Penstemon parryi and Orthocarpus purpurascens; members of the acanthus, Acanthaceae, family, including Anisacanthus thurberi, Justicia californica and Ruellia nudiflora; members of the four o'clock, Nyctaginaceae, family, such as Allionia incarnata, Abronia villosa and Mirabilis multiflora; members of the geranium, Geraniaceae, family, including Erodium cicutarium; members of the waterleaf, Hydrophyllaceae, family, such as Nama demissum, Phacelia bombycina and Ph. distans; members of the bignonia, Bignoniaceae, family, such as Chilopsis linearis; members of the vervain, Verbenaceae, family, including Glandularia gooddugii and Verbena neomexicana; members of the mint, Labiatae, family, such as Hyptis emoryi and Salvia columbariae; members of the broomrape, Orobanchaceae, family, such as Orobanche cooperi; members of the portulaca, Portulaceae, family, such as Talinum auriantiacum; members of the carpet-weed, Aizoaceae, family, such as Sesuvium verrucosum; members of the flax, Linaceae, family, such as Linum lewisii; members of the potato, Solanaceae, family, including Nicotiana trigonophylla and Physalis lobata; and members of the cochlospermum, Cochlospermaceae, family, such as Amoreuxia palmatifida.

In accordance with one embodiment of the present invention, the potential plant is selected from the group comprising: Allium tuberosum; Althacea officinalis; Amaranthus candathus; Ambrosia artemisiifolia; Angelica sinensis; Aronia×prunifolia; Asarum europaeum; Begonia Hannii; Begonia polygonoides; Brassica oleracea; Brassica napus; Brassica oleracea; Bromus inermis; Chenopodium quinoa; Citrullus lanatus; Conyza canadensis; Cynara cardunculus subsp. Cardunculus; Daucus carota; Dolichos lablab; Foeniculum vulgare; Hypomyces lactifluorum; Iberis sempervirens; Lotus corniculatus; Lunaria annua; Manihot esculenta; Matricaria recutita; Melilotus albus; Phaseolus vulgaris; Physostegia virginiana; Pisum sativum; Raphanus raphanistrum; Rheum rhabarbarum; Ribes sylvestre; Rubus occidentalis; Rumex crispus; Rumex scutatus; Salvia officinalis; Solidago canadensis; Solidago sp.; Solidago×hybrida; Tamarindus indica; Tanacetum cinerariifolium; Taraxacum officinale; Tropaeolum majus; Tsuga canadensis; Tsuga diversifolia; Vaccinium angustifolium; Zea mays; Zingiber officinale.

### Pre-Harvest Treatment

Once a potential plant has been chosen, a pre-harvest treatment is selected, wherein the treatment can be water or water in combination with one or more stressor, elicitor, or inducer. A pre-harvest treatment comprises contacting or treating a potential plant, or material from a potential plant, with one or more stressor, elicitor, or inducer. Examples of stressors, elicitors and inducers include, but are not limited to, chemical compounds, for example organic and inorganic acids, fatty acids, glycerides, phospholipids, glycolipids, organic solvents, amino acids and peptides, monosaccharides, oligosaccharides, polysaccharides and lipopolysaccharides, phenolics, alkaloids, terpenes and terpenoids, antibiotics, detergents, polyamines, peroxides, ionophores, etc.; subjection of the plant material to a physical treatment, such as ultraviolet radiation, low and high temperature stress, osmotic stress induced by salt or sugars, nutritional stress defined as depriving the plant of essential nutrients (e.g. nitrogen, phosphorus or potassium), in order to induce or elicit increased production of one or more chemicals. The one or more stressor (i.e. chemical compound or physical treatment) may be applied continuously or intermittently to the plant material. In one embodiment, such treatment may be accomplished by contacting the plant material with a solution containing the elicitor or by irradiating the plant material or exposing the plant material to other environmental stresses such as temperature stresses.

One skilled in the art would understand that a potential plant can be subjected to a variety of pre-harvest treatments and an extract prepared after each treatment. For example, the treatment can be with water and then with one or a series of stressors. The extracts are then tested to determine whether they become an extract of the invention. Thus, it is possible that, of several extracts prepared from the same potential plant subjected to different pre-harvest treatment, only some may become extracts of the invention.

In one embodiment, the potential plant is subjected to a pre-harvest treatment comprising stressing the plant through the use of chemical elicitors, which act as stressor agent, and/or mechanical wounding, drought, heat, or cold, which activate plant defense pathways, before tissue collection and extraction.

In another embodiment, the stressor employed involves exposing a potential plant to a solution of one or more chemical elicitors to induce defense metabolic pathways and secondary metabolites prior to collection of plant tissues. Known chemical elicitors reported in the literature include ozone, hydrogen peroxide, jasmonic acid and its derivatives, arachidonic acid, salicylic acid and ester derivatives, alpha- and gamma-linolenic acids, volicitin, peptides, oligopeptides, saccharides, oligosaccharides such as chitosan, and synthetic chemicals such as benzo-1,2,3-thiadiazole-7-carbathioic acid S-methyl ester (BTH).

A stressor may be one or more organic compound. Some exemplary compounds that may be used as stressors include jasmonic acid, jasmonic acid lower alkyl esters, αlinolenic acid, α-linolenic acid lower alkyl esters, γ-linolenic acid, γ-linolenic acid lower alkyl esters, arachidonic acid, arachidonic acid lower alkyl esters, salicylic acid.

In one embodiment of the present invention, the stressor is γ-linolenic acid, γ-linolenic acid lower alkyl esters, arachidonic acid, arachidonic acid lower alkyl esters, or a combination thereof.

A stressor may be able to induce abiotic stresses in plants. Thus, for example, plants can be treated with one or more mechanical or chemical stress prior to tissue collection.

Mechanical stress can be performed, for example, between about twelve hours to about ten days prior to tissue collection. In one embodiment of the present invention, a potential plant can be subjected to one or more mechanical stress between about one day to about three days prior to tissue collection. In another embodiment, a potential plant can be subjected to one or more mechanical stress between about three to about six days prior to tissue collection. In a further embodiment, a potential plant can be subjected to one or more mechanical stress between about four to about eight days prior to tissue collection. In another embodiment a potential plant can be subjected to one or more mechanical stress between about six to about ten days prior to tissue collection.

Chemical stress can be induced in a potential plant by spraying plant material once, or more than once, with an aqueous or alcoholic solution of one or more chemical elicitor. Chemical stress can also be induced by feeding a potential plant with an aqueous or alcoholic solution of one or more chemical elicitor. Similarly, a potential plant can be subjected to a chemical stress by means of contact with an airborne transport of one or more chemical elicitor. Chemical stress can be performed, for example, between about one hour to about 10 days prior to tissue collection. In one embodiment of the present invention, a potential plant can be subjected to one or more chemical stress between about ten hours and about one day prior to harvesting the plant tissue. In another embodiment, a potential plant can be treated with one or more chemical by spray one day before harvesting. In a further embodiment, a potential plant can be subjected to one or more chemical stress between about one day to about three days prior to harvesting the plant tissue. In other embodiments, a potential plant can be subjected to one or more chemical stress between about two to about four days and between about five to about ten days prior to harvesting the plant tissue.

Various combinations of the above-mentioned stressors and treatment regimes can be employed to induce or enhance the production of one or more extracellular proteases in the plant material. One skilled in the art would be able to determine from the results of the assay against the panel of extracellular proteases whether it is desirable to follow one or more than one of the stressor regimes.

### Harvesting the Plant Material for Extraction and Optional Storage Treatment

The plant material may be used immediately after pre-harvest treatment, or it may be desirable to store the plant material for a period of time prior to performing the extraction procedure(s). If desired, the plant material can be treated prior to storage, for example, by drying, freezing, lyophilising, or some combination thereof.

Following treatment to prepare the plant material for storage, the plant material may be stored for a period of time prior to being contacted with a first solvent. The storage time may be of various durations, for example, the storage period may be between a few days and a few years. In one embodiment of the invention, the plant material is stored for a period of less than one week. In another embodiment, the plant material is stored for a period between one week to one month. In a further embodiment, the plant material is stored for a period of between one month to six months. In other embodiments, the plant material is stored for periods of between four months to one year and for a period over one year in duration.

### The Extraction Process

In accordance with the embodiment depicted in FIG. 1, three basic extraction processes can be performed in sequence to generate potential pre-extracts. In other embodiments of the present invention, greater of fewer extraction processes are contemplated. Regardless of the number of extraction processes, the procedure for each extraction process entails contacting the solid plant material with a solvent with adequate mixing and for a period of time sufficient to ensure adequate exposure of the solid plant material to the solvent such that inhibitory activity present in the plant material can be taken up by the solvent. Typically, the extraction procedures are conducted over a period of time between about 10 minutes and about 24 hours at a temperature between about 4° C. and about 50° C. Adequate contact of the solvent with the plant material can be encouraged by shaking the suspension for 15 minutes to 24 hours at a temperature between about 4° C. and about 50° C.

The liquid fraction is then separated from the solid (insoluble) matter resulting in the generation of two fractions: a liquid fraction, which is a potential pre-extract, and a solid fraction. In accordance with the embodiment depicted in FIG. 1, the extraction process is then repeated with a second and a third solvent, to yield three potential pre-extracts.

Separation of the liquid and solid fractions can be achieved by one or more standard processes known to those skilled in the art. For example, the solid material can be separated from the solvent by centrifugation, filtration (regular or suction), or other means known in the art to separate solids from a solution. In addition, when an alcoholic or organic solvent is used, the potential pre-extract can be dried to remove the solvent and then re-suspended or dissolved in an aqueous solvent prior to testing against a panel of extracellular proteases. The alcoholic or organic solvent can be removed by standard methods including, for example, by distillation or by the use of a lyophilizer, a speedvac, a rotary evaporator, or a vacuum pump and then further dried under vacuum, if necessary in order to remove any remaining solvent.

The dried extract can be dissolved can be dissolved in an aqueous buffer, or in a mixture of an aqueous buffer and a suitable solvent (such as dimethylsulfoxide) prior to analysing its activity against a panel of extracellular proteases. An example of an aqueous buffer is Tris-HCl buffer at a suitable pH, such as between pH 6 and pH 8. In one embodiment, Tris-HCl buffer at pH 7 is used.

Solvents A, B and C in FIG. 1 generally represent separate classes of solvents, for example, aqueous, alcoholic and organic. The solvents can be applied in specific order, for example, a polar to non-polar order or in a non-polar to polar order. Alternatively, the solvents can be applied in a random sequence. In all cases, however, the solid matter should be dried prior to contact with the subsequent solvent.

The term “liquid” is used to denote matter that is distinct from the solid, insoluble matter. Thus, a liquid, which may be converted to a gas or function in a gaseous form (as in the case with steam, for example), can serve as a solvent. Likewise, other non-solid solvents may be used such as highly viscous liquids or other gaseous solvents, some of which can then be converted into a liquid phase. A liquid solvent may also indicate a composition or a mixture of solvents. Common examples include a buffered aqueous solution, such as a TRIS-HCl buffer, an ethanol/methanol combination and combinations of an alcoholic solvent and a co-solvent, such as methanol or water.

The plant material employed in the extraction process can be the entire potential plant, or it can be one or more distinct tissues from a plant, for example, leaves, seeds, roots, stems, flowers, or various combinations thereof. The plant material can be fresh, dried or frozen. If desired, the plant material can be treated prior to the extraction process in order to facilitate the extraction of the inhibitory activity. Typically such treatment results in the plant material being fragmented by some means such that a greater surface area is presented to the solvent. For example, the plant material can be crushed or sliced mechanically, using a grinder or other device to fragment the plant parts into small pieces or particles, or the plant material can be frozen liquid nitrogen and then crushed or fragmented into smaller pieces.

In one embodiment of the present invention, plant material is first fragmented and then extracted with a first solvent comprising an aqueous TRIS-HCl buffer at pH 6-8 for a period of between 30 minutes to 8 hours at a temperature between about 4 to about 50° C. In an alternative embodiment, aqueous buffer has a pH of about 7. In another embodiment, extraction takes place over about 30 min to 2 hours. In a further embodiment, the extraction takes place at a temperature between about 4 to about 25° C. In another embodiment, the extraction takes place at a temperature between about 4 to about 10° C. In another embodiment, the extraction is performed at a temperature of about 4° C. for about 30 minutes.

In one embodiment of the invention, ethanol is used as an alcoholic solvent either alone or in combination with a co-solvent. In another embodiment, a combination of ethanol and methanol is used as the alcoholic solvent, wherein the range of ethanol:methanol is between about 50:50 and about 85:15. In a further embodiment, the plant material is contacted with an alcoholic solvent for a time period between about 10 minutes to one hour at a temperature between about 4 to about 25° C. In another embodiment, the plant material is contacted with an alcoholic solvent for a time period between about 15 and about 30 minutes. In other embodiments, the plant material is contacted with an alcoholic solvent at a temperature between about 4 to about 10° C. and at about 4° C.

In one embodiment of the present invention, diethylether, hexane, dichloromethane, or ethylacetate extract is used as the organic solvent. In another embodiment, the residual solid plant material is shaken for one to twenty-four hours with the organic solvent. In a further embodiment, the residual solid plant material is shaken for one to fifteen hours. In other embodiments, the residual solid plant material is shaken for one to eight hours and for one to four hours with the organic solvent. In another embodiment, dichloromethane is used as the organic solvent and the extraction is performed at room temperature for about 2 hours.

The present invention contemplates that the extraction process may be carried out on various scales including known large, medium and small-scale methods of preparing extracts.

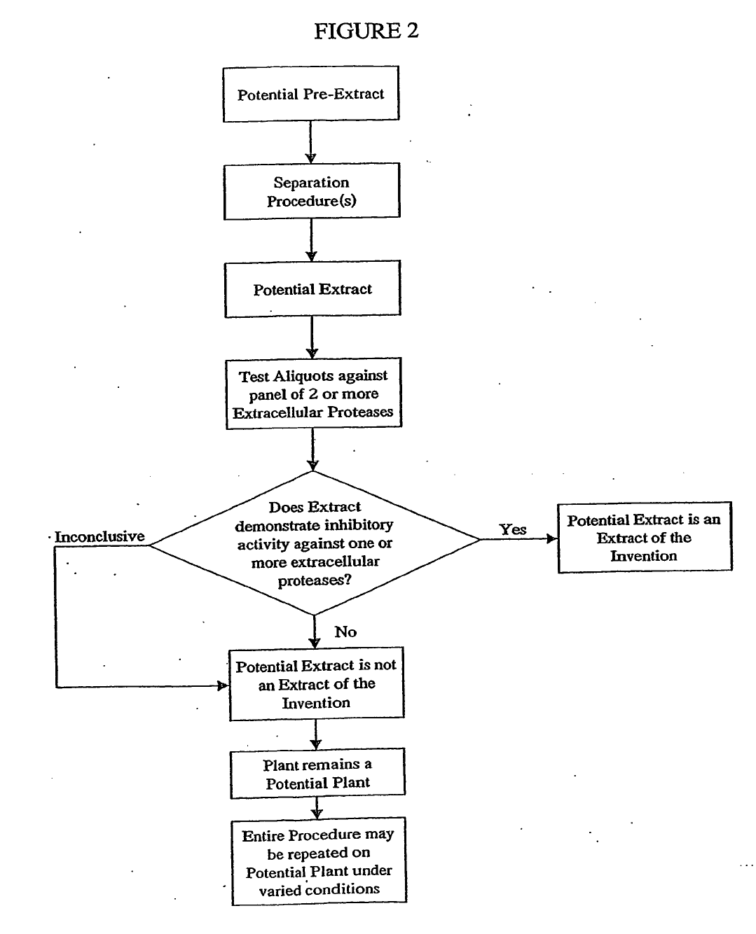

Once the potential pre-extracts have been isolated, they can be tested directly for their ability to inhibit extracellular protease activity, or they may be subjected to further separation procedures to generate a potential extract as described below and outlined in FIG. 2.

### Determination of Extracellular Protease Inhibitory Activity in an Extract

In accordance with the present invention, the plant extracts are capable of inhibiting the activity of at least one extracellular protease. In the context of the present invention, a plant extract that decreases the activity of an extracellular protease by at least 20% when measured according to one of the assays described herein is considered to be capable of inhibiting the activity of that protease.

Extracellular proteases that may be used to test the ability of the extract to inhibit extracellular protease activity include, but are not limited to, matrix metalloproteases (MMPs), cathepsins, elastase, plasmin, TPA, uPA, kallikrein, ADAMS family members, neprilysin, gingipain, clostripain, thermolysin, serralysin, and other bacterial and viral proteases.

It is contemplated that for some purposes, it may be desirable to determine the ability of the potential pre-extract/extract to inhibit a certain set or group of extracellular proteases. For example, it may be useful to determine which potential pre-extracts/extracts are capable of inhibiting at least one human extracellular protease. In this case a panel of extracellular proteases may be designed that comprises those proteases of particular interest. In one embodiment of the present invention, the ability of a potential pre-extract/extract to inhibit at least one extracellular protease is determined using a panel of proteases comprising: MMP-1, MMP-2, MMP-3, MMP-9, cathepsin B, cathepsin D, cathepsin G, cathepsin L, cathepsin K, human leukocyte elastase (HLE), clostripain and subtilisin. In another embodiment, the ability of a potential pre-extract/extract to inhibit at least one extracellular protease is determined using a panel of proteases comprising: MMP-1, MMP-2, MMP-3, MMP-9 and cathepsin B.

One skilled in the art would appreciate that there are numerous methods and techniques for measuring qualitatively and/or quantitatively the ability of the potential pre-extracts and/or potential extracts to inhibit the activity of extracellular protease(s).

For example, there are currently several assays to measure the activity of MMPs, elastase and cathepsins (for a review of these methods, see Murphy and Crabbe, In Barrett (ed.) Methods in Enzymology. Proteolytic Enzymes: Aspartic Acid and Metallopeptidases, New York: Academic Press, 1995, 248: 470), including the gelatinolytic assay (which is based on the degradation of radio-labelled type I collagen), the zymography assay (which is based on the presence of negatively-stained bands following electrophoresis through substrate-impregnated SDS polyacrylamide gels) and a microtitre plate assay developed by Pacmen et al., (Biochem. Pharm. (1996) 52:105-111).

Other methods include those that employ auto-quenched fluorogenic substrates, which do not have some of the drawbacks associated with the above methods, such as the use of radioisotopes, labour-intensiveness, long incubation times and/or low sensitivity. Many fluorogenic substrates have been designed for quantification of the activity of MMPs, elastase, and cathepsins through fluorescent level variation measuring (reviewed by Nagase and Fields (1996) Biopolymers 40: 399-416).

Fluorescence polarization assays are based on the principle that when fluorescent molecules are excited with plane polarized light, they will emit light in the same polarized plane provided that the molecule remains stationary throughout the excited state. However, if the excited molecule rotates or tumbles during the excited state, then light is emitted in a plane different from the excitation plane. If vertically polarized light is used to excite the fluorophore, the emission light intensity can be monitored in both the original vertical plane and also the horizontal plane. The degree to which the emission intensity moves from the vertical to horizontal plane is related to the mobility of the fluorescently labelled molecule. If fluorescently labelled molecules are very large, they move very little during the excited state interval, and the emitted light remains highly polarized with respect to the excitation plane. If fluorescently labelled molecules are small, they rotate or tumble faster, and the resulting emitted light is depolarized relative to the excitation plane. Therefore, FP can be used to follow any biochemical reaction that results in a change in molecular size of a fluorescently labelled molecule (e.g. protein-DNA interactions; immunoassays; receptor-ligand interactions; degradation reactions). (Adapted from Bolger R. Checovich W. (1994) Biotechniques 17(3):585-9.).

Another method of measuring extracellular protease activity makes use of the fluorescent activated substrate conversion (FASC) assay described in Canadian Patent No. 2,189,486 (1996) and in St-Pierre et al., (1996) Cytometry 25: 374-380.

Various formats known in the art may be employed to test the ability of the potential pre-extracts and potential extracts to inhibit the activity of extracellular proteases. For example, the potential pre-extract/extract may be tested against one or more proteases in a sequential fashion or it may be tested against a plurality of proteases, such as an array of extracellular proteases, simultaneously. The assays may be adapted to high throughput in order to facilitate the simultaneous testing of a potential pre-extract/extract against a plurality of proteases. High throughput techniques are constantly being developed and the use of such techniques to adapt the assays in the future is also considered to be within the scope of the present invention.

In one embodiment of the present invention, a potential pre-extract or potential extract is selected for further testing when it demonstrates inhibitory activity against one extracellular protease. In another embodiment, a potential pre-extract or potential extract is selected for further testing when it demonstrates inhibitory activity against two or more extracellular proteases. In a further embodiment, a potential pre-extract or potential extract is selected for further testing when it demonstrates inhibitory activity against three or more extracellular proteases. In another embodiment, a potential pre-extract or potential extract is selected for further testing when it demonstrates inhibitory activity against four or more extracellular proteases.

### Determination of the Ability of the Extract to Modulate Cellular Activity

In accordance with the present invention, extracts are selected by their ability to inhibit one or more extracellular protease and to modulate one or more cellular activity. In one embodiment, extracts are selected by their ability to slow down, inhibit or prevent cell migration.

There are a number of assays known to one skilled in the art, which can be used to test an extract for the ability to modulate cellular activity. For example, various cell migration assays can be used to test the extracts, such as those described herein in Example IV.

In general, the ability of an extract to inhibit migration of endothelial and/or neoplastic cells can be assessed in vitro using standard cell migration assays. Typically, such assays are conducted in multi-well plates, the wells of the plate being separated by a suitable membrane into top and bottom sections. The membrane is coated with an appropriate compound, the selection of which is dependent on the type of cell being assessed and can be readily determined by one skilled in the art. Examples include collagen or gelatine for endothelial cells and Matrigel for neoplastic cell lines. An appropriate chemo-attractant, such as EGM-2, IL-8, aFGF, bFGF and the like, is added to the bottom chamber as a chemo-attractant. An aliquot of the test cells together with the potential pre-extract/extract are added to the upper chamber, typically various dilutions of the potential pre-extract/extract are tested. After a suitable incubation time, the membrane is rinsed, fixed and stained. The cells on the upper side of the membrane are wiped off, and then randomly selected fields on the bottom side are counted.

Various cell lines can be used in cell migration assays. Examples of suitable endothelial cell lines include, but are not limited to, human umbilical vein endothelial cells (HUVECs), bovine aortic endothelial cells (BAECs), human coronary artery endothelial cells (HCAECs), bovine adrenal gland capillary endothelial cells (BCE) and vascular smooth muscle cells. HUVECs can be isolated from umbilical cords using standard methods (see, for example, Jaffe et al. (1973) J. Clin. Invest. 52: 2745), or they can be obtained from the ATCC or various commercial sources, as can other suitable endothelial cell lines. Examples of suitable neoplastic cell lines include those that are available from the American Type Culture Collection (ATCC), which currently provides 950 cancer cell lines, and other commercial sources.

In accordance with one embodiment of the present invention, a potential pre-extract/extract that demonstrates the ability to decrease cell migration by about 10% when used at a concentration of about 10 mg/ml in at least one of the above-described assays is selected as an extract of the invention.

In accordance with another embodiment, a potential pre-extract/extract that demonstrates the ability to decrease cell migration by about 10% when used at a concentration of about 2.5× in at least one of the above-described assays is selected as an extract of the invention, wherein 1× corresponds to the concentration of the potential pre-extract/extract required to inhibit the activity of a selected extracellular protease by at least 50% (i.e. the IC_{>50}).

### In Vivo Testing

As an alternative, or in addition, to the above-described in vitro tests, the ability of the potential pre-extracts/extracts or extracts of the invention to inhibit cell migration in vivo can be assessed using various standard techniques. For example, the ability of the potential pre-extracts/extracts to inhibit endothelial cell migration can be determined using the chick chorioallantoic membrane (CAM) assay, Matrigel plug assay and/or corneal micropocket assay.

The CAM assay can be used to evaluate the ability of an extract to inhibit growth of blood vessels into various tissues, i.e. both angiogenesis and neovascularization (see Brooks et al., in Methods in Molecular Biology, Vol. 129, pp. 257-269 (2000), ed. A. R. Howlett, Humana Press Inc., Totowa, N.J.; Ausprunk et al., (1975) Am. J. Pathol., 79:597-618; Ossonski et al., (1980) Cancer Res., 40:2300-2309). The CAM assay measures neovascularization of whole tissue, wherein chick embryo blood vessels grow into the CAM or into the tissue transplanted on the CAM, and is, therefore, a well-recognised assay model for in vivo angiogenesis. In addition, the assay provides an internal toxicity control in that the chick embryo is exposed to the potential pre-extract/extract over the course of the assay. The health of the embryo can, therefore, provide an indication of the cytotoxicity of the extract.

The Matrigel plug assay is also a standard method for evaluating the anti-angiogenic properties of compounds in vivo (see, for example, Passaniti, et al., (1992) Lab. Invest. 67:519-528). In this assay, an extract is introduced into cold liquid Matrigel which, after subcutaneous injection into a suitable animal model, solidifies and permits penetration by host cells and the formation of new blood vessels. After a suitable period of time, the animal is sacrificed and the Matrigel plug is recovered, usually together with the adjacent subcutaneous tissues. Assessment of angiogenesis in the Matrigel plug is achieved either by measuring haemoglobin or by scoring selected regions of histological sections for vascular density, for example by immunohistochemistry techniques identifying specific factors such as hemagglutinin (HA), CD31 (platelet endothelial cell adhesion molecule-1) or Factor VIII. Modifications of this assay have also been described (see, for example, Akhtar et al., (2002) Angiogenesis 5:75-80; Kragh et al., (2003) Int J Oncol. 22:305-11).

The corneal micropocket assay is usually conducted in mice, rats or rabbits and has been described in detail by others (see D'Amato, et al., (1994) Proc. Natl, Acad. Sci. USA, 91:4082-4085; Koch et al., (1991) Agents Actions, 34:350-7; Kenyon, et al., (1996) Invest. Opthalmol. Vis. Sci. 37:1625-1632). Briefly, pellets for implantation are prepared from sterile hydron polymer containing a suitable amount of the extract. The pellets are surgically implanted into corneal stromal micropockets created at an appropriate distance medial to the lateral corneal limbus of the animal. Angiogenesis can be quantitated at various times after pellet implantation through the use of stereomicroscopy. Typically, the length of neovessels generated from the limbal vessel ring toward the centre of the cornea and the width of the neovessels are measured.

Similarly to the CAM assay both the Matrigel plug assay and the corneal micropocket assay provide some indication of the toxicity of the extract as the test animal is exposed to the extract. The overall health of the animal, therefore, can provide an indication of toxicity.

The ability of the extract to inhibit the migration of neoplastic cells in vivo can be determined using various models of experimental metastasis known in the art. Typically, this involves the treatment of neoplastic cells with the extract ex vivo and subsequent injection or implantation of the cells into a suitable test animal. The spread of the neoplastic cells from the site of injection, for example spread to the lungs and/or lymphoid nodes, is then monitored over a suitable period of time by standard techniques.

### Additional Tests

In addition to the above tests, potential pre-extracts/extracts or extracts of the invention may be submitted to other standard tests, such as those for the assessment of cytotoxicity, stability, bioavailability and the like. Such tests may be conducted prior to testing potential pre-extracts/extracts for their ability to modulate cellular activity or they may be conducted once an extract of the invention has been selected. As will be readily apparent to one skilled in the art, a selected extract will need to meet certain criteria in order to be suitable for in vivo use and to meet regulatory requirements. Conducting such tests, therefore, allows the suitability of an extract for in vivo use to be assessed. Similarly, once an extract has been found to be suitable for animal administration, its efficacy may be determined by standard in vivo tests and clinical trials.

### Commercial Processes for Preparing Extracts of the Invention

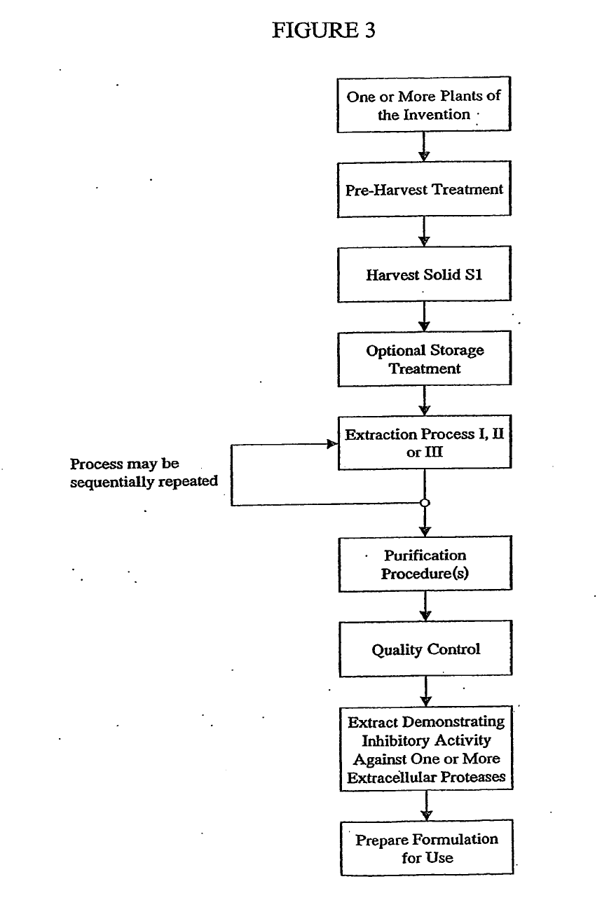

The present invention contemplates the large-scale preparation of selected extracts of the invention. Such extracts can be prepared on a commercial scale by repeating the extraction process that lead to the isolation of the extract of interest. One embodiment of this aspect of the invention is presented in FIG. 3. In this embodiment, the small-scale extraction procedure is simply scaled-up and additional steps of quality control are included to ensure reproducible results for the resulting extracts.

Also contemplated by the present invention are modifications to the small-scale procedure that may be required during scale-up for industrial level production of the extract. Such modifications include, for example, alterations to the solvent being used or to the extraction procedure employed in order to compensate for variations that occur during scale-up and render the overall procedure more amenable to industrial scale production, or more cost effective. Modifications of this type are standard in the industry and would be readily apparent to those skilled in the art.

Purification/Fractionation of Extracts and Active Ingredients from Extracts of the Invention

The present invention also provides for active ingredients from the extracts of the inventions, and for purified or concentrated extracts. The present invention further provides for methods of purifying one or more active ingredient from the extracts of the invention. In the context of the present invention an “active ingredient” is a compound or molecule that is capable of inhibiting one or more extracellular protease and that demonstrates the ability to modulate one or more cellular activity. The active ingredient may be either proteinaceous or non-proteinaceous. “Purifying” an active ingredient or extract indicates that the active ingredient or purified extract can be obtained by purification, partial purification, and/or fractionation of an extract of the invention.

There are a number of techniques well known in the art for isolating active components from mixtures. For example, purification, partial purification, and/or fractionation can be performed using solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilisation, evaporation, precipitation with various “carriers” (including PVPP, carbon, antibodies, etc.), or various combinations thereof. One skilled in the art, would appreciate how to use such options, in a sequential fashion, in order to enrich each successive fraction in the activity of interest by following its activity throughout the purification procedure. Typically, the activity is the inhibitory activity against an extracellular protease of interest and can be measured using assays such as those described above.

Solid-liquid extraction means include the use of various solvents in the art, and includes the use of supercritical solvents, soxhlet extractors, vortex shakers, ultrasounds and other means to enhance extraction, as well as recovery by filtration, centrifugation and related methods as described in the literature (see, for example, R. J. P. Cannell, Natural Products Isolation, Humana Press, 1998). Examples of solvents that may be used include, but are not limited to, hydrocarbon solvents, chlorinated solvents, organic esters, organic ethers, alcohols, water, and mixtures thereof. In the case of supercritical fluid extraction, the invention also covers the use of modifiers such as those described in V. H. Bright (Supercritical Fluid Technology, ACS Symp. Ser. Vol. 488, ch. 22, 1999). Liquid-liquid extraction means include the use of various mixtures of solvents known in the art, including solvents under supercritical conditions. Typical solvents include, but are not limited to, hydrocarbon solvents, chlorinated solvents, organic esters, organic ethers, alcohols, water, various aqueous solutions, and mixtures thereof. The liquid-liquid extraction can be effected manually, or it can be semi-automated or completely automated, and the solvent can be removed or concentrated by standard techniques in the art (see, for example, S. Ahuja, Handbook of Bioseparations, Academic Press, 2000).

Solid-phase extraction (SPE) techniques include the use of cartridges, columns or other devices known in the art. The sorbents that may be used with such techniques include, but are not limited to, silica gel (normal phase), reverse-phase silica gel (modified silica gel), ion-exchange resins, and fluorisil. The invention also includes the use of scavenger resins or other trapping reagents attached to solid supports derived from organic or inorganic macromolecular materials to remove selectively active ingredients or other constituents from the extracts.

Membrane, reverse osmosis and ultrafiltration means include the use of various types of membranes known in the art, as well as the use of pressure, vacuum, centrifugal force, and/or other means that can be utilised in membrane and ultrafiltration processes (see, for example, S. Ahuja, Handbook of Bioseparations, Academic Press, 2000).

Dialysis means include membranes having a molecular weight cut-off varying from less than about 0.5 KDa to larger than about 50 KDa. The invention also covers the recovery of purified and/or fractionated extracts from either the dialysate or the retentate by various means known in the art including, but not limited to, evaporation, reduced pressure evaporation, distillation, vacuum distillation, and lyophilization.

Chromatographic means include various means of carrying out chromatography known by those skilled in the art and described in the literature (see, for example, G. Sofer, L. Hagel, Handbook of Process Chromatography, Academic Press, 1997). Examples include, but are not limited to, regular column chromatography, flash chromatography, high performance liquid chromatography (HPLC), medium pressure liquid chromatography (MPLC), supercritical fluid chromatography (SFC), countercurrent chromatography (CCC), moving bed chromatography, simulated moving bed chromatography, expanded bed chromatography, and planar chromatography. With each chromatographic method, examples of sorbents that may be used include, but are not limited to, silica gel, alumina, fluorisil, cellulose and modified cellulose, various modified silica gels, ion-exchange resins, size exclusion gels and other sorbents known in the art (see, for example, T. Hanai, HPLC: A Practical Guide, RSC Press, UK 1999). The present invention also includes the use of two or more solvent gradients to effect the fractionation, partial purification, and/or purification of said active extracts by chromatographic methods. Examples of solvents that may be utilised include, but are not limited to, hexanes, pentane, petroleum ethers, cyclohexane, heptane, diethyl ether, methanol, ethanol, isopropanol, propanol, butanol, isobutanol, tert-butanol, water, dichloromethane, dichloroethane, ethyl acetate, tetrahydrofuran, dioxane, tert-butyl methyl ether, acetone, and 2-butanone. When water or an aqueous phase is used, it may contain varying amounts of inorganic or organic salts, and/or the pH may be adjusted to different values with an acid or a base such that fractionation and/or purification is enhanced.

In the case of planar chromatography, the present invention includes the use of various forms of this type of chromatography including, but not limited to, one- and two dimension thin-layer chromatography (1D- and 2D-TLC), high performance thin-layer chromatography (HPTLC), and centrifugal thin-layer chromatography (centrifugal TLC).

In the case of countercurrent chromatography (CCC), the present invention includes the use of manual, semi-automated, and automated systems, and the use of various solvents and solvent combinations necessary to effect fractionation and/or purification of active ingredients or extracts (see, for example, W. D. Conway, R. J. Petroski, Modern Countercurrent Chromatography, ACS Symp. Ser. Vol. 593, 1995). Solvent removal and/or concentration can be effected by various means known in the art including, but not limited to, reduced pressure evaporation, evaporation, reduced pressure distillation, distillation, and lyophilization.

The present invention includes the fractionation, partial purification, and purification of active ingredients or extracts by expanded bed chromatography, moving and simulated moving bed chromatography, and other related methods known in the art (see, for example, G. Sofer, L. Hagel, Handbook of Process Chromatography, Academic Press, 1997 and S. Ahuja, Handbook of Bioseparations, Academic Press, 2000).

Selective precipitation means includes the use of various solvents and solvent combinations, the use of temperature changes, the addition of precipitant and/or modifiers, and/or modification of the pH by addition of base or acid to effect a selective precipitation of active ingredients or other constituents.

The invention also includes the fractionation, partial purification, and/or purification of active ingredients and extracts by steam distillation, hydrodistillation, or other related methods of distillation known in the art (see, for example, L. M. Harwood, C. J. Moody, Experimental Organic Chemistry, Blackwell Scientific Publications, UK, 1989).

The process of purifying the active ingredients or extracts also includes the concentration of purified or partially purified active ingredients or extracts by solvent removal of the original extract and/or fractionated extract, and/or purified extract. The techniques of solvent removal are known to those skilled in the art and include, but are not limited to, rotary evaporation, distillation (normal and reduced pressure), centrifugal vacuum evaporation (speed-vac), and lyophilization.

Purified, partially purified and/or concentrated active ingredients and extracts can be tested for their ability to inhibit one or more extracellular protease and to modulate cellular activity according to the one or more of the procedures described above.

### Formulations and Pharmaceutical Compositions

The present invention further provides for formulations and pharmaceutical compositions comprising one or more extract of the invention, one or more active ingredient, or a combination thereof.

The formulations and pharmaceutical compositions of the invention comprise extracts and/or active ingredients capable of inhibiting one or more extracellular protease and modulating one or more cellular activity. In one embodiment of the invention, the formulations and pharmaceutical compositions comprise extracts and/or active ingredients capable of slowing down, inhibiting or preventing endothelial or neoplastic cell migration. In general, the extract or active ingredient has the capacity to inhibit at least one of the active proteases involved in the physiological process being targeted, i.e. preventing endothelial or neoplastic cell migration, with a good inhibition constant (Kᵢ). The formulations and pharmaceutical compositions must also have acceptable toxicity and stability. In addition, if the formulation is administered by different means other than topically (e.g. via oral, intraperitoneal, intravenous, subcutaneous, intramuscular etc. routes), then the extract and/or active ingredient must demonstrate acceptable hepatotoxicity and must be sufficiently resistant to degradation to allow the site of action to be reached. Finally, the formulation or pharmaceutical composition must be formulated in a manner to enable administration to the animal in need of treatment. Testing for the above parameters and formulation of appropriate compositions and formulations can be readily achieved by one skilled in the art.

The formulation or pharmaceutical composition may be in a solid or liquid form, for example, a cream, gel or ointment (for a topical application), or gel-cap, tablet or capsule (for oral administration), or other formulation suitable for administration to an animal.

Criteria which must be considered in the preparation of a formulation include, but are not limited to, the physicochemical and biochemical characteristics (bioavailability, toxicity, stability, etc.) of the extracts and/or active ingredients which make up the formulation. In particular, the formulation is prepared so as to preserve, as much as possible, the maximum inhibitory activity of the active components upon administration, without being harmful to the animal. In one embodiment, the overall capacity for inhibition of proteolytic activity in the formulation correlates with the proteolytic overactivity profile of the biological condition being targeted, i.e. cell migration.

Pharmaceutical compositions may be formulated by mixing the extracts and/or active ingredients together with a physiologically acceptable carrier, excipient, binder, diluent, etc. Alternatively, the extracts and/or active ingredients can be formulated independently and the respective formulations can then be extemporaneously admixed using a diluent or the like and administered, or can be administered independently of each other, either concurrently or at staggered times to the same subject.

One embodiment of the invention relates to the preparation of pharmaceutical compositions comprising a therapeutically effective amount of the above said active material or mix of active materials and a pharmaceutically acceptable carrier, diluent, vehicle, or excipient. The pharmaceutical compositions according to the invention may be adapted for oral (capsules tablets, phials, etc.), parenteral, rectal, inhalation, or topical administration, including creams, gels, etc. and may be in unit dosage form. Also, the composition may be adapted for slow release in vivo as known in the art.

The pharmaceutical compositions of the invention may be used in conventional formulations including, but not limited to, solutions, syrups, emulsions, injectables, tablets, capsules, suppositories, hydrophobic and hydrophilic creams and lotions.

In another embodiment, the invention relates to the preparation of herbal and nutraceutical formulations comprising extracts and/or active ingredients or solid parts of the plant(s) from with the extracts were obtained. For nutraceutical formulations comprising solid parts of plant(s), the plant(s) must be an edible plant. The extracts and/or active ingredients or plant parts can be used in these herbal remedies and nutraceutical compositions as solutions, purified solutions, or dry powders after treatments such as those described below.

The formulations and compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. One or more extract and/or active ingredient may be present in association with one or more non-toxic pharmaceutically acceptable carriers and/or diluents and/or adjuvants and, if desired, other active ingredients. The pharmaceutical compositions containing one or more extract and/or active ingredient may be in a form suitable for oral use, for example, as tablets, troches, lozenges, aqueous or oily suspensions, dispersible powders or granules, emulsion hard or soft capsules, or syrups or elixirs.

Formulations intended for oral use may be prepared according to methods known in the art for the manufacture of pharmaceutical compositions and such compositions may contain one or more agents such as sweetening agents, flavouring agents, colouring agents and preserving agents in order to provide pharmaceutically elegant and palatable preparations. Tablets contain the extracts and/or active ingredients in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate: granulating and disintegrating agents for example, corn starch, or alginic acid: binding agents, for example starch, gelatine or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium, for example peanut oil, liquid paraffin or olive oil.

Aqueous suspensions contain extracts and/or active ingredients in admixture with excipients suitable for the manufacture of aqueous suspensions. Such excipients are suspending agents, for example, sodium carboxymethylcellulose, methyl cellulose, hydropropylmethylcellulose, sodium alginate, polyvinylpyrrolidone, gum tragacanth and gum acacia: dispersing or wetting agents may be a naturally-occurring phosphatide, for example, lecithin, or condensation products of an alkylene oxide with fatty acids, for example polyoxyethyene stearate, or condensation products of ethylene oxide with long chain aliphatic alcohols, for example hepta-decaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the extracts and/or active ingredients in a vegetable oil, for example, arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavouring agents may be added to provide palatable oral preparations. These compositions may be preserved by the addition of an anti-oxidant such as ascorbic acid.

Dispersible powders and granules suitable for preparation of an aqueous suspension by the addition of water provide the extracts and/or active ingredients in admixture with a dispersing or wetting agent, suspending agent and one or more preservatives. Suitable dispersing or wetting agents and suspending agents are exemplified by those described above. Additional excipients, for example, sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example liquid paraffin or mixtures of these. Suitable emulsifying agents may be naturally-occurring gums, for example, gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative and flavouring and colouring agents. The pharmaceutical compositions may be in the form of a sterile injectable aqueous or oleaginous suspension. This suspension may be formulation according to methods known in the art using suitable dispersing or wetting agents and suspending agents such as those mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

### Use

The present invention further provides for the in vivo use of the extracts of the invention and/or active ingredients derived from the extracts, and formulations and pharmaceutical compositions comprising extracts and/or active ingredients. Thus, the extracts, active ingredients, formulations or pharmaceutical compositions can be administered to an animal in order to slow down, inhibit or prevent undesirable migration of endothelial and/or neoplastic cells and to ameliorate conditions associated therewith. For example, the extracts, active ingredients, formulations or pharmaceutical compositions can be administered to an animal in order to slow down angiogenesis, neovascularisation or tumour metastasis.

As is known in the art, a variety of tissues, or organs comprised of organised tissues, can support angiogenesis including skin, muscle, gut, connective tissue, joints, bones and the like in which blood vessels can invade upon angiogenic stimuli. In addition, a variety of tumour types are known to be capable of metastasizing. The extracts, active ingredients, formulations or pharmaceutical compositions are, therefore, useful in slowing down the migration or invasion of endothelial or neoplastic cells in a variety of animal tissues.

To gain a better understanding of the invention described herein, the following examples are set forth. It should be understood that these examples are for illustrative purposes only. Therefore, they should not limit the scope of this invention in any way.

### EXAMPLES

### Example I

### Preparation of Stressed and Non-Stressed Plant Extracts

Pre-Harvest Treatment: Aerial parts of a living plant are sprayed with an aqueous solution of gamma linolenic acid (6,9,12-Octadecatrienoic acid, Sigma L-2378) (stress G) or arachidonic acid (5,8,11,14-Eicosatetraenoic acid, Sigma A-3925) (stress A) (400 μM in water with 0.125% (v/v) Triton X-100) to completely cover the leaves. Twenty to twenty-four hours after the stress, plants are harvested.

### Harvest Solid S1 and Optional Storage Treatment

Twenty to twenty-four hours after the stress, more than 4 grams of leaves, stems, fruit, flowers, seeds or other plant parts are harvested and frozen immediately in dry ice, then transferred as soon as possible to a −20° C. freezer until use. Plant materials may be stored at −20 C for a long period of time, more than a year, without losing inhibitory activity. Temperature is monitored to ensure a constant condition.

Stressed and non-stressed plant specimens are collected as wet samples and stored at −20° C. for various periods of time, and are submitted to a process which generates 3 subfractions: aqueous, ethanolic and organic fractions. The complete extraction process is performed in a continuous cycle using the following steps. An initial 5 g of plant specimen is homogenized in liquid nitrogen with a blender. The resulting powder is weighed.

### Extraction Process I: Aqueous Extraction

To each 4.5 grams of plant powder, 12 ml of a cold solution of 100 mM Tris, pH 7.0 is added. The mixture is thoroughly vortexed for 2 minutes. The mixture is kept on ice for 30 minutes and vortexed after each 10 minute period of time. The sample is centrifuged in a Corex™ 30 ml tube for 5 minutes at 4500 rpm. The resulting supernatant is decanted in a 15 ml tube after filtration with a Miracloth™ filter. This extract is therefore referred as the Potential Pre-Extract A. The pellet, referred as Solid S2, is kept for ethanolic extraction.

The aqueous extract (Potential Pre-Extract A) is further purified in order to determine its extracellular protease inhibition capability. The Potential Pre-Extract A is purified by size-exclusion chromatography, wherein the aqueous extract is chromatographed on a calibrated Sephadex G-25 column (1×10 cm) using a 20 mM Tris-HCl, 150 mM NaCl, pH 7.5 buffer as eluant. Fractions corresponding to compounds that seem to have a molecular weight (MW) less than 1500 daltons (D) are pooled to constitute the purified aqueous extract that is tested for inhibitory activity in an assay as described in Example II.

Prior to this analysis, the extract is treated with 10% gelatin-Sepharose (Pharmacia Biotech, Uppsala, Sw.) in order to remove unspecific enzyme ligands. To 1 mL of extract, 100 μL of gelatin-Sepharose resin is added in a microassay tube, the solution in the tube is mixed, kept on ice for 30 minutes, and then centrifuged 5 minutes at 5,000 rpm. The supernatant is removed and used directly for assays.

### Extraction Process II: Alcoholic Extraction

To the pellet, Solid S2, collected from the previous aqueous extraction, 12 ml of cold ethanol:methanol (85:15) is added and the mixture is thoroughly vortexed for 2 minutes. The mixture is kept on ice for 30 minutes and vortexed every 10 minutes. The sample is centrifuged in a Corex™ 30 ml tube for 5 minutes at 4,500 rpm. The resulting supernatant is decanted in a 15 ml tube after filtration with a Miracloth™ filter. The pellet, referred as Solid S3 is kept for the subsequent organic extraction. This extract is therefore referred as the Potential Pre-Extract B.

The ethanolic extract, Potential Pre-Extract B, is purified by liquid/liquid extraction prior to analysis by enzymatic assay. For this purpose, 1 ml of ethanolic extract is evaporated under vacuum, dissolved in 150 μl of dimethylsulfoxide (DMSO), and completed to a final volume of 1.5 ml with Tris buffer (final concentration: Tris-HCl 20 mM; pH 7.5). Four ml of hexane is added to the Tris phase in a glass tube and the tube is thoroughly vortexed, then allowed to form a biphasic liquid. The organic phase is removed and the extract is submitted to a second round of liquid/liquid extraction. The aqueous phase is removed and treated with 10% gelatin-Sepharose (Pharmacia Biotech, Uppsala, Sw) to remove unspecific enzyme ligands prior to conducting subsequent assays. To 1 ml of extract, 1001 L of gelatin-Sepharose resin is added in a microassay tube, the tube is mixed, kept on ice for 30 minutes, and then centrifuged 5 minutes at 5,000 rpm. Supernatant is removed and used directly for assays as described in Example II.

### Extraction Process III: Organic Extraction

To the pellet, Solid S3, collected from previous ethanolic extraction, 12 ml of cold dichloromethane is added and the mixture is thoroughly vortexed for 2 minutes. The mixture is kept on ice for 30 minutes and vortexed after each 10 minutes period. The sample is centrifuged in a Corex™ 30 ml tube for 5 minutes at 4,500 rpm. The resulting supernatant is decanted in a 15 ml glass tube after filtration with a Miracloth™ filter. The final pellet is discarded. The organic solvent is evaporated under vacuum and the phase is dissolved with dimethylsulfoxide (DMSO). This extract is therefore referred as the Potential Pre-Extract C, which was further purified by solid phase extraction prior to analysis by enzymatic assay.

In order to assay the Potential Pre-Extract C, the organic extract is diluted 1:10 in a solution of DMSO:Methanol:Tris (20 mM, pH 7.5) (10 :50 :40) (Solution A), i.e., 220 μl of extract is added to 2.0 ml of solution A. After 10 seconds of vigorous vortex, the mix is sonicated for 10 seconds. Dissolved extracts are subsequently applied to a solid phase extraction plate (Discovery SPE-96, Sigma Chemical Co, St-Louis, Mo.). After initial conditioning of the columns with 1 ml of methanol, columns are equilibrated with solution A, and extract samples are deposited on the columns. Elution is completed with solution A (final volume of 2 ml) and this fraction is used directly in assays as described in Example II.

### Example II

### In vitro Enzyme Inhibition Assays

The inhibitory activity of sample compositions towards human MMP-1, human MMP-2, human MMP-3, human MMP-9, human cathepsin-B, human cathepsin-D, human cathepsin-G, human cathepsin-L, human cathepsin-K, human leukocyte elastase (HLE), bacteria clostripain and bacteria subtilisin can be determined using either fluorogenic substrates or the FASC assay.

Measurement of Human MMP-1, -2, -3 and -9 Activity with Fluorogenic Peptidic Substrates

MMP-1, -2, -9 are purified from natural sources (human immortalized cell lines: 8505C (Deutsche Sammlung von Mikroorganismen und Zellkulturen GmbH) for MMP-1, HT-1080 (ATCC, Manassas, Va.) for MMP-2 and THP-1 (ATCC, Manassas, Va.) for MMP-9) as described in literature and based on protocols found in I. M. Clark: <<Matrix metalloproteinases protocols>>, Humana Press (2001). Recombinant human MMP-3 is overexpressed in E. coli and purified according to Windsor L J, Steele D L (2001), Methods Mol Biol 151:191-205. Proteolytic activity of these proteases is evaluated with the assay based on the cleavage of auto-quenched peptide substrate: (MCA-Pro-Leu-Gly-Leu-Dpa-Ala-Arg-NH₂.TFA [Dpa ═N-3-(2,4-dinitrophenyl)-L-2,3-diaminopropionyl]) for MMP-1, -2, and -9; and, MCA-Arg-Pro-Lys-Pro-Val-Glu-Nva-Trp-Arg-Lys(DNP)—NH₂ (DNP=2,4-dinitrophenyl; Nva=L-norvaline) for MMP-3 (Calbiochem, San Diego, Calif.). In the intact peptide, Dpa or DNP quenches the MCA fluorescence. Cleavage of the peptide causes release of the fluorescent MCA group which is then quantitated on a fluorometer (Gemini XS, Molecular Devices, Sunnyvale, Calif.). The assay is performed in TNCZ assay buffer (20 mM Tris-HCl; NaCl 150 mM; CaCl₂ 5 mM; ZnCl₂ 0.5 mM; pH 7.5) with human purified proteases (I. M. Clark: Matrix metalloproteinases protocols, Humana Press (2001)). The substrate, primarily dissolved in DMSO is then redissolved in TNCZ buffer for the assay. In a typical assay, 10 μl of purified enzyme (1-50 ng) and 5 μl of dissolved substrate (final concentration of 10 μM) is mixed in a final volume of 75 μl (completed with TNCZ). All assays were performed in 96 well plate and the reaction is started by the addition of substrate. Assays are measured (excitation 325 nm, emission 392 nm) for 20, 40 and 60 minutes.

Measurement of Human Cathepsin L and K Activity with Fluorogenic Peptidic Substrate.

Human recombinant cathepsins L and K are overexpressed in P. pastoris according to the protocol described by Krupa and Mort (Anal Biochem (2000), 283(1):99-103). The assay is similar to that described above except for the auto-quenched peptidic substrate: Z-Arg-Phe-AMC, 2HCl (Bachem California, Torrance, Calif.) and reaction buffer. Assays for Cathepsin L are performed in 20 mM acetate pH 5.5, 1 mM EDTA buffer and assays for Cathepsin K in 20 mM acetate pH 4.2, 1 mM EDTA. Assays are monitored with fluorometer settled at excitation 380 nm/emission 460 nm wavelengths (Krupa J C, Mort J S. (2000), Anal Biochem 283(1):99-103).

### Measurement of Human MMP-9, Cathepsin B, Cathepsin G, and Human Leukocyte Elastase (HLE) Activity Using the FASC Assay

Human Cathepsin B and G and human leukocyte elastase are obtained from Calbiochem (San Diego, Calif.). Human MMP-9 is purified as previously described. The assay is based on the method described in Canadian Patent No. 2,189,486 (1996) and by St-Pierre et al., (Cytometry (1996) 25:374-380. For the assay, 5 μl of the purified enzyme (1-100 ng), 5 μl of concentrated buffer solution (20 mM Tris-HCl; NaCl 150 mM; CaCl₂ 5 mM; ZnCl₂ 0.5 mM; pH 7.5), and 5 μl of gelatin-FITC beads are typically used in a final volume of 100 μl. The assay is performed by incubation of the reaction mixture for 90 minutes at 37° C. The reaction is stopped by the transfer of the mix in 0.5 ml of 20 mM Tris, 150 mM NaCl; pH 9.5 buffer. This tube is analyzed in a flow cytometer (Epics MCL, Beckman Coulter, Mississauga, Ontario) as described in Canadian Patent No. 2,189,486 (1996).

Measurement of Human Cathepsin D, Cathepsin B, Cathepsin G and HLE Activity with a Fluorogenic Proteic Substrate

Cathepsin D is purified from human MCF-7 cells according to the method described by Stewart et al., (Int J Cancer (1994) 57(5):715-8. Cathepsin B, Cathepsin G and HLE are obtained as previously described. The activities of Cathepsin D, Cathepsin B, Cathepsin G and HLE are measured by an assay based on the increase of fluorescence of a proteic substrate (Haemoglobin in the case of Cathepsin D and B and beta-casein in the case of Cathepsin G and HLE) heavily labelled with Alexa-488 dye (Molecular Probes, Eugene, Or). The substrate, when highly labelled with the dye, will almost quench the dye fluorescence. Cleavage of the substrate will result in an increase of the fluorescence which can be measured with a spectrofluorometer, and which is proportional to protease activity. Typically, 10 μl of purified human Cathepsin D, Cathepsin B, Cathepsin G or HLE (10-50 ng) and 10 μL of Hemoglobin-Alexa488 or beta-casein-Alexa488 (100 ng) are assayed in final volume of 75 μl adjusted with 20 mM citrate pH 3.3 buffer in the case of Cathepsins D and B or TNCZ buffer in the case of Cathepsin G and HLE. The reaction is performed as already described except that the fluorescence is read at excitation 488 nm/emission 525 nm wavelengths.

### Subtilisin Assay

Subtilisin (isolated from B. subtilis) is purchased from Fluka. Assays are performed with a fluorogenic peptide (Z-Gly-Gly-Leu-AMC, Bachem California, Torrance, Calif.) as already described for MMPs with the following modification: the assay is buffered with 20 mM Tris, 150 mM NaCl; pH 7.5 and the results are read at excitation 380 nm/emission 460 nm wavelengths.

### Clostripain Assay

Clostripain from Clostridium histolyticum (Worthington Lakewood, N.J.) is prepared and activated as described by manufacturer's protocol. The activity is determined by using Z-Arg-Arg-AMC, 2HCl (Calbiochem, San Diego, Calif.) as a fluorogenic peptidic substrate and the incubation buffer is 75 mM phosphate, pH 7.6. The reaction is performed as already described except that the fluorescence is read at excitation 380 nm/emission 460 nm wavelengths.

### Extract Inhibition Assay

Before a typical assay, aqueous extracts prepared as described in Example I are preincubated with 1:10 of gelatin-Sepharose 4B™ for 30 minutes to remove fluorescence quenching. For the ethanolic extract, an initial hexane extraction is performed and samples are treated with 1:10 of gelatin-Sepharose 4B™ to remove quenching.

In a typical fluorescent assay, 10 μl of purified enzyme at concentrations previously mentioned for the enzymatic assay, 5 μl of dissolved fluorogenic peptide or 10 μl of dissolved fluorescent proteic substrate (final concentration of 10 μM) and 40 μL of the aqueous, ethanolic or organic extract to be tested and prepared as described in Example I are mixed in a final volume of 75 μl (completed with TNCZ for fluorogenic peptide substrate assay or 20 mM citrate pH 3.3 buffer for fluorescent protein substrate assay). All assays are performed in 96 well plate and the reaction is started by the addition of substrate. Assays are measured (excitation 325 nm, emission 392 nm for peptide and excitation 488 nm/emission 525 nm wavelengths for protein) for 20, 40 and 60 minutes. Activity and inhibition values are determined from the increase in fluorescence

For the FASC assay, 35 μl of the treated extract prepared as described in Example 1, 5 μl of the purified enzyme prepared as described previously, 5 μl of concentrated buffer solution (TNCZ), and 5 μl of gelatin-FITC beads are typically used. The initial step of the assay is the incubation of the reaction without beads for a 30 minutes period on ice to allow the binding of inhibitors to enzyme. Fluorescent beads are added and the reaction mix is incubated for 90 minutes at 37° C. The reaction is stopped by transfer of the mix in 0.5 ml of 20 mM Tris, 150 mM NaCl; pH 9.5 buffer. This tube is analyzed in the flow cytometer (Epics MCL, Beckman Coulter, Mississauga, Ontario) as described in Canadian Patent Application No. 2,189,486 (1996).

Results of the inhibition studies are shown in Tables 1-12 for aqueous (A), ethanolic (R) and organic (S) extracts from exemplary stressed (A and G) and non-stressed (T) plant sources. The inhibition is reported as percentage (%) of inhibition of substrate degradation as compared with the degradation without extract.

Table 1: inhibition of human MMP-1.
Table 2: inhibition of human MMP-2.
Table 3: inhibition of human MMP-3.
Table 4: inhibition of human MMP-9.
Table 5: inhibition of human Cathepsin B.
Table 6: inhibition of human Cathepsin D.
Table 7: inhibition of human Cathepsin G.
Table 8: inhibition of human Cathepsin L.
Table 9: inhibition of human Cathepsin K.
Table 10: inhibition of HLE.
Table 11: inhibition of bacterial subtilisin.
Table 12: inhibition of bacterial clostripain.

### Example III

### Exemplary Purification of Inhibitory Activity Found in an Extract

Extracts were separated by HPLC on an Agilent 1100 system (San Fernando, Calif.). Briefly, 100 μL of a crude extract prepared as described in Example I was applied on a C18 reverse-phase column (Purospher RP-18 5 μm, 4.0×125 mm (HP), Agilent, San Fernando, Calif.). Elution of compounds was achieved with a linear gradient of 10-85% acetonitrile. Fractions were collected, evaporated, resuspended in aqueous buffer and then reanalysed for their inhibition activity on specific enzymes as already described. Fractions of interest (demonstrating a biological activity) where then reisolated at a larger scale for further analysis and characterisation.

### Example IV

### Effect of Plant Extracts on Cell Migration

Plant extracts were prepared as described in Example I and underwent further testing to ascertain that they contain stable, orally bioavailable, non-cytotoxic molecules that are appropriate for product development. Stability is ascertained by recovery of protease inhibition over time under various conditions, including physiological conditions. Potential for oral bioavailability is ascertained by an in vitro test using Caco-2 cells and cytotoxicity is ascertained by incubation of the extracts with various cell types, including those indicated below.

### Methods for Determination of Anti-Angiogenic and Anti-Invasive Effects of Plant Extracts

In order to test the effect of various plant extracts that are also validated protease inhibitors on cellular migration, the following cellular assays were used: a cellular migration assay coupled with a cord formation assay using endothelial cells; and a cellular migration assay using one of 2 neoplastic cell lines. The experimental details are provided below and the results of the tests are set forth in Tables 13 and 14. Concentrations of plant extracts are expressed as a function of the IC₅₀ concentration determined for protease inhibition, which is termed 1×. The extracts are, therefore, capable of decreasing the activity of at least one extracellular protease by at least 50% when measured according to one of the assays described herein. The 1× concentration can vary depending on the plant and the solvent used in the preparation of the extract. The average concentration of a 1× aqueous extract is about 1.6 mg/ml, whereas the average concentration of a 1× alcoholic extract is about 4 mg/ml. For each extract tested in the assays described below, 4 different concentrations were used (0.31×, 0.62×, 1.25× and 2.5×) in duplicate.

### Cell Migration Assays

Migration was assessed using a multi-well system (Falcon 1185, 24-well format), separated by a PET membrane (8 μm pore size) into top and bottom sections. Depending on the cells that are used in the assay, the membrane was coated with 10 μg/ml rat tail collagen (for HUVECs) or with 80 μg/cm² of Matrigel growth factor (BD Biosciences) (for cancer cell lines) and allowed to dry. All solutions used in top sections were prepared in DMEM-0.1% BSA, whereas all solutions used in the bottom sections were DMEM, or other media, containing 10% fetal calf serum. For HUVECs (Clonetics), EGM-2 (700 μl) was added to the bottom chamber as a chemo-attractant. HUVEC (100 μl of 10⁶ cells/ml) and buffer containing the plant extract at the appropriate dilution were added to the upper chamber (duplicate wells of each plant extract at each dilution). After 5 h incubation at 37° C. in a 5% CO₂ atmosphere, the membrane was rinsed with PBS, fixed and stained. The cells on the upper side of the membrane were wiped off, three randomly selected fields were counted on the bottom side.

The percent inhibition of migration is calculated as follows:

[(A−B)/A]×100,

where A is the average number of cells per field in the control well and B is the average number of cells per field in the treated wells.

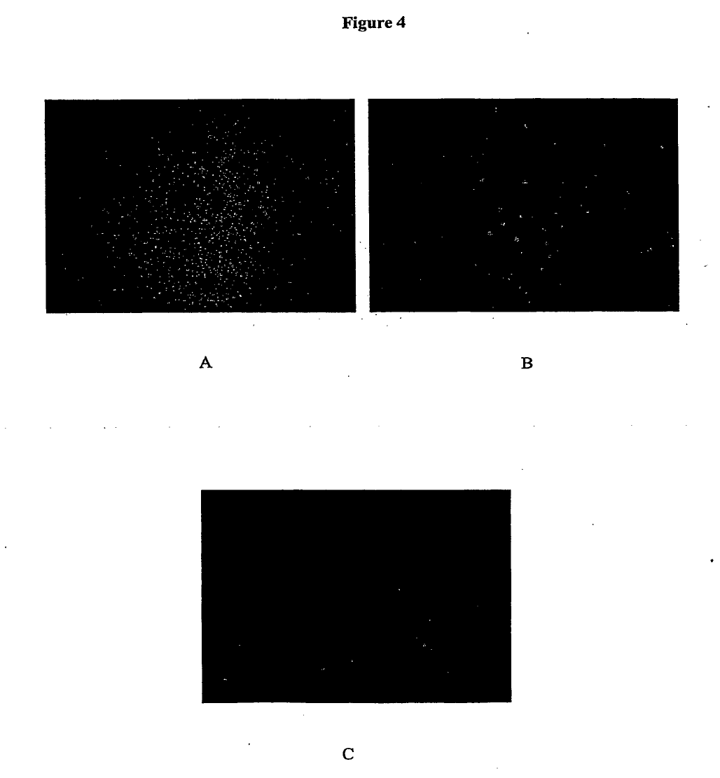

For cancer cell lines, prior to starting the experiment, the Matrigel impregnated filter was rehydrated with 200 μl of DMEM. A mixture of cells (100 μl of 2.5×10⁵/ml HT1080 or MDA-MB-231 cells, both from ATCC) and plant extracts were pipetted into the upper wells and 700 μl of DMEM-5% SVF was added to the bottom wells. The cells were incubated for 48 hours (HT1080 cells) or 72 hours (MDA-MB-231 cells), after which the membrane was treated as described above and inhibition of migration was determined as described above (see also FIG. 4, which shows the results using an extract from Iberis sempervirens).

### Cord Formation Assay

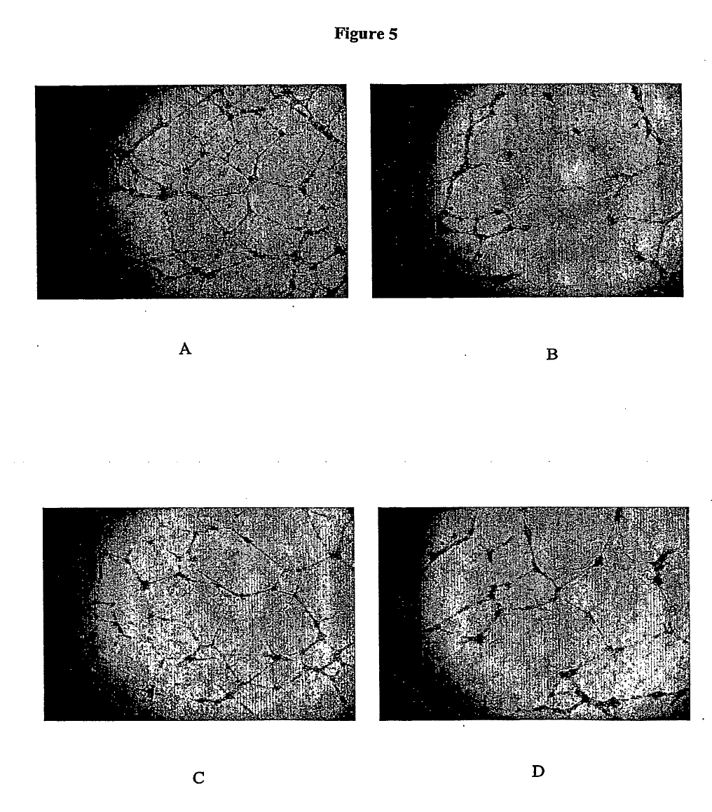

Matrigel (60 μl of 10 mg/ml) was added to a 96-well plate flat bottom plate (Costar 3096) and incubated for 30 minutes at 37° C. in a 5% CO₂ atmosphere. A mixture of HUVECs and plant extract, or positive controls (Fumagillin and GM6001) were added to each well. HUVECs were prepared as suspensions of 2.5×10⁵ cells per ml in EGM-2, then 500 μl of HUVECs preparation was mixed with 500 μl of 2× of the desired dilution of plant extract or control drug and 200 μl were added to each well. Four dilutions of each extract were tested in duplicate. After 18-24 hours at 37° C. in 5% CO₂, the cells had migrated and organized into cords (see FIG. 5, which shows the results using an extract from Rheum rhabarbaram). The number of cell junctions were counted in 3 randomly selected fields and the inhibition of cord formation is calculated as follows:

[(A−B)/A]×100,

where A is the average number of cell junctions per field in the control well and B is the average number of cell junctions per field in the treated wells.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

TABLE I
MMP-1 Inhibition

| Nom latin | Stress | Extrait | Inhibition (%) |
|---|---|---|---|
| Achillea millefolium | A | O | 22.2 |
| Acorus calamus | A | O | 100.0 |
| Actinidia arguta | A | O | 56.4 |
| Agastache foeniculum | A | S | 30.4 |
| Alchemilla mollis | A | 4 | 36.4 |
| Allium cepa | A | O | 61.4 |
| Allium grande | A | R | 46.5 |
| Allium porrum | A | R | 25.0 |
| Allium porrum | A | O | 98.9 |
| Allium sativum | A | O | 42.5 |
| Allium sativum | A | R | 98.7 |
| Allium schoenoprasum | A | R | 22.3 |
| Allium Tuberosum | A | R | 29.9 |
| Allium Tuberosum | A | O | 100.0 |
| Althaea officinalis | A | S | 21.6 |
| Angelica archangelica | A | S | 45.9 |
| Anthemis nobilis | A | R | 34.5 |
| Aralia nudicaulis | A | O | 100.0 |
| Armoracia rusticana | A | O | 31.2 |
| Armoracia rusticana | A | S | 39.7 |
| Aronia melanocarpa | A | R | 39.8 |
| Aster sp | A | O | 67.6 |
| Beckmannia eruciformis | A | O | 24.1 |
| Beta vulgaris | A | R | 41.2 |
| Beta vulgaris spp. Maritima | A | O | 44.1 |
| Brassica napus | A | O | 26.3 |
| Brassica oleracea | A | S | 28.6 |
| Brassica oleracea | A | R | 33.8 |
| Brassica Oleracea | A | O | 100.0 |
| Brassica rapa | A | R | 61.4 |
| Calamintha nepeta | A | R | 40.2 |
| Camellia sinensis | A | O | 39.3 |
| Capsicum annuum | A | R | 34.3 |
| Capsicum annuum | A | O | 88.3 |
| Capsicum frutescens | A | R | 39.4 |
| Chenopodium bonus-henricus | A | O | 100.0 |
| Chenopodium bonus-henricus | A | R | 37.3 |
| Chenopodium quinoa | A | O | 66.3 |
| Chrysanthenum coronarium | A | R | 37.4 |
| Cichorium intybus | A | R | 22.0 |
| Cichorium intybus | A | S | 66.9 |
| Citrullus lanatus | A | O | 41.9 |
| Cornus canadensis | A | S | 73.0 |
| Crataegus sp | A | O | 100.0 |
| Cucumis Anguria | A | S | 34.2 |
| Cucurbita moschata | A | O | 27.3 |
| Cucurbita pepo | A | O | 84.9 |
| Cymbopogn citratus | A | O | 100.0 |
| Cymbopogon citratus | A | R | 22.1 |
| Cyperus esculentus | A | R | 25.8 |
| Cyperus esculentus | A | O | 28.1 |
| Dactylis glomerata | A | O | 25.5 |
| Daucus carota | A | O | 43.4 |
| Daucus carota | A | R | 100.0 |
| Dipsacus sativus | A | O | 35.3 |
| Dirca palustris | A | S | 47.9 |
| Eruca vesicaria | A | R | 33.7 |
| Eschscholzia californica | A | O | 61.1 |
| Eschscholzia californica | A | R | 74.1 |
| Filipendula rubra | A | O | 51.7 |
| Foeniculum vulgare | A | O | 86.2 |
| Fragaria x ananassa | A | O | 23.7 |
| Fragaria X ananassa | A | S | 40.6 |
| Fragaria x ananassa | A | R | 28.3 |
| Galinsoga ciliata | A | R | 29.7 |
| Gallium odoratum | A | 6 | 48.8 |
| Gaultheria hispidula | A | R | 23.9 |
| Glycine max | A | R | 24.7 |
| Glycine max | A | S | 29.6 |
| Glycine max | A | O | 100.0 |
| Guizotia abyssinica | A | S | 39.4 |
| Hamamelis virginiana | A | R | 49.1 |
| Helianthus Tuberosus | A | O | 95.9 |
| Heliotropium arborescens | A | R | 25.0 |
| Hordeum hexastichon | A | O | 100.0 |
| Hordeum vulgare | A | O | 46.2 |
| Hordeum vulgare subsp. Vulgare | A | O | 43.8 |
| Inula helenium | A | O | 25.8 |
| Lathyrus sativus | A | 0 | 27.1 |
| Leonurus cardiaca | A | O | 34.4 |
| Levisticum officinale | A | R | 31.7 |
| Lolium multiflorum | A | O | 39.0 |
| Lotus corniculatus | A | O | 100.0 |
| Malva sylvestris | A | R | 22.8 |
| Matricaria recutita | A | O | 25.1 |
| Matteucia pensylvanica | A | R | 48.1 |
| Medicago sativa | A | R | 25.1 |
| Melissa officinalis | A | O | 100.0 |
| Mentha piperita | A | O | 60.1 |
| Mentha suaveolens | A | O | 35.1 |
| Nepeta cataria | A | O | 100.0 |
| Nicotiana rustica | A | R | 20.7 |
| Origanum vulgare | A | R | 60.5 |
| Origanum vulgare | A | O | 73.2 |
| Perilla frutescens | A | R | 74.4 |
| Perilla frutescens | A | O | 92.4 |
| Petroselinum crispum | A | R | 77.4 |
| Phacelia tanacetifolia | A | R | 52.8 |
| Phaseolus coccineus | A | R | 20.9 |
| Phaseolus coccineus | A | S | 34.2 |
| Phaseolus Vulgaris | A | S | 29.2 |
| Phaseolus vulgaris | A | R | 56.1 |
| Phaseolus Vulgaris | A | R | 60.0 |
| Phaseolus Vulgaris | A | O | 100.0 |
| Phlo x paniculata | A | O | 100.0 |
| Pimpinella anisum | A | S | 100.0 |
| Pimpinella anisum | A | R | 72.2 |
| Plantago coronopus | A | R | 23.7 |
| Plectranthus sp. | A | O | 25.0 |
| Poa compressa | A | O | 31.5 |
| Potentilla anserina | A | R | 71.2 |
| Pysalis ixocarpa | A | R | 32.1 |
| Raphanus raphanistrum | A | O | 31.5 |
| Raphanus sativus | A | O | 100.0 |
| Raphanus sativus | A | O | 30.2 |
| Rheum officinale | A | O | 79.1 |
| Rheum rhabarbarum | A | R | 22.9 |
| Rheum rhabarbarum | A | R | 32.8 |
| Ribes nigrum | A | O | 100.0 |
| Ribes nigrum | A | R | 100.0 |
| Ribes salivum | A | R | 48.6 |
| Ribes sylvestre | A | S | 26.5 |
| Ribes uva-crispa | A | R | 100.0 |
| Rubus canadensis | A | R | 46.1 |
| Rubus canadensis | A | R | 53.1 |
| Rubus idaeus | A | R | 100.0 |
| Salvia officianalis | A | O | 100.0 |
| Salvia sclarea | A | S | 43.8 |
| Satureja montana | A | R | 100.0 |
| Solanum dulcamara | A | S | 43.8 |
| Solanum melanocerasum | A | R | 37.2 |
| Solanum tuberosum | A | R | 100.0 |
| Sorghum dochna | A | O | 100.0 |
| Stachys byzantina | A | S | 28.9 |
| Stellaria media | A | S | 33.1 |
| Tanacetum parthenium | A | O | 28.9 |
| Tanacetum vulgare | A | R | 76.0 |
| Taraxacum officinale | A | O | 65.7 |
| Thymus praeco x subsp arcticus | A | O | 64.2 |
| Thymus praeco x subsp arcticus | A | R | 88.2 |
| Thymus vulgaris | A | R | 42.7 |
| Thymus x citriodorus | A | O | 34.7 |
| Trichosanthes kirilowii | A | R | 31.8 |
| Trifolium hybridum | A | R | 96.0 |
| Trifolium incarnatum | A | R | 100.0 |
| Trifolium pannonicum | A | R | 27.7 |
| Trifolium repens | A | R | 79.5 |
| Vaccinum augustifolium | A | R | 52.5 |
| Vaccinum macrocarpon | A | O | 64.5 |
| Vicia sativa | A | O | 60.8 |
| Vicia sativa | A | R | 28.6 |
| Vicia villosa | A | R | 64.7 |
| Vicia villosa | A | O | 57.3 |
| Vigna sesquipedalis | A | O | 33.0 |
| Vigna sesquipedalis | A | R | 24.4 |
| Vigna unguiculata | A | R | 20.6 |
| Vitia spp | A | R | 72.6 |
| Vitia spp | A | O | 100.0 |
| Zea Mays | A | R | 99.2 |
| Zea Mays | A | O | 100.0 |
| Abelmochus esculentus | G | R | 37.6 |
| Aconitum napellus | G | O | 100.0 |
| Allium ampeloprasum | G | R | 33.4 |
| Allium ascalonicum | G | R | 31.5 |
| Allium cepa | G | O | 34.4 |
| Allium cepa | G | R | 36.4 |
| Allium sativum | G | R | 53.2 |
| Allium tuberosum | G | R | 68.3 |
| Althaea officianalis | G | O | 47.7 |
| Althaea officinalis | G | S | 30.7 |
| Althaea officinalis | G | S | 44.3 |
| Althea officinalis | G | R | 83.6 |
| Anethum graveolens | G | S | 44.3 |
| Apium graveolens | G | R | 27.7 |
| Armoracia rusticana | G | O | 51.8 |
| Armoracia rusticana | G | S | 47.1 |
| Aronia melanocarpa | G | S | 66.5 |
| Artemisia dracunculus | G | S | 79.0 |
| Artemisia dracunculus | G | R | 50.3 |
| Asparagus officinalis | G | O | 96.4 |
| Bellis perennis | G | R | 44.1 |
| Beta vulgaris spp. Maritima | G | R | 43.7 |
| Beta vulgaris spp. Maritima | G | O | 34.9 |
| Betula glandulosa | G | S | 40.8 |
| Borago officinalis | G | O | 30.3 |
| Borago officinalis | G | R | 29.7 |
| Brassica cepticepa | G | R | 21.9 |
| Brassica oleracea | G | O | 33.6 |
| Brassica oleracea | G | O | 100.0 |
| Brassica rapa | G | O | 42.5 |
| Brassica rapa | G | R | 40.2 |
| Calamintha nepeta | G | O | 28.7 |
| Calendula officinalis L. | G | O | 100.0 |
| Camellia sinensis | G | O | 46.4 |
| Campanula rapunculus | G | R | 27.2 |
| Capsella bursa-pastoris | G | R | 24.1 |
| Capsicum annum | G | O | 36.0 |
| Chaerophyllum bulbosum | G | R | 38.9 |
| Chenopodium quinoa | G | O | 100.0 |
| Cichorium intybus | G | S | 44.6 |
| Circium arvense | G | R | 30.3 |
| Citrullus lanatus | G | R | 21.2 |
| Cucurbita pepo | G | O | 59.5 |
| Cucurbita Pepo | G | O | 40.2 |
| Cuminum cyminum | G | R | 25.5 |
| Cymbopogon citratus | G | R | 33.7 |
| Datura stramonium | G | O | 73.5 |
| Daucus carota | G | O | 86.0 |
| Daucus carota | G | O | 27.9 |
| Dryopteris filix-mas | G | O | 21.9 |
| Erysimum perofskianum | G | O | 24.4 |
| Fagopyrum esculentum | G | O | 100.0 |
| Foeniculum vulgare | G | O | 28.0 |
| Foeniculum vulgare | G | R | 57.3 |
| Gaultheria hispidula | G | O | 44.2 |
| Gaultheria procumbens | G | R | 94.8 |
| Glechoma hederacea | G | O | 25.5 |
| Glycine max | G | S | 100.0 |
| Glycyrrhiza glabra | G | O | 24.9 |
| Guizotia abyssinica | G | R | 30.3 |
| Helenium hoopesii | G | 0 | 28.6 |
| Helianthus annuus | G | O | 33.6 |
| Helianthus tuberosus | G | O | 54.4 |
| Hordeum vulgare | G | O | 28.8 |
| Hordeum vulgare subsp. Vulgare | G | R | 28.1 |
| Hypericum henryl | G | R | 80.0 |
| Iberis amara | G | O | 44.6 |
| Lactuca sativa | G | R | 25.3 |
| Lathyrus sylvestris | G | O | 90.2 |
| Lavandula angustifolia | G | R | 22.5 |
| Lepidium Sativum | G | S | 29.5 |
| Levisticum officinale | G | O | 100.0 |
| Lolium multiflorum | G | O | 24.9 |
| Lolium multiflorum | G | R | 27.1 |
| Lotus corniculatus | G | O | 52.2 |
| Lycopersicon esculentum | G | R | 24.4 |
| Lycopersicon pimpinellifolium | G | R | 30.3 |
| Malus hupehensis | G | R | 65.8 |
| Malva verticillata | G | R | 43.1 |
| Matricaria recutita | G | S | 100.0 |
| Matteucia pensylvanica | G | R | 57.5 |
| Melissa officinalis | G | O | 28.5 |
| Mentha piperita | G | O | 36.0 |
| Mentha spicata | G | S | 20.3 |
| Mentha spicata | G | S | 26.0 |
| Mentha suaveolens | G | O | 60.5 |
| Nepeta cataria | G | O | 24.1 |
| Nicotiana rustica | G | R | 28.1 |
| Nicotiana tabacum | G | R | 40.6 |
| Oenothera biennis | G | R | 28.4 |
| Oenothera biennis | G | O | 100.0 |
| Origanum vulgare | G | S | 100.0 |
| Origanum vulgare | G | O | 20.1 |
| Origanum vulgare | G | O | 85.4 |
| Oryza Sativa | G | R | 53.3 |
| Pana x quinquefolius | G | S | 100.0 |
| Panicum miliaceum | G | S | 100.0 |
| Passiflora caerula | G | O | 20.9 |
| Pastinaca sativa | G | R | 68.4 |
| Pastinaca sativa | G | O | 100.0 |
| Pennisetum alopecuroides | G | R | 100.0 |
| Petroselinum crispum | G | R | 73.0 |
| Phalaris canariensis | G | O | 100.0 |
| Phaseolus coccineus | G | R | 29.9 |
| Phaseolus coccineus | G | R | 67.6 |
| Phaseolus coccineus | G | O | 32.4 |
| Phaseolus vulgaris | G | R | 33.4 |
| Phaseolus vulgaris | G | R | 60.2 |
| Phaseolus vulgaris | G | R | 22.3 |
| Phaseolus vulgaris | G | O | 87.7 |
| Phlo x paniculata | G | O | 89.3 |
| Physalis pruinosa | G | O | 37.0 |
| Plantago coronopus | G | R | 48.1 |
| Plantago major | G | O | 47.0 |
| Plectranthus sp. | G | O | 97.2 |
| Potentilla anserina | G | R | 22.0 |
| Prunella vulgaris | G | O | 21.2 |
| Raphanus Raphanistrum | G | O | 95.9 |
| Raphanus sativus | G | O | 67.7 |
| Reseda odorata | G | O | 40.6 |
| Rheum officinale | G | O | 82.1 |
| Rheum rhabarbarum | G | R | 48.1 |
| Ribes Nigrum | G | R | 100.0 |
| Ribes Sylvestre | G | O | 42.9 |
| Ricinus communis | G | O | 73.5 |
| Rubus Phoenicalasius | G | R | 31.4 |
| Ruta graveolens | G | R | 100.0 |
| Salvia officinalis | G | R | 100.0 |
| Santolina | G | R | 28.1 |
| Satureja hortensis | G | R | 100.0 |
| Satureja repandra | G | O | 57.1 |
| Scrophularia nodosa | G | R | 41.6 |
| Scutelaria lateriflora | G | S | 72.1 |
| Sium sisarum | G | O | 99.7 |
| Solanum dulcamara | G | R | 65.4 |
| Solanum melanocerasum | G | R | 32.4 |
| Solanum melorgena | G | O | 100.0 |
| Solanum tuberosum | G | S | 46.4 |
| Sorghum caffrorum | G | R | 100.0 |
| Sorghum dochna | G | R | 51.4 |
| Sorghum dochna | G | R | 39.6 |
| Sorghum sudanense | G | O | 97.4 |
| Stachys byzantina | G | O | 41.4 |
| Stellaria media | G | O | 33.8 |
| Symphytum officinale | G | O | 52.0 |
| Tanacetum parthenium | G | O | 79.1 |
| Tanacetum vulgare | G | O | 100.0 |
| Taraxacum officinale | G | S | 25.9 |
| Teucrium chamaedrys | G | O | 100.0 |
| Teucrium chamaedrys | G | R | 48.0 |
| Thymus praeco x subsp arcticus | G | R | 73.1 |
| Thymus x citriodorus | G | O | 52.2 |
| Trichosanthes kirilowii | G | O | 35.9 |
| Trifolium hybridum | G | R | 76.0 |
| Trifolium incarnatum | G | R | 73.4 |
| Trifolium pannonicum | G | R | 24.8 |
| Trifolium repens | G | R | 48.5 |
| Triticosecale spp. | G | R | 48.5 |
| Triticum spelta | G | R | 22.9 |
| Tropaeolum majus | G | S | 23.4 |
| Urtica dioica | G | O | 96.4 |
| Vaccinium corymbosum | G | S | 60.7 |
| Vaccinium corymbosum | G | R | 61.4 |
| Vaccinum angustifolium | G | R | 54.7 |
| Vicia sativa | G | R | 68.8 |
| Vicia sativa | G | O | 31.5 |
| Vicia villosa | G | O | 100.0 |
| Vicia villosa | G | R | 35.5 |
| Vigna sesquipedalis | G | R | 23.0 |
| Vitia spp | G | R | 36.9 |
| Withania somnifera | G | O | 44.0 |
| Xanthium strumarium | G | R | 37.6 |
| Zea mays | G | O | 100.0 |
| Aconitum napellus | T | R | 100.0 |
| Agaricus bisporus | T | R | 58.9 |
| Agaricus bisporus | T | O | 100.0 |
| Allium ampeloprasum | T | R | 43.3 |
| Allium ascalonicum | T | R | 34.5 |
| Allium cepa | T | R | 53.5 |
| Allium cepa | T | O | 45.8 |
| Allium grande | T | R | 43.2 |
| Allium schoenoprasum | T | R | 47.1 |
| Allium tuberosum | T | R | 74.6 |
| Allium tuberosum | T | O | 33.6 |
| Aloe vera | T | R | 34.1 |
| Althaea officinalis | T | S | 47.8 |
| Amelanchier alnitolia | T | R | 59.1 |
| Ananas comosus | T | O | 100.0 |
| Anthemis nobilis | T | O | 22.7 |
| Anthriscus cerefolium | T | O | 56.8 |
| Apium graveolens | T | R | 29.8 |
| Aralia nudicaulis | T | O | 100.0 |
| Armoracia rusticana | T | O | 58.9 |
| Artemisia dracunculus | T | O | 100.0 |
| Asparagus officinalis | T | R | 25.2 |
| Atriple x hortensis | T | R | 44.7 |
| Bellis perennis | T | R | 58.1 |
| Beta vulgaris | T | R | 37.3 |
| Betula glandulosa | T | O | 23.5 |
| Boletus edulis | T | S | 64.2 |
| Brassica juncea | T | R | 35.6 |
| Brassica napus | T | O | 100.0 |
| Brassica oleracea | T | R | 33.2 |
| Brassica oleracea | T | O | 49.7 |
| Camellia sinensis | T | O | 24.7 |
| Camellia sinensis | T | R | 45.7 |
| Canna edulis | T | R | 26.2 |
| Carum carvi | T | O | 100.0 |
| Chaerophyllum bulbosum | T | R | 40.9 |
| Chrysanthemun coronarium (Chp suey) | T | R | 48.1 |
| Chrysanthenum coronarium | T | R | 29.9 |
| Chrysanthenum coronarium | T | R | 100.0 |
| Cichorium endivia | T | R | 20.5 |
| Cichorium endivia | T | R | 21.9 |
| Cichorium intybus | T | S | 50.6 |
| Cichorium intybus | T | R | 31.7 |
| Cichorium intybus | T | R | 52.9 |
| Citrullus lanatus | T | O | 100.0 |
| Citrus paradisi | T | O | 40.6 |
| Cocos nucifera | T | O | 27.2 |
| Cornus canadensis | T | S | 44.9 |
| Crithmum maritimum | T | R | 32.3 |
| Cucumis anguria | T | O | 22.6 |
| Cucurbita moschata | T | O | 33.5 |
| Cucurbita moschata (Early Butternut) | T | R | 32.3 |
| Cucurbita pepo | T | O | 89.0 |
| Cuminum cyminum | T | R | 54.3 |
| Curcuma zedoaria | T | S | 100.0 |
| Cymbopogon citratus | T | O | 42.6 |
| Datura metel | T | O | 24.8 |
| Datura metel | T | R | 25.5 |
| Dioscorea batatas | T | R | 100.0 |
| Dipsacus sativus | T | O | 85.0 |
| Dryopteris filix-mas | T | O | 46.4 |
| Erigeron canadensis | T | O | 100.0 |
| Eruca vesicaria | T | R | 30.9 |
| Erysimum perofskianum | T | O | 23.0 |
| Eschscholzia californica | T | O | 37.8 |
| Eschscholzia californica | T | R | 20.8 |
| Fagopyrum esculentum | T | O | 100.0 |
| Fagopyrum tartaricum | T | R | 78.5 |
| Foeniculum vulgare | T | O | 63.4 |
| Foeniculum vulgare | T | O | 27.2 |
| Forsythia x intermedia | T | S | 32.0 |
| Fragaria x ananassa | T | S | 33.0 |
| Galinsoga ciliata | T | R | 25.8 |
| Gaultheria procumbens | T | O | 46.8 |
| Hedeoma pulegioides | T | O | 73.6 |
| Helianthus tuberosus | T | O | 39.3 |
| Hordeum vulgare | T | O | 32.4 |
| Humulus lupulus | T | O | 21.1 |
| Hypericum henryi | T | R | 29.3 |
| Hypericum perforatum | T | R | 42.7 |
| Iberis amara | T | O | 29.5 |
| Ipomea aquatica | T | R | 22.9 |
| Lathyrus Sativus | T | R | 69.4 |
| Laurus nobilis | T | O | 70.2 |
| Lavandula latifolia | T | O | 100.0 |
| Lens culinaris subsp. Culinaris | T | O | 70.2 |
| Lepidium sativum | T | O | 100.0 |
| Levisticum officinale | T | O | 100.0 |
| Lolium multiflorum | T | O | 35.1 |
| Lunaria annua | T | O | 100.0 |
| Lycopersicon pimpinellifolium | T | R | 24.4 |
| Malus hupehensis | T | R | 73.1 |
| Malus sp. | T | R | 80.9 |
| Malva sylvestris | T | R | 34.7 |
| Malva sylvestris | T | O | 100.0 |
| Manihot esculenta | T | R | 33.0 |
| Melissa officinalis | T | O | 100.0 |
| Melissa officinalis | T | O | 100.0 |
| Mentha suaveolens | T | S | 39.7 |
| Nigella sativa | T | R | 58.9 |
| Nigella sativa | T | R | 100.0 |
| Ocimum Basilicum | T | R | 100.0 |
| Origanum majorana | T | O | 41.5 |
| Origanum vulgare | T | O | 29.8 |
| Origanum vulgare | T | R | 33.1 |
| Pana x quinquefolius | T | R | 75.2 |
| Passiflora spp. | T | S | 32.0 |
| Pastinaca sativa | T | R | 20.8 |
| Perroselinum crispum | T | R | 55.4 |
| Petroselinum crispum | T | R | 76.1 |
| Petroselinum crispum | T | O | 24.1 |
| Peucedanum oreaselinum | T | O | 21.0 |
| Phacelia tanacetifolia | T | R | 48.6 |
| Phalaris canariensis | T | O | 56.4 |
| Phaseolus coccineus | T | R | 22.7 |
| Phaseolus mungo | T | R | 47.4 |
| Phaseolus vulgaris | T | R | 40.0 |
| Phaseolus vulgaris | T | O | 29.4 |
| Phoeni x dactylifera | T | R | 46.3 |
| Physalis ixocarpa goldie ou pourpre | T | R | 28.9 |
| Phytolacca americana | T | O | 100.0 |
| Plectranthus sp. | T | O | 73.8 |
| Pleurotus spp. | T | O | 100.0 |
| Poa compressa | T | O | 22.3 |
| Poa pratensis | T | O | 73.1 |
| Populus Tremula | T | O | 100.0 |
| Prunella vulgaris | T | O | 38.0 |
| Psoralea corylifolia | T | S | 96.4 |
| Pteridium aquilinum | T | R | 100.0 |
| Raphanus raphanistrum | T | O | 100.0 |
| Raphanus sativus | T | R | 33.7 |
| Raphanus sativus | T | R | 28.0 |
| Raphanus sativus | T | O | 100.0 |
| Reseda luteola | T | S | 69.6 |
| Reseda odorata | T | O | 51.8 |
| Rheum officinale | T | O | 46.7 |
| Rheum officinale | T | S | 100.0 |
| Ribes nigrum | T | R | 30.0 |
| Ribes Sativum | T | R | 61.7 |
| Ribes Sylvestre | T | R | 75.4 |
| Ricinus communis | T | S | 100.0 |
| Rosmarinus officinalis | T | R | 29.0 |
| Rubus canadensis | T | R | 86.1 |
| Sabal serrulata | T | R | 100.0 |
| Salvia officinalis | T | O | 100.0 |
| Sambucus canadensis | T | O | 24.8 |
| Satureja montana | T | R | 100.0 |
| Satureja repandra | T | S | 27.2 |
| Satureja repandra | T | O | 36.4 |
| Satureja repandra | T | R | 42.0 |
| Scrophularia nodosa | T | R | 68.8 |
| Secale cereale | T | O | 100.0 |
| Setaria italica | T | R | 23.2 |
| Silybum marianum | T | O | 73.5 |
| Solanum melongena | T | R | 20.1 |
| Solanum tuberosum | T | S | 24.4 |
| Solidago virgaurea | T | R | 71.4 |
| Sorghum dochna | T | O | 22.5 |
| Stachys byzantina | T | O | 39.2 |
| Stellaria media | T | O | 43.3 |
| Symphytum officinale | T | O | 58.7 |
| Tanacetum parthenium | T | O | 100.0 |
| Tanacetum vulgare | T | O | 32.5 |
| Taraxacum officinale | T | S | 27.8 |
| Teucrium chamaedrys | T | R | 62.9 |
| Teucrium chamaedrys | T | O | 100.0 |
| Thalpsi arvense | T | O | 21.2 |
| Thymus praeco x subsp arcticus | T | R | 60.9 |
| Tragopogon porrifolium | T | R | 24.6 |
| Trifolium incarnatum | T | R | 33.7 |
| Trifolium pannonicum | T | R | 72.4 |
| Trifolium repens | T | R | 72.4 |
| Triticosecale spp. | T | R | 33.7 |
| Tropaeolum majus | T | R | 100.0 |
| Tropaeolum majus | T | O | 31.5 |
| Vaccinium angustifolium | T | O | 100.0 |
| Vaccinium angustifolium | T | S | 42.1 |
| Vaccinium macrocarpon | T | S | 30.9 |
| Vicia villosa | T | R | 35.5 |
| Vigna sesquipedalis | T | R | 24.0 |
| Vigna unguiculata | T | R | 31.6 |
| Vinca minor | T | O | 28.7 |
| Withania somnifera | T | O | 26.9 |
| Xanthium strumarium | T | O | 30.9 |
| Zea mays | T | R | 20.1 |
| Zea mays | T | O | 32.2 |

TABLE 2
MMP-2

| Nom latin | Stress | Extrait | Inhibition (%) |
|---|---|---|---|
| Achillea millefolium | A | S | 21.9 |
| Achillea millefolium | A | O | 63.0 |
| Achillea millefolium | A | R | 100.0 |
| Aconitum napellus | A | R | 71.0 |
| Alcea rosea | A | R | 67.9 |
| Alchemilla mollis | A | O | 64.4 |
| Allium ascalonicum | A | R | 20.9 |
| Allium cepa | A | R | 84.3 |
| Allium grande | A | R | 36.7 |
| Allium porrum | A | O | 100.0 |
| Allium porum | A | S | 51.9 |
| Allium porum | A | R | 66.7 |
| Allium sativum | A | R | 100.0 |
| Allium schoenoprasum | A | R | 73.5 |
| Allium Tuberosum | A | S | 24.3 |
| Allium Tuberosum | A | O | 83.6 |
| Allium Tuberosum | A | R | 89.3 |
| Aloe vera | A | R | 69.7 |
| Althaea officinalis | A | S | 27.6 |
| Althaea officinalis | A | R | 64.7 |
| Amaranthus gangeticus | A | S | 29.4 |
| Anethum graveolens | A | O | 100.0 |
| Apium graveolens | A | S | 25.1 |
| Apium graveolens | A | R | 52.1 |
| Aralia cordata | A | S | 66.4 |
| Aralia cordata | A | R | 92.2 |
| Aralia nudicaulis | A | O | 29.4 |
| Arctium minus | A | S | 28.4 |
| Armoracia rusticana | A | S | 20.2 |
| Armoracia rusticana | A | O | 55.0 |
| Arrhenatherum elatius | A | S | 40.2 |
| Artemisia dracunculus | A | S | 39.7 |
| Asparagus officinalis | A | S | 29.3 |
| Atriple x hortensis | A | R | 33.6 |
| Avena sativa | A | R | 37.2 |
| Beta vulgaris | A | S | 45.4 |
| Beta vulgaris | A | R | 95.9 |
| Beta vulgaris spp. Maritima | A | R | 100.0 |
| Brassica chinensis | A | R | 49.6 |
| Brassica napus | A | O | 28.5 |
| Brassica Napus | A | S | 52.4 |
| Brassica Napus | A | R | 82.4 |
| Brassica nigra | A | O | 29.2 |
| Brassica oleracea | A | R | 31.2 |
| Brassica Oleracea | A | R | 31.4 |
| Brassica oleracea | A | R | 64.0 |
| Brassica oleracea | A | S | 68.7 |
| Brassica oleracea | A | R | 75.3 |
| Brassica oleracea | A | O | 100.0 |
| Brassica rapa | A | S | 27.6 |
| Brassica rapa | A | R | 33.4 |
| Brassica rapa | A | O | 57.6 |
| Brassica rapa | A | R | 58.1 |
| Brassica rapa | A | R | 84.5 |
| Calamintha nepeta | A | O | 65.0 |
| Camellia sinensis | A | S | 21.9 |
| Camellia sinensis | A | R | 26.5 |
| Camellia sinensis | A | O | 79.0 |
| Cana edulis | A | R | 45.5 |
| Canna edulis | A | S | 20.2 |
| Capsella bursa-pastoris | A | S | 35.5 |
| capsicum annuum | A | S | 61.5 |
| Capsicum annuum | A | O | 89.8 |
| Capsicum annuum | A | R | 100.0 |
| Capsicum frutescens | A | S | 66.6 |
| Capsicum frutescens | A | R | 100.0 |
| Carthamus tinctorius | A | R | 21.3 |
| Carthamus tinctorius | A | R | 21.5 |
| Chaerophyllum bulbosom | A | R | 57.2 |
| Chelidonium majus | A | S | 34.4 |
| Chenopodium bonus-henricus | A | R | 43.5 |
| Chenopodium bonus-henricus | A | O | 100.0 |
| Chenopodium bonus-henricus | A | R | 76.4 |
| Chenopodium quinoa | A | O | 92.0 |
| Chrysanthemum coronarium | A | R | 48.6 |
| Chrysanthemum coronarium | A | O | 49.7 |
| Chrysanthemum coronarium | A | R | 47.3 |
| Chrysanthemum coronarium | A | R | 26.7 |
| Cicer arietinum | A | S | 22.0 |
| Cicer arietinum | A | O | 23.6 |
| Cichorium intybus | A | S | 21.1 |
| Cichorium intybus | A | R | 100.0 |
| Citrullus lanatus | A | S | 65.5 |
| Citrullus lanatus | A | R | 96.3 |
| Citrullus lanatus | A | O | 100.0 |
| Coi x Lacryma-Jobi | A | O | 32.2 |
| Cornus canadensis | A | S | 52.8 |
| Cosmos sulphureus | A | R | 72.5 |
| Crataegus spp | A | O | 100.0 |
| Cryptotaenia canadensis | A | R | 50.6 |
| Cryptotaenia canadensis | A | O | 51.3 |
| Cucumis anguria | A | S | 53.4 |
| Cucumis Anguria | A | R | 84.9 |
| Cucumis melo | A | R | 91.7 |
| Cucurbita Maxima | A | S | 34.9 |
| Cucurbita Maxima | A | R | 41.7 |
| Cucurbita moschata | A | R | 36.8 |
| Cucurbita moschata | A | S | 37.4 |
| Cucurbita pepo | A | S | 48.1 |
| Cucurbita pepo | A | R | 85.7 |
| Curcuma zedoaria | A | S | 21.0 |
| Curcuma zedoaria | A | R | 32.1 |
| Curcurbita maxima | A | S | 27.0 |
| Cymbopogon citratus | A | R | 34.5 |
| Cymbopogon citratus | A | O | 100.0 |
| Cymbopogon martinii | A | S | 47.4 |
| Dactylis glomerata | A | S | 20.6 |
| Dactylis glomerata | A | O | 75.0 |
| Daucus carota | A | S | 44.5 |
| Daucus carota | A | R | 70.5 |
| Dipsacus sativus | A | O | 40.4 |
| Dirca palustris | A | S | 27.2 |
| Dolichos Lablab | A | S | 54.2 |
| Dryopteris filix-mas | A | R | 76.3 |
| Echinacea purpurea | A | R | 42.9 |
| Eleusine coracana | A | S | 37.5 |
| Eleusine coracana | A | O | 100.0 |
| Erigeron canadensis | A | O | 45.7 |
| Eruca vesicaria | A | R | 80.2 |
| Eschscholzia californica | A | S | 42.4 |
| Eschscholzia californica | A | O | 75.0 |
| Eschscholzia californica | A | R | 88.8 |
| Fagopyrum esculentum | A | O | 100.0 |
| Fagopyrum tartaricum | A | R | 38.6 |
| Fagopyrum tartaricum | A | S | 40.3 |
| Galinsoga ciliata | A | R | 54.0 |
| Galium odoratum | A | O | 34.3 |
| Galium odoratum | A | O | 100.0 |
| Gaultheria hispidula | A | S | 35.8 |
| Gaultheria hispidula | A | R | 100.0 |
| Glau x maritima | A | R | 46.5 |
| Glycine max | A | S | 27.0 |
| Glycine Max | A | R | 43.1 |
| Glycine max | A | O | 100.0 |
| Guizotia abyssinica | A | S | 29.8 |
| Guizotia abyssinica | A | R | 32.5 |
| Hamamelis virginiana | A | R | 75.7 |
| Helianthus annuus | A | R | 69.0 |
| Helianthus Tuberosus | A | R | 22.2 |
| Helianthus tuberosus | A | R | 69.7 |
| Helianthus Tuberosus | A | O | 100.0 |
| Hordeum hexastichon | A | R | 22.3 |
| Hordeum hexastichon | A | R | 34.9 |
| Hordeum hexastichon | A | O | 86.9 |
| Hordeum vulgare | A | O | 74.8 |
| Hordeum vulgare subsp. Vulgare | A | S | 34.5 |
| Hordeum vulgare subsp. Vulgare | A | O | 74.2 |
| Hyssopus officinalis | A | O | 57.5 |
| Inula helenium | A | S | 26.8 |
| Ipomoea Batatas | A | S | 20.1 |
| Lathyrus sativus | A | S | 28.7 |
| Lathyrus sativus | A | 0 | 100.0 |
| Lathyrus sylvestris | A | R | 42.4 |
| Lavandula latifolia | A | O | 39.1 |
| Lepidium sativum | A | O | 20.1 |
| Lepidium sativum | A | S | 49.0 |
| Levisticum officinale | A | S | 23.0 |
| Levisticum officinale | A | O | 29.8 |
| Linum usitatissimum | A | R | 56.9 |
| Lolium multiflorum | A | S | 41.5 |
| Lolium multiflorum | A | O | 92.3 |
| Lotus corniculatus | A | O | 95.5 |
| Lotus tetragonolobus | A | R | 76.7 |
| Lycopersicon esculentum | A | S | 35.3 |
| Lycopersicon esculentum | A | R | 78.1 |
| Lycopersicon esculentum | A | R | 85.6 |
| Lycopersicon pimpinollifolium | A | R | 74.9 |
| Malva moschata | A | S | 21.5 |
| Malva moschata | A | O | 44.5 |
| Malva verticillata | A | R | 22.0 |
| Matricaria recutita | A | S | 40.9 |
| Matricaria recutita | A | O | 67.3 |
| Melaleuca alternifolia | A | O | 65.0 |
| Melilotus albus | A | S | 50.7 |
| Melilotus albus | A | O | 100.0 |
| Melissa officinalis | A | O | 42.4 |
| Mentha pulegium | A | O | 88.3 |
| Mentha spicata | A | O | 94.8 |
| Mentha suaveolens | A | O | 82.9 |
| Nepeta cataria | A | O | 100.0 |
| Nicotiana rustica | A | S | 24.0 |
| Nicotiana rustica | A | R | 100.0 |
| Nicotiana tabacum | A | S | 42.5 |
| Nicotiana tabacum | A | R | 61.1 |
| Nigella sativa | A | R | 81.7 |
| Ocimum tenuiflorum | A | R | 23.1 |
| Oenothera biennis | A | R | 28.6 |
| Origanum majorana | A | O | 52.9 |
| Origanum majorana | A | R | 100.0 |
| Origanum vulgare | A | O | 66.8 |
| Pana x quinquefolius | A | S | 31.8 |
| Pastinaca sativa | A | S | 27.7 |
| Pastinaca sativa | A | R | 33.8 |
| Petasites japonicus | A | S | 26.2 |
| Petroselinum crispum | A | R | 69.1 |
| Phalaris canariensis | A | S | 28.4 |
| Phalaris canariensis | A | R | 29.7 |
| Phalaris canariensis | A | O | 94.3 |
| Phaseolus coccineus | A | S | 30.8 |
| Phaseolus coccineus | A | R | 79.5 |
| Phaseolus coccineus | A | O | 80.9 |
| Phaseolus mungo | A | R | 59.8 |
| Phaseolus vulgaris | A | S | 47.3 |
| Phaseolus Vulgaris | A | R | 74.4 |
| Phaseolus vulgaris | A | R | 83.2 |
| Phaseolus Vulgaris | A | O | 100.0 |
| Phlo x paniculata | A | O | 23.7 |
| Phlo x paniculata | A | R | 81.7 |
| Physalis alkekengi | A | R | 23.5 |
| Physalis ixocarpa | A | O | 85.8 |
| Physalis ixocarpa | A | R | 91.5 |
| Physalis Pruinosa | A | R | 25.7 |
| Physalis Pruinosa | A | O | 83.5 |
| Phytolacca decandra | A | O | 31.5 |
| Phytolacca decandra | A | S | 38.5 |
| Pimpinella anisum | A | S | 100.0 |
| Pimpinella anisum | A | R | 100.0 |
| Plantago coronopus | A | R | 36.0 |
| Plantago coronopus | A | R | 38.4 |
| Plantago coronopus | A | O | 53.6 |
| Plantago major | A | R | 65.3 |
| Plectranthus sp. | A | O | 74.2 |
| Poa compressa | A | S | 37.3 |
| Poa compressa | A | R | 49.8 |
| Poa compressa | A | O | 100.0 |
| Polygonum pensylvanicum | A | R | 63.5 |
| Polygonum pensylvanicum | A | O | 72.9 |
| Polygonum persicaria | A | S | 27.5 |
| Polygonum persicaria | A | O | 43.0 |
| Poterium sanguisorba | A | R | 100.0 |
| Poterium Sanguisorba | A | O | 84.2 |
| Pteridium aquilinum | A | O | 45.1 |
| Pteridium aquilinum | A | R | 100.0 |
| Pysalis ixocarpa | A | R | 87.3 |
| Raphanus raphanistrum | A | S | 32.2 |
| Raphanus sativus | A | R | 25.3 |
| Raphanus sativus | A | S | 47.5 |
| Raphanus sativus | A | R | 83.5 |
| Raphanus sativus | A | R | 84.7 |
| Raphanus Sativus | A | O | 100.0 |
| Rheum officinale | A | O | 44.0 |
| Ribes nigrum | A | O | 100.0 |
| Ribes nigrum | A | R | 100.0 |
| Ricinus communis | A | O | 100.0 |
| Rosa rugosa | A | R | 25.2 |
| Rosa rugosa | A | S | 26.6 |
| Rosa rugosa | A | O | 83.2 |
| Rosmarinus officinalis | A | R | 68.2 |
| Rubus idaeus | A | O | 81.9 |
| Rubus ideaus | A | R | 73.4 |
| Rume x Acetosa | A | S | 24.2 |
| Rume x Acetosa | A | R | 85.5 |
| Rume x Acetosa | A | O | 100.0 |
| Rume x crispus | A | 0 | 46.7 |
| Rume x crispus | A | R | 100.0 |
| Ruta graveolens | A | O | 100.0 |
| Saccharum officinarum | A | R | 80.8 |
| Sali x purpurea | A | S | 56.7 |
| Salvia officinalis | A | S | 24.1 |
| Salvia officinalis | A | O | 91.8 |
| Salvia sclarea | A | O | 99.7 |
| Santolina chamaecyparissus | A | O | 83.8 |
| Satureja hortensis | A | O | 79.1 |
| Satureja hortensis | A | R | 100.0 |
| Satureja montana | A | R | 60.4 |
| Satureja montana | A | O | 76.1 |
| Scorzonera hispanica | A | S | 22.1 |
| Secale cereale | A | R | 47.2 |
| Secale cereale | A | O | 67.2 |
| Senecio vulgaris | A | S | 23.2 |
| Senecio vulgaris | A | R | 76.6 |
| Sesamum indicum | A | R | 100.0 |
| Sesamum indicum | A | S | 100.0 |
| Solanum dulcamara | A | R | 54.5 |
| Solanum melanocerasum | A | S | 45.4 |
| Solanum melanocerasum | A | R | 85.2 |
| Solanum melanocerasum | A | O | 88.7 |
| Solanum melongena | A | S | 42.5 |
| Solanum melongena | A | R | 85.9 |
| Sonchus oleraceus | A | R | 25.6 |
| Sorghum caffrorum | A | R | 39.6 |
| Sorghum dochna | A | S | 30.0 |
| Sorghum dochna | A | R | 48.0 |
| Sorghum dochna | A | O | 62.0 |
| Sorghum durra | A | R | 72.1 |
| Sorghum durra | A | O | 94.6 |
| Sorghum sudanense | A | O | 100.0 |
| Spinacia oleracea | A | S | 23.6 |
| Stachys affinis | A | R | 74.4 |
| Stachys byzantina | A | R | 48.4 |
| Stachys byzantina | A | O | 100.0 |
| Stellaria graminea | A | S | 20.8 |
| Stellaria graminea | A | R | 37.5 |
| Stellaria media | A | R | 49.0 |
| Stellaria media | A | S | 50.7 |
| Symphytum officinale | A | R | 44.2 |
| Tanacetum cinerariifolium | A | R | 100.0 |
| Tanacetum parthenium | A | S | 30.4 |
| Tanacetum vulgare | A | S | 28.6 |
| Tanacetum vulgare | A | R | 100.0 |
| Taraxacum officinale | A | R | 59.1 |
| Thymus praeco x subsp arcticus | A | R | 43.5 |
| Thymus vulgaris | A | S | 30.1 |
| Thymus x citriodorus | A | R | 100.0 |
| Trichosanthes kirilowii | A | S | 29.2 |
| Trichosanthes kirilowii | A | O | 42.1 |
| Trigonella foenumgraecum | A | O | 53.4 |
| Triticosecal spp. | A | R | 44.8 |
| Triticum aestivum | A | R | 65.5 |
| Triticum durum | A | O | 53.9 |
| Triticum spelta | A | R | 26.4 |
| Triticum spelta | A | S | 36.7 |
| Triticum spelta | A | O | 51.9 |
| Tropaeolum majus | A | R | 25.8 |
| Urtica dioica | A | O | 22.9 |
| Urtica dioica | A | S | 30.6 |
| Vaccinium Corymbosum | A | R | 100.0 |
| Veratrum viride | A | R | 33.2 |
| Verbascum thapsus | A | S | 22.9 |
| Veronica beccabunga | A | R | 52.8 |
| Veronica officinalis | A | R | 84.2 |
| Vicia sativa | A | R | 100.0 |
| Vicia villosa | A | S | 32.9 |
| Vicia villosa | A | R | 100.0 |
| Vigna angularis | A | R | 54.0 |
| Vigna sesquipedalis | A | S | 48.3 |
| Vigna sesquipedalis | A | R | 73.0 |
| Vigna sesquipedalis | A | O | 96.6 |
| Vigna unguiculata | A | R | 70.7 |
| Vinca minor | A | S | 22.1 |
| Vinca minor | A | R | 88.4 |
| Vitis sp. | A | S | 20.9 |
| Vitis sp. | A | R | 30.4 |
| Xanthium sibiricum | A | S | 39.2 |
| Xanthium sibiricum | A | R | 47.8 |
| Xanthium sibiricum | A | O | 70.1 |
| Zea mays | A | R | 100.0 |
| Zea Mays | A | O | 100.0 |
| Abelmochus esculentus | G | S | 21.6 |
| Abelmochus esculentus | G | R | 79.3 |
| Achillea millefolium | G | O | 62.7 |
| Aconitum napellus | G | O | 82.0 |
| Acorus calamus | G | S | 100.0 |
| Ageratum conyzoides | G | S | 49.3 |
| Alcea rosea | G | R | 64.4 |
| Alchemilla mollis | G | S | 21.5 |
| Alchemilla mollis | G | R | 30.2 |
| Alchemilla mollis | G | O | 55.7 |
| Allium ampeloprasum | G | O | 36.1 |
| Allium ampeloprasum | G | R | 52.8 |
| Allium ascalonicum | G | O | 68.9 |
| Allium cepa | G | S | 40.2 |
| Allium cepa | G | R | 66.4 |
| Allium cepa | G | O | 100.0 |
| Allium grande | G | R | 36.4 |
| Allium sativum | G | S | 29.5 |
| Allium sativum | G | R | 68.4 |
| Allium sativum | G | O | 100.0 |
| Allium schoenoprasum | G | S | 47.1 |
| Allium schoenoprasum | G | R | 61.7 |
| Allium tuberosum | G | S | 23.8 |
| Allium tuberosum | G | O | 54.5 |
| Allium tuberosum | G | R | 85.9 |
| Aloe vera | G | R | 53.6 |
| Althaea officinalis | G | S | 37.4 |
| Altheaa officinalis | G | S | 42.4 |
| Amaranthus caudathus | G | S | 30.9 |
| Amaranthus caudathus | G | O | 56.7 |
| Amaranthus gangeticus | G | S | 23.1 |
| Anethum graveolens | G | S | 23.9 |
| Angelica archangelica | G | S | 22.0 |
| Angelica archangelica | G | S | 24.9 |
| Apium graveolens | G | O | 33.0 |
| Apium graveolens | G | R | 44.8 |
| Apium graveolens | G | S | 54.1 |
| Apium graveolens | G | R | 84.1 |
| Aralia nudicaulis | G | R | 51.8 |
| Arctium minus | G | S | 25.4 |
| Armoracia rusticana | G | O | 52.1 |
| Aronia melanocarpa | G | S | 22.5 |
| Aronia melanocarpa | G | R | 82.3 |
| Artemisia dracunculus | G | R | 53.6 |
| Artemisia dracunculus | G | R | 58.8 |
| Artemisia dracunculus | G | S | 100.0 |
| Artemisia dracunculus | G | O | 100.0 |
| Asclepias incarnata | G | S | 26.9 |
| Asparagus officinalis | G | S | 24.0 |
| Asparagus officinalis | G | R | 65.9 |
| Asparagus officinalis | G | O | 95.0 |
| Aster spp | G | O | 48.4 |
| Beckmannia eruciformis | G | O | 24.8 |
| Bellis perennis | G | O | 52.6 |
| Beta vulgaris | G | S | 45.3 |
| Beta vulgaris | G | R | 100.0 |
| Beta vulgaris spp. Maritima | G | R | 100.0 |
| Brassica cepticepa | G | R | 52.9 |
| Brassica chinensis | G | R | 41.9 |
| Brassica juncea | G | R | 22.8 |
| Brassica Napus | G | S | 22.9 |
| Brassica oleracea | G | R | 45.5 |
| Brassica oleracea | G | R | 47.1 |
| Brassica oleracea | G | S | 62.9 |
| Brassica oleracea | G | R | 77.9 |
| Brassica oleracea | G | O | 100.0 |
| Brassica rapa | G | S | 26.5 |
| Brassica rapa | G | R | 38.9 |
| Brassica Rapa | G | R | 53.6 |
| Calamintha nepeta | G | S | 20.4 |
| Calamintha nepeta | G | O | 78.0 |
| Camellia sinensis | G | O | 100.0 |
| Campanula rapunculus | G | R | 60.6 |
| Canna edulis | G | O | 78.1 |
| Capsella bursa-pastoris | G | S | 30.7 |
| Capsella bursa-pastoris | G | R | 60.6 |
| Capsicum annuum | G | S | 70.8 |
| Capsicum annuum | G | O | 80.0 |
| Capsicum annuum | G | R | 100.0 |
| Capsicum frutescens | G | S | 63.2 |
| Capsicum frutescens | G | R | 100.0 |
| Carthamus tinctorius | G | R | 100.0 |
| Centaurea solstitialis | G | S | 46.4 |
| Cerastium tomentosum | G | R | 52.3 |
| Chenopodium bonus-henricus | G | S | 22.0 |
| Chenopodium quinoa | G | S | 31.0 |
| Chenopodium quinoa | G | O | 53.4 |
| Chrysanthemun coronarium | G | R | 76.2 |
| Chrysanthenum coronarium | G | R | 54.2 |
| Cicer arietinum | G | S | 23.1 |
| Cichorium endivia subsp endivia | G | S | 28.7 |
| Cichorium endivia subsp endivia | G | O | 68.7 |
| Cichorium intybus | G | S | 41.4 |
| Cichorium intybus | G | O | 62.1 |
| Circium arvense | G | S | 25.3 |
| Circium arvense | G | R | 59.3 |
| Citrullus lanatus | G | S | 24.8 |
| Citrullus lanatus | G | R | 41.1 |
| Citrullus lanatus | G | R | 100.0 |
| Cosmos sulphureus | G | R | 77.9 |
| Cosmos sulphureus | G | S | 79.4 |
| Cucumis sativus | G | S | 39.9 |
| Cucumis sativus | G | S | 39.9 |
| Cucurbita maxima | G | S | 33.9 |
| Cucurbita maxima | G | R | 43.4 |
| Cucurbita maxima | G | O | 100.0 |
| Cucurbita moschata | G | S | 41.3 |
| Cucurbita pepo | G | S | 42.8 |
| Cucurbita pepo | G | S | 45.4 |
| Cucurbita pepo | G | R | 83.0 |
| Cuminum cyminum | G | O | 66.2 |
| Curcuma zedoaria | G | R | 33.9 |
| Cymbopogon citratus | G | R | 65.8 |
| Cymbopogon martinii motia | G | S | 41.4 |
| Cymbopogon martinii motia | G | O | 60.5 |
| Dactylis glomerata | G | S | 21.9 |
| Dactylis glomerata | G | O | 61.2 |
| Datura stramonium | G | S | 27.0 |
| Daucus carota | G | O | 21.3 |
| Daucus carota | G | S | 31.0 |
| Daucus carota | G | R | 100.0 |
| Digitalis purpurea | G | S | 30.9 |
| Dipsacus sativus | G | O | 63.6 |
| Dirca palustris | G | O | 23.1 |
| Dolichos Lablab | G | S | 33.0 |
| Dryopteris filix-mas | G | R | 100.0 |
| Echinacea purpurea | G | R | 93.4 |
| Eleusine coracana | G | S | 30.0 |
| Erigeron speciosus | G | S | 28.9 |
| Errhenatherum elatius | G | S | 55.6 |
| Eruca vesicaria | G | R | 54.7 |
| Eschscholzia californica | G | S | 47.9 |
| Eschscholzia californica | G | O | 75.9 |
| Fagopyrum tartaricum | G | O | 41.1 |
| Filipendula rubra | G | R | 38.5 |
| Foeniculum vulgare | G | R | 70.0 |
| Foeniculum Vulgare | G | S | 100.0 |
| Galinsoga ciliata | G | S | 34.6 |
| Galinsoga ciliata | G | R | 48.2 |
| Gaultheria hispidula | G | R | 60.5 |
| Gaultheria hispidula | G | 0 | 100.0 |
| Gaultheria hispidula | G | S | 100.0 |
| Glau x maritima | G | R | 59.3 |
| Glycine max | G | R | 21.1 |
| Glycine max | G | S | 24.4 |
| Glycine max | G | O | 28.1 |
| Guizotia abyssinica | G | S | 26.0 |
| Guizotia abyssinica | G | R | 36.8 |
| Guizotia abyssinica | G | O | 100.0 |
| Hedeoma pulegioides | G | O | 94.6 |
| Helianthus annuus | G | S | 35.5 |
| Helianthus annuus | G | O | 75.0 |
| Helianthus annuus | G | R | 79.9 |
| Helianthus strumosus | G | O | 100.0 |
| Helianthus tuberosus | G | R | 64.2 |
| Helichrysum thianschanicum | G | O | 61.1 |
| Helleborus niger | G | R | 48.0 |
| Hordeum hexastichon | G | S | 26.8 |
| Hordeum vulgare | G | O | 65.4 |
| Hordeum vulgare subsp. Vulgare | G | O | 75.8 |
| Humulus lupulus | G | S | 26.0 |
| Hypericum henryi | G | R | 20.2 |
| Hypericum henryi | G | O | 71.1 |
| Hyssopus officinalis | G | O | 100.0 |
| Iberis amara | G | S | 21.2 |
| Inula helenium | G | S | 24.3 |
| Lactuca sativa | G | R | 100.0 |
| Lactuca serriola | G | R | 69.3 |
| Laportea canadensis | G | R | 100.0 |
| Lathyrus sylvestris | G | O | 39.6 |
| Lavandula angustifolia | G | O | 70.0 |
| Lavandula latifolia | G | S | 22.7 |
| Lepidium Sativum | G | R | 30.6 |
| Lepidium sativum | G | S | 53.3 |
| Levisticum officinale | G | O | 80.7 |
| Lolium multiflorum | G | O | 34.5 |
| Lotus corniculatus | G | S | 32.9 |
| Lotus corniculatus | G | O | 100.0 |
| Lotus tetragonolobus | G | R | 79.9 |
| Lycopersicon esculentum | G | S | 28.2 |
| Lycopersicon esculentum | G | R | 75.4 |
| Lycopersicon pimpinellifolium | G | R | 81.4 |
| Malus hupehensis | G | R | 32.5 |
| Malus hupehensis | G | S | 41.2 |
| Malva moschata | G | O | 47.1 |
| Malva sylvestris | G | S | 23.1 |
| Malva verticillata | G | R | 39.9 |
| Matricaria recutita | G | O | 30.0 |
| Matricaria recutita | G | S | 71.3 |
| Melaleuca alternifolia | G | O | 58.3 |
| Melilotus alba | G | S | 41.1 |
| Melilotus albus | G | 0 | 88.8 |
| Melilotus albus | G | R | 100.0 |
| Melissa officinalis | G | O | 47.8 |
| Mentha arvensis | G | R | 33.9 |
| Mentha arvensis | G | O | 63.3 |
| Mentha piperita | G | S | 32.3 |
| Mentha piperita | G | O | 85.9 |
| Mentha piperita | G | R | 100.0 |
| Mentha spicata | G | S | 28.9 |
| Mentha spicata | G | R | 37.5 |
| Mentha suaveolens | G | R | 25.6 |
| Mentha suaveolens | G | O | 70.3 |
| Momordica charantia | G | R | 52.9 |
| Monarda didyma | G | S | 22.0 |
| Monarda didyma | G | O | 100.0 |
| Monarda fistulosa | G | O | 26.0 |
| Nepeta cataria | G | S | 23.4 |
| Nicotiana tabacum | G | S | 45.2 |
| Nigella sativa | G | R | 94.7 |
| Ocimum basilicum | G | S | 23.0 |
| Ocimum basilicum | G | O | 100.0 |
| Ocimum tenuiflorum | G | R | 45.3 |
| Oerothera biennis | G | R | 54.3 |
| Origanum majorana | G | O | 100.0 |
| Origanum majorana | G | R | 100.0 |
| Origanum vulgare | G | R | 93.3 |
| Origanum vulgare | G | O | 93.5 |
| Origanum vulgare | G | S | 97.4 |
| Oxalis Deppei | G | S | 28.7 |
| Oxalis Deppei | G | R | 87.2 |
| Oxalis Deppei | G | O | 100.0 |
| Oxyria digyna | G | R | 54.5 |
| Panicum miliaceum | G | 0 | 71.1 |
| Panicum miliaceum | G | R | 100.0 |
| Panicum miliaceum | G | S | 100.0 |
| Passiflora caerula | G | S | 26.3 |
| Passiflora caerula | G | R | 72.1 |
| Pastinaca sativa | G | S | 24.3 |
| Pastinaca sativa | G | R | 90.2 |
| Petroselinum crispum | G | R | 87.6 |
| Petroselinum crispum | G | O | 100.0 |
| Phalaris canariensis | G | R | 100.0 |
| Phalaris canariensis | G | O | 100.0 |
| Phaseolus acutifolius | G | R | 79.6 |
| Phaseolus coccineus | G | S | 28.3 |
| Phaseolus coccineus | G | R | 80.4 |
| Phaseolus mungo | G | R | 37.2 |
| Phaseolus vulgaris | G | R | 54.3 |
| Phaseolus vulgaris | G | S | 59.0 |
| Phaseolus vulgaris | G | O | 73.7 |
| Phaseolus vulgaris | G | R | 100.0 |
| Phlo x paniculata | G | R | 37.7 |
| Phlo x paniculata | G | O | 77.0 |
| Phlo x paniculata | G | R | 80.8 |
| Physalis ixocarpa | G | S | 30.5 |
| Physalis ixocarpa | G | R | 78.3 |
| Physalis ixocarpa | G | R | 80.9 |
| Physalis pruinosa | G | O | 63.2 |
| Phytolacca americana | G | S | 36.1 |
| Phytolacca americana | G | O | 100.0 |
| Pimpinella anisum | G | S | 26.1 |
| Pimpinella anisum | G | R | 30.0 |
| Pisum sativum | G | S | 28.4 |
| Plantago coronopus | G | R | 27.8 |
| Plantago coronopus | G | O | 51.1 |
| Plantago coronopus | G | R | 67.5 |
| Plantago major | G | S | 30.3 |
| Plantago major | G | O | 64.6 |
| Poa compressa | G | O | 63.0 |
| Poa compressa | G | S | 67.4 |
| Poa compressa | G | R | 89.0 |
| Poa pratensis | G | S | 28.2 |
| Polygonum aviculare | G | R | 100.0 |
| Polygonum pensylvanicum | G | S | 27.7 |
| Polygonum pensylvanicum | G | O | 54.1 |
| Polygonum persicaria | G | S | 32.0 |
| Polygonum persicaria | G | O | 35.7 |
| Polygonum persicaria | G | R | 100.0 |
| Portulaca oleracera | G | R | 51.5 |
| Poterium sanguisorba | G | O | 89.9 |
| Poterium sanguisorba | G | R | 100.0 |
| Poterium sanguisorba | G | S | 23.7 |
| Prunella vulgaris | G | S | 26.7 |
| Prunus cerasifera | G | R | 95.3 |
| Raphanus Raphanistrum | G | R | 41.7 |
| Raphanus Raphanistrum | G | S | 43.5 |
| Raphanus sativus | G | R | 41.0 |
| Raphanus sativus | G | S | 44.6 |
| Raphanus sativus | G | R | 50.5 |
| Raphanus sativus | G | R | 86.1 |
| Raphanus sativus | G | O | 100.0 |
| Reseda odorata | G | O | 58.3 |
| Rheum officinale | G | O | 30.7 |
| Ribes nigrum | G | O | 54.3 |
| Ribes nigrum | G | R | 63.8 |
| Ribes Sylvestre | G | R | 100.0 |
| Ricinus communis | G | R | 41.5 |
| Ricinus communis | G | O | 100.0 |
| Rosmarinus officinalis | G | R | 90.0 |
| Rubus idaeus | G | S | 37.1 |
| Rubus ideaus | G | R | 26.6 |
| Rubus occidentalis | G | R | 35.1 |
| Rume x crispus | G | R | 30.3 |
| Rume x crispus | G | S | 100.0 |
| Rume x patientia | G | R | 41.0 |
| Rume x patientia | G | S | 41.9 |
| Ruta graveolens | G | S | 47.9 |
| Ruta graveolens | G | R | 82.1 |
| Saccharum officinarum | G | R | 100.0 |
| Salvia elegens | G | O | 100.0 |
| Salvia officinalis | G | S | 35.3 |
| Salvia officinalis | G | O | 100.0 |
| Salvia officinalis | G | R | 100.0 |
| Sambucus ebulus | G | R | 53.9 |
| Santolina chamaecyparissus | G | S | 36.4 |
| Santolina chamaecyparissus | G | 0 | 69.5 |
| Santolina chamaecyparissus | G | R | 100.0 |
| Saponaria officinalis | G | S | 29.8 |
| Satureja hortensis | G | O | 97.4 |
| Satureja hortensis | G | R | 100.0 |
| Satureja montana | G | O | 59.2 |
| Satureja repandra | G | S | 35.3 |
| Satureja repandra | G | O | 66.2 |
| Scorzonera hispanica | G | S | 24.5 |
| Scrophularia nodosa | G | S | 24.5 |
| Scrophularia nodosa | G | O | 30.0 |
| Scrophularia nodosa | G | R | 55.6 |
| Scutellaria lateriflora | G | S | 20.3 |
| Scutellaria lateriflora | G | R | 83.1 |
| Secale cereale | G | O | 51.1 |
| Senecio vulgaris | G | R | 42.5 |
| Sesamum indicum | G | S | 34.3 |
| Sesamum indicum | G | R | 44.5 |
| Silene vulgaris | G | S | 34.1 |
| Sium sisarum | G | O | 100.0 |
| Solanum melanocerasum | G | S | 40.6 |
| Solanum melanocerasum | G | R | 85.4 |
| solanum melongena | G | S | 58.2 |
| solanum melongena | G | O | 83.0 |
| solanum melongena | G | R | 85.6 |
| Solanum tuberosum | G | O | 40.2 |
| Sonchus oleraceus | G | R | 41.1 |
| Sorghum dochna | G | S | 25.0 |
| Sorghum dochna | G | O | 64.3 |
| Sorghum dochna | G | R | 100.0 |
| sorghum durra | G | R | 60.1 |
| Sorghum durra | G | O | 100.0 |
| Sorghum sudanense | G | O | 98.0 |
| Spinacia oleracea | G | S | 24.9 |
| Spinacia oleracea | G | O | 100.0 |
| Stachys byzantina | G | R | 78.8 |
| Stellaria graminea | G | S | 29.3 |
| Stellaria media | G | S | 33.4 |
| Stellaria media | G | R | 45.4 |
| Symphytum officinale | G | O | 57.5 |
| Tanacetum cinerariifolium | G | R | 100.0 |
| Tanacetum parthenium | G | R | 28.2 |
| Tanacetum vulgare | G | S | 25.2 |
| Tanacetum vulgare | G | R | 39.3 |
| Tanacetum vulgare | G | O | 81.2 |
| Taraxacum officinale | G | R | 51.1 |
| Thymus fragantissimus | G | S | 29.9 |
| Thymus fragantissimus | G | O | 55.3 |
| Thymus praeco x subsp arcticus | G | S | 27.7 |
| Thymus serpyllum | G | R | 74.9 |
| Thymus vulgaris | G | S | 23.3 |
| Thymus vulgaris | G | R | 86.4 |
| Thymus x citriodorus | G | R | 97.6 |
| Tragopogon porrifolius | G | R | 76.2 |
| Trichosanthes kirilowii | G | O | 87.7 |
| Trigonella foenumgraecum | G | S | 31.0 |
| Trigonella foenumgraecum | G | O | 84.0 |
| Triticosecale spp | G | S | 26.5 |
| Triticosecale spp | G | O | 73.5 |
| Triticum aestivum | G | R | 62.4 |
| Triticum durum | G | O | 51.9 |
| Triticum spelta | G | S | 24.5 |
| Triticum spelta | G | O | 32.9 |
| Triticum turgidum | G | O | 25.1 |
| Tropaeolum majus | G | S | 21.3 |
| Tropaeolum majus | G | R | 45.6 |
| Urtica dioica | G | S | 21.3 |
| Urtica dioica | G | O | 100.0 |
| Valerianella locusta | G | O | 32.2 |
| Veratrum viride | G | R | 77.7 |
| Verbascum thapsus | G | S | 34.0 |
| Veronica beccabunga | G | R | 44.1 |
| Veronica officinalis | G | S | 38.8 |
| Veronica officinalis | G | R | 87.5 |
| Viburnum trilobum | G | O | 62.6 |
| Vicia faba | G | S | 22.2 |
| Vicia sativa | G | 0 | 74.8 |
| Vicia sativa | G | R | 100.0 |
| Vicia villosa | G | R | 100.0 |
| Vigna angularis | G | R | 65.2 |
| Vigna sesquipedalis | G | S | 35.1 |
| Vigna sesquipedalis | G | R | 73.8 |
| Vigna sesquipedalis | G | O | 100.0 |
| Vigna unguiculata | G | S | 65.9 |
| Vigna unguiculata | G | R | 84.5 |
| Vinca minor | G | S | 22.1 |
| Vitis sp. | G | R | 40.1 |
| Vitis sp. | G | O | 74.7 |
| Withania somnifera | G | S | 37.3 |
| Withania somnifera | G | O | 91.0 |
| Xanthium sibiricum | G | S | 38.4 |
| Xanthium sibiricum | G | O | 100.0 |
| Xanthium strumarium | G | S | 37.7 |
| Xanthium strumarium | G | O | 39.6 |
| Xanthium strumarium | G | R | 40.0 |
| Zea mays | G | S | 43.3 |
| Zea mays | G | O | 64.4 |
| Zea mays | G | R | 68.3 |
| Abies lasiocarpa | T | S | 20.2 |
| Abies lasiocarpa | T | R | 59.1 |
| Achillea millefolium | T | O | 84.7 |
| Aconitum napellus | T | O | 22.0 |
| Aconitum napellus | T | R | 100.0 |
| Adiantum pedatum | T | R | 100.0 |
| Agaricus bisporus | T | R | 52.1 |
| Agaricus bisporus | T | R | 65.6 |
| Ageratum conyzoides | T | S | 26.7 |
| Agropyron repens | T | S | 30.2 |
| Agrostis Stolonifera | T | O | 100.0 |
| Alcea rosea | T | R | 63.7 |
| Alchemilla mollis | T | R | 28.6 |
| Allium ampeloprasum | T | R | 55.9 |
| Allium ampeloprasum | T | O | 60.4 |
| Allium ascalonicum | T | S | 20.4 |
| Allium ascalonicum | T | O | 73.4 |
| Allium cepa | T | S | 33.8 |
| Allium cepa | T | S | 35.6 |
| Allium cepa | T | R | 48.0 |
| Allium cepa | T | R | 78.6 |
| Allium grande | T | R | 32.4 |
| Allium schoenoprasum | T | R | 67.7 |
| Allium tuberosum | T | S | 38.8 |
| Allium tuberosum | T | O | 82.5 |
| Allium tuberosum | T | R | 85.2 |
| Aloe vera | T | R | 74.6 |
| Althaea officianalis | T | S | 37.7 |
| Althaea officinalis | T | O | 55.3 |
| Althaea officinalis | T | R | 72.3 |
| Amaranthus caudathus | T | O | 53.5 |
| Amaranthus gangeticus | T | S | 28.1 |
| Ananas comosus | T | R | 37.9 |
| Ananas comosus | T | O | 100.0 |
| angelica archangelica | T | R | 41.3 |
| Anthemis nobilis | T | O | 100.0 |
| Anthemis nobilis | T | R | 100.0 |
| Anthriscus cerefolium | T | S | 21.9 |
| Anthriscus cerefolium | T | O | 67.1 |
| Apium graveolens | T | R | 35.5 |
| Apium graveolens | T | R | 52.1 |
| Aralia cordata | T | R | 100.0 |
| Aralia nudicaulis | T | R | 31.2 |
| Arctium minus | T | S | 31.3 |
| Arctium minus | T | O | 73.7 |
| Armoracia rusticana | T | O | 49.9 |
| Arrhenatherum elatius | T | O | 100.0 |
| Artemisia dracunlus | T | S | 100.0 |
| Asclepias incarnata | T | S | 32.3 |
| Asparagus officinalis | T | S | 48.2 |
| Atriple x hortensis | T | R | 28.4 |
| Avena sativa | T | R | 31.3 |
| Avena sativa | T | O | 70.6 |
| Avena sativa | T | R | 100.0 |
| Averrhoa carambola | T | R | 44.0 |
| Bellis perennis | T | R | 82.0 |
| Beta vulgaris | T | S | 33.7 |
| Beta vulgaris | T | R | 100.0 |
| Betula glandulosa | T | O | 53.5 |
| Boletus edulis | T | S | 21.8 |
| Borago officinalis | T | S | 42.3 |
| Borago officinalis | T | R | 78.5 |
| Brassica hirta | T | R | 53.1 |
| Brassica hirta | T | O | 68.9 |
| Brassica Napus | T | S | 45.1 |
| Brassica Napus | T | R | 82.9 |
| Brassica oleracea | T | R | 38.8 |
| Brassica oleracea | T | R | 49.7 |
| Brassica oleracea | T | O | 75.5 |
| Brassica oleracea | T | R | 77.0 |
| Brassica oleracea | T | S | 77.2 |
| Brassica rapa | T | R | 25.4 |
| Brassica rapa | T | O | 37.9 |
| Brassica rapa | T | S | 47.7 |
| Brassica rapa | T | R | 64.7 |
| Brassica rapa | T | R | 81.8 |
| Calamintha nepeta | T | O | 57.6 |
| Calendula officinalis | T | S | 32.6 |
| Camellia sinensis | T | S | 21.0 |
| Camellia sinensis | T | R | 43.8 |
| Camellia sinensis | T | O | 66.2 |
| Canna edulis | T | O | 100.0 |
| Cantharellus cibarias | T | S | 26.0 |
| Capsicum annuum | T | S | 54.6 |
| Capsicum annuum | T | R | 100.0 |
| Capsicum frutescens | T | S | 60.9 |
| Capsicum frutescens | T | R | 100.0 |
| Care x morrowii | T | R | 24.4 |
| Carica papaya | T | S | 20.8 |
| Carthamus tinctorius | T | R | 39.6 |
| Carya cordiformis | T | R | 100.0 |
| Cerastium tomentosum | T | R | 54.8 |
| Chaerophyllum bulbosum | T | S | 42.2 |
| Chaerophyllum bulbosum | T | R | 74.3 |
| Chelidonium majus | T | S | 20.3 |
| Chenopodium quinoa | T | O | 76.0 |
| Chrysanthemum coronarium | T | S | 30.6 |
| Chrysanthemum parthenium | T | R | 57.2 |
| chrysanthemun coronarium | T | R | 56.5 |
| Chrysanthenum coronarium | T | R | 81.6 |
| Cicer arietinum | T | O | 32.2 |
| Cichorium endivia subsp endivia | T | R | 27.1 |
| Cichorium endivia subsp. Endivia | T | S | 26.9 |
| Cichorium endivia subsp. Endivia | T | O | 64.5 |
| Cichorium intybus | T | S | 22.7 |
| Cichorium intybus | T | R | 53.5 |
| Cimicifuga racemosa | T | S | 41.1 |
| Cimicifuga racemosa | T | R | 68.4 |
| Circium arvense | T | S | 42.5 |
| Circium arvense | T | R | 64.5 |
| Citrullus lanatus | T | S | 72.4 |
| Citrullus lanatus | T | O | 92.2 |
| Citrullus lanatus | T | R | 100.0 |
| Citrus limettoides | T | O | 77.1 |
| Citrus limon | T | R | 43.6 |
| Citrus paradisi | T | S | 21.8 |
| Citrus paradisi | T | R | 90.9 |
| Citrus sinensis | T | R | 46.7 |
| Colocasia sp | T | R | 43.4 |
| Colocasia sp | T | O | 84.3 |
| Corchorus olitorius | T | R | 22.7 |
| Coriandrum sativum | T | S | 20.4 |
| Cornus canadensis | T | S | 66.0 |
| Cosmos sulphureus | T | R | 47.1 |
| Crataegus submollis | T | S | 21.2 |
| Crataegus submollis | T | O | 94.3 |
| Cucumis anguria | T | S | 49.4 |
| Cucumis anguria | T | R | 84.1 |
| Cucumis melo | T | S | 56.6 |
| Cucumis melo | T | R | 92.4 |
| Cucumis melo | T | O | 100.0 |
| Cucumis metuliferus | T | S | 29.5 |
| Cucumis sativus | T | S | 28.3 |
| Cucurbita maxima | T | S | 26.7 |
| Cucurbita maxima | T | O | 34.7 |
| Cucurbita maxima | T | R | 62.1 |
| Cucurbita moschata | T | R | 30.7 |
| Cucurbita moschata | T | S | 33.4 |
| Cucurbita moschata | T | S | 48.3 |
| Cucurbita moschata | T | R | 98.8 |
| Cucurbita moschata | T | O | 100.0 |
| Cucurbita pepo | T | S | 45.8 |
| Cucurbita pepo | T | R | 80.2 |
| Erysimum perofskianum | T | S | 28.2 |
| Erysimum perofskianum | T | R | 85.2 |
| Eschscholzia californica | T | S | 49.9 |
| Eschscholzia californica | T | O | 74.5 |
| Fagopyrum esculentum | T | O | 52.9 |
| Fagopyrum tartaricum | T | S | 25.6 |
| Fagopyrum tartaricum | T | R | 68.4 |
| Fagopyrum tartaricum | T | O | 100.0 |
| Festuca rubra | T | O | 51.6 |
| Festuca rubra | T | S | 56.6 |
| Festuca rubra | T | R | 71.7 |
| Foeniculum vulgare | T | S | 36.5 |
| Foeniculum vulgare | T | R | 41.4 |
| Foericulum vulgare | T | 0 | 100.0 |
| Fortunella spp | T | R | 53.9 |
| Fragaria x ananassa | T | R | 28.1 |
| Galinsoga ciliata | T | S | 43.2 |
| Galinsoga ciliata | T | R | 73.3 |
| Galium odoratum | T | S | 42.0 |
| Galium odoratum | T | O | 94.2 |
| Glau x Maritima | T | R | 24.8 |
| Glycine max | T | R | 37.2 |
| Glycine max | T | O | 100.0 |
| Glycine max | T | R | 100.0 |
| Glycine max | T | S | 100.0 |
| Gossypium herbaceum | T | R | 48.7 |
| Guizotia abyssinica | T | S | 26.8 |
| Guizotia abyssinica | T | R | 100.0 |
| Hedeoma pulegioides | T | R | 20.3 |
| Hedeoma pulegioides | T | O | 72.7 |
| Helianthus annuus | T | R | 56.1 |
| Helianthus strumosus | T | O | 100.0 |
| Helianthus tuberosus | T | S | 25.3 |
| Helianthus tuberosus | T | R | 28.1 |
| Helianthus tuberosus | T | O | 78.6 |
| Helianthus tuberosus | T | R | 91.5 |
| Helichrysum angustifolium | T | R | 83.4 |
| Helichrysum angustifolium | T | S | 88.3 |
| Helichrysum thianschanicum | T | O | 26.0 |
| Heliotropium arborescens | T | R | 100.0 |
| Helleborus niger | T | R | 23.0 |
| Hibiscus cannabinus | T | R | 37.9 |
| Hordeum vulgare | T | O | 75.9 |
| Hordeum vulgare supsp vulgare | T | S | 20.5 |
| Hordeum vulgare supsp vulgare | T | O | 62.3 |
| Humulus lupulus | T | S | 44.7 |
| Humulus lupulus | T | O | 70.6 |
| Hypericum henryi | T | O | 76.8 |
| Hypericum henryi | T | R | 99.8 |
| Hypericum perforatum | T | R | 38.8 |
| Hyssopus officinalis | T | O | 100.0 |
| Iberis amara | T | O | 100.0 |
| Juniperus communis | T | S | 100.0 |
| Kochia scoparia | T | S | 25.2 |
| Koeleria glauca | T | S | 23.1 |
| Lactuca sativa | T | R | 70.5 |
| Lactuca serriola | T | R | 34.1 |
| Laportea canadensis | T | R | 61.3 |
| Lathyrus sylvestris | T | R | 48.6 |
| Laurus nobilis | T | O | 73.6 |
| Lavandula angustifolia | T | R | 35.0 |
| Lavandula angustifolia | T | O | 100.0 |
| Lavandula latifolia | T | O | 77.1 |
| Lepidium sativum | T | S | 35.2 |
| Lepidium sativum | T | R | 48.1 |
| Lepidium sativum | T | O | 72.9 |
| Levisticum officinale | T | S | 38.7 |
| Levisticum officinale | T | O | 60.3 |
| Linum usitatissimum | T | R | 24.7 |
| Lolium multiflorum | T | S | 39.8 |
| Lolium multiflorum | T | O | 74.1 |
| Lonicera ramosissima | T | S | 34.4 |
| Lonicera ramosissima | T | O | 80.5 |
| Lonicera syringantha | T | R | 58.4 |
| Lotus corniculatus | T | S | 36.0 |
| Lotus corniculatus | T | O | 100.0 |
| Lotus tetragonolobus | T | R | 76.1 |
| Lunaria annua | T | R | 47.4 |
| Lycopersicon esculentum | T | R | 69.7 |
| Lycopersicon pimpinellifolium | T | R | 58.7 |
| Malus hupehensis | T | R | 53.1 |
| Malus hupehensis | T | S | 100.0 |
| Malus sp. | T | R | 72.6 |
| Malva moschata | T | O | 96.7 |
| Malva verticillata | T | R | 35.8 |
| Manihot esculenta | T | R | 53.7 |
| Melaleuca alternifolia | T | S | 21.5 |
| Melaleuca alternifolia | T | O | 78.7 |
| Melilotus albus | T | R | 79.7 |
| Melilotus officinalis | T | S | 34.6 |
| Melilotus officinalis | T | R | 100.0 |
| Melissa officinalis | T | O | 100.0 |
| Mentha piperita | T | S | 24.5 |
| Mentha pulegium | T | O | 100.0 |
| Mentha suaveolens | T | O | 20.9 |
| Miscanthus sinensis Andress | T | S | 69.1 |
| Momordica charantia | T | R | 54.9 |
| Monarda didyma | T | S | 31.3 |
| Monarda fistulosa | T | S | 21.3 |
| Monarda fistulosa | T | O | 100.0 |
| Montia perfoliata | T | R | 67.2 |
| Musa paradisiaca | T | R | 47.3 |
| nasturtium officinale | T | S | 55.7 |
| Nepeta cataria | T | S | 20.7 |
| Nepeta cataria | T | S | 69.0 |
| Nepeta cataria | T | O | 100.0 |
| Nicotiana rustica | T | S | 52.8 |
| Nicotiana rustica | T | R | 88.1 |
| Nicotiana tabacum | T | S | 50.3 |
| Nicotiana tabacum | T | R | 91.5 |
| Nigella sativa | T | R | 34.2 |
| Nigella sativa | T | R | 90.3 |
| Nigella sativa | T | R | 100.0 |
| Ocimum Basilicum | T | S | 21.6 |
| Ocimum Basilicum | T | O | 100.0 |
| Ocimum tenuiflorum | T | R | 44.5 |
| Oenothera biennis | T | R | 48.2 |
| Onobrychis viciifolia | T | S | 34.4 |
| Onobrychis viciifolia | T | O | 35.6 |
| Opuntia sp. | T | S | 23.5 |
| Origanum vulgare | T | S | 20.7 |
| Origanum vulgare | T | R | 76.7 |
| Origanum vulgare | T | O | 100.0 |
| Oryza sativa | T | R | 60.8 |
| Oxalis Deppei | T | S | 22.2 |
| Oxalis Deppei | T | R | 81.4 |
| Passiflora caerulea | T | S | 36.9 |
| Passiflora caerulea | T | R | 87.0 |
| Passiflora spp | T | R | 54.6 |
| Pastinaca sativa | T | S | 24.8 |
| Pastinaca sativa | T | R | 74.7 |
| Perilla frutescens | T | R | 100.0 |
| Perroselinum crispum | T | R | 85.2 |
| Perroselinum crispum | T | O | 100.0 |
| Persea americana | T | R | 43.1 |
| Petasites Japonicus | T | S | 21.9 |
| Petroselinum crispum | T | R | 52.8 |
| Peucedanum oreaselinum | T | R | 41.9 |
| Phalaris canariensis | T | R | 41.1 |
| Phalaris canariensis | T | O | 100.0 |
| Phaseolus acutifolius | T | R | 88.2 |
| Phaseolus coccineus | T | S | 22.2 |
| Phaseolus coccineus | T | R | 36.4 |
| Phaseolus coccineus | T | R | 86.7 |
| Phaseolus coccineus | T | O | 100.0 |
| Phaseolus mungo | T | S | 43.0 |
| Phaseolus vulgaris | T | S | 62.9 |
| Phaseolus vulgaris | T | R | 71.9 |
| Phaseolus vulgaris | T | R | 73.0 |
| Phaseolus vulgaris | T | O | 100.0 |
| Phlo x paniculata | T | R | 23.1 |
| Phlo x paniculata | T | R | 92.8 |
| Physalis alkekengi | T | R | 39.5 |
| Physalis ixocarpa | T | R | 36.7 |
| Physalis ixocarpa | T | R | 75.9 |
| Physalis pruinosa | T | R | 65.6 |
| Physalis pruinosa | T | R | 71.0 |
| Physalis pruinosa | T | O | 100.0 |
| Physalis pruinosa | T | O | 100.0 |
| Phytolacca decandra | T | S | 39.3 |
| Phytolacca decandra | T | O | 42.0 |
| Pimpinella anisum | T | S | 27.9 |
| Pimpinella anisum | T | R | 35.8 |
| Pimpinella anisum | T | O | 49.9 |
| Pimpinella anisum | T | R | 55.5 |
| Pisum sativum | T | S | 22.3 |
| Plantago coronopus | T | R | 35.2 |
| Plantago coronopus | T | R | 46.0 |
| Plantago coronopus | T | O | 73.5 |
| Plantago major | T | S | 22.3 |
| Plectranthus sp. | T | S | 59.2 |
| Pleurotus spp | T | R | 26.6 |
| Poa compressa | T | S | 33.4 |
| Poa compressa | T | R | 75.7 |
| Poa compressa | T | O | 100.0 |
| Poa pratensis | T | S | 25.4 |
| Polygonum pensylvanicum | T | O | 66.8 |
| Polygonum pensylvanicum | T | R | 73.3 |
| Polygonum persicaria | T | S | 27.1 |
| Polygonum persicaria | T | O | 50.8 |
| Populus incrassata | T | O | 74.3 |
| Populus incrassata | T | S | 100.0 |
| Prunus armeniaca | T | R | 55.0 |
| Prunus cerasus | T | O | 100.0 |
| Prunus persica | T | S | 26.0 |
| Prunus persica | T | R | 46.2 |
| Psoralea corylifolia | T | S | 47.4 |
| Pteridium aquilinum | T | R | 100.0 |
| Pyrus communis | T | R | 42.9 |
| Raphanus raphanistrum | T | S | 24.4 |
| Raphanus raphanistrum | T | R | 56.9 |
| Raphanus raphanistrum | T | O | 62.1 |
| Raphanus raphanistrum | T | O | 100.0 |
| Raphanus sativus | T | R | 48.9 |
| Raphanus sativus | T | S | 59.8 |
| Raphanus sativus | T | R | 81.6 |
| Reseda odorata | T | O | 71.3 |
| Rhamnus frangula | T | O | 44.6 |
| Rhamnus frangula | T | R | 74.4 |
| Rheum officinale | T | O | 73.9 |
| Rheum officinale | T | S | 100.0 |
| Ricinus communis | T | O | 100.0 |
| Rosmarinus officinalis | T | O | 100.0 |
| Rosmarinus officinalis | T | R | 100.0 |
| Rubus ideaus | T | R | 78.1 |
| Rume x acetosella | T | R | 42.2 |
| Rume x crispus | T | O | 73.1 |
| Rume x patientia | T | S | 52.0 |
| Ruta graveolens | T | S | 34.7 |
| Ruta graveolens | T | O | 100.0 |
| Saccharum officinarum | T | S | 59.6 |
| Saccharum officinarum | T | R | 66.1 |
| Salvia elegans | T | S | 36.3 |
| Salvia elegans | T | O | 44.3 |
| Salvia officinalis | T | S | 28.2 |
| Salvia officinalis | T | O | 100.0 |
| Salvia sclarea | T | R | 38.6 |
| Sambucus canadensis | T | S | 36.3 |
| Sambucus canadensis | T | R | 64.5 |
| Sambucus canadensis | T | O | 100.0 |
| Sanguisorba minor | T | O | 73.1 |
| Sanguisorba minor | T | R | 100.0 |
| Santolina chamaecyparissus | T | O | 27.7 |
| Santolina chamaecyparissus | T | R | 100.0 |
| Saponaria officinalis | T | R | 100.0 |
| Satureja hortensis | T | O | 62.2 |
| Satureja hortensis | T | R | 100.0 |
| Satureja montana | T | S | 34.7 |
| Satureja montana | T | O | 36.3 |
| Satureja montana | T | R | 100.0 |
| Satureja repandra | T | O | 47.0 |
| Satureja repandra | T | S | 47.6 |
| Satureja repandra | T | R | 84.6 |
| Scolymus hispanicus | T | R | 35.8 |
| Scorzorera hipanica | T | R | 99.4 |
| Scrophularia nodosa | T | S | 29.1 |
| Scrophularia nodosa | T | R | 90.1 |
| Scrophularia nodosa | T | O | 100.0 |
| Scutellaria lateriflora | T | S | 30.9 |
| Scutellaria lateriflora | T | R | 63.9 |
| Secale cereale | T | O | 100.0 |
| Senecio vulgaris | T | S | 24.7 |
| Senecio vulgaris | T | R | 32.2 |
| Sesamum indicum | T | R | 100.0 |
| Silene vulgaris | T | S | 25.6 |
| Sium sisarum | T | O | 81.4 |
| Sium sisarum | T | O | 100.0 |
| Solanum melanocerasum | T | S | 28.0 |
| Solanum melanocerasum | T | R | 78.8 |
| Solanum melanocerasum | T | R | 99.6 |
| Solanum melongena | T | S | 70.5 |
| Sorghum caffrorum | T | S | 28.1 |
| Sorghum dochna | T | R | 40.6 |
| Sorghum dochna | T | O | 100.0 |
| Sorghum durra | T | R | 29.7 |
| Sorghum durra | T | O | 78.9 |
| Sorghum sudanense | T | R | 74.6 |
| Sorghum sudanense | T | O | 100.0 |
| Spinacia oleracea | T | S | 28.5 |
| Spinacia oleracea | T | O | 62.7 |
| Stachys byzantina | T | R | 66.9 |
| Stachys byzantina | T | O | 100.0 |
| Stellaria media | T | S | 21.4 |
| Stellaria media | T | R | 87.1 |
| Stipa capillata | T | R | 37.5 |
| Symphytum officinale | T | O | 58.5 |
| Tanacetum cinerariifolium | T | O | 100.0 |
| Tanacetum cinerariifolium | T | R | 100.0 |
| Tanacetum parthenium | T | R | 100.0 |
| Tanacetum vulgare | T | R | 20.8 |
| Taraxacum officinale | T | R | 76.3 |
| Teucrium chamaedrys | T | O | 75.6 |
| Thalpsi arvense | T | O | 64.1 |
| Thymus fragantissimus | T | S | 21.4 |
| Thymus praeco x subsp arcticus | T | S | 36.4 |
| Thymus pseudolanuginosus | T | S | 21.1 |
| Thymus pseudolanuginosus | T | O | 75.4 |
| Thymus serpyllum | T | O | 64.2 |
| Thymus vulgaris | T | R | 71.5 |
| Thymus X citriodorus | T | S | 27.6 |
| Tragopogon porrifolium | T | S | 44.8 |
| Tragopogon porrifolius | T | O | 39.1 |
| Tragopogon porrifolius | T | R | 57.9 |
| Tragopogon sp. | T | R | 20.0 |
| Trifolium repens | T | R | 79.7 |
| Trigonella foenum graecum | T | O | 28.4 |
| Trigonella foenum graecum | T | S | 34.8 |
| Triticosecale spp | T | S | 28.5 |
| Triticosecale spp | T | O | 100.0 |
| Triticum aestivum | T | R | 32.9 |
| Triticum aestivum | T | O | 67.7 |
| Triticum durum | T | O | 47.7 |
| Triticum spelta | T | O | 37.1 |
| Triticum turgidumm | T | O | 41.2 |
| Tropaeolum majus | T | S | 42.7 |
| Tropaeolum majus | T | R | 77.6 |
| Tsuga diversifolia | T | R | 53.4 |
| Typha latifolia | T | S | 29.2 |
| Urtica dioica | T | S | 29.5 |
| Vaccinium angustifolium | T | R | 59.4 |
| Vaccinium angustifolium | T | R | 100.0 |
| Vaccinium macrocarpon | T | S | 51.1 |
| Vaccinium macrocarpon | T | O | 64.7 |
| Valerianella locusta | T | S | 22.7 |
| Valerianella locusta | T | O | 24.8 |
| Veronica beccabunga | T | R | 33.3 |
| Veronica officinalis | T | R | 59.2 |
| Veronica officinalis | T | O | 100.0 |
| Viburnum trilobum | T | O | 71.2 |
| Vicia faba | T | S | 25.5 |
| Vicia faba | T | R | 27.0 |
| Vicia sativa | T | O | 56.6 |
| Vicia villosa | T | R | 100.0 |
| Vigna angularis | T | R | 49.2 |
| Vigna sesquipedalis | T | R | 77.4 |
| Vigna sesquipedalis | T | O | 100.0 |
| Vigna unguiculata | T | S | 27.2 |
| Vigna unguiculata | T | R | 59.0 |
| Vinca minor | T | R | 39.2 |
| Vitis sp. | T | R | 31.9 |
| Vitis sp. | T | S | 36.3 |
| Vitis sp. | T | O | 72.2 |
| Weigela coraeensis | T | S | 32.9 |
| Weigela coraeensis | T | R | 61.5 |
| Withania somnifera | T | S | 36.1 |
| Withania somnifera | T | O | 83.3 |
| Xanthium sibiricum | T | S | 32.1 |
| Xanthium sibiricum | T | R | 33.2 |
| Xanthium sibiricum | T | O | 62.4 |
| Xanthium strumarium | T | S | 47.2 |
| Xanthium strumarium | T | O | 74.3 |
| Zea mays | T | R | 55.7 |
| Zea mays | T | O | 100.0 |
| Zingiber officinale | T | R | 79.0 |

TABLE 3
MMP-3

| Nom latin | Stress | Extrait | Inhibition (%) |
|---|---|---|---|
| Achillea millefolium | A | O | 21.4 |
| Allium Tuberosum | A | S | 32.5 |
| Anethum graveolens | A | S | 26.0 |
| Anthemis nobilis | A | R | 20.3 |
| Anthemis tinctoria | A | R | 58.0 |
| Apium graveolens | A | R | 34.1 |
| Arctium minus | A | R | 53.9 |
| Arctium minus | A | O | 100.0 |
| Arctostaphylos uva-ursi | A | S | 58.6 |
| Aronia melanocarpa | A | R | 32.2 |
| Artemisia Absinthium | A | O | 100.0 |
| Artemisia dracunculus | A | R | 23.4 |
| Artemisia dracunculus | A | S | 63.0 |
| Aster sp | A | O | 42.4 |
| Atropa belladonna | A | O | 23.8 |
| Beta vulgaris | A | S | 24.1 |
| Beta vulgaris | A | O | 42.9 |
| Beta vulgaris | A | O | 94.3 |
| Beta vulgaris | A | R | 97.9 |
| Beta vulgaris var. condivata | A | O | 21.2 |
| Brassica napus | A | S | 25.0 |
| Brassica napus | A | O | 100.0 |
| Brassica oleracea | A | S | 39.9 |
| Canna edulis | A | S | 39.6 |
| Capsicum annuum | A | S | 35.4 |
| Capsicum frutescens | A | S | 27.2 |
| Cichorium intybus | A | O | 20.2 |
| Cichorium intybus | A | R | 26.5 |
| Cichorium intybus | A | S | 28.2 |
| Citrullus lanatus | A | S | 21.7 |
| Citrullus lanatus | A | O | 27.8 |
| Citrullus lanatus | A | R | 34.4 |
| Coix Lacryma-Jobi | A | S | 37.3 |
| Coix Lacryma-Jobi | A | O | 78.1 |
| Cosmos sulphureus | A | R | 26.8 |
| Crataegus submollis | A | S | 22.3 |
| Crataegus submollis | A | R | 61.6 |
| Cucumis anguria | A | S | 27.8 |
| Cucurbita Maxima | A | S | 28.9 |
| Cucurbita moschata | A | S | 32.9 |
| Cucurbita pepo | A | S | 50.9 |
| Datisca cannabina | A | R | 43.3 |
| Datisca cannabina | A | S | 100.0 |
| Digitalis purpurea | A | R | 20.0 |
| Dipsacus sativus | A | R | 64.8 |
| Dirca palustris | A | S | 29.6 |
| Dryopteris filix-mas | A | R | 22.0 |
| Dryopteris filix-mas | A | O | 32.8 |
| Echinacea purpurea | A | O | 100.0 |
| Fagopyrum tataricum | A | R | 28.3 |
| Fagopyrum tataricum | A | O | 29.7 |
| Filipendula rubra | A | S | 43.7 |
| Filipendula rubra | A | R | 63.2 |
| Fragaria x ananassa | A | R | 41.5 |
| Fragaria x ananassa | A | S | 67.1 |
| Fragaria x ananassa | A | O | 99.6 |
| Fragaria x ananassa | A | R | 31.7 |
| Gaultheria hispidula | A | R | 50.5 |
| Glycyrrhiza glabra | A | R | 56.2 |
| Hedeoma pulegioides | A | O | 51.7 |
| Helianthus tuberosus | A | O | 22.9 |
| Hordeum vulgare subsp vulgare | A | S | 36.0 |
| Hypericum henryi | A | R | 67.2 |
| Hypericum perforatum | A | R | 31.7 |
| Hyssopus officinalis | A | R | 21.6 |
| Iris versicolor | A | R | 53.6 |
| Isatis tinctoria | A | S | 32.9 |
| Levisticum officinale | A | O | 46.7 |
| Lotus tetragonolobus | A | R | 26.2 |
| Matricaria recutita | A | S | 43.5 |
| Matteucia pensylvanica | A | R | 24.7 |
| Melissa officinalis | A | S | 30.3 |
| Mentha suaveolens | A | R | 91.7 |
| Nepeta cataria | A | S | 30.3 |
| Nigella sativa | A | O | 26.0 |
| Ocinum tenuiflorum | A | O | 33.0 |
| Ocinum tenuiflorum | A | R | 49.8 |
| Perilla frutescens | A | R | 34.8 |
| Petasites japonicus | A | R | 38.0 |
| Phaseolus mungo | A | O | 62.6 |
| Phaseolus vulgaris | A | S | 21.2 |
| Phaseolus vulgaris | A | O | 50.6 |
| Phaseolus Vulgaris | A | R | 100.0 |
| Phlox paniculata | A | S | 46.4 |
| Physalis alkekengi | A | O | 37.5 |
| Plantago major | A | O | 27.3 |
| Polygonum aviculare linné | A | S | 24.8 |
| Polygonum persicaria | A | S | 59.1 |
| Potentilla anserina | A | R | 40.1 |
| Poterium sanguisorba | A | R | 75.7 |
| Prunus cerasitera | A | R | 80.0 |
| Ptaridium aquilinus | A | R | 39.6 |
| Raphanus raphanistrum | A | S | 28.2 |
| Raphanus sativus | A | S | 64.4 |
| Ribes nigrum | A | O | 47.6 |
| ribes uva-crispa | A | R | 21.0 |
| ribes uva-crispa | A | O | 100.0 |
| Rosa rugosa | A | S | 21.4 |
| Rosmarinus officinalis | A | R | 27.3 |
| Rubus allegheniensis | A | R | 81.0 |
| Rubus arcticus | A | R | 51.0 |
| Rubus canadensis | A | R | 48.8 |
| Rubus idaeus | A | S | 28.5 |
| Rubus idaeus | A | R | 35.1 |
| Rubus pubescens | A | O | 50.4 |
| Rubus thibetanus | A | O | 39.1 |
| Rumex patientia | A | S | 24.8 |
| Ruta graveolens | A | O | 56.1 |
| Salvia officinalis | A | R | 43.2 |
| Santolina chamaecyparissus | A | R | 27.0 |
| Scutellaria lateriflora | A | R | 53.5 |
| Solanum melongena | A | S | 21.8 |
| Solidago canadensis | A | S | 27.4 |
| Stachys affinis | A | S | 100.0 |
| Stellaria media | A | O | 24.4 |
| Tanacetum vulgare | A | R | 62.1 |
| Thymus praecox subsp arcticus | A | S | 28.4 |
| Thymus praecox subsp arcticus | A | O | 31.8 |
| Trichosanthes kirilowii | A | S | 23.2 |
| Vaccinium Corymbosum | A | R | 100.0 |
| Vaccinium macrocarpon | A | S | 48.6 |
| Vaccinium augustifolium | A | R | 56.6 |
| Vigna angularia | A | O | 23.1 |
| Vigna sesquipedalis | A | O | 37.8 |
| Vigna unguiculata | A | S | 52.5 |
| Vinca minor | A | O | 23.2 |
| Vitis sp. | A | S | 20.8 |
| Vitis sp. | A | O | 21.5 |
| Vitis sp. | A | R | 33.6 |
| Xanthium sibiricum | A | S | 27.3 |
| Aconitum napellus | G | O | 59.0 |
| Agropyron repens | G | O | 69.4 |
| Alchemilla mollis | G | S | 30.6 |
| Alchemilla mollis | G | O | 73.3 |
| Allium grande | G | O | 33.4 |
| Anethum graveolens | G | S | 40.5 |
| Aronia melanocarpa | G | O | 100.0 |
| Artemisia absinthium | G | S | 31.3 |
| Artemisia absinthium | G | O | 67.9 |
| Artemisia dracunculus | G | S | 100.0 |
| Atropa belladonna | G | S | 41.2 |
| Bellis perennis | G | S | 48.4 |
| Brassica oleracea | G | S | 26.4 |
| Brassica oleracea | G | O | 40.6 |
| Brassica rapa | G | S | 21.4 |
| Capsicum annuum | G | S | 35.0 |
| Capsicum annuum | G | S | 35.7 |
| Capsicum frutescens | G | S | 27.5 |
| Chelidonium majus | G | O | 34.7 |
| Cichorium intybus | G | R | 34.4 |
| Coix Lacryma-Jobi | G | S | 20.2 |
| Cosmos sulphureus | G | O | 32.9 |
| Crataegus submollis | G | S | 25.6 |
| Crataegus submollis | G | R | 28.6 |
| Cucumis anguria | G | S | 33.6 |
| Cucurbita maxima | G | S | 44.6 |
| Cucurbita moschata | G | S | 33.4 |
| Cucurbita pepo | G | S | 25.3 |
| Cymbopogon citratus | G | S | 30.3 |
| Cymbopogon martinii | G | S | 61.1 |
| Daucus carota | G | O | 30.0 |
| Dryopteris filix-mas | G | S | 26.0 |
| Dryopteris filix-mas | G | R | 45.3 |
| Echinacea purpurea | G | O | 51.8 |
| Echinochloa frumentacea | G | S | 30.3 |
| Fagopyrum esculentum | G | R | 50.9 |
| Fagopyrum tartaricum | G | O | 44.0 |
| Fagopyrum tartaricum | G | R | 46.0 |
| Filipendula rubra | G | S | 53.1 |
| Filipendula rubra | G | R | 58.7 |
| Forsythia intermedia | G | O | 52.9 |
| Fragaria x ananassa | G | R | 40.7 |
| Fragaria x ananassa | G | R | 28.1 |
| Gaultheria hispidula | G | R | 72.8 |
| Gaultheria hispidula | G | O | 100.0 |
| Gaultheria procumbens | G | R | 24.1 |
| Glycine max | G | S | 31.2 |
| Glycyrrhiza glabra | G | R | 37.1 |
| Guizotia abyssinica | G | R | 35.4 |
| Hamamelis virginiana | G | S | 29.1 |
| Hamamelis virginiana | G | R | 67.1 |
| Helenium hoopesii | G | R | 39.8 |
| Helianthus tuberosus | G | O | 32.8 |
| Hordeum hexastichon | G | S | 60.9 |
| Humulus lupulus | G | R | 61.2 |
| Humulus lupulus | G | S | 90.5 |
| Hypericum henryi | G | R | 100.0 |
| Hypericum perforatum | G | R | 43.4 |
| Hyssopus officinalis | G | S | 25.1 |
| Hyssopus officinalis | G | O | 48.2 |
| Iris versicolor | G | R | 47.0 |
| Isatis tinctoria | G | S | 32.1 |
| Lavandula angustifolia | G | S | 43.9 |
| Levisticum officinale | G | O | 51.4 |
| Malus hupehensis | G | S | 24.2 |
| Malus hupehensis | G | R | 37.2 |
| Malva sylvestris | G | O | 73.7 |
| Matricaria recutita | G | S | 31.5 |
| Melaleuca alternifolia | G | S | 21.5 |
| Melissa officinalis | G | S | 32.8 |
| Melissa officinalis | G | R | 44.8 |
| Melissa officinalis | G | O | 82.4 |
| Mentha piperita | G | R | 77.3 |
| Mentha pulegium | G | R | 41.1 |
| Monarda didyma | G | S | 31.8 |
| Nepeta cataria | G | R | 25.8 |
| Nepeta cataria | G | O | 84.9 |
| Nigella sativa | G | O | 44.9 |
| Ocinum tenuiflorum | G | R | 23.7 |
| Oenothera biennis | G | S | 25.6 |
| Origanum vulgare | G | S | 28.6 |
| Origanum vulgare | G | R | 31.2 |
| Pennisetum alopecuroides | G | S | 49.9 |
| Petroselinum crispum | G | S | 31.5 |
| Peucedanum oreaselinum | G | R | 68.3 |
| Phaseolus acutifolius | G | R | 25.4 |
| Phaseolus acutifolius | G | O | 61.8 |
| Phaseolus vulgaris | G | O | 24.4 |
| Phaseolus vulgaris | G | S | 35.6 |
| Phlox paniculata | G | S | 27.2 |
| Physalis alkekengi | G | R | 26.1 |
| Physalis alkekengi | G | O | 54.9 |
| Plantago major | G | O | 55.9 |
| Plectranthus sp. | G | R | 23.0 |
| Polygonum persicaria | G | S | 41.1 |
| Potentilla anserina | G | R | 55.4 |
| Poterium sanguisorba | G | R | 76.4 |
| Prunus cerasifera | G | R | 55.3 |
| Ptaridium aquilinus | G | R | 44.5 |
| Rhaphanus sativus | G | O | 98.1 |
| Rheum x cultorum | G | R | 27.0 |
| Ribes nidigrolaria | G | R | 22.0 |
| Ribes Silvestris | G | R | 88.8 |
| Rosmarinus officinalis | G | R | 39.4 |
| Rubus idaeus | G | S | 100.0 |
| Rubus ideaus | G | O | 37.0 |
| Rubus Phoenicalasius | G | R | 24.9 |
| Rubus pubescens | G | O | 23.0 |
| Rubus thibetanus | G | O | 41.2 |
| Rumex patientia | G | S | 36.2 |
| Salvia officinalis | G | O | 34.5 |
| Salvia officinalis | G | R | 89.5 |
| Sanguisorba officinalis | G | S | 46.8 |
| Santolina chamaecyparissus | G | R | 33.7 |
| Secale cereale | G | S | 24.4 |
| Senecio vulgaris | G | R | 37.6 |
| Solanum melongena | G | S | 21.1 |
| Solanum tuberosum | G | S | 27.6 |
| Sorghum dochna | G | S | 23.7 |
| Sorghum dochna | G | R | 56.3 |
| Symphytum officinale | G | S | 25.2 |
| Teucrium chamaedrys | G | S | 75.4 |
| Thymus praecox subsp arcticus | G | S | 28.4 |
| Thymus praecox subsp arcticus | G | O | 52.1 |
| Thymus x citriodorus | G | R | 25.3 |
| Triticum durum | G | S | 21.9 |
| Triticum turgidum | G | O | 80.2 |
| Vaccinium angustifolium | G | R | 47.6 |
| Vaccinium angustifolium | G | R | 48.1 |
| Vaccinium angustifolium | G | R | 71.0 |
| Vaccinium corymbosum | G | R | 60.6 |
| Vaccinium corymbosum | G | R | 61.7 |
| Vaccinium corymbosum | G | O | 99.4 |
| Vaccinium macrocarpon | G | R | 100.0 |
| Vaccinum angustifolium | G | O | 24.4 |
| Vaccinum angustifolium | G | R | 41.5 |
| Valeriana officinalis | G | R | 33.5 |
| Veronica officinalis | G | S | 27.0 |
| Vicia faba | G | O | 31.2 |
| Vicia faba | G | R | 44.7 |
| Vigna angularia | G | O | 40.8 |
| Vigna angularis | G | S | 39.4 |
| Vigna unguiculata | G | O | 26.1 |
| Vitis sp. | G | R | 62.4 |
| Vitis sp. | G | S | 63.3 |
| Vitis sp. | G | O | 82.0 |
| Withania somnifera | G | S | 22.4 |
| Xanthium strumarium | G | S | 20.7 |
| Zea mays | G | S | 26.1 |
| Zea mays | G | R | 67.5 |
| Abies lasiocarpa | T | R | 46.2 |
| Acorus calamus | T | R | 21.8 |
| Actinidia arguta | T | R | 64.6 |
| Agropyron repens | T | O | 48.3 |
| Alchemilla mollis | T | R | 100.0 |
| Alchemilla mollis | T | O | 100.0 |
| Allium cepa | T | R | 39.8 |
| Allium cepa | T | O | 45.2 |
| Allium tuberosum | T | R | 28.2 |
| Allium tuberosum | T | S | 28.8 |
| Alpinia officinarum | T | S | 26.4 |
| Amelanchier alnitolia | T | R | 78.3 |
| Amelanchier sanguinea x A. laevis | T | R | 66.5 |
| angelica archangelica | T | S | 25.2 |
| Apium graveolens | T | R | 43.3 |
| Aralia cordata | T | S | 31.5 |
| Aralia nudicaulis | T | S | 37.7 |
| Aralia nudicaulis | T | R | 48.5 |
| Aronia melanocarpa | T | S | 26.0 |
| Aronia melanocarpa | T | O | 53.3 |
| Aronia prunifolia | T | R | 79.2 |
| Artemisia absinthium | T | O | 100.0 |
| Artemisia dracunlus | T | S | 42.0 |
| Ayperus esculentus | T | O | 67.8 |
| Beta vulgaris | T | R | 27.9 |
| Beta vulgaris | T | S | 33.2 |
| Beta vulgaris | T | O | 53.0 |
| Borago officinalis | T | O | 55.7 |
| Brassica Napus | T | O | 71.9 |
| Brassica oleracea | T | O | 37.0 |
| Brassica oleracea | T | S | 46.9 |
| Brassica rapa | T | S | 36.7 |
| Bromus inermis | T | R | 42.8 |
| Calendula officinalis L. | T | S | 28.4 |
| Camellia sinensis syn. Thea sinensis | T | R | 86.4 |
| Capsicum annus | T | S | 29.7 |
| Capsicum annus | T | R | 43.7 |
| Capsicum frutescens (tabasco) | T | S | 22.0 |
| Carya cordiformis | T | R | 27.5 |
| Chaerophyllum bulbosum | T | S | 27.1 |
| Chaerophyllum bulbosum | T | O | 100.0 |
| Chelidonium majus | T | O | 54.0 |
| Chrysanthemum parthenium | T | S | 50.4 |
| Chrysanthenum coronarium | T | S | 25.8 |
| Cichorium inlybus | T | R | 23.9 |
| Citrullus lanatus | T | S | 33.2 |
| Citrullus lanatus (Garden baby) | T | S | 21.4 |
| Citrus limettoides | T | O | 39.2 |
| Citrus limon | T | O | 60.4 |
| Corchorus olitorius | T | S | 28.6 |
| Cornus canadensis L. | T | O | 50.0 |
| Cornus canadensis L. | T | R | 80.6 |
| Cosmos sulphureus | T | R | 20.5 |
| Cosmos sulphureus | T | S | 27.0 |
| Crataegus sp | T | S | 43.9 |
| Crataegus submollis | T | O | 24.2 |
| Crataegus submollis | T | R | 55.1 |
| Cucumis anguria | T | S | 33.2 |
| Cucumis sativus Fanfare | T | S | 35.4 |
| Cucurbita moschata | T | S | 30.4 |
| Cucurbita pepo | T | R | 23.8 |
| Cucurbita pepo | T | S | 46.6 |
| Cuminum cyminum | T | S | 23.1 |
| Curcuma zedoaria | T | S | 20.8 |
| Cymbopogon citratus | T | S | 39.7 |
| Dolichus lablab | T | S | 25.8 |
| Dryopteris filix-mas | T | O | 54.0 |
| Echinacea purpurea | T | S | 20.4 |
| Eriobotrya japonica | T | O | 34.8 |
| Eriobotrya japonica | T | S | 42.9 |
| Foericulum vulgare | T | O | 33.1 |
| Fragaria x ananassa | T | S | 20.3 |
| Fragaria x ananassa | T | R | 42.8 |
| Glycine max | T | O | 26.3 |
| Glycine max | T | O | 30.5 |
| Gossypium herbaceum | T | R | 22.5 |
| Guizotia abyssinica | T | R | 46.6 |
| Hamamelis virginiana | T | S | 33.1 |
| Hamamelis virginiana | T | S | 33.1 |
| Hamamelis virginiana | T | R | 44.8 |
| Hedeoma pulegiodes | T | O | 46.8 |
| Helenium hoopesii | T | R | 27.9 |
| Helianthus annus | T | S | 22.7 |
| Helianthus strumosus | T | O | 30.0 |
| Heliotropium arborescens | T | O | 53.7 |
| Helleborus niger | T | S | 40.5 |
| Hibiscus cannabinus | T | O | 34.0 |
| Hordeum vulgare subsp. Vulgare | I | O | 100.0 |
| Humulus lupulus | T | S | 24.9 |
| Humulus lupulus | T | R | 55.1 |
| Humulus lupulus | T | R | 77.6 |
| Humulus lupulus | T | S | 79.1 |
| Humulus lupulus | T | S | 100.0 |
| Humulus lupulus | T | R | 100.0 |
| Humulus lupulus | T | S | 100.0 |
| Hypericum henryl | T | R | 100.0 |
| Hypericum perforatum | T | O | 99.3 |
| Hypomyces lactiflorum | T | O | 20.5 |
| Iris versicolor | T | R | 48.5 |
| Juniperus communis | T | R | 33.8 |
| Lactuca serriola | T | R | 21.5 |
| Laportea canadensis | T | S | 37.7 |
| Lavendula angustifolia | T | S | 91.7 |
| Lepidium sativum | T | R | 24.7 |
| Levisticum officinale | T | O | 24.9 |
| Lolium perenne | T | S | 22.3 |
| Lonicera ramosissima | T | R | 42.5 |
| Lonicera syringantha | T | R | 21.1 |
| Malus | T | O | 53.1 |
| Malus hupehensis (Pamp.) Rehd. | T | R | 76.5 |
| Malus sp. | T | R | 39.8 |
| Malus sp. | T | R | 45.7 |
| Malva moschata | T | S | 22.8 |
| Malva sylvestris | T | O | 57.6 |
| Matteucia pensylvanica | T | R | 20.1 |
| Melissa officinalis | T | O | 55.0 |
| Mentha piperita | T | R | 35.5 |
| Mentha piperita | T | O | 43.9 |
| Mentha piperita | T | R | 56.6 |
| Mentha pulegium | T | O | 33.3 |
| Mentha pulegium | T | R | 56.2 |
| Mentha spicata | T | O | 43.4 |
| Mentha spicata | T | O | 58.0 |
| Nicotiana tabacum | T | R | 27.3 |
| Nigella sativa | T | R | 25.1 |
| Ocimum Basilicum | T | R | 20.2 |
| Ocnothera bienris | T | S | 37.8 |
| Origanum marjonara | T | R | 45.2 |
| Origanum vulgare | T | S | 21.3 |
| Origanum vulgare | T | O | 23.3 |
| Origanum vulgare | T | R | 23.6 |
| Origanum vulgare | T | O | 37.2 |
| Panicum miliaceum | T | S | 20.6 |
| Panicum miliaceum | T | S | 30.7 |
| Pastinaca saliva | T | R | 26.1 |
| Pastinaca sativa | T | O | 100.0 |
| Peucedanum oreaselinum | T | S | 39.6 |
| Peucedanum oreaselinum | T | R | 53.4 |
| Phaseolus vulgaris | T | S | 21.8 |
| Phaseolus vulgaris | T | O | 23.6 |
| Phaseolus vulgaris | T | O | 59.8 |
| Physalis alkekengi | T | O | 55.5 |
| Physalis pruinosa | T | S | 24.8 |
| Plantago major | T | O | 77.1 |
| Poa compressa | T | R | 54.4 |
| Polygonium chinense | T | O | 36.3 |
| Polygonium chinense | T | R | 61.4 |
| Polygonum persicaria | T | S | 21.3 |
| Populus incrassata | T | S | 50.7 |
| Populus incrassata | T | S | 50.7 |
| Populus x petrowskyana | T | R | 66.7 |
| Prunus cerasifera | T | O | 26.1 |
| Prunus cerasifera | T | R | 64.2 |
| Psidium guajaba | T | S | 22.9 |
| Ptaridium aquilinus | T | R | 43.0 |
| Pyrus pyrifolia | T | S | 28.2 |
| Rahmnus frangula | T | R | 25.9 |
| Raphanus sativus | T | R | 21.4 |
| Raphanus sativus | T | O | 36.9 |
| Rhamnus frangula | T | O | 43.2 |
| Rheum rhabarbarum | T | O | 28.5 |
| Rheum X cultorum | T | R | 28.2 |
| Rianus communis | T | S | 32.4 |
| Ribes nidigrolaria | T | S | 28.5 |
| Ribes nigrum | T | R | 49.9 |
| Rosa rugosa | T | S | 29.1 |
| Rosmarinum officinalis | T | R | 48.2 |
| Rubus arcticus | T | R | 59.1 |
| Rubus ideaus | T | O | 21.5 |
| Rubus pubescens | T | O | 51.8 |
| Rubus thibetanus | T | O | 33.7 |
| Rumex patientia | T | S | 34.4 |
| Ruta graveolens | T | O | 24.3 |
| Salvia (elegens) | T | O | 37.2 |
| Salvia (elegens) | T | R | 42.9 |
| Salvia officinalis | T | R | 67.3 |
| Sambucus canadensis | T | S | 30.2 |
| Sanguisorba minor | T | R | 21.0 |
| Sanguisorba minor | T | R | 29.9 |
| Sanguisorba minor | T | R | 30.8 |
| Sanguisorba minor | T | R | 44.5 |
| Santolina | T | R | 43.8 |
| Sarratula tinctoria | T | S | 37.7 |
| Satureja montana | T | R | 45.0 |
| Satureja repandra | T | S | 46.3 |
| Scorzorera hipanica | T | R | 25.7 |
| Scuttellaria lateriflora | T | S | 41.2 |
| Setaria italica | T | S | 33.4 |
| Solidago canadensis | T | S | 78.5 |
| Stachys affinis | T | S | 100.0 |
| Stachys byzantina | T | O | 100.0 |
| Stellaria media (linné) Cyrillo | T | O | 51.2 |
| Tanacetum vulgare | T | R | 30.5 |
| Tepary | T | R | 31.7 |
| Tepary | T | O | 39.7 |
| Thymus serpyllum | T | O | 29.9 |
| Thymus serpyllum | T | R | 32.8 |
| Thymus x citriodorus | T | S | 22.1 |
| Tiarella cordifolia | T | R | 46.8 |
| Tragopogon porrifolium | T | R | 26.3 |
| Tragopogon porrifolium | T | R | 29.8 |
| Tragopogon porrifolium | T | O | 58.0 |
| Triticale sp. | T | O | 25.3 |
| Tropaeolum majus | T | O | 46.9 |
| Tropaeolum majus | T | O | 55.8 |
| Tropaeolum majus | T | R | 64.7 |
| Tsuga can0adensis | T | R | 39.2 |
| Vaccinium angustifolium | T | R | 28.0 |
| Vaccinium angustifolium | T | S | 29.6 |
| Vaccinium angustifolium | T | R | 33.3 |
| Vaccinium angustifolium Ait. | T | R | 100.0 |
| Vaccinium macrocarpon | T | S | 25.1 |
| Vaccinium macrocarpon | T | R | 27.4 |
| Vaccinium macrocarpon | T | O | 35.4 |
| Vaccinium macrocarpon | T | R | 80.5 |
| Vaccinium macrocarpon | T | O | 90.5 |
| Valeriana officinalis | T | O | 33.0 |
| Veratrum viride | T | S | 46.8 |
| Verbascum thapsus | T | O | 33.4 |
| Vicia faba | T | R | 26.6 |
| Vicia faba | T | O | 35.8 |
| Vigna angularia | T | S | 29.3 |
| Vigna angularia | T | O | 54.0 |
| Vigna sesquipedalis | T | O | 100.0 |
| Vigna unguiculata | T | S | 49.5 |
| Vitia sp. | T | O | 99.6 |
| Vitis sp | T | R | 50.9 |
| Vitis sp. | T | R | 75.8 |
| Weigela coracensis | T | S | 22.8 |
| Weigela coracensis | T | S | 22.8 |
| Weigela hortensis | T | R | 54.9 |
| Zea mays | T | O | 74.3 |

TABLE 4
MMP-9

| | | | Inhibition |
|---|---|---|---|
| Nom latin | Stress | Extrait | (%) |
| Abelmochus esculentus | A | S | 26.8 |
| Achillea millefolium | A | S | 41.6 |
| Aconitum napellus | A | O | 47.7 |
| Acorus calamus | A | O | 83.2 |
| Actinidia arguta | A | S | 26.8 |
| Adiantum pedatum | A | O | 20.7 |
| Agastache foeniculum | A | S | 100.0 |
| Agrimonia eupatoria | A | W | 21.4 |
| Agropyron cristatum | A | R | 51.4 |
| Agropyron repens | A | S | 27.3 |
| Agrostis alba | A | R | 40.6 |
| Agrostis Stofonifera | A | R | 35.4 |
| Alcea rosea | A | S | 45.8 |
| Alkanna tinctoria | A | S | 42.5 |
| Allium cepa | A | O | 49.7 |
| Allium grande | A | R | 71.4 |
| Allium porrum | A | S | 28.0 |
| Allium porrum | A | O | 82.0 |
| Allium sativum | A | S | 23.7 |
| Allium schoenoprasum | A | O | 45.5 |
| Allium tuberosum | A | V | 20.1 |
| Allium Tuberosum | A | O | 91.5 |
| Althaea officinalis | A | S | 29.6 |
| Amaranthus gangeticus | A | O | 25.1 |
| Amaranthus gangeticus | A | R | 31.1 |
| Amaranthus gangeticus | A | S | 73.2 |
| Amaranthus retroflexus | A | S | 20.4 |
| Ambrosia artemisiifolia | A | R | 50.1 |
| Amelanchier sanguinea | A | W | 37.6 |
| Anthemis nobilis | A | O | 40.4 |
| Anthemis nobilis | A | R | 66.7 |
| Anthemis tinctorium | A | S | 30.3 |
| Apium graveolens | A | R | 71.2 |
| Arachis hypogaea | A | V | 23.5 |
| Aralia cordata | A | S | 21.2 |
| Aralia cordata | A | S | 56.3 |
| Arctium minus | A | R | 31.1 |
| Arctostaphylos uva-ursi | A | S | 31.2 |
| Arctostaphylos uva-ursi | A | O | 31.2 |
| Arctostaphylos uva-ursi | A | R | 59.7 |
| Armoracia rusticana | A | W | 25.1 |
| Armoracia rusticana | A | S | 56.2 |
| Aronia melanocarpa | A | S | 26.8 |
| Aronia melanocarpa | A | S | 41.3 |
| Aronia melanocarpa | A | O | 44.8 |
| Aronia melanocarpa | A | W | 47.7 |
| Aronia melanocarpa | A | R | 55.7 |
| Aronia melanocarpa | A | V | 100.0 |
| Arrhenatherum elatius | A | R | 40.4 |
| Artemisia dracunculus | A | S | 51.1 |
| Asparagus officinalis | A | S | 20.9 |
| Asparagus officinalis | A | S | 32.6 |
| Aster sp | A | O | 29.5 |
| Aster sp | A | R | 80.0 |
| Atropa belladonna | A | S | 47.4 |
| Beta vulgaris | A | S | 25.3 |
| Beta vulgaris | A | R | 26.6 |
| Beta vulgaris | A | W | 34.0 |
| Beta vulgaris | A | O | 42.0 |
| Beta vulgaris | A | V | 44.0 |
| Beta vulgaris spp. Maritima | A | R | 44.0 |
| Beta vulgaris var. condivata | A | R | 35.4 |
| Brassica napus | A | S | 24.6 |
| Brassica napus | A | R | 53.1 |
| Brassica napus | A | O | 100.0 |
| Brassica nigra | A | S | 24.2 |
| Brassica oleracea | A | R | 33.0 |
| Brassica oleracea | A | R | 36.0 |
| Brassica oleracea | A | W | 36.2 |
| Brassica oleracea | A | S | 73.1 |
| Brassica Oleracea | A | O | 100.0 |
| Brassica rapa | A | R | 31.0 |
| Brassica rapa | A | W | 38.6 |
| Brassica rapa | A | V | 42.8 |
| Brassica rapa | A | R | 48.8 |
| Brassica rapa | A | S | 68.2 |
| Brassica rapa | A | O | 89.2 |
| Bromus inermis | A | R | 51.4 |
| Campanula rapunculus | A | O | 25.1 |
| Canna edulis | A | S | 31.1 |
| Canna edulis | A | O | 47.6 |
| Canna edulis | A | R | 68.9 |
| Capsella bursa-pastoris | A | R | 32.5 |
| Capsicum annuum | A | O | 22.0 |
| Capsicum annuum | A | R | 24.0 |
| capsicum annuum | A | S | 55.7 |
| Capsicum frutescens | A | S | 30.3 |
| Capsicum frutescens | A | O | 34.7 |
| Carthamus tinctorius | A | R | 28.5 |
| Carum carvi | A | S | 38.6 |
| Chelidonium majus | A | O | 27.9 |
| Chenopodium bonus-henricus | A | R | 47.4 |
| Chenopodium bonus-henricus | A | O | 20.7 |
| Chenopodium bonus-henricus | A | W | 23.2 |
| chenopodium bonus-henricus | A | S | 62.8 |
| Chenopodium quinoa | A | V | 23.1 |
| Chenopodium quinoa | A | W | 34.7 |
| Chrysanthemum leucanthemum | A | O | 20.6 |
| Chrysanthemum leucanthemum | A | R | 30.9 |
| Chrysanthemun coronarium (Chp Suey) | A | R | 26.4 |
| Chrysanthenum coronarium | A | S | 66.6 |
| Cichorium intybus | A | S | 44.7 |
| Citrullus lanatus | A | S | 62.1 |
| Citrullus lanatus | A | O | 70.6 |
| Cornus canadensis | A | S | 48.5 |
| Cosmos sulphureus | A | S | 23.4 |
| Cosmos sulphureus | A | O | 37.0 |
| Crataegus sp | A | V | 32.4 |
| Crataegus sp | A | S | 45.5 |
| Crataegus sp | A | R | 100.0 |
| Crataegus submollis | A | S | 45.5 |
| Cryptotaenia canadensis | A | W | 26.4 |
| Cucumis Anguria | A | R | 27.2 |
| Cucumis anguria | A | S | 36.6 |
| Cucumis anguria | A | O | 38.5 |
| Cucumis melo | A | O | 59.2 |
| Cucumis sativus | A | R | 39.8 |
| Cucumis sativus | A | O | 49.4 |
| Cucumis sativus | A | S | 54.4 |
| Cucurbita Maxima | A | O | 46.7 |
| Cucurbita moschata | A | S | 32.1 |
| Cucurbita pepo | A | O | 37.0 |
| Curburbita pepo | A | R | 41.0 |
| Curburbita pepo | A | S | 43.9 |
| Curcuma zedoaria | A | S | 67.6 |
| Curcurbita maxima | A | S | 25.8 |
| Cymbopogon citratus | A | O | 26.7 |
| Dactylis glomerata | A | R | 27.2 |
| Datisca cannabina | A | S | 26.9 |
| Datisca cannabina | A | O | 38.0 |
| Daucus carota | A | R | 30.8 |
| Daucus carota | A | O | 31.9 |
| Dirca palustris | A | O | 27.3 |
| Dirca palustris | A | S | 34.2 |
| Dolicos Lablab | A | S | 22.0 |
| Dolicos Lablab | A | R | 25.3 |
| Dryopteris filix-mas | A | S | 24.9 |
| Dryopteris filix-mas | A | R | 40.6 |
| Eleusine coracana | A | S | 20.2 |
| Eleusine coracana | A | R | 20.9 |
| Eleusine coracana | A | O | 71.1 |
| Elymus junceus | A | R | 45.4 |
| Erigeron canadensis | A | S | 35.7 |
| Eruca vesicaria | A | R | 59.9 |
| Fagopyrum esculentum | A | V | 20.7 |
| Fagopyrum tartaricum | A | W | 30.3 |
| Fagopyrum tartaricum | A | O | 33.2 |
| Festuca rubra | A | R | 31.8 |
| Foeniculum Vulgare | A | W | 27.4 |
| Foeniculum vulgare | A | O | 50.6 |
| Forsythia intermedia | A | O | 100.0 |
| Fragaria x ananassa | A | V | 30.0 |
| Fragaria x ananassa | A | S | 36.3 |
| Galium odoratum | A | R | 26.9 |
| Gaultheria hispidula | A | R | 28.4 |
| Gaultheria hispidula | A | S | 40.7 |
| Gentiana lutea | A | R | 34.7 |
| Glechoma hederacea | A | S | 37.6 |
| Glycine max | A | R | 38.1 |
| Glycine Max | A | O | 56.4 |
| Glycine max | A | S | 71.4 |
| Glycyrrhiza glabra | A | S | 62.6 |
| Glycyrrhiza glabra | A | W | 100.0 |
| Guizotia abyssinica | A | R | 91.9 |
| Hamamelis virginiana | A | S | 41.0 |
| Hamamelis virginiana | A | R | 74.6 |
| Hedeoma pulegioides | A | O | 22.0 |
| Helianthus tuberosus | A | W | 21.2 |
| Helianthus tuberosus | A | W | 51.5 |
| Helichrysum angustifolium | A | V | 21.0 |
| Heliotropium arborescens | A | S | 54.1 |
| Helleborus niger | A | S | 37.8 |
| Hordeum hexastichon | A | W | 38.0 |
| Hyssopus officinalis | A | O | 25.1 |
| Inula helenium | A | S | 29.7 |
| Isatis tinctoria | A | S | 41.5 |
| Lactuca serrila | A | R | 41.3 |
| Lactuca serriola | A | S | 46.6 |
| Laportea canadensis | A | S | 26.3 |
| Lathyrus sativus | A | O | 22.2 |
| Lathyrus sativus | A | R | 50.2 |
| Lathyrus sylvestris | A | V | 31.3 |
| Lathyrus sylvestris | A | W | 31.8 |
| Laurus nobilis | A | S | 25.7 |
| Laurus nobilis | A | V | 30.0 |
| Lavandula latifolia | A | S | 40.3 |
| Leonurus cardiaca | A | R | 27.0 |
| Lepidium sativum | A | S | 41.8 |
| Levisticum officinale | A | S | 29.0 |
| Levisticum officinale | A | O | 44.9 |
| Linaria vulgaris miller | A | O | 23.6 |
| Linum usitatissimum | A | R | 33.3 |
| Lolium multiflorum | A | S | 29.0 |
| Lolium perenne | A | R | 52.0 |
| Lotus corniculatus | A | R | 62.9 |
| Lotus tetragonolobus | A | S | 62.9 |
| Lycopersicon esculentum | A | S | 26.1 |
| Lycopersicon esculentum | A | W | 33.0 |
| Malva moschata | A | S | 31.8 |
| Malva sylvestris | A | S | 21.4 |
| Malva verticillata | A | R | 43.4 |
| Matteucia pensylvanica | A | R | 26.9 |
| Medicago sativa | A | V | 20.4 |
| Melilotus albus | A | R | 53.9 |
| Melissa officinalis | A | S | 21.4 |
| Melissa officinalis | A | O | 36.8 |
| Melissa officinalis | A | R | 53.7 |
| Mentha piperita | A | S | 57.7 |
| Mentha pulegium | A | S | 66.1 |
| Mentha spicata | A | S | 67.7 |
| Mentha suaveolens | A | S | 51.8 |
| Momordica charantia | A | R | 29.7 |
| Momordica charantia | A | S | 72.1 |
| Nicotiana rustica | A | O | 30.3 |
| Nicotiana rustica | A | S | 59.1 |
| Nicotiana tabacum | A | S | 39.0 |
| Nicotiana tabacum | A | W | 47.6 |
| Nicotiana tabacum | A | O | 100.0 |
| Nigella sativa | A | R | 59.4 |
| Oenothera biennis | A | O | 21.3 |
| Oenothera biennis | A | O | 36.7 |
| Origanum vulgare | A | W | 21.3 |
| Origanum vulgare | A | V | 42.7 |
| Oryza sativa | A | W | 56.5 |
| Oxyria digyna | A | W | 35.1 |
| Oxyria digyna | A | V | 76.4 |
| Pastinaca sativa | A | V | 20.3 |
| Pastinaca sativa | A | W | 23.2 |
| Pastinaca sativa | A | O | 42.1 |
| Pastinaca sativa | A | R | 46.9 |
| Phalaris canariensis | A | R | 20.3 |
| Phalaris canariensis | A | O | 80.5 |
| Phaseolus mungo | A | O | 51.3 |
| Phaseolus mungo | A | S | 74.1 |
| Phaseolus vulgaris | A | V | 23.0 |
| Phaseolus vulgaris | A | O | 51.4 |
| Phaseolus vulgaris | A | S | 62.6 |
| Phlox paniculata | A | O | 41.0 |
| Physalis alkekengi | A | R | 31.6 |
| Physalis ixocarpa | A | S | 45.2 |
| Physalis ixocarpa | A | O | 65.3 |
| Physalis Pruinosa | A | O | 87.3 |
| Phytolacca americana | A | S | 49.6 |
| Phytolacca americana | A | O | 89.8 |
| Pimpinella anisum | A | S | 100.0 |
| Plantago coronopus | A | S | 48.3 |
| Plantago coronopus | A | O | 89.3 |
| Plantago major | A | S | 21.8 |
| Poa compressa | A | R | 22.4 |
| Poa compressa | A | S | 49.3 |
| Poa pratensis | A | R | 22.4 |
| Polygonum pensylvanicum | A | S | 43.3 |
| Polygonum persicaria | A | O | 21.6 |
| Polygonum persicaria | A | S | 38.5 |
| Potentilla anserina | A | S | 26.3 |
| Potentilla anserina | A | O | 31.2 |
| Poterium Sanquisorba | A | S | 29.2 |
| Pteridium aquilinum | A | S | 27.3 |
| Raphanus sativus | A | W | 22.7 |
| Raphanus sativus | A | R | 30.8 |
| Raphanus sativus | A | R | 40.2 |
| Raphanus sativus | A | S | 71.5 |
| Raphanus sativus | A | O | 100.0 |
| Rheum rhabarbarum | A | S | 21.3 |
| Rheum rhabarbarum | A | V | 67.9 |
| Rheum rhabarbarum | A | W | 72.4 |
| Ribes nidigrolaria | A | W | 32.6 |
| Ribes nidigrolaria | A | V | 64.6 |
| Ribes nigrum | A | W | 23.6 |
| Ribes nigrum | A | V | 27.2 |
| Ribes nigrum | A | S | 41.0 |
| Ribes nigrum | A | O | 65.8 |
| Ribes Nigrum | A | W | 100.0 |
| Ribes Salivum | A | R | 75.4 |
| Ribes Sylvestre | A | V | 27.7 |
| Ribes Sylvestre | A | W | 100.0 |
| ribes uva-crispa | A | S | 24.4 |
| Ribes Uva-crispa | A | W | 36.6 |
| Ricinus communis | A | R | 21.6 |
| Rosa rugosa | A | V | 30.6 |
| Rosa rugosa | A | S | 36.2 |
| Rosa rugosa | A | W | 39.3 |
| Rosmarinus officinalis | A | W | 27.2 |
| Rosmarinus officinalis | A | R | 45.7 |
| Rubus allegheniensis | A | S | 53.7 |
| Rubus canadensis | A | V | 27.0 |
| Rubus canadensis | A | S | 41.0 |
| Rubus canadensis | A | W | 41.2 |
| Rubus canadensis | A | S | 45.1 |
| Rubus idaeus | A | V | 24.3 |
| Rubus idaeus | A | S | 39.7 |
| Rubus idaeus | A | W | 62.2 |
| Rubus ideaus | A | R | 37.0 |
| Rumex acetosella | A | V | 75.8 |
| Rumex acotosa | A | W | 25.5 |
| Rumex crispus | A | R | 73.3 |
| Rumex crispus | A | O | 60.5 |
| Rumex patientia | A | O | 49.4 |
| Rumex patientia | A | S | 65.8 |
| Rumex Scutatus | A | W | 25.5 |
| Rumex Scutatus | A | V | 61.9 |
| Rumex Scutatus | A | O | 93.8 |
| Ruta graveolens | A | S | 25.8 |
| Ruta graveolens | A | W | 27.1 |
| Salix purpurea | A | S | 22.1 |
| Salix purpurea | A | R | 33.8 |
| Salvia elegans | A | W | 23.7 |
| Salvia officinalis | A | V | 20.8 |
| Salvia officinalis | A | S | 31.4 |
| Salvia sclarea | A | S | 28.0 |
| Satureja montana | A | W | 21.7 |
| Scuttellaria lateriflora | A | S | 54.1 |
| Secale cereale | A | V | 22.6 |
| Secale cereale | A | S | 22.9 |
| Secale cereale | A | W | 26.9 |
| Sesamum indicum | A | O | 21.2 |
| Setaria italica | A | O | 27.0 |
| Sium Sisarum | A | R | 32.6 |
| Sium Sisarum | A | O | 42.7 |
| Solanum dulcamara | A | S | 43.3 |
| Solanum dulcamara | A | O | 48.6 |
| Solanum melanocerasum | A | O | 21.3 |
| Solanum melongena | A | R | 20.5 |
| Solanum melongena | A | V | 35.6 |
| Solanum melongena | A | O | 49.4 |
| Solanum melongena | A | S | 65.2 |
| Solidago sp | A | R | 32.7 |
| Spinacia oleracea | A | S | 41.0 |
| Stachys affinis | A | R | 22.5 |
| Stachys affinis | A | S | 43.9 |
| Stachys affinis | A | O | 92.0 |
| Symphytum officinale | A | S | 28.0 |
| Tanacetum cinerariifolium | A | O | 20.3 |
| Tanacetum cinerariifolium | A | R | 69.7 |
| Tanacetum vulgare | A | O | 20.2 |
| Tanacetum vulgare | A | S | 84.2 |
| Teucrium chamaedrys | A | O | 20.4 |
| Teucrium chamaedrys | A | R | 20.4 |
| Thymus serpyllum | A | W | 24.3 |
| Thymus vulgaris | A | S | 42.5 |
| Thymus x citriodorus | A | W | 27.4 |
| Tragopogon porrifolius | A | W | 21.9 |
| Tragopogon porrifolius | A | V | 26.2 |
| Trifolium hybridum | A | R | 30.9 |
| Trifolium pannonicum | A | R | 41.0 |
| Trifolium repens | A | R | 51.3 |
| Trigonella foenum graecum | A | S | 44.2 |
| Triticum spelta | A | S | 30.0 |
| Triticum turgidum | A | S | 31.3 |
| Typha latifolia | A | S | 57.7 |
| Urtica dioica | A | O | 26.5 |
| Urtica dioica | A | S | 50.2 |
| Vaccinium Corymbosum | A | W | 39.9 |
| Vaccinium Corymbosum | A | S | 64.8 |
| Vaccinum augustifolium | A | R | 44.8 |
| Vaccinum macrocarpon | A | S | 100.0 |
| Veratrum viride | A | S | 29.1 |
| Veratrum viride | A | O | 31.8 |
| Verbascum thapsus | A | S | 42.6 |
| Verbascum thapsus | A | O | 75.2 |
| Viburnum trilobum | A | V | 97.4 |
| Vicia sativa | A | R | 53.3 |
| Vicia villosa | A | R | 48.9 |
| Vigna unguiculata | A | R | 27.0 |
| Vigna unguiculata | A | O | 44.8 |
| Vigna unguiculata | A | S | 55.5 |
| Vinca minor | A | S | 35.1 |
| Vitis sp. | A | V | 52.2 |
| Vitis sp. | A | S | 59.6 |
| Vitis sp. | A | R | 87.8 |
| Xanthium sibiricum | A | S | 57.1 |
| Zea mays | A | V | 26.1 |
| Zea mays | A | W | 32.1 |
| Zea Mays | A | O | 38.7 |
| Achillea millefolium | G | S | 45.5 |
| Aconitum napellus | G | S | 24.0 |
| Aconitum napellus | G | O | 53.9 |
| Acorus calamus | G | O | 87.6 |
| Acorus calamus | G | S | 100.0 |
| Actinidia arguta | G | S | 33.8 |
| Adiantum pedatum | G | R | 31.6 |
| Adiantum pedatum | G | S | 31.7 |
| Ageratum conyzoides | G | S | 23.1 |
| Agropyron cristatum | G | R | 64.1 |
| Agropyron repens | G | S | 29.2 |
| Agropyron repens | G | O | 32.6 |
| Agrostis Stolonifera | G | R | 34.4 |
| Alcea rosea | G | S | 22.7 |
| Alchemilla mollis | G | S | 30.5 |
| Alchemilla mollis | G | W | 33.2 |
| Allium ampeloprasum | G | O | 53.4 |
| Allium cepa | G | S | 22.5 |
| Allium cepa | G | O | 60.7 |
| Allium schoenoprasum | G | S | 21.1 |
| Allium schoenoprasum | G | O | 60.4 |
| Allium tuberosum | G | S | 38.8 |
| Allium tuberosum | G | O | 74.4 |
| Althaea officianalis | G | S | 54.9 |
| Amaranthus candathus | G | O | 42.6 |
| Amaranthus caudathus | G | W | 27.1 |
| Amaranthus gangeticus | G | S | 56.8 |
| Amaranthus gangeticus | G | S | 74.4 |
| Ambrosia artemisiifolia | G | R | 49.0 |
| Amelanchier sanguinea | G | W | 45.2 |
| Angelica archangelica | G | S | 20.9 |
| Anthemis nobilis | G | R | 58.9 |
| Apium graveolens | G | O | 30.4 |
| Apium graveolens | G | S | 36.4 |
| Apium graveolens | G | R | 60.6 |
| Arachis hypogaea | G | W | 26.0 |
| Aralia cordata | G | S | 66.0 |
| Arctium minus | G | O | 26.6 |
| Arctium minus | G | R | 30.8 |
| Arctostaphylos uva-ursi | G | S | 29.3 |
| Arctostaphylos uva-ursi | G | O | 38.8 |
| Arctostaphylos uva-ursi | G | R | 80.2 |
| Armoracia rusticana | G | S | 62.7 |
| Aronia melanocarpa | G | O | 26.7 |
| Aronia melanocarpa | G | V | 100.0 |
| Aronia melanocarpa | G | R | 100.0 |
| Aronia melanocarpa (Michx.) Ell. | G | W | 39.1 |
| Artemisia dracunculus | G | O | 44.3 |
| Artemisia dracunculus | G | S | 65.4 |
| Asclepias incarnata | G | R | 20.3 |
| Asparagus officinalis | G | O | 22.3 |
| Asparagus officinalis | G | S | 26.6 |
| Asparagus officinalis | G | W | 28.7 |
| Aster sp | G | O | 34.3 |
| Aster sp | G | R | 62.6 |
| Atropa belladonna | G | S | 34.9 |
| Beta vulgaris | G | R | 28.3 |
| Beta vulgaris | G | R | 42.2 |
| Beta vulgaris | G | O | 47.0 |
| Beta vulgaris spp. Maritima | G | O | 46.7 |
| Brassica cepticepa | G | R | 26.7 |
| Brassica cepticepa | G | S | 68.3 |
| Brassica juncea | G | O | 45.0 |
| Brassica juncea | G | S | 66.1 |
| Brassica Napus | G | S | 27.5 |
| Brassica Napus | G | R | 37.6 |
| Brassica napus | G | O | 94.8 |
| Brassica nigra | G | S | 36.4 |
| Brassica oleracea | G | R | 38.7 |
| Brassica oleracea | G | W | 39.0 |
| Brassica oleracea | G | R | 49.4 |
| Brassica oleracea | G | S | 76.1 |
| Brassica oleracea | G | O | 100.0 |
| Brassica rapa | G | R | 21.1 |
| Brassica rapa | G | S | 64.0 |
| Brassica rapa | G | O | 100.0 |
| Bromus inermis | G | R | 36.7 |
| Campanula rapunculus | G | O | 59.9 |
| Canna edulis | G | O | 20.8 |
| Canna edulis | G | O | 83.1 |
| Capsicum annuum | G | R | 20.2 |
| Capsicum annuum | G | S | 29.6 |
| Capsicum annuum | G | O | 51.5 |
| Capsicum annuum | G | S | 60.8 |
| Capsicum frutescens | G | S | 32.8 |
| Carthamus tinctorius | G | R | 29.8 |
| Carum carvi | G | S | 30.4 |
| Chelidonium majus | G | O | 39.9 |
| Chenopodium bonus-henricus | G | O | 63.0 |
| Chenopodium quinoa | G | O | 34.1 |
| Chenopodium quinoa | G | W | 42.8 |
| Chenopodium quinoa | G | V | 46.1 |
| Chichorium endivia subsp endivia | G | W | 22.0 |
| Chichorium endivia subsp endivia | G | S | 22.9 |
| Chrysanthemum coronarium | G | R | 23.2 |
| Chrysanthemum coronarium | G | S | 68.4 |
| Chrysanthemum leucanthemum | G | R | 20.5 |
| Cicer arietinum | G | S | 25.7 |
| Cichorium intybus | G | W | 51.1 |
| Cichorium intybus | G | S | 53.4 |
| Citrullus lanatus | G | S | 36.5 |
| Citrullus lanatus | G | O | 71.5 |
| Coix Lacryma-Jobi | G | O | 21.0 |
| Cornus canadensis | G | S | 34.8 |
| Crataegus sp | G | W | 54.0 |
| Crataegus submollis | G | S | 31.3 |
| Cryptotaenia canadensis | G | W | 32.1 |
| Cucumis anguria | G | S | 27.3 |
| Cucumis anguria | G | O | 32.5 |
| Cucumis sativus | G | O | 39.4 |
| Cucumis sativus | G | S | 69.4 |
| Cucurbita maxima | G | O | 34.1 |
| Cucurbita maxima | G | S | 42.6 |
| Cucurbita moschata | G | S | 32.0 |
| Cucurbita moschata | G | O | 39.2 |
| Cucurbita pepo | G | S | 28.8 |
| Cucurbita pepo | G | O | 32.6 |
| Curcuma zedoaria | G | O | 23.3 |
| Curcuma zedoaria | G | S | 57.6 |
| Cymbopogon citratus | G | O | 70.1 |
| Cynara scolymus | G | S | 20.2 |
| Cynara scolymus | G | O | 37.5 |
| Cynara scolymus | G | R | 88.7 |
| Cyperus esculentus | G | S | 66.7 |
| Datura metel | G | S | 29.2 |
| Datura stramonium | G | O | 27.6 |
| Daucus carota | G | O | 24.2 |
| Daucus carota | G | R | 29.3 |
| Dipsacus sativus | G | S | 48.7 |
| Dirca palustris | G | O | 29.9 |
| Dirca palustris | G | S | 36.4 |
| Dolichos Lablab | G | S | 35.8 |
| Dolichos Lablab | G | R | 74.5 |
| Dryopteris filix-mas | G | S | 27.9 |
| Dryopteris filix-mas | G | R | 42.6 |
| Echinochloa frumentacea | G | O | 68.4 |
| Eleusine coracana | G | O | 47.8 |
| Elymus junceus | G | R | 42.7 |
| Erigeron canadensis | G | S | 37.8 |
| Erigeron speciosus | G | R | 34.6 |
| Errhenatherum elatius | G | R | 34.4 |
| Fagopyrum tartaricum | G | W | 31.4 |
| Foeniculum vulgare | G | W | 28.0 |
| Foeniculum vulgare | G | S | 44.6 |
| Foeniculum vulgare | G | O | 68.9 |
| Foeniculum Vulgare | G | R | 100.0 |
| Forsythia intermedia | G | O | 100.0 |
| Forsythia x intermedia | G | O | 79.5 |
| Galium odoratum | G | S | 32.4 |
| Galium odoratum | G | R | 100.0 |
| Gaultheria hispidula | G | R | 48.4 |
| Gaultheria hispidula | G | S | 80.4 |
| Gaultheria hispidula | G | O | 100.0 |
| Gaultheria procumbens | G | S | 26.9 |
| Gaultheria procumbens | G | W | 54.3 |
| Glechoma hederacea | G | S | 26.6 |
| Glycine max | G | R | 52.5 |
| Glycine max | G | O | 67.9 |
| Glycine max | G | O | 75.8 |
| Glycyrrhiza glabra | G | R | 21.4 |
| Glycyrrhiza glabra | G | V | 21.6 |
| Glycyrrhiza glabra | G | W | 100.0 |
| Guizotia abyssinica | G | R | 91.4 |
| Hamamelis virginiana | G | O | 39.8 |
| Hamamelis virginiana | G | R | 78.8 |
| Hamamelis virginiana | G | S | 96.6 |
| Hedeoma pulegioides | G | S | 45.4 |
| Helenium hoopesii | G | S | 22.6 |
| Helenium hoopesii | G | O | 52.8 |
| Helianthus annuus | G | R | 22.0 |
| Helianthus annuus | G | S | 31.6 |
| Helianthus strumosus | G | R | 30.5 |
| Helianthus strumosus | G | O | 71.7 |
| Helianthus tuberosus | G | W | 21.2 |
| Helianthus tuberosus | G | S | 50.7 |
| Helianthus tuberosus L. | G | R | 24.9 |
| Heliotropium arborescens | G | S | 40.0 |
| Heliotropium arborescens | G | O | 45.6 |
| Helleborus niger | G | S | 38.0 |
| Hordeum vulgare | G | S | 21.5 |
| Humulus lupulus | G | O | 35.1 |
| Hypericum sp | G | W | 26.1 |
| Hyssopus officinalis | G | S | 74.5 |
| Iberis amara | G | O | 20.9 |
| Iberis amara | G | S | 21.7 |
| Inula helenium | G | S | 27.6 |
| Ipomoea batatas | G | S | 37.5 |
| Isatis tinctoria | G | S | 48.0 |
| Lachica serrola | G | R | 53.0 |
| Lactuca sativa | G | W | 24.5 |
| Laportea canadensis | G | S | 36.0 |
| Laportea canadensis | G | O | 81.7 |
| Lathyrus sativus | G | W | 37.8 |
| Lathyrus sylvestris | G | R | 40.7 |
| Lathyrus sylvestris | G | O | 79.1 |
| Laurus nobilis | G | S | 22.7 |
| Lavandula angustifolia | G | S | 31.7 |
| Lavandula latifolia | G | O | 27.2 |
| Ledum groenlandicum | G | S | 61.1 |
| Leonurus cardiaca | G | O | 22.6 |
| Lepidium sativum | G | S | 23.3 |
| Levisticum officinale | G | S | 23.1 |
| Levisticum officinale | G | W | 27.5 |
| Levisticum officinale | G | O | 41.3 |
| Linum usitatissimum | G | R | 21.4 |
| Lolium perenne | G | R | 32.7 |
| Lotus corniculatus | G | R | 54.2 |
| Malus hupehensis | G | R | 26.4 |
| Malva verticillata | G | R | 37.9 |
| Matricaria recutita | G | O | 50.3 |
| Medicago sativa | G | W | 29.1 |
| Melilotus albus | G | R | 52.1 |
| Melissa officinalis | G | O | 22.7 |
| Melissa officinalis | G | S | 35.9 |
| Melissa officinalis | G | R | 38.6 |
| Mentha piperita | G | S | 64.4 |
| Mentha suaveolens | G | W | 22.5 |
| Momordica charantia | G | R | 29.3 |
| Momordica charantia | G | S | 90.6 |
| Nepeta cataria | G | R | 50.5 |
| Nicotiana rustica | G | O | 35.3 |
| Nicotiana rustica | G | S | 100.0 |
| Nicotiana tabacum | G | S | 31.6 |
| Nicotiana tabacum | G | O | 100.0 |
| Nigella sativa | G | R | 24.2 |
| Ocimum basilicum | G | S | 30.6 |
| Oenothera biennis | G | O | 48.0 |
| Oenothera biennis | G | R | 76.6 |
| Origanum vulgare | G | V | 41.3 |
| Oryza Sativa | G | O | 22.1 |
| Oxyria digyna | G | O | 26.5 |
| Oxyria digyna | G | V | 70.3 |
| Panicum miliaceum | G | O | 94.4 |
| Pastinaca sativa | G | R | 29.4 |
| Pastinaca sativa | G | S | 79.2 |
| Pennisetum alopecuroides | G | O | 22.0 |
| Petasites japonicus | G | S | 29.2 |
| Peucedanum oreaselinum | G | O | 21.3 |
| Phacelia tanacetifolia | G | R | 23.5 |
| Phalaris arundinacea | G | R | 47.5 |
| Phalaris canariensis | G | R | 23.1 |
| Phalaris canariensis | G | O | 100.0 |
| Phaseolus coccineus | G | O | 37.0 |
| Phaseolus coccineus | G | R | 74.1 |
| Phaseolus mungo | G | O | 42.2 |
| Phaseolus mungo | G | S | 52.2 |
| Phaseolus vulgaris | G | V | 35.5 |
| Phaseolus vulgaris | G | S | 48.0 |
| Phaseolus vulgaris | G | O | 58.1 |
| Phlox paniculata | G | S | 32.2 |
| Phlox paniculata | G | O | 40.1 |
| Physalis-ixocarpa | G | O | 20.6 |
| Physalis pruinosa | G | O | 80.0 |
| Phytolacca americana | G | S | 62.0 |
| Phytolacca americana | G | O | 100.0 |
| Pimpinella anisum | G | S | 37.3 |
| Pisum sativum | G | W | 34.4 |
| Pisum sativum | G | O | 63.3 |
| Plantago coronopus | G | O | 42.7 |
| Plantago coronopus | G | S | 46.4 |
| Plantago major | G | O | 28.3 |
| Plantago major | G | S | 41.4 |
| Plectranthus sp. | G | S | 29.3 |
| Poa compressa | G | R | 22.1 |
| Poa compressa | G | S | 45.5 |
| Poa pratensis | G | R | 35.7 |
| Polygonum pensylvanicum | G | S | 38.3 |
| Polygonum persicaria | G | S | 31.0 |
| Potentilla anserina | G | O | 46.8 |
| Poterium sanquisorba | G | S | 24.7 |
| Poterium sanquisorba | G | W | 30.6 |
| Prunus cerasifera | G | R | 45.9 |
| Pteridium aquilinum | G | S | 22.4 |
| Raphanus Raphanistrum | G | S | 36.5 |
| Raphanus Raphanistrum | G | O | 75.0 |
| Raphanus sativus | G | R | 20.8 |
| Raphanus sativus | G | R | 27.5 |
| Raphanus sativus | G | S | 35.4 |
| Rheum rhabarbarum | G | S | 27.0 |
| Ribes Grossularia | G | W | 33.7 |
| Ribes nidigrolaria | G | S | 30.7 |
| Ribes nidigrolaria | G | V | 40.5 |
| Ribes nigrum | G | V | 35.9 |
| Ribes nigrum | G | W | 58.6 |
| Ribes Silvestris | G | V | 26.9 |
| Ribes Silvestris | G | W | 100.0 |
| Ricinus communis | G | R | 21.8 |
| Rosmarinus officinalis | G | S | 24.7 |
| Rosmarinus officinalis | G | W | 30.9 |
| Rosmarinus officinalis | G | R | 60.3 |
| Rubus ideaus | G | O | 32.5 |
| Rubus ideaus | G | S | 47.0 |
| Rubus occidentalis | G | S | 39.4 |
| Rubus occidentalis | G | R | 74.1 |
| Rumex acetosa | G | W | 45.6 |
| Rumex acetosella | G | W | 22.8 |
| Rumex acetosella | G | V | 31.5 |
| Rumex crispus | G | O | 25.9 |
| Rumex crispus | G | R | 70.3 |
| Rumex patientia | G | O | 39.8 |
| Rumex patientia | G | S | 54.2 |
| Rumex scutatus | G | W | 23.8 |
| Rumex scutatus | G | V | 69.9 |
| Rumex scutatus | G | O | 78.8 |
| Ruta graveolens | G | R | 30.7 |
| Ruta graveolens | G | S | 61.5 |
| Salvia elagens | G | W | 25.4 |
| Salvia elegans | G | S | 31.1 |
| Sambucus canadensis | G | W | 80.6 |
| Sambucus ebulus | G | W | 26.1 |
| Sambucus ebulus | G | V | 34.4 |
| Sambucus ebulus | G | S | 37.8 |
| Sanguisorba officinalis | G | R | 100.0 |
| Santolina chamaecyparissus | G | R | 21.7 |
| Santolina chamaecyparissus | G | S | 25.2 |
| Satureja montana | G | O | 21.2 |
| Scutiellaria lateriflora | G | S | 37.0 |
| Secale cereale | G | S | 26.7 |
| Secale cereale | G | W | 27.3 |
| Serratula tinctoria | G | S | 36.2 |
| Serratula tinctoria | G | O | 70.3 |
| Sesamum indicum | G | O | 27.6 |
| Sesamum indicum | G | S | 44.3 |
| Silybum marianum | G | S | 34.7 |
| Sium sisarum | G | O | 79.0 |
| Solanum dulcamara | G | R | 25.2 |
| Solanum dulcamara | G | S | 64.6 |
| solanum melongena | G | S | 36.6 |
| solanum melongena | G | O | 40.1 |
| solanum melongena | G | V | 50.0 |
| solanum melongena | G | S | 74.9 |
| Solanum tuberosum | G | S | 39.1 |
| Solanum tuberosum | G | O | 39.2 |
| Solidago sp | G | R | 30.7 |
| Sorghum caffrorum | G | O | 87.9 |
| Sorghum dochna | G | W | 20.6 |
| Sorghum dochna | G | O | 20.6 |
| Sorghum dochna | G | S | 34.1 |
| Sorghum dochna | G | O | 97.0 |
| Sorghum durra | G | O | 30.6 |
| sorghum durra | G | S | 30.6 |
| sorghum durra | G | O | 48.0 |
| Sorghum sudanense | G | S | 21.7 |
| Sorghum sudanense | G | O | 24.6 |
| Sorghum sudanense | G | V | 32.1 |
| Spinacia oleracea | G | S | 53.2 |
| Stachys Affinis | G | S | 25.0 |
| Stachys Affinis | G | R | 27.8 |
| Stachys-Affinis | G | O | 100.0 |
| Symphytum officinale | G | W | 21.7 |
| Symphytum officinale | G | O | 25.2 |
| Symphytum officinale | G | S | 34.6 |
| Tanacetum cinerariifolium | G | R | 52.4 |
| Tanacetum vulgare | G | R | 27.1 |
| Tanacetum vulgare | G | S | 72.7 |
| Teucrium chamaedrys | G | R | 24.6 |
| Teucrium chamaedrys | G | O | 52.8 |
| Thymus fragantissumus | G | R | 100.0 |
| Thymus vulgaris | G | V | 24.2 |
| Thymus x citriodorus | G | S | 23.7 |
| Tiarella cordifolia | G | S | 20.8 |
| Tiarella cordifolia | G | O | 30.8 |
| Tragopogon porrifolius | G | O | 22.8 |
| Trifolium hybridum | G | R | 24.7 |
| Trifolium pannonicum | G | R | 65.5 |
| Trifolium repens | G | R | 57.5 |
| Trigonella foenumgraecum | G | S | 37.6 |
| Triticum furgidum | G | S | 56.5 |
| Triticum spelta | G | S | 40.8 |
| Tropaeolum majus | G | O | 76.1 |
| Typha latifolia | G | S | 43.3 |
| Urtica dioica | G | S | 40.3 |
| Vaccinium angustifolium | G | S | 42.4 |
| Vaccinium corymbosum | G | S | 61.5 |
| Vaccinium macrocarpon | G | S | 43.7 |
| Vaccinium angustifolium | G | R | 23.1 |
| Veratrum viride | G | S | 43.6 |
| Verbascum thapsus | G | S | 37.8 |
| Verbascum thapsus | G | O | 87.0 |
| Veronica officinalis | G | S | 30.5 |
| Viburnum trilobum | G | S | 49.4 |
| Viburnum trilobum | G | R | 100.0 |
| Viburnum trilobum | G | V | 100.0 |
| Vicia faba | G | R | 50.5 |
| Vicia sativa | G | R | 42.4 |
| Vicia villosa | G | R | 89.2 |
| Vigna angularia | G | R | 28.1 |
| Vigna angularia | G | S | 71.5 |
| Vigna unguiculata | G | R | 21.0 |
| Vigna unguiculata | G | O | 38.7 |
| Vigna unguiculata | G | S | 61.1 |
| Vinca minor | G | O | 33.6 |
| Vinca minor | G | S | 34.3 |
| Vitis sp. | G | O | 29.0 |
| Vitis sp. | G | W | 50.2 |
| Vitis sp. | G | S | 53.3 |
| Vitis sp. | G | V | 63.0 |
| Vitis sp. | G | R | 86.6 |
| Withania somnifera | G | S | 20.3 |
| Xanthium sibiricum | G | S | 34.7 |
| Xanthium strumarium | G | S | 23.2 |
| Zea mays | G | V | 20.1 |
| Zea mays | G | S | 45.9 |
| Zea mays | G | O | 97.5 |
| Abelmochus esculentus | T | S | 24.8 |
| Abies lasiocarpa | T | W | 44.7 |
| Achillea millefolium | T | O | 24.1 |
| Achillea millefolium | T | S | 59.2 |
| Aconitum napellus | T | S | 40.6 |
| Aconitum napellus | T | O | 41.6 |
| Acorus calamus | T | O | 47.1 |
| Actinidia arguta | T | S | 21.8 |
| Adiantum pedatum | T | S | 26.8 |
| Adiantum pedatum | T | O | 45.8 |
| Adiantum pedatum | T | R | 86.0 |
| Agaricus bisporus | T | S | 26.3 |
| Agaricus bisporus | T | O | 29.8 |
| Agaricus bisporus | T | W | 36.9 |
| Agaricus bisporus | T | W | 44.0 |
| Agaricus bisporus | T | S | 46.0 |
| Agastache foeniculum | T | S | 70.0 |
| Ageratum conyzoides | T | S | 31.7 |
| Agropyron cristatum | T | R | 86.9 |
| Agropyron repens | T | O | 49.6 |
| Agrostis alba | T | R | 21.9 |
| Agrostis Stolonifera | T | R | 35.8 |
| Alcea rosea | T | S | 35.2 |
| Alchemilla mollis | T | S | 37.9 |
| Allium ampeloprasum | T | O | 48.0 |
| Allium ascalonicum | T | S | 26.2 |
| Allium ascalonicum | T | O | 77.2 |
| Allium cepa | T | O | 92.6 |
| Allium grande | T | R | 60.4 |
| Allium schoenoporasum | T | O | 65.8 |
| Allium schoenoprasum | T | W | 31.0 |
| Allium tuberosum | T | S | 22.8 |
| Allium tuberosum | T | O | 99.7 |
| Althaea officianalis | T | S | 22.8 |
| Althaea officinalis | T | O | 22.1 |
| Amaranthus candathus | T | W | 43.9 |
| Amaranthus gangeticus | T | O | 30.3 |
| Amaranthus gangeticus | T | S | 66.0 |
| Ambrosia artemisiifolia | T | R | 58.7 |
| Amelanchier alnitolia | T | R | 70.5 |
| Amelanchier sanguinea | T | W | 37.3 |
| Ananas comosus | T | W | 23.8 |
| Ananas comosus | T | V | 95.0 |
| Ananas comosus | T | O | 99.6 |
| angelica archangelica | T | S | 30.5 |
| angelica archangelica | T | R | 38.9 |
| Anthemis nobilis | T | O | 41.4 |
| Anthemis nobilis | T | R | 72.8 |
| Anthemis tinctorium | T | S | 27.3 |
| Anthriscus cerefolium | T | W | 35.8 |
| Apium graveolens | T | S | 31.7 |
| Apium graveolens | T | W | 32.4 |
| Apium graveolens | T | R | 56.6 |
| Aralia cordata | T | R | 29.2 |
| Aralia cordata | T | S | 45.0 |
| Arctium minus | T | R | 25.8 |
| Arctostaphylos uva-ursi | T | O | 31.0 |
| Arctostaphylos uva-ursi | T | S | 35.2 |
| Arctostaphylos uva-ursi | T | R | 58.6 |
| Armoracia rusticana | T | W | 24.9 |
| Armoracia rusticana | T | S | 52.9 |
| Aronia melanocarpa | T | W | 40.0 |
| Aronia melanocarpa | T | V | 91.9 |
| Aronia prunifolia | T | W | 100.0 |
| Arrhenatherum elatius | T | R | 22.8 |
| Artemisia draculus | T | S | 74.9 |
| Artemisia dracunculus | T | S | 47.8 |
| Asclepias incarnata | T | R | 20.5 |
| Asctinidia chinensis | T | V | 43.4 |
| Asctinidia chinensis | T | O | 66.4 |
| Asparagus officinalis | T | O | 91.3 |
| Asparagus officiralis | T | R | 23.3 |
| Asparagus officiralis | T | S | 44.7 |
| Aster Linné | T | S | 47.5 |
| Aster sp | T | R | 62.0 |
| Atriplex hortensis | T | R | 54.6 |
| Atropa belladonna | T | R | 20.1 |
| Atropa belladonna | T | S | 51.0 |
| Avena sativa | T | R | 24.8 |
| Avena sativa | T | W | 26.4 |
| Averrhoa carambola | T | W | 23.4 |
| Ayperus esculentus | T | S | 46.2 |
| Beta vulgaris | T | R | 28.2 |
| Beta vulgaris | T | S | 30.4 |
| Beta vulgaris | T | O | 56.8 |
| Beta vulgaris spp. Maritima | T | R | 23.6 |
| Betula glandulosa | T | O | 22.2 |
| Betula glandulosa | T | V | 22.2 |
| Betula glandulosa | T | S | 25.7 |
| Betula glandulosa | T | W | 32.9 |
| Boletus edulis | T | S | 36.2 |
| Boletus edulis | T | O | 90.2 |
| Borago officinalis | T | S | 27.9 |
| Borago officinalis | T | O | 76.1 |
| Brassica cepticepa | T | O | 65.4 |
| Brassica cepticepa | T | S | 71.5 |
| Brassica Chineusis | T | R | 27.1 |
| Brassica juncea | T | O | 51.0 |
| Brassica juncea | T | R | 66.0 |
| Brassica juncea | T | S | 74.1 |
| Brassica Napus | T | S | 22.0 |
| Brassica Napus | T | R | 34.0 |
| Brassica Napus | T | O | 100.0 |
| Brassica nigra | T | S | 26.7 |
| Brassica nigra | T | O | 27.4 |
| Brassica nigra | T | R | 82.5 |
| Brassica oleracea | T | O | 21.2 |
| Brassica oleracea | T | S | 22.1 |
| Brassica oleracea | T | W | 26.2 |
| Brassica oleracea | T | R | 27.2 |
| Brassica oleracea | T | O | 31.3 |
| Brassica oleracea | T | W | 46.5 |
| Brassica oleracea | T | S | 71.2 |
| Brassica oleracea | T | O | 93.5 |
| Brassica rapa | T | R | 25.6 |
| Brassica rapa | T | R | 33.9 |
| Brassica rapa | T | R | 56.0 |
| Brassica rapa | T | S | 69.7 |
| Brassica rapa | T | O | 100.0 |
| Bromus inermis | T | R | 57.3 |
| Campanula rapunculus | T | O | 77.5 |
| Canna edulis | T | O | 75.6 |
| Cantharellus ciparium | T | O | 52.5 |
| Capsella bursa-pastoris | T | O | 35.9 |
| Capsicum annus | T | S | 43.9 |
| Capsicum annuum | T | S | 50.1 |
| Capsicum frutescens | T | S | 28.9 |
| Carica papaya | T | W | 31.1 |
| Carthamus tinctorius | T | R | 37.3 |
| Carum carvi | T | S | 30.1 |
| Castanea spp. | T | W | 21.7 |
| Chaerophyllum bulbosum | T | S | 46.0 |
| Chamaemelum nobile | T | W | 36.8 |
| Chamaemelum nobile | T | W | 48.4 |
| Chelidonium majus | T | O | 46.6 |
| Chenapodium bonus-henricus | T | R | 22.4 |
| Chenopodium bonus-henricus | T | S | 57.6 |
| Chenopodium quinoa | T | V | 35.5 |
| Chenopodium quinoa | T | W | 54.4 |
| Chrysanthemum leucanthemum | T | R | 26.5 |
| Chrysanthemun coronarium (Chp suey) | T | R | 48.4 |
| Chrysanthenum coronarium | T | R | 38.2 |
| Chrysanthenum coronarium | T | S | 63.9 |
| Cicer arietinum | T | S | 20.0 |
| Cichorium endivia | T | S | 25.6 |
| Cichorium endivia crispa | T | O | 38.4 |
| Cichorium intybus | T | S | 30.2 |
| Cimicifuga racemosa | T | S | 33.7 |
| Citrullus colocynthus | T | S | 20.4 |
| Citrullus lanatus | T | O | 68.3 |
| Citrullus lanatus | T | S | 31.9 |
| Citrus limettoides | T | W | 20.4 |
| Citrus limettoides | T | V | 37.5 |
| Citrus limon | T | V | 47.7 |
| Citrus limon | T | O | 72.4 |
| Citrus paradisi | T | W | 23.8 |
| Citrus paradisi | T | V | 33.4 |
| Citrus reticulata | T | V | 20.4 |
| Citrus reticulata | T | V | 20.9 |
| Citrus reticulata | T | W | 26.0 |
| Citrus reticulata | T | S | 40.4 |
| Citrus reticulata | T | O | 50.0 |
| Citrus reticulata | T | O | 79.2 |
| Citrus sinensis | T | W | 25.3 |
| Citrus sinensis | T | V | 59.8 |
| Coix Lacryma-Jobi | T | W | 20.0 |
| Corchorus olitorius | T | S | 38.9 |
| Cornus canadensis | T | S | 35.6 |
| Cosmos sulphureus | T | S | 51.4 |
| Crataegus sp | T | V | 28.0 |
| Crataegus sp | T | R | 60.9 |
| Crataegus submollis | T | O | 25.5 |
| Crithmum maritima | T | S | 50.6 |
| Cryptotaenia canadensis | T | O | 21.2 |
| Cryptotaenia canadensis | T | W | 26.0 |
| Cryptotaenia canadensis | T | V | 40.0 |
| Cucumis anguria | T | S | 38.7 |
| Cucumis anguria | T | O | 46.6 |
| Cucumis melo | T | S | 30.3 |
| Cucumis melo | T | O | 46.2 |
| Cucumis meluliferus | T | W | 32.0 |
| Cucumis sativus Fanfare | T | O | 40.3 |
| Cucurbita maxima | T | S | 23.6 |
| Cucurbita maxima | T | S | 33.1 |
| Cucurbita maxima | T | O | 55.2 |
| Cucurbita moschata | T | S | 20.1 |
| Cucurbita moschata | T | S | 26.7 |
| Cucurbita moschata | T | O | 41.7 |
| Cucurbita pepo | T | S | 41.9 |
| Cucurbita pepo | T | O | 82.9 |
| Curcuma zedoaria | T | S | 100.0 |
| Cydonia oblonga | T | W | 42.9 |
| Cynara scolymus | T | R | 51.6 |
| Cynara scolymus | T | S | 60.9 |
| Dactilis Glomerata | T | R | 25.7 |
| Datura stramonium | T | R | 21.9 |
| Daucus carota | T | R | 25.9 |
| Dioscorea batatas | T | O | 47.6 |
| Dioscorea batatas | T | O | 83.1 |
| Diospiros Kaki | T | W | 34.9 |
| Dirca palustris | T | S | 27.6 |
| Dirca palustris | T | O | 90.4 |
| Dolichus lablab | T | R | 66.4 |
| Dolichus lablab | T | O | 85.3 |
| Dryopteris filix-mas | T | S | 21.9 |
| Dryopteris filix-mas | T | R | 77.9 |
| Echinacea purpurea | T | S | 48.6 |
| Eleusine coracana | T | O | 45.2 |
| Elymus junceus | T | R | 41.0 |
| Erigeron canadensis | T | S | 31.4 |
| Eriobotrya japonica | T | W | 28.3 |
| Eruca vesicaria | T | R | 44.9 |
| Fagopyrum escutentum | T | W | 76.7 |
| Fagopyrum tartaricum | T | W | 42.6 |
| Festuca rubra | T | R | 29.6 |
| Festuca rubra | T | S | 42.9 |
| Foeniculum vulgare | T | V | 22.1 |
| Foericulum vulgare | T | S | 21.6 |
| Foericulum vulgare | T | O | 84.8 |
| Forsythia intermedia | T | O | 70.8 |
| Forsythia x intermedia | T | O | 60.2 |
| Fortunella spp | T | S | 35.7 |
| Fortunella spp | T | W | 50.7 |
| Fortunella spp | T | O | 74.5 |
| Fragaria | T | W | 24.8 |
| Fragaria | T | V | 52.4 |
| Fragaria | T | O | 100.0 |
| Fragaria x ananassa | T | S | 29.3 |
| Galium odoratum | T | R | 26.0 |
| Gaultheria hispidula | T | W | 40.3 |
| Ginkgo biloba | T | V | 27.0 |
| Ginkgo biloba | T | W | 68.9 |
| Glechoma hederacea | T | R | 20.4 |
| Glechoma hederacea | T | S | 30.4 |
| Glycine max | T | O | 26.6 |
| Glycine max | T | R | 47.4 |
| Glycine max | T | S | 82.0 |
| Glycyrrhiza glabra | T | S | 35.4 |
| Glycyrrhiza glabra | T | O | 40.5 |
| Glycyrrhiza glabra | T | W | 100.0 |
| Gossypium herbaceum | T | S | 36.1 |
| Guizotia abyssinica | T | R | 28.9 |
| Guizotia abyssinica | T | S | 40.4 |
| Hamamelis virginiana | T | O | 52.4 |
| Hamamelis virginiana | T | S | 67.5 |
| Hamamelis virginiana | T | R | 84.1 |
| Hedeoma pulegiodes | T | S | 57.4 |
| Helenium hoopesii | T | O | 33.7 |
| Helenium hoopesii | T | S | 49.0 |
| Helianthus annus | T | S | 53.4 |
| Helianthus strumosus | T | R | 20.3 |
| Helianthus strumosus | T | O | 71.7 |
| Helianthus tuberosa | T | W | 22.8 |
| Helianthus tuberosus L. | T | V | 22.6 |
| Helianthus tuberosus L. | T | S | 55.0 |
| Helichrysum angustifolium | T | S | 67.0 |
| Heliotropium arborescens | T | S | 58.9 |
| Helleborus niger | T | S | 31.9 |
| Hibiscus cannabinus | T | S | 48.9 |
| Hordeum vulgare | T | S | 29.2 |
| Humulus lupulus | T | W | 22.4 |
| Humulus lupulus | T | R | 39.1 |
| Humulus lupulus | T | O | 63.1 |
| Humulus lupulus | T | S | 100.0 |
| Hydrastis canadensis | T | S | 20.2 |
| Hydrastis canadensis | T | W | 31.0 |
| Hyoscyamus niger | T | O | 56.8 |
| Hypericum henryi | T | O | 48.8 |
| Hypericum perforatum | T | S | 48.1 |
| Hypericum perforatum | T | O | 63.7 |
| Hypomyces lactiflorum | T | S | 44.8 |
| Hypomyces lactiflorum | T | O | 60.9 |
| Hyssops officinalis | T | W | 22.9 |
| Inula helenium | T | S | 24.6 |
| Juniperus communis | T | S | 33.0 |
| Juniperus communis | T | O | 38.2 |
| Lactuca sativa | T | S | 44.5 |
| Lactuca sativa | T | R | 50.7 |
| Laportea canadensis | T | S | 30.2 |
| Lathyrus Sativus | T | O | 20.4 |
| Lathyrus Sativus | T | R | 52.5 |
| Lathyrus sylvestris | T | W | 27.7 |
| Lathyrus sylvestris | T | O | 36.8 |
| Laurus nobilis | T | S | 52.0 |
| Lavendula angustifolia | T | W | 26.4 |
| Lavendula angustifolia | T | S | 53.2 |
| Lavendula latifolia | T | S | 51.3 |
| Ledum groenlandicum | T | S | 44.4 |
| Lentinus edodes | T | W | 42.1 |
| Lentinus edodes | T | O | 100.0 |
| Lepidium sativum | T | S | 44.2 |
| Levisticum officinale | T | S | 20.8 |
| Levisticum officinale | T | O | 39.4 |
| Linum usitatissimum | T | R | 42.3 |
| Litchi chinensis | T | W | 25.7 |
| Lolium multiflorum | T | S | 20.6 |
| Lolium perenne | T | R | 28.7 |
| Lonicera ramosissima | T | S | 26.3 |
| Lonicera ramosissima | T | O | 40.4 |
| Lonicera ramosissima | T | W | 53.2 |
| Lonicera syringantha | T | W | 95.8 |
| Lotus corniculatus | T | R | 100.0 |
| Lotus tetragonolubus | T | S | 65.4 |
| Lunaria annua | T | O | 55.7 |
| Lunaria annua | T | S | 67.3 |
| Lycopersicon esculentum | T | R | 37.6 |
| Malus | T | W | 31.8 |
| Malus | T | V | 44.4 |
| Malus hupehensis (Pamp.) Rehd. | T | R | 26.3 |
| Malus hupehensis (Pamp.) Rehd. | T | S | 67.0 |
| Malus sp. | T | R | 65.3 |
| Malva moschata | T | S | 41.1 |
| Malva sylvestris | T | S | 36.4 |
| Malva sylvestris | T | O | 47.4 |
| Malva verticillata | T | R | 42.7 |
| Mangifera indica | T | O | 30.5 |
| Manihot esculenta syn. M. utilissima | T | W | 38.3 |
| Manihot esculenta syn. M. utilissima | T | S | 50.4 |
| Manihot esculenta syn. M. utilissima | T | O | 86.5 |
| Melilotus alba | T | R | 30.4 |
| Melilotus officinalis | T | R | 68.1 |
| Melissa officinalis | T | S | 33.7 |
| Melissa officinalis | T | O | 34.7 |
| mentha arvensis | T | R | 53.7 |
| Mentha suaveolens | T | S | 26.8 |
| Menyanthes trifoliata | T | S | 32.8 |
| Miscanthus sinensis Andress | T | R | 22.7 |
| Momordica charantia | T | S | 55.5 |
| Monarda didyma | T | S | 26.8 |
| Monarda fistulosa | T | S | 21.5 |
| Montia perfoliata | T | R | 26.6 |
| Musa paradisiaca | T | W | 29.0 |
| nasturtium officinale | T | S | 35.4 |
| Nepeta cataria | T | W | 26.5 |
| Nepeta cataria | T | O | 27.5 |
| Nepeta cataria | T | S | 41.9 |
| Nephelium longana ou Euphoria longana | T | W | 43.4 |
| Nicotiana rustica | T | O | 26.0 |
| Nicotiana rustica | T | S | 32.7 |
| Nicotiana tabacum | T | S | 25.1 |
| Nicotiana tabacum | T | O | 77.7 |
| Nigella sativa | T | R | 59.3 |
| Nigella sativa | T | R | 100.0 |
| Ocimum Basilicum | T | W | 20.2 |
| Ocimum Basilicum | T | V | 20.2 |
| Ocimum Basilicum | T | S | 32.8 |
| Oenothera biennis linné | T | R | 100.0 |
| Onobrychis viciafolia | T | R | 45.0 |
| Optunia sp. | T | W | 33.4 |
| Origanum marjonara | T | O | 20.5 |
| Origanum vulgare | T | O | 20.8 |
| Origanum vulgare | T | W | 21.6 |
| Oryza sativa | T | W | 42.4 |
| oxyria digyna | T | O | 57.0 |
| oxyria digyna | T | V | 77.9 |
| Panax quinquefolius L. | T | O | 23.5 |
| Panicum miliaceum | T | W | 36.5 |
| Passiflora spp | T | S | 35.8 |
| Passiflora spp | T | V | 38.3 |
| Passiflora spp | T | W | 46.2 |
| Passiflora spp | T | O | 100.0 |
| Pastinaca sativa | T | O | 21.7 |
| Pastinaca sativa | T | R | 38.6 |
| Pastinaca sativa | T | S | 39.2 |
| Persea americana | T | V | 32.5 |
| Persea americana | T | O | 38.6 |
| Petasites Japonicus | T | S | 26.2 |
| Phalaris canariensis | T | O | 80.0 |
| Phaseolus coccineus | T | S | 44.4 |
| Phaseolus coccineus | T | R | 79.1 |
| Phaseolus mungo | T | S | 27.0 |
| Phaseolus mungo | T | O | 37.9 |
| Phaseolus vulgaris | T | R | 20.1 |
| Phaseolus vulgaris | T | S | 51.9 |
| Phaseolus vulgaris | T | O | 61.7 |
| Phlox paniculata | T | S | 22.9 |
| Phlox paniculata | T | O | 44.5 |
| Phoenix dactylifera | T | O | 29.6 |
| Physalis alkekengi | T | R | 32.9 |
| Physalis ixocarpa | T | R | 26.6 |
| Physalis ixocarpa | T | O | 28.3 |
| Physalis pruinosa | T | S | 27.3 |
| Physalis pruinosa | T | R | 47.8 |
| Physalis pruinosa | T | O | 93.1 |
| Physalis sp | T | W | 39.1 |
| Physalis sp | T | V | 60.8 |
| Phytolacca americana | T | S | 41.8 |
| Phytolacca americana | T | O | 100.0 |
| Phytolacca decandra syn. P. americana | T | O | 85.9 |
| Pimpinella anisum | T | S | 20.2 |
| Pimpinella anisum | T | O | 68.4 |
| Pisum sativum | T | W | 20.1 |
| Pisum sativum | T | S | 25.8 |
| Pisum sativum | T | V | 27.0 |
| Pisum sativum | T | O | 51.8 |
| Plantago coronopus | T | R | 21.9 |
| Plantago coronopus | T | O | 48.6 |
| Plantago coronopus | T | S | 66.8 |
| Plantago major | T | S | 35.1 |
| Pleurotus spp | T | W | 25.3 |
| Pleurotus spp | T | S | 59.3 |
| Pleurotus spp | T | O | 85.2 |
| Poa compressa | T | R | 26.2 |
| Poa pratensis | T | O | 21.5 |
| Poa pratensis | T | R | 30.0 |
| Podophyllum peltatum | T | O | 33.9 |
| Podophyllum peltatum | T | S | 50.2 |
| Polygonum aviculare linné | T | R | 31.0 |
| Polygonum pennsylvanicum | T | S | 56.6 |
| Polygonum persicaria | T | S | 20.1 |
| Populus incrassata | T | W | 54.9 |
| Populus Tremula | T | W | 31.0 |
| Populus x petrowskyana | T | W | 100.0 |
| Potentilla anserina | T | S | 22.1 |
| Potentilla anserina | T | O | 41.1 |
| Prunus cerasus | T | V | 30.1 |
| Prunus persica | T | W | 26.6 |
| Prunus persica | T | V | 38.5 |
| Prunus spp | T | S | 24.0 |
| Prunus spp | T | V | 49.1 |
| Psidium guajaba | T | V | 22.5 |
| Psidium guajaba | T | W | 44.3 |
| Psidium guajaba | T | O | 95.4 |
| Psidium spp | T | S | 36.6 |
| Psidium spp | T | W | 47.6 |
| Psidium spp | T | O | 87.6 |
| Pteridium aquilinum | T | R | 22.0 |
| Punica granalum | T | V | 52.1 |
| Pyrus communis | T | V | 39.5 |
| Pyrus pyrifolia | T | W | 33.7 |
| Raphanus raphanistrum | T | O | 24.5 |
| Raphanus raphanistrum | T | S | 44.8 |
| Raphanus raphanistrum | T | S | 46.1 |
| Raphanus sativus | T | V | 25.4 |
| Raphanus sativus | T | R | 32.1 |
| Raphanus sativus | T | W | 38.1 |
| Raphanus sativus | T | S | 63.6 |
| Raphanus sativus | T | O | 93.4 |
| Reseda luteola | T | S | 22.5 |
| Rhamnus frangula | T | S | 34.2 |
| Rhamnus frangula | T | R | 39.5 |
| Rheum officinale | T | S | 100.0 |
| Rheum palmatum | T | W | 20.2 |
| Rheum rhabarbarum | T | S | 33.8 |
| Rianus communis | T | S | 20.9 |
| Ribes nidigrolaria | T | W | 44.5 |
| Ribes nidigrolaria | T | V | 53.1 |
| Ribes nigrum | T | S | 40.7 |
| Ribes nigrum L. | T | W | 50.0 |
| Ribes nigrum L. | T | V | 60.1 |
| Ribes sativam syme | T | W | 47.9 |
| Ribes Sativum | T | R | 48.2 |
| Ribes Silvestre | T | V | 26.3 |
| Ribes Silvestre | T | W | 100.0 |
| Ribes uva-crispa | T | O | 57.5 |
| Rosa rugosa | T | S | 27.8 |
| Rosa rugosa thunb. | T | W | 37.5 |
| Rosa rugosa thunb. | T | V | 45.7 |
| Rosmarinum officinalis | T | R | 44.2 |
| Rosmarinum officinalis | T | W | 65.9 |
| Rubus canadensis | T | S | 45.5 |
| Rubus idaeus | T | W | 31.4 |
| Rubus idaeus | T | V | 57.2 |
| Rubus ideaus | T | S | 28.5 |
| Rubus ideaus | T | O | 38.0 |
| Rubus occidentalis | T | O | 21.4 |
| Rubus occidentalis | T | S | 36.5 |
| Rubus occidentalis | T | R | 60.2 |
| Rumes scutatus | T | O | 84.5 |
| Rumex crispus linné | T | O | 52.5 |
| Rumex crispus linné | T | R | 100.0 |
| Rumex patientia | T | O | 23.1 |
| Rumex patientia | T | S | 65.8 |
| Ruta graveolens | T | S | 37.2 |
| Sabal serrulata syn. Serenoa repens | T | V | 34.4 |
| Sabal serrulata syn. Serenoa repens | T | S | 44.6 |
| Salix purpurea | T | R | 67.8 |
| Salvia (elegens) | T | O | 51.1 |
| Sambucus canadensis | T | S | 44.8 |
| Sambucus canadensis | T | O | 72.4 |
| Sambucus canadensis L. | T | W | 67.8 |
| Sambucus ebulus | T | V | 44.3 |
| Sanguisorba officinalis | T | R | 100.0 |
| Santolina | T | R | 37.9 |
| Satureja montana | T | S | 20.0 |
| Satureja montana | T | O | 21.3 |
| Satureja repandra | T | S | 36.3 |
| Scorzorera hipanica | T | R | 27.1 |
| Scorzorera hipanica | T | S | 31.7 |
| Scuttellaria lateriflora | T | S | 44.3 |
| Secale cereale | T | S | 24.2 |
| Secale cereale | T | W | 31.1 |
| Sechium edule | T | S | 37.8 |
| Sesamum indicum | T | S | 59.2 |
| Setaria italica | T | W | 33.0 |
| Silybum marianum | T | O | 92.4 |
| Sium sisarum | T | O | 32.7 |
| Sium sisarum | T | S | 33.1 |
| Sium sisarum | T | O | 81.3 |
| Solanum melogena | T | O | 21.9 |
| solanum melogena | T | V | 26.1 |
| Solanum melogena | T | R | 34.0 |
| Solanum melogena | T | S | 67.1 |
| Solanum Tuberosum | T | O | 68.6 |
| Solidago canadensis | T | S | 48.4 |
| Solidago sp | T | R | 31.4 |
| Solidago virgaurea | T | S | 56.2 |
| Sorghum caffrorum | T | O | 23.3 |
| Sorghum dochna bicolor gr technicum | T | W | 20.8 |
| Sorghum dochna Snowdrew | T | S | 21.4 |
| Sorghum dochna Snowdrew | T | O | 27.7 |
| Spinacia oleracea | T | V | 25.0 |
| Spinacia oleracea | T | W | 32.1 |
| Spinacia oleracea | T | S | 47.6 |
| Spinacia oleracea | T | O | 63.1 |
| Stachys affinis | T | R | 31.7 |
| Stachys affinis | T | O | 100.0 |
| Stachys byzantina | T | W | 30.9 |
| Stipa capillata L. | T | R | 20.1 |
| Symphytum officinale | T | S | 24.1 |
| Tanacetum cinerarifolium | T | O | 24.2 |
| Tanacetum cinerarifolium | T | R | 84.4 |
| Tanacetum vulgare | T | R | 25.7 |
| Tanacetum vulgare | T | S | 75.6 |
| Taraxacum officinale (Red ribe) | T | S | 21.1 |
| Tepary | T | R | 56.7 |
| Teucrium chamaedrys L. | T | R | 27.3 |
| Thalpsi arvense | T | S | 61.4 |
| Thymus fragantissumus | T | R | 100.0 |
| Thymus herba-barona | T | W | 22.0 |
| Thymus pseudolanuginosus | T | R | 36.8 |
| Thymus pseudolanuginosus | T | S | 37.1 |
| Thymus serpyllum | T | S | 26.0 |
| Thymus serpyllum | T | W | 42.7 |
| Thymus x citriodorus | T | O | 22.7 |
| Tiarella cordifolia | T | R | 100.0 |
| Tragopogon porrifolius | T | V | 26.8 |
| Tragopogon porrifolius | T | O | 28.4 |
| Tragopogon porrifolius | T | S | 42.1 |
| Tragopogon sp. | T | O | 20.3 |
| Tragopogon sp. | T | S | 32.0 |
| Tragopogon sp. | T | W | 66.3 |
| Trichosanthes kirilowii | T | O | 66.5 |
| Trifolium incarnatum | T | R | 47.9 |
| Trifolium repens | T | R | 81.7 |
| Trigonella foenum graecum | T | S | 39.6 |
| Triticale sp. | T | O | 64.1 |
| Triticum aestivum | T | W | 24.5 |
| Triticum aestivum | T | S | 29.4 |
| Triticum furgidumm | T | S | 35.8 |
| Triticum spelta | T | S | 34.7 |
| Tropaeolum majus | T | O | 90.3 |
| Tropaeolum malus | T | W | 20.1 |
| Tsuga can0adensis | T | O | 21.5 |
| Tsuga can0adensis | T | W | 64.4 |
| Tsuga diversifolia | T | O | 45.9 |
| Tsuga diversifolia | T | W | 100.0 |
| Tsuga F. macrophylla | T | W | 28.1 |
| Typha latifolia L. | T | S | 30.6 |
| Urtica dioica | T | O | 31.4 |
| Urtica dioica | T | R | 36.9 |
| Urtica dioica | T | S | 41.7 |
| Vaccinium angustifolium | T | V | 25.2 |
| Vaccinium angustifolium | T | R | 34.6 |
| Vaccinium angustifolium | T | O | 59.6 |
| Vaccinium angustifolium | T | R | 65.7 |
| Vaccinium macrocarpon | T | O | 30.2 |
| Vaccinium macrocarpon | T | S | 39.0 |
| Vaccinium macrocarpon | T | S | 56.9 |
| Vaccinum macrocarpon | T | V | 39.2 |
| Vaccinum macrocarpon | T | W | 42.3 |
| Veratrum viride | T | O | 20.5 |
| Veratrum viride | T | S | 33.1 |
| Verbascum thapsus | T | S | 43.1 |
| Verbascum thapsus | T | O | 70.2 |
| Veronica officinalis | T | O | 20.5 |
| Viburnum trilobum Marsh. | T | S | 40.6 |
| Vicia taba | T | R | 61.5 |
| Vicia sativa | T | R | 30.1 |
| Vigna angularia | T | R | 32.6 |
| Vigna angularia | T | S | 64.2 |
| Vigna unguiculata | T | R | 32.4 |
| Vigna unguiculata | T | O | 47.4 |
| Vigna unguiculata | T | S | 51.0 |
| Vinca minor | T | S | 21.3 |
| Vitis sp. | T | V | 28.3 |
| Vitis sp. | T | O | 29.4 |
| Vitis sp. | T | S | 45.4 |
| Vitis sp. | T | V | 50.7 |
| Vitis sp. | T | W | 61.6 |
| Vitis sp. | T | R | 100.0 |
| Weigela coracensis | T | W | 35.5 |
| Withania somnifera | T | S | 35.5 |
| Xanthium sibiricum | T | S | 38.6 |
| Xanthium strumarium | T | S | 33.5 |
| Zea mays | T | S | 37.1 |
| Zea mays | T | O | 65.5 |
| Zingiber officinale | T | S | 20.1 |
| Zingiber officinale | T | W | 58.9 |
| Zingiber officinale | T | O | 75.9 |

TABLE 5
Cath B

| Nom latin | Stress | Extrait | Inhibition (%) |
|---|---|---|---|
| Achillea millefolium | A | O | 61.9 |
| Achillea tomentosa | A | O | 60.8 |
| Aconitum | A | O | 38.6 |
| Aconitum napellus | A | O | 61.1 |
| Alchemilla mollis | A | R | 26.7 |
| Allium | A | R | 43.0 |
| Allium cepa gr. Cepa | A | O | 49.9 |
| Allium cepa gr. Cepa | A | O | 70.1 |
| Allium cepa gr. Cepa | A | R | 45.8 |
| Allium sativum | A | O | 25.6 |
| Allium Tuberosum | A | O | 91.5 |
| Allium Tuberosum | A | O | 75.0 |
| Allium victorialis | A | O | 31.1 |
| Amaranthus gangeticus | A | O | 26.1 |
| Amaranthus gangeticus | A | O | 29.0 |
| Amelanchier canadensis | A | R | 28.7 |
| Anthemis tinctoria | A | O | 26.8 |
| Anthemis tinctoria | A | R | 32.4 |
| Anthoxanthum odoratum | A | O | 24.9 |
| Apium graveolens | A | O | 31.1 |
| Apium graveolens | A | O | 20.6 |
| Aralia cordata | A | R | 52.3 |
| Arctium lappa | A | O | 33.7 |
| Arctium lappa | A | R | 33.0 |
| Aronia melanocarpa (Michx.) Ell. | A | R | 41.2 |
| Aronia melanocarpa (Michx.) Ell. | A | O | 21.6 |
| Asarum europaeum | A | O | 24.9 |
| Athaea officinalis | A | O | 57.7 |
| Athyrium asperum | A | O | 27.3 |
| Atropa belladonna | A | O | 37.7 |
| Begonia convolvulacea | A | O | 26.0 |
| Begonia eminii | A | O | 34.2 |
| Begonia glabra | A | O | 38.9 |
| Begonia Hannii | A | O | 52.9 |
| Begonia polygonoides | A | O | 67.3 |
| Berberis vulgaris | A | O | 54.6 |
| Beta vulgaris | A | R | 39.9 |
| Beta vulgaris | A | R | 30.4 |
| Beta vulgaris | A | O | 61.9 |
| Beta vulgaris | A | O | 43.0 |
| Beta vulgaris | A | R | 91.0 |
| Beta vulgaris | A | O | 46.7 |
| Beta vulgaris | A | R | 65.3 |
| Beta vulgaris | A | R | 33.4 |
| Beta vulgaris | A | O | 54.3 |
| Beta vulgaris | A | O | 38.2 |
| Beta vulgaris | A | R | 55.9 |
| Beta vulgaris | A | R | 28.5 |
| Beta vulgaris | A | O | 40.1 |
| Beta vulgaris spp. Maritima | A | O | 33.4 |
| Brassica juncea | A | O | 21.3 |
| Brassica Oleracea | A | O | 27.5 |
| Brassica Oleracea | A | O | 48.2 |
| Brassica rapa | A | O | 20.8 |
| Calendula officinalis | A | O | 35.6 |
| Camellia sinensis syn. Thea sinensis | A | R | 24.4 |
| Cana edulis | A | R | 100.0 |
| Capsicum annuum | A | O | 25.0 |
| Capsicum frutescens | A | O | 29.6 |
| Chrysanthemum balsamita | A | O | 89.3 |
| Chrysanthemun balsamina | A | O | 55.0 |
| Chrysanthemun coronarium (Chp Suey) | A | O | 30.1 |
| Chrysanthemun coronarium (Chp Suey) | A | O | 36.4 |
| Cichorium intybus | A | R | 100.0 |
| Citrullus lanatus | A | O | 24.4 |
| Convallaria maialis | A | O | 57.0 |
| Coriandrum sativum | A | R | 20.8 |
| Cryptotaenia canadensis | A | O | 20.4 |
| Cucumis Anguria | A | O | 26.8 |
| Cucumis sativus | A | R | 45.6 |
| Curburbita pepo | A | O | 30.8 |
| Daucus carota | A | R | 68.8 |
| Daucus carota | A | O | 20.3 |
| Daucus carota | A | R | 72.5 |
| Daucus carota | A | O | 22.6 |
| Daucus carota | A | O | 25.6 |
| Daucus carota | A | R | 65.9 |
| Daucus carota | A | R | 77.3 |
| Daucus carota | A | R | 41.6 |
| Dirca palustris | A | R | 100.0 |
| Eruca vesicaria | A | O | 41.4 |
| Filipendula rubra | A | R | 65.0 |
| Forsythia intermedia | A | R | 100.0 |
| Forsythia x intermedia | A | R | 100.0 |
| Geum rivale | A | O | 26.4 |
| Glycyrrhiza glabra | A | R | 86.8 |
| Heliotropium arborescens | A | O | 29.5 |
| Humulus Lupulus | A | O | 65.4 |
| Humulus Lupulus | A | R | 100.0 |
| Hylotelephium | A | R | 23.7 |
| Hypericum henryi | A | R | 44.4 |
| Iberis sempervirens | A | O | 84.6 |
| Jeffersonia diphylla | A | O | 35.4 |
| Ligularia dentata | A | O | 30.3 |
| Lonicera ramosissima | A | R | 48.7 |
| Miscanthus sacchariflorus | A | O | 50.9 |
| Nicotiana tabacum | A | O | 40.0 |
| Nicotiana tabacum | A | O | 56.8 |
| Nicotiana tabacum | A | O | 55.2 |
| Nigella sativa | A | O | 40.3 |
| Origanum majorana | A | O | 49.7 |
| Origanum vulgare | A | O | 67.0 |
| Origanum vulgare | A | O | 39.9 |
| Panax quinquefolius L. | A | O | 24.0 |
| Pastinaca sativa | A | R | 33.5 |
| Petroselinum crispum | A | O | 70.2 |
| Peucedanum cervaria | A | O | 21.5 |
| Phaseolus Vulgaris | A | O | 67.9 |
| Philadelphus coronarius | A | O | 24.0 |
| Physostegia virginiana | A | O | 56.9 |
| Phytolacca americana | A | O | 100.0 |
| Plantago major | A | O | 31.2 |
| Plectranthus fruticosus | A | O | 32.1 |
| Polygonum pennsylvanicum | A | R | 70.1 |
| Pulmonaria saccharata | A | O | 31.1 |
| Raphanus sativus | A | O | 21.5 |
| Raphanus sativus | A | O | 50.5 |
| Raphanus sativus | A | O | 58.9 |
| Ribes nigrum L. | A | O | 53.1 |
| Rubus Allegheniensis | A | O | 56.7 |
| Rubus ideaus | A | R | 89.0 |
| Rumex crispus linne | A | R | 65.2 |
| Salvia elegens | A | O | 32.6 |
| Salvia nemorosa | A | O | 26.2 |
| Salvia officianalis | A | O | 26.3 |
| Salvia sclarea | A | R | 51.6 |
| Salvia sclarea | A | O | 21.5 |
| Saponaria officinalis | A | O | 68.5 |
| Satureja montana | A | O | 47.6 |
| Scorzonera hispanica | A | O | 29.9 |
| Sesamum indicum | A | O | 84.8 |
| Solanum dulcamara | A | O | 51.3 |
| Solidago canadensis | A | O | 95.3 |
| Solidago hybrida | A | O | 94.5 |
| Solidago hybrida | A | O | 99.5 |
| Solidago sp ? | A | O | 60.9 |
| Stellaria graminea linne | A | O | 40.2 |
| Tamarindus indica | A | O | 59.2 |
| Taraxacum officinale | A | O | 88.6 |
| Thalictrum aquilegiifolium | A | O | 65.2 |
| Thalictrum Aquilegiifolium | A | O | 44.5 |
| Thuja occidentalis | A | O | 50.6 |
| Thymus praecox subsp arctitus | A | O | 23.9 |
| Tiarella | A | R | 34.4 |
| Vaccinum augustifolium | A | R | 67.2 |
| Vaccinum macrocarpon | A | R | 37.1 |
| Vitia sp. | A | R | 93.7 |
| Xanthium strumarium | A | O | 83.2 |
| Yucca filamentosa | A | O | 34.5 |
| Zea mays | A | O | 29.7 |
| Zea mays | A | O | 93.2 |
| Achillea tomentosa | G | O | 41.0 |
| Adiantum tenerum | G | R | 30.2 |
| Alcea rosea | G | O | 37.7 |
| Alchemilla mollis | G | R | 32.8 |
| Allium schoenoporasum | G | O | 49.3 |
| Allium tuberosum | G | O | 79.1 |
| Allium tuberosum | G | O | 77.4 |
| Allium victorialis | G | O | 45.5 |
| Althaea officinalis | G | O | 67.2 |
| amaranthus gangeticus | G | O | 23.5 |
| Anaphalis margaritacea | G | R | 34.7 |
| Angelica dahurica | G | R | 27.9 |
| Anthemis nobilis | G | O | 42.3 |
| Apium graveolens | G | O | 25.7 |
| Apium graveolens | G | O | 27.4 |
| Arotostaphylos uva-ursi | G | R | 94.5 |
| Aronia melanocarpa | G | R | 74.5 |
| Aronia melanocarpa | G | O | 21.3 |
| Aronia melanocarpa (Michx.) Ell. | G | R | 79.9 |
| Aronia melanocarpa (Michx.) Ell. | G | R | 28.3 |
| Asarum europaeum | G | O | 55.4 |
| Atropa belladonna | G | O | 58.9 |
| Begonia eminii | G | O | 24.7 |
| Begonia glabra | G | O | 42.9 |
| Begonia manii | G | O | 32.1 |
| Begonia polygonoides | G | O | 38.2 |
| Berberis vulgaris | G | O | 42.3 |
| Beta vulgaris | G | R | 75.3 |
| Beta vulgaris | G | O | 28.7 |
| Beta vulgaris | G | O | 21.7 |
| Beta vulgaris | G | R | 40.0 |
| Beta vulgaris spp. Maritima | G | O | 31.4 |
| Betula glandulosa | G | R | 38.5 |
| Calendula officinalis | G | O | 36.2 |
| Capsicum annus | G | O | 49.9 |
| Chrysanthemum balsamita | G | O | 100.0 |
| Chrysanthemun balsamina | G | O | 33.1 |
| Cynara scolymus | G | O | 51.9 |
| Daucus carota | G | O | 81.3 |
| Daucus carota | G | O | 27.2 |
| Dirca palustris | G | R | 100.0 |
| Echinacea purpurea | G | O | 22.9 |
| Equisetum hyemale | G | O | 100.0 |
| Erigeron canadensis | G | O | 73.3 |
| Erigeron speciosus (Lindl.) D.C. | G | O | 22.9 |
| Eruca vesicaria | G | O | 29.2 |
| Erysimum perofskianum Fish. S. | G | O | 89.8 |
| Fenouil bronze | G | R | 23.7 |
| Filipendula rubra | G | R | 93.2 |
| Filipendula rubra | G | R | 100.0 |
| Filipendula ulmaria | G | O | 20.5 |
| Filipendula vulgaris | G | O | 26.2 |
| Forsythia intermedia | G | R | 100.0 |
| Forsythia x intermedia | G | R | 100.0 |
| Galium odoratum | G | O | 21.0 |
| Gaultheria hispidula (L.) Muhl | G | R | 39.3 |
| Gaultheria procumbens | G | R | 43.4 |
| Geum rivale | G | O | 21.7 |
| Glycine max | G | O | 64.2 |
| Glycyrrhiza glabra | G | R | 53.4 |
| Hamamelis virginiana | G | R | 88.4 |
| Heliotropium arborescens | G | O | 23.0 |
| Humulus lupulus | G | R | 100.0 |
| Humulus lupulus | G | O | 90.2 |
| Hydrastis canadensis | G | O | 30.9 |
| Hylotelephium | G | R | 43.8 |
| Hypericum henryi | G | R | 50.3 |
| Iberis sempervirens | G | O | 87.7 |
| Lathyrus sativus | G | R | 25.9 |
| Ligularia dentata | G | O | 31.5 |
| Lunaria annua | G | O | 59.7 |
| Lythrum salicaire | G | R | 33.1 |
| Melissa officinalis | G | O | 27.6 |
| Miscanthus sacchariflorus | G | O | 30.7 |
| Nicotiana rustica | G | O | 54.8 |
| Nicotiana tabacum | G | O | 36.2 |
| Nigella sativa | G | O | 40.3 |
| Origan | G | O | 98.8 |
| Origanum majorana | G | O | 48.9 |
| Panax quinquefolius L. | G | O | 21.1 |
| Panicum miliaceum | G | R | 100.0 |
| Passiflora caerula | G | O | 66.2 |
| Petroselinum crispum | G | O | 65.0 |
| Phaseolus vulgaris | G | R | 40.3 |
| Physostegia virginiana | G | O | 74.0 |
| Phytolacca americana | G | O | 100.0 |
| Plantago major | G | O | 60.9 |
| Plectranthus fruticosus | G | O | 29.2 |
| Polygonum aviculare linne | G | R | 45.6 |
| Pongamia pinnata | G | O | 41.7 |
| Pulmonaria officinalis | G | O | 36.9 |
| Pulmonaria saccharata | G | O | 24.7 |
| Raphanus sativus | G | O | 38.9 |
| Raphanus sativus | G | O | 86.4 |
| Rhus aromatica | G | O | 49.1 |
| Ribes nigrum L. | G | O | 20.6 |
| Rubus ideaus | G | R | 56.9 |
| Rubus occidentalis | G | R | 61.3 |
| Saponaria officinalis | G | O | 48.3 |
| Sarriette vivace | G | O | 44.6 |
| Satureja repandra | G | O | 72.3 |
| Sesamum indicum | G | O | 46.8 |
| Sidalcea | G | O | 55.2 |
| Silene vulgaris | G | O | 35.5 |
| Solanum dulcamara | G | O | 56.9 |
| Solidago canadensis | G | O | 99.8 |
| Solidago canadensis | G | O | 100.0 |
| Solidago~sp ? | G | O | 71.8 |
| Sorghum caffrorum | G | O | 34.5 |
| Tamarindus indica | G | O | 65.4 |
| Taraxacum officinale | G | O | 82.7 |
| taraxacum officinale | G | O | 42.7 |
| Tetradenia riparia | G | O | 32.5 |
| Thalictrum aquilegiifolium | G | O | 62.1 |
| Thuja occidentalis | G | O | 57.7 |
| Thymus vulgaris “Argenteus” | G | O | 40.7 |
| Tiarella | G | R | 39.0 |
| Tropaeolum majus | G | O | 36.6 |
| Tussilago farfara | G | O | 26.8 |
| Vaccinium angustifolium | G | R | 26.4 |
| Vaccinium angustifolium | G | R | 89.1 |
| Vaccinum macrocarpon | G | R | 33.9 |
| Vitia sp. | G | R | 100.0 |
| Vitia sp. | G | R | 90.9 |
| Vitis sp. | G | O | 37.1 |
| Achillea millefolium | T | O | 44.1 |
| Aconitum napelius | T | O | 27.4 |
| Aesculus hippocastanum | T | R | 84.2 |
| Aesculus hippocastanum | T | O | 47.3 |
| Alcea rosea “Nigra” | T | O | 24.3 |
| Alchemilla moliis | T | R | 24.9 |
| Allium ascalonicum | T | O | 31.1 |
| Allium cepa gr. Cepa | T | O | 39.4 |
| Allium cepa gr. Cepa | T | R | 23.2 |
| Allium cepa gr. Cepa | T | O | 45.5 |
| Allium fistulosum | T | O | 21.9 |
| Allium grande | T | O | 39.5 |
| Allium tuberosum | T | O | 26.6 |
| Allium tuberosum | T | O | 33.1 |
| Allium tuberosum | T | O | 72.3 |
| Allium tuberosum | T | R | 22.6 |
| Allium victorialis | T | O | 42.3 |
| Alpinia oficinarum | T | O | 57.4 |
| Alpinia oficinarum | T | R | 88.9 |
| Althacea officinalis | T | O | 51.5 |
| Althaea officianalis | T | O | 25.2 |
| Amelanchier canadensis | T | O | 20.8 |
| Amelanchier canadensis | T | R | 42.1 |
| Amsonia tabernaemontana | T | O | 30.2 |
| Ananas comosus | T | R | 36.2 |
| Anaphalis margaritacea | T | R | 33.9 |
| Angelica dahurica | T | R | 40.7 |
| Angelica sinensis syn, A. polymorpha | T | O | 91.0 |
| Anthriscus cerefolium | T | R | 23.3 |
| Anthriscus cerefolium | T | O | 21.7 |
| Aralia cordata | T | R | 44.1 |
| Aronia melanocarpa | T | R | 33.1 |
| Aronia melanocarpa | T | R | 100.0 |
| Aronia melanocarpa (Michx.) Ell. | T | R | 35.0 |
| Aronia prunifolia | T | R | 50.4 |
| Artemisia draculus | T | O | 42.5 |
| Asarum europaeum | T | O | 39.4 |
| Asclepias incarnata L. | T | O | 48.7 |
| Asclepias tuberosa | T | O | 21.5 |
| Asctinidia chinensis | T | O | 24.9 |
| Atriplex hortensis | T | O | 22.4 |
| Atropa belladonna | T | O | 94.1 |
| Aubepine, hawthorne | T | R | 72.7 |
| Begonia convolvulacea | T | O | 32.1 |
| Begonia eminii | T | O | 40.4 |
| Begonia glabra | T | O | 84.3 |
| Begonia manii | T | O | 64.2 |
| Berberus vulgaris | T | O | 35.4 |
| Beta vulgaris | T | O | 34.1 |
| Beta vulgaris | T | R | 86.7 |
| Beta vulgaris | T | O | 23.8 |
| Beta vulgaris | T | R | 79.4 |
| Beta vulgaris | T | O | 34.2 |
| Beta vulgaris | T | R | 20.8 |
| Beta vulgaris | T | R | 37.0 |
| Beta vulgaris spp. Maritima | T | R | 83.6 |
| Betula glandulosa | T | R | 62.5 |
| Borago officinalis | T | O | 23.5 |
| Brassica Napus | T | O | 27.6 |
| Brassica oleracea | T | O | 21.8 |
| Brassica oleracea | T | O | 22.3 |
| Butomus umbellatus | T | O | 20.8 |
| Canna edulis | T | R | 100.0 |
| cannelle | T | R | 99.5 |
| Carica papaya | T | R | 100.0 |
| Chrysanthemum balsamita | T | O | 89.3 |
| Chrysanthemum parthenium | T | R | 44.6 |
| chrysanthemun coronarium (Chp Suey) | T | O | 28.7 |
| chrysanthemun coronarium (Chp Suey) | T | O | 59.2 |
| Citrus paradisi | T | R | 100.0 |
| Citrus sinensis | T | R | 100.0 |
| Cocos nucifera | T | R | 100.0 |
| Cocos nucifera | T | O | 71.9 |
| Convallaria majalis | T | O | 67.1 |
| Corchorus olitorius | T | R | 26.0 |
| Crataegus sanguinea | T | O | 33.1 |
| Cryptotaenia canadensis | T | R | 23.1 |
| Cucumis anguria | T | O | 26.4 |
| Cucumis sativus (Fanfare) | T | O | 25.7 |
| Cydonia oblonga | T | R | 23.6 |
| Datura stramonium | T | O | 61.4 |
| Daucus carota | T | R | 21.1 |
| Diospiros Kaki | T | R | 100.0 |
| Echinacea purpurea | T | O | 27.8 |
| Eriobotrya japonica | T | R | 25.2 |
| Eruca vesicaria | T | O | 34.5 |
| Erysimum perofskianum Fish. S. | T | O | 91.0 |
| Fragaria x ananassa | T | R | 37.5 |
| Fucus vesiculosis | T | R | 87.1 |
| Fumaria officinalis | T | O | 44.4 |
| Gaultheria procumbens | T | R | 74.8 |
| Gentiana macrophylla | T | O | 44.5 |
| Glyceria maxima | T | O | 37.6 |
| Glycine max Envy | T | O | 40.3 |
| Glycyrrhiza glabra | T | R | 37.7 |
| Hamamelis virginiana | T | R | 78.3 |
| Helichrysum angustifolium | T | R | 21.8 |
| Heliotropium arborescens | T | O | 26.8 |
| Humulus lupulus | T | R | 84.7 |
| Humulus lupulus | T | O | 39.2 |
| Humulus lupulus | T | O | 100.0 |
| Humulus lupulus | T | R | 100.0 |
| Hydrastis canadensis | T | I | 42.7 |
| Hypericum henryi | T | R | 51.8 |
| Hypericum perforatum | T | O | 52.3 |
| Hypomyces lactiflorum | T | O | 30.1 |
| Iberis sempervirens | T | O | 90.8 |
| Jeffersonia diphylla | T | O | 43.0 |
| Juglans nigra | T | R | 66.7 |
| Kochia scoparia (L.) Schrad. | T | O | 38.4 |
| Krameria Triandra | T | R | 63.6 |
| Lentinus edodes | T | R | 100.0 |
| Lentinus edodes | T | R | 26.2 |
| Ligularia dentata | T | O | 34.9 |
| Ligustrum vulgare | T | O | 29.5 |
| Lunaria annua | T | O | 72.3 |
| Lunaria annua | T | R | 51.1 |
| Lupinus polyphyllus lindl. | T | O | 47.4 |
| Lychnis chalcedonica | T | O | 34.4 |
| Lythrum salicaire | T | R | 53.8 |
| Mangifera indica | T | R | 100.0 |
| Mangifera indica | T | O | 29.3 |
| Nigella sativa | T | O | 26.1 |
| Nil | T | O | 73.6 |
| Nil | T | R | 25.4 |
| Nil | T | R | 24.6 |
| Nil | T | R | 49.8 |
| Nil | T | O | 43.6 |
| Nil | T | R | 28.4 |
| Optunia sp. | T | R | 100.0 |
| Panax quinquefolius L. | T | O | 27.4 |
| Passiflora caerula | T | O | 39.8 |
| Pastinaca sativa | T | O | 20.5 |
| Perroselinum crispum | T | O | 60.9 |
| Phaseolus vulgaris | T | O | 37.5 |
| Physostegia virginiana | T | O | 64.2 |
| Phytolacca americana | T | O | 51.9 |
| Phytolacca americana | T | O | 100.0 |
| Plectranthus fruticosus | T | O | 23.4 |
| Polygonatum odoratum | T | O | 100.0 |
| Polygonium chinense | T | R | 33.6 |
| Pontederia cordata | T | O | 26.2 |
| Portulacea oleracea | T | O | 20.7 |
| Primula veris | T | O | 58.2 |
| Prunus persica | T | R | 100.0 |
| Prunus persica (hybrids de la peche) | T | R | 100.0 |
| Pulmonaria officinalis | T | O | 22.8 |
| Punica granatum | T | R | 100.0 |
| Pyrus pyrifolia | T | R | 22.4 |
| Radix Paeonia rubra | T | O | 39.8 |
| Rahmnus frangula | T | R | 25.3 |
| Raphanus sativus | T | O | 45.8 |
| Rhus trilobata | T | O | 20.2 |
| Ribes uva-crispa | T | R | 34.2 |
| Rosa Rugosa “Alba” | T | O | 45.4 |
| Rubus idaeus | T | R | 31.2 |
| Rubus idaeus L. | T | O | 42.7 |
| Rubus ideaus | T | R | 74.2 |
| Rubus occidentalis | T | R | 68.1 |
| Rumex crispus linne | T | R | 37.9 |
| Salvia nemorosa | T | O | 38.2 |
| Sambucus canadensis | T | O | 27.5 |
| Sambucus nigra | T | O | 30.8 |
| Sanguisorba minor | T | R | 78.3 |
| Saponaria officinalis | T | O | 68.7 |
| Saponaria officinalis L. | T | O | 44.2 |
| Satureja hortensis | T | O | 62.1 |
| Sechium edule | T | O | 34.4 |
| Sesamum indicum | T | O | 78.6 |
| Sidalcea | T | O | 42.9 |
| Silene vulgaris | T | O | 51.3 |
| Solidago hybrida | T | O | 92.8 |
| Solidago Hybrida | T | O | 100.0 |
| Solidago Hybrida | T | R | 100.0 |
| Solidago sp ? | T | O | 39.6 |
| Tamarindus indica | T | O | 64.2 |
| Tanacetum balsamila | T | O | 100.0 |
| Tanacetum vulgare | T | O | 23.3 |
| Taraxacum officinale | T | O | 90.9 |
| Taraxacum officinale (Red ribe) | T | O | 34.5 |
| Thuja occidentalis | T | O | 37.6 |
| Thymus serpyllum | T | O | 20.6 |
| Tiarella | T | R | 35.6 |
| Tragopogon sp. | T | R | 21.1 |
| Trigonella foenum graecum | T | R | 97.3 |
| Tropaeolum majus | T | O | 58.8 |
| Tropaeolum majus | T | R | 28.6 |
| Tropaeolum majus | T | O | 36.7 |
| Tsuga diversifolia | T | R | 64.0 |
| Vaccinium angustifolium | T | R | 72.2 |
| Vaccinium angustifolium | T | R | 50.7 |
| Vaccinium macrocarpon | T | R | 52.6 |
| Vitia sp. | T | O | 35.1 |
| Vitia sp. | T | R | 98.9 |
| Vitis sp. | T | R | 32.6 |
| Weigela coracensis | T | R | 24.6 |
| Zea mays | T | R | 100.0 |
| Zea mays | T | R | 48.1 |

TABLE 6
Cath D

| Nom latin | Stress | Extrait | Inhibition (%) |
|---|---|---|---|
| Agastache foeniculum | A | O | 91.6 |
| Agropyron cristatum | A | O | 24.5 |
| Agropyron repens | A | O | 75.2 |
| Agrostis Stofonifera | A | O | 94.7 |
| Alchemilla mollis | A | O | 39.0 |
| Allium sativum | A | R | 100.0 |
| Allium schoenoprasum | A | R | 40.0 |
| Althaea officinalis | A | O | 96.5 |
| Amaranthus gangeticus | A | R | 67.4 |
| Amaranthus gangeticus | A | O | 74.3 |
| Amaranthus retroflexus | A | O | 100.0 |
| Ambrosia artemisiifolia | A | O | 75.4 |
| Anethum graveolens | A | O | 48.7 |
| Angelica archangelica | A | O | 27.6 |
| Anthemis nobilis | A | O | 56.2 |
| Anthemis tinctoria | A | S | 42.3 |
| Aralia cordata | A | R | 100.0 |
| Aralia nudicaulis | A | R | 44.9 |
| Arctium minus | A | O | 93.2 |
| Arctium minus | A | O | 100.0 |
| Aronia melanocarpa | A | O | 22.8 |
| Artemisia abrotanum | A | O | 31.3 |
| Artemisia abrotanum | A | O | 43.6 |
| Artemisia absinthium | A | O | 58.3 |
| Artemisia Absinthium | A | O | 71.4 |
| Artemisia dracunculus | A | O | 70.5 |
| Artemisis Ludoviciana | A | O | 74.4 |
| Artemisis Ludoviciana | A | O | 100.0 |
| Asparagus officinalis | A | O | 61.9 |
| Aster sp | A | O | 100.0 |
| Aster sp | A | O | 100.0 |
| Atropa belladonna | A | O | 100.0 |
| Beckmannia eruciformis | A | R | 22.1 |
| Beckmannia eruciformis | A | O | 48.3 |
| Beta vulgaris | A | R | 21.2 |
| Beta vulgaris | A | R | 100.0 |
| Beta vulgaris spp. Maritima | A | O | 30.8 |
| Betta vulgaris | A | O | 100.0 |
| Brassica napus | A | R | 63.6 |
| Brassica oleracea | A | R | 33.3 |
| Brassica rapa | A | R | 23.8 |
| Brassica rapa | A | O | 26.1 |
| Bromus inermis | A | O | 59.6 |
| Calamintha nepeta | A | R | 24.0 |
| Campanula rapunculus | A | O | 41.6 |
| Canna edulis | A | O | 100.0 |
| Capsella bursa-pastoris | A | O | 36.7 |
| Capsicum annuum | A | R | 25.8 |
| Capsicum annuum | A | R | 28.2 |
| Capsicum annuum | A | O | 64.7 |
| Capsicum annuum | A | R | 76.9 |
| Capsicum frutescens | A | O | 44.1 |
| Carthamus tinctorius | A | O | 42.9 |
| Carum carvi | A | R | 28.6 |
| Chaerophyllum bulbosom | A | O | 100.0 |
| Chelidonium majus | A | R | 100.0 |
| chenopodium bonus-henricus | A | O | 54.3 |
| Chenopodium quinoa | A | R | 22.2 |
| Chrysanthemum coronarium | A | O | 96.8 |
| Cichorium endivia susp. Endivia | A | R | 36.0 |
| Cichorium endivia susp. Endivia | A | O | 78.4 |
| Cichorium intybus | A | O | 100.0 |
| Citrullus lanatus | A | O | 22.7 |
| Citrullus lanatus | A | R | 26.7 |
| Citrullus lanatus | A | R | 35.9 |
| Citrullus lanatus | A | O | 76.5 |
| Coix Lacryma-Jobi | A | O | 20.9 |
| Coix Lacryma-Jobi | A | O | 93.2 |
| Cornus canadensis | A | O | 30.9 |
| Cuburbita pepo | A | O | 21.9 |
| Cucumis melo | A | O | 44.1 |
| Cucumis sativus | A | O | 21.3 |
| Cucumis sativus | A | R | 33.3 |
| Cucurbita Maxima | A | R | 100.0 |
| Cucurbita moschata | A | R | 20.5 |
| Cucurbita pepo | A | O | 31.9 |
| Cucurbita pepo | A | R | 40.9 |
| Cucurbita pepo | A | O | 41.2 |
| Curcuma zedoaria | A | O | 26.3 |
| Cymbopogon martinii | A | O | 77.8 |
| Daucus carota | A | O | 55.1 |
| Daucus carota | A | R | 100.0 |
| Dipsacus sativus | A | O | 21.1 |
| Elymus junceus | A | O | 27.7 |
| Eschscholzia californica | A | O | 44.4 |
| Foeniculum vulgare | A | O | 81.8 |
| Forsythia intermedia | A | O | 40.4 |
| Forsythia intermedia | A | R | 100.0 |
| Fragaria x ananassa | A | R | 38.5 |
| Galinsoga ciliata | A | O | 46.7 |
| Galium odoratum | A | O | 21.6 |
| Galium odoratum | A | R | 22.7 |
| Gaultheria hispidula | A | R | 71.9 |
| Gaultheria hispidula | A | O | 90.2 |
| Gentiana lutea | A | R | 100.0 |
| Glechoma hederacea | A | O | 32.7 |
| Glycine max | A | S | 55.1 |
| Glycine max | A | R | 100.0 |
| Glycyrrhiza glabra | A | R | 100.0 |
| Guizotia abyssinica | A | O | 73.8 |
| Hedeoma pulegioides | A | O | 100.0 |
| Helianthus tuberosus | A | O | 37.2 |
| Hordeum hexastichon | A | R | 34.6 |
| Hordeum hexastichon | A | O | 63.6 |
| Hordeum vulgare | A | O | 66.7 |
| Hordeum vulgure subsp. Vulgare | A | O | 33.3 |
| Hypericum henryi | A | O | 66.7 |
| Hyssopus officinalis | A | O | 100.0 |
| Ipomoea Batatas | A | O | 55.1 |
| Iris versicolor | A | R | 24.1 |
| Iris versicolor | A | O | 30.8 |
| Lathyrus sativus | A | O | 20.6 |
| Laurus nobilis | A | O | 33.3 |
| Levisticum officinale | A | O | 87.6 |
| Linum usitatissimum | A | R | 21.4 |
| Linum usitatissimum | A | O | 44.4 |
| Lolium perenne | A | O | 30.9 |
| Lotus corniculatus | A | O | 23.4 |
| Lycopersicon esculentum | A | R | 40.0 |
| Matricaria recutita | A | S | 56.4 |
| Medicago sativa | A | R | 20.5 |
| Melissa officinalis | A | O | 100.0 |
| Mentha piperita | A | O | 22.7 |
| Mentha piperita | A | R | 100.0 |
| Mentha suaveolens | A | O | 53.2 |
| Nepeta cataria | A | O | 100.0 |
| Nicotiana tabacum | A | O | 37.7 |
| Nicotiana tabacum | A | R | 44.3 |
| Oenothera biennis | A | O | 23.8 |
| Oenothera biennis | A | O | 40.0 |
| Oenothera biennis | A | R | 100.0 |
| Origanum vulgare | A | O | 94.7 |
| Panax quinquefolius | A | O | 29.8 |
| Panax quinquefolius | A | O | 35.1 |
| Panax quinquefolius | A | O | 40.4 |
| Pastinaca sativa | A | O | 74.4 |
| Perilla frutescens | A | O | 86.7 |
| Perilla frutescens | A | R | 100.0 |
| Petasites japonicus | A | O | 43.5 |
| Petroselinum crispum | A | O | 100.0 |
| Phalaris arundinacea | A | O | 21.3 |
| Phalaris canariensis | A | O | 22.0 |
| Phaseolus coccineus | A | O | 68.8 |
| Phaseolus mungo | A | S | 58.5 |
| Phaseolus mungo | A | O | 100.0 |
| Phaseolus vulgaris | A | O | 33.3 |
| Phaseolus vulgaris | A | O | 80.3 |
| Phleum pratense | A | O | 20.2 |
| Physalis ixocarpa | A | R | 100.0 |
| Pimpinella anisum | A | O | 86.7 |
| Plantago major | A | O | 99.0 |
| Plectranthus sp. | A | R | 50.0 |
| Plectranthus sp. | A | O | 64.0 |
| Polygonum aviculare | A | O | 55.7 |
| Poterium sanguisorba | A | R | 100.0 |
| Poterium Sanquisorba | A | O | 23.4 |
| Prunus Tomentosa | A | O | 27.6 |
| Raphanus Sativus | A | O | 36.8 |
| Raphanus sativus | A | R | 100.0 |
| Rheun rhabarbarum | A | R | 33.0 |
| Ribes nigrum | A | R | 21.1 |
| Ribes nigrum | A | O | 32.6 |
| Ribes rubrum | A | O | 24.5 |
| Ribes Sylvestre | A | O | 21.1 |
| Ribes Sylvestre | A | R | 30.3 |
| Rosa rugosa | A | R | 21.1 |
| Rosa rugosa | A | O | 36.6 |
| Rosa rugosa | A | O | 40.2 |
| Rosmarinus officinalis | A | O | 95.7 |
| Rubus canadensis | A | R | 25.8 |
| Rubus canadensis | A | O | 31.7 |
| Rubus idaeus | A | O | 85.9 |
| Rubus ideaus | A | R | 66.7 |
| Rumex acetosella | A | O | 27.4 |
| Rumex crispus | A | O | 25.0 |
| Rumex Scutatus | A | O | 21.3 |
| Salvia officinalis | A | O | 21.3 |
| Salvia officinalis | A | O | 85.1 |
| Salvia officinalis | A | R | 100.0 |
| Salvia sclarea | A | O | 29.9 |
| Sanguisorba officinalis | A | O | 23.1 |
| Sanguisorba officinalis | A | R | 48.3 |
| Santolina chamaecyparissus | A | O | 52.9 |
| Satureja montana | A | O | 87.4 |
| Scorzonera hispanica | A | O | 30.8 |
| Secale cereale | A | R | 21.2 |
| Senecio vulgaris | A | O | 42.6 |
| Sesamum indicum | A | O | 27.3 |
| Silybum marianum | A | O | 25.2 |
| Sium sisarum | A | O | 34.4 |
| Solanum dulcamara | A | R | 21.4 |
| Solanum melanocerasum | A | S | 44.6 |
| Solanum melanocerasum | A | R | 60.0 |
| Solanum tuberosum | A | O | 29.2 |
| Solidago sp | A | O | 98.4 |
| Spinacia oleracea | A | O | 40.5 |
| Spinacia oleracea | A | S | 57.7 |
| Stachys affinis | A | O | 23.8 |
| Stachys byzantina | A | O | 96.1 |
| Stellaria graminea | A | O | 34.4 |
| Stellaria media | A | O | 24.6 |
| Symphytum officinale | A | O | 87.7 |
| Symphytum officinale | A | O | 100.0 |
| Tanacetum cinerariifolium | A | O | 70.7 |
| Tanacetum parthenium | A | R | 40.0 |
| Tanacetum parthenium | A | O | 74.7 |
| Tanacetum parthenium | A | R | 100.0 |
| Tanacetum vulgare | A | O | 26.7 |
| Tanacetum vulgare | A | R | 32.7 |
| Tanacetum vulgare | A | O | 98.4 |
| Tanacetum vulgare | A | O | 100.0 |
| Taraxacum officinale | A | R | 22.7 |
| Taraxacum officinale | A | O | 100.0 |
| Teucrium chamaedrys | A | O | 100.0 |
| Thymus praecox subsp arcticus | A | O | 75.6 |
| Thymus praecox subsp arcticus | A | O | 100.0 |
| Thymus serpyllum | A | O | 78.1 |
| Thymus vulgaris | A | O | 90.9 |
| Trichosanthes kirilowii | A | O | 100.0 |
| Trifolium incarnatum | A | S | 76.9 |
| Trifolium pannonicum | A | O | 72.6 |
| Trifolium pratense | A | O | 100.0 |
| Trifolium repens | A | O | 100.0 |
| Triticum durum | A | R | 22.7 |
| Triticum spelta | A | R | 24.0 |
| Triticum spelta | A | O | 32.4 |
| Typha latifolia | A | O | 52.1 |
| Vaccinium Corymbosum | A | R | 53.3 |
| Vaccinium macrocarpon | A | R | 44.3 |
| Valefiana officinalis | A | O | 23.1 |
| Verbascum thapsus | A | O | 65.6 |
| Vitis sp. | A | O | 33.7 |
| Vitis sp. | A | R | 93.3 |
| Zea mays | A | R | 25.0 |
| Zea mays | A | R | 50.0 |
| Achillea millefolium | G | O | 47.7 |
| Agropyron repens | G | O | 93.3 |
| Alchemilla mollis | G | O | 32.1 |
| Allium ascalonicum | G | O | 29.7 |
| Allium sativum | G | R | 100.0 |
| Allium schoenoprasum | G | R | 100.0 |
| Allium tuberosum | G | R | 100.0 |
| Althaea officinalis | G | O | 95.6 |
| Amaranthus caudathus | G | O | 95.3 |
| Amaranthus gangeticus | G | O | 45.7 |
| Amaranthus retroflexus | G | O | 78.3 |
| Ambrosia arternisiifolia | G | O | 73.8 |
| Amelanchier alnifolius | G | O | 50.5 |
| Anethum graveofens | G | O | 100.0 |
| Anthemis nobilis | G | O | 94.3 |
| Apium graveolens | G | O | 21.9 |
| Arctium minus | G | O | 65.9 |
| Arctium minus | G | O | 71.7 |
| Arctostaphylos uva-ursi | G | O | 84.8 |
| Aronia melanocarpa | G | O | 31.5 |
| Arrhenatherum elatius | G | S | 50.8 |
| Artemisia abrotanum | G | O | 52.1 |
| Artemisia absinthium | G | O | 59.7 |
| Artemisia absinthium | G | O | 72.9 |
| Artemisia Ludoviciana | G | O | 64.1 |
| Artemisia Ludoviciana | G | O | 90.7 |
| Artemisia vulgaris | G | O | 55.2 |
| Artemisia vulgaris | G | O | 83.3 |
| Asclepias incarnata | G | O | 38.9 |
| Asclepias incarnata | G | O | 75.6 |
| Asparagus officinalis | G | R | 27.8 |
| Aster sp | G | O | 33.3 |
| Atropa belladonna | G | O | 96.6 |
| Beta vulgaris | G | O | 92.1 |
| Beta vulgaris | G | R | 100.0 |
| Beta vulgaris spp. Maritima | G | R | 100.0 |
| Borago officinalis | G | O | 100.0 |
| Brassica napus | G | R | 40.9 |
| Brassica oleracea | G | R | 66.7 |
| Bromus inermis | G | O | 38.3 |
| Calamintha nepeta | G | R | 25.3 |
| Campanula rapunculus | G | S | 50.8 |
| Campanula rapunculus | G | O | 68.8 |
| Campanula rapunculus | G | O | 69.9 |
| Canna edulis | G | S | 50.8 |
| Capsella bursa-pastoris | G | O | 30.0 |
| Capsicum annuum | G | O | 27.9 |
| Capsicum annuum | G | R | 33.3 |
| Capsicum annuum | G | R | 35.9 |
| Capsicum annuum | G | R | 41.0 |
| Capsicum annuum | G | S | 43.1 |
| Capsicum annuum | G | O | 56.9 |
| Capsicum frutescens | G | O | 60.8 |
| Carthamus tinctorius | G | O | 30.2 |
| Carum carvi | G | O | 28.6 |
| Chaerophyllum bulbosum | G | O | 88.9 |
| Chrysanthemum coronarium | G | O | 82.5 |
| Cicer arietinum | G | R | 31.8 |
| Cichorium endivia subsp endivia | G | O | 100.0 |
| Cichorium intybus | G | O | 100.0 |
| Circium arvense | G | S | 53.8 |
| Circium arvense | G | O | 63.3 |
| Citrullus lanatus | G | O | 40.9 |
| Citrullus lanatus | G | O | 56.9 |
| Coix Lacryma-Jobi | G | O | 100.0 |
| Cornus canadensis | G | O | 20.2 |
| Cornus canadensis | G | O | 35.1 |
| Cucumis anguria | G | R | 40.0 |
| Cucurbita maxima | G | O | 31.4 |
| Cucurbita maxima | G | R | 40.9 |
| Cucurbita moschata | G | O | 23.0 |
| Cucurbita moschata | G | R | 31.8 |
| Cucurbita moschata | G | S | 47.7 |
| Cucurbita pepo | G | O | 29.8 |
| Cucurbita pepo | G | R | 53.3 |
| Cymbopogon martinii | G | O | 100.0 |
| Cynara scolymus | G | O | 27.3 |
| Datura metel | G | O | 54.1 |
| Daucus carota | G | O | 28.6 |
| Daucus carota | G | R | 100.0 |
| Digitalis purpurea | G | R | 100.0 |
| Dirca palustris | G | R | 24.5 |
| Elymus junceus | G | O | 38.3 |
| Erigeron speciosus | G | O | 73.7 |
| Foeniculum vulgare | G | O | 100.0 |
| Forsythia intermedia | G | R | 100.0 |
| Forsythia x intermedia | G | O | 42.1 |
| Galium odoratum | G | R | 63.6 |
| Galium odoratum | G | O | 64.7 |
| Gaultheria hispidula | G | R | 63.4 |
| Gaultheria hispidula | G | O | 69.6 |
| Glechoma hederacea | G | O | 50.5 |
| Glechoma hederacea | G | R | 100.0 |
| Glycine max | G | O | 27.9 |
| Glycine max | G | R | 100.0 |
| Guizotia abyssinica | G | R | 33.3 |
| Guizotia abyssinica | G | O | 83.6 |
| Helianthus annuus | G | R | 100.0 |
| Helianthus strumosus | G | R | 28.9 |
| Helianthus strumosus | G | O | 52.2 |
| Helianthus tuberosus | G | O | 29.3 |
| Helianthus tuberosus | G | O | 54.9 |
| Helichrysum thianschanicum | G | O | 30.5 |
| Heliotropium arborescens | G | R | 29.1 |
| Hysopus officinalis | G | O | 100.0 |
| Ipomoea batatas | G | O | 45.8 |
| Lactuca sativa | G | O | 26.6 |
| Lathyrus sativus | G | O | 72.7 |
| Lathyrus sylvestris | G | O | 33.3 |
| Lathyrus sylvestris | G | R | 56.8 |
| Lavandula angustifolia | G | R | 100.0 |
| Lavandula angustifolia | G | O | 100.0 |
| Lavandula latifolia | G | O | 100.0 |
| Leonurus cardiaca | G | O | 100.0 |
| Levisticum officinale | G | O | 98.1 |
| Levisticum officinale | G | R | 100.0 |
| Linum usitatissimum | G | O | 42.9 |
| Lolium perenne | G | O | 25.5 |
| Lotus tetragonolobus | G | R | 49.2 |
| Lupinus polyphyllus | G | O | 33.3 |
| Lycopersicon esculentum | G | O | 29.5 |
| Lycopersicon esculentum | G | R | 43.3 |
| Lycopersicon pimpinellifolium | G | R | 100.0 |
| Malva moschata | G | O | 100.0 |
| Medicago sativa | G | O | 32.6 |
| Melissa officinalis | G | O | 100.0 |
| Mentha piperita | G | O | 40.3 |
| Mentha suaveolens | G | O | 79.2 |
| Monarda didyma | G | R | 100.0 |
| Nepeta cataria | G | O | 100.0 |
| Ocimum basilicum | G | O | 80.5 |
| Oenothera biennis | G | O | 41.7 |
| Oenothera biennis | G | R | 100.0 |
| Origanum majorana | G | O | 67.4 |
| Origanum vulgare | G | O | 100.0 |
| Oxalis Deppei | G | O | 22.2 |
| Oxalis Deppei | G | S | 44.6 |
| Oxyria digyna | G | O | 21.3 |
| Panax quinquefolius | G | O | 25.5 |
| Panax quinquefolius | G | O | 38.3 |
| Panicum miliaceum | G | R | 83.3 |
| Pennisetum alopecuroides | G | R | 21.5 |
| Petasites japonicus | G | O | 40.6 |
| Petroselinum crispum | G | O | 100.0 |
| Peucedanum cervaria | G | O | 42.9 |
| Phaseolus mungo | G | O | 100.0 |
| Phaseolus vulgaris | G | O | 54.8 |
| Phaseolus vulgaris | G | O | 67.2 |
| Plantago major | G | O | 95.2 |
| Plectranthus sp. | G | R | 100.0 |
| Plectranthus sp. | G | O | 100.0 |
| Poa compressa | G | O | 20.2 |
| Portulaca oleracera | G | O | 60.0 |
| Potentilla anserina | G | R | 100.0 |
| Poterium sanguisorba | G | O | 21.3 |
| Poterium sanguisorba | G | R | 100.0 |
| Prunella vulgaris | G | O | 70.3 |
| Raphanus Raphanistrum | G | O | 33.3 |
| Raphanus Raphanistrum | G | R | 80.0 |
| Raphanus sativus | G | O | 52.6 |
| Raphanus sativus | G | R | 100.0 |
| Ribes nigrum | G | O | 42.1 |
| Ribes Sylvestre | G | R | 32.0 |
| Ricinus communis | G | R | 100.0 |
| Rosa rugosa | G | O | 52.4 |
| Rosa rugosa | G | O | 90.2 |
| Rosmarinus officinalis | G | O | 100.0 |
| Rubus ideaus | G | O | 34.8 |
| Rubus occidentalis | G | R | 60.0 |
| Rubus occidentalis | G | O | 65.3 |
| Rumex crispus | G | O | 43.3 |
| Ruta graveolens | G | O | 23.0 |
| Salvia officinalis | G | O | 100.0 |
| Salvia officinalis | G | R | 100.0 |
| Sambucus canadensis | G | O | 80.6 |
| Sambucus ebulus | G | R | 21.1 |
| Sambucus ebulus | G | O | 36.8 |
| Sanguisorba officinalis | G | O | 43.6 |
| Santolina chamaecyparissus | G | O | 50.6 |
| Saponaria officinalis | G | O | 85.6 |
| Satureja hortensis | G | R | 36.8 |
| Satureja hortensis | G | O | 68.4 |
| Senecio vulgaris | G | O | 31.1 |
| Sesamum indicum | G | O | 27.3 |
| Sium sisarum | G | O | 20.8 |
| Sium sisarum | G | O | 47.8 |
| Solanum melanocerasum | G | O | 23.5 |
| Solanum melongens | G | O | 28.6 |
| solanum melongens | G | R | 41.2 |
| Solidago sp | G | O | 72.1 |
| Sonchus oleraceus | G | O | 95.1 |
| Stachys Affinis | G | O | 38.1 |
| Stachys byzantina | G | O | 28.6 |
| Stellaria graminea | G | O | 39.3 |
| Stellaria media | G | O | 21.3 |
| Symphytum officinale | G | R | 37.8 |
| Symphytum officinale | G | S | 43.1 |
| Symphytum officinale | G | O | 92.6 |
| Symphytum officinale | G | O | 100.0 |
| Tanacetum cinerariifolium | G | O | 91.3 |
| Tanacetum parthenium | G | R | 60.0 |
| Tanacetum parthenium | G | O | 86.7 |
| Tanacetum vulgare | G | O | 44.4 |
| Tanacetum vulgare | G | O | 67.9 |
| Tanacetum vulgare | G | O | 85.7 |
| taraxacum officinale | G | R | 40.9 |
| taraxacum officinale | G | O | 100.0 |
| Teucrium chamaedrys | G | R | 33.3 |
| Teucrium chamaedrys | G | O | 66.7 |
| Thymus fragantissimus | G | O | 24.1 |
| Thymus praecox subsp arcticus | G | R | 25.0 |
| Thymus praecox subsp arcticus | G | O | 92.7 |
| Thymus praecox subsp arcticus | G | O | 100.0 |
| Thymus serpyllum | G | O | 100.0 |
| Thymus vulgaris | G | O | 64.4 |
| Thymus x citriodorus | G | O | 72.7 |
| Tiarella cordifolia | G | O | 92.4 |
| Trifolium hybridum | G | O | 29.5 |
| Trifolium pannonicum | G | O | 54.7 |
| Trifolium pratense | G | O | 92.9 |
| Trifolium repens | G | O | 100.0 |
| Triticum spelta | G | R | 37.3 |
| Triticum turgidum | G | O | 59.5 |
| Typha latifolia | G | O | 23.4 |
| Vaccinium corymbosum | G | O | 26.5 |
| Vaccinum angustifolium | G | O | 27.7 |
| Vaccinum macrocarpon | G | R | 33.0 |
| Valeriana officinalis | G | R | 27.6 |
| Valeriana officinalis | G | O | 51.3 |
| Verbascum thapsus | G | O | 21.3 |
| Vinca minor | G | O | 28.6 |
| Vitis sp. | G | R | 40.0 |
| Vitis sp. | G | O | 42.6 |
| Zea mays | G | R | 26.9 |
| Zea mays | G | R | 100.0 |
| Abies lasiocarpa | T | O | 25.6 |
| Agastache foeniculum | T | O | 100.0 |
| Agropyron cristatum | T | O | 20.2 |
| Agrostis alba | T | O | 24.5 |
| Alchemilla mollis | T | O | 33.3 |
| Alchemilla mollis | T | S | 49.2 |
| Alchemilla mollis | T | O | 66.2 |
| Allium ampeloprasum | T | O | 100.0 |
| Allium ascalonicum | T | O | 29.7 |
| Allium ascalonicum | T | R | 38.7 |
| Allium cepa | T | R | 100.0 |
| Allium tuberosum | T | R | 100.0 |
| Alpinia officinarum | T | R | 50.0 |
| Althaea officinalis | T | O | 58.6 |
| Amaranthus candathus | T | R | 22.9 |
| Amaranthus candatus | T | O | 93.2 |
| Amaranthus caudathus | T | O | 100.0 |
| Amaranthus gangeticus | T | O | 57.1 |
| Amaranthus retroflexus | T | O | 100.0 |
| Ambrosia artemisiifolia | T | O | 86.9 |
| Amelanchier alnifolia | T | O | 50.5 |
| Anthemis nobilis | T | O | 100.0 |
| Anthriscus cerefolium | T | O | 100.0 |
| Aralia cordata | T | R | 100.0 |
| Arctium minus | T | O | 68.3 |
| Aronia melanocarpa | T | O | 50.0 |
| Aronia prunifolia | T | O | 44.7 |
| Arrhenatherum elatius | T | O | 78.7 |
| Artemisia absinthium | T | O | 58.4 |
| Artemisia dracunculus | T | R | 28.6 |
| Artemisia dracunculus | T | O | 86.3 |
| Artemisia Ludoviciana | T | O | 48.8 |
| Artemisia vulgaris | T | O | 50.0 |
| Artemisia vulgaris | T | O | 82.8 |
| Asclepias incarnata | T | O | 72.9 |
| Asparagus officinalis | T | O | 69.8 |
| Aster sp | T | O | 35.0 |
| Avena sativa | T | O | 31.8 |
| Baptisia tinctoria | T | O | 33.8 |
| Beta vulgaris | T | O | 25.5 |
| Beta vulgaris | T | O | 28.6 |
| Beta vulgaris | T | R | 34.6 |
| Beta vulgaris | T | S | 43.6 |
| Beta vulgaris | T | O | 54.5 |
| Beta vulgaris | T | R | 100.0 |
| Beta vulgaris spp. Maritima | T | R | 100.0 |
| Brassica nigra | T | R | 45.5 |
| Brassica oleracea | T | O | 50.0 |
| Brassica oleracea | T | R | 100.0 |
| Bromus inermis | T | O | 30.9 |
| Calamagrostis arundiflora | T | O | 85.6 |
| Calendula officinalis | T | O | 23.7 |
| Campanula rapunculus | T | O | 25.0 |
| Canna edulis | T | O | 26.3 |
| Capsella bursa-pastoris | T | O | 21.7 |
| Capsicum annum | T | O | 46.1 |
| Capsicum annuum | T | R | 20.5 |
| Capsicum annuum | T | O | 23.3 |
| Capsicum annuum | T | R | 41.0 |
| Capsicum frutescens | T | O | 58.8 |
| Carthamus tinctorius | T | O | 36.5 |
| Carum carvi | T | O | 88.6 |
| Chaerophyllum bulbosum | T | O | 25.0 |
| Chaerophyllum bulbosum | T | O | 95.2 |
| Chelidonium majus | T | O | 27.1 |
| Chelidonium majus | T | R | 50.0 |
| Chenopodium bonus-henricus | T | O | 60.0 |
| Chenopodium quinoa | T | R | 31.5 |
| Chenopodium quinoa | T | O | 50.0 |
| Chrysanthemum coronarium | T | R | 65.5 |
| Chrysanthemum coronarium | T | O | 100.0 |
| Cicer arietinum | T | R | 27.3 |
| Cichorium endivia subsp endivia | T | R | 27.3 |
| Cichorium endivia subsp endivia | T | O | 97.3 |
| Cichorium intybus | T | O | 100.0 |
| Cimicifuga racemosa | T | R | 22.2 |
| Circium arvense | T | O | 78.3 |
| Citrullus lanatus | T | R | 26.7 |
| Citrullus lanatus | T | O | 45.5 |
| Citrullus lanatus | T | O | 62.7 |
| Coix Lacryma-Jobi | T | O | 77.3 |
| Coriandrum sativum | T | O | 90.0 |
| Cornus canadensis | T | O | 29.3 |
| Cucumis anguria | T | R | 50.0 |
| Cucumis anguria | T | O | 70.1 |
| Cucumis melo | T | R | 20.5 |
| Cucumis melo | T | O | 51.0 |
| Cucumis sativus | T | O | 23.4 |
| Cucurbita maxima | T | O | 50.0 |
| Cucurbita moschata | T | O | 84.9 |
| Cucurbita pepo | T | R | 20.5 |
| Cucurbita pepo | T | O | 39.2 |
| Cucurbita pepo | T | S | 53.8 |
| Curcuma zedoaria | T | O | 24.6 |
| Cymbopogon citratus | T | O | 100.0 |
| Cynara scolymus | T | R | 33.3 |
| Dactilis Glomerata | T | O | 20.2 |
| Datura metel | T | O | 37.8 |
| Datura stramonium | T | R | 50.0 |
| Daucus carota | T | R | 21.1 |
| Daucus carota | T | O | 30.3 |
| Daucus carota | T | O | 49.3 |
| Daucus carota | T | S | 52.3 |
| Dipsacus sativus | T | O | 73.7 |
| Dirca palustris | T | O | 88.5 |
| Eleusine coracana | T | S | 49.2 |
| Elymus junceus | T | O | 35.1 |
| Erigeron speciosus | T | O | 67.8 |
| Fagopyrum esculentum | T | O | 27.3 |
| Foeniculum vulgare | T | R | 80.0 |
| Forsythia intermedia | T | O | 50.9 |
| Forsythia x intermedia | T | O | 57.9 |
| Fucus vesiculosus | T | O | 83.7 |
| Fucus vesiculosus | T | R | 100.0 |
| Galinsoga ciliata | T | O | 56.7 |
| Galium aparine | T | O | 60.5 |
| Galium odoratum | T | R | 31.8 |
| Gaultheria hispidula | T | O | 33.7 |
| Gaultheria procumbens | T | O | 25.0 |
| Gentiana lutea | T | O | 98.1 |
| Gentiana macrophylla | T | O | 100.0 |
| Glechoma hederacea | T | O | 62.6 |
| Glycine max | T | O | 26.2 |
| Glycyrrhiza glabra | T | R | 50.0 |
| Glycyrrhiza glabra | T | S | 51.3 |
| Guizotia abyssinica | T | O | 39.3 |
| Guizotia abyssinica | T | R | 100.0 |
| Hedeoma pulegioides | T | O | 100.0 |
| Helianthus annus | T | O | 75.8 |
| Helianthus strumosus | T | R | 55.6 |
| Helianthus tuberosus | T | O | 22.1 |
| Helichrysum angustifolium | T | O | 96.1 |
| Helichrysum thianschanicum | T | O | 70.5 |
| Heliotropium arborescens | T | O | 83.2 |
| Helleborus niger | T | O | 24.1 |
| Herba Schizonepetae | T | O | 60.5 |
| Hibiscus cannabinus | T | S | 52.6 |
| Hordeum vulgare | T | O | 77.8 |
| Hydrastis canadensis | T | O | 64.9 |
| Hypericum henryi | T | O | 100.0 |
| Hypericum perforatum | T | R | 31.0 |
| Hyssopus officinalis | T | O | 100.0 |
| Inula helenium | T | O | 100.0 |
| Ipomoea batalas | T | O | 91.5 |
| Iris versicolor | T | O | 35.9 |
| Juniperus communis | T | O | 83.8 |
| Krameria Triandra | T | O | 25.6 |
| Lactuca sativa | T | O | 100.0 |
| Lathyrus Sativus | T | R | 27.3 |
| Lathyrus Sativus | T | O | 33.3 |
| Lathyrus sylvestris | T | O | 20.3 |
| Lathyrus sylvestris | T | R | 100.0 |
| Laurus nobilis | T | R | 23.8 |
| Laurus nobilis | T | O | 26.0 |
| Lavandula latifolia | T | R | 100.0 |
| Lavandula latifolia | T | O | 100.0 |
| Lens culinaris subsp culinaris | T | O | 21.3 |
| Leonorus cardiaca | T | O | 57.9 |
| Lepidium sativum | T | O | 31.6 |
| Levisticum officinale | T | O | 90.5 |
| Levisticum officinale | T | R | 100.0 |
| Linum usitatissimum | T | O | 23.8 |
| Lonicera syringantha | T | O | 79.5 |
| Lotus couniculatus | T | R | 46.7 |
| Lupinus polyphyllus lindl. | T | O | 36.6 |
| Lycopersicon esculentum | T | R | 60.0 |
| Malus hupehensis | T | R | 100.0 |
| Malva sylvestris | T | O | 100.0 |
| Matricaria spp. | T | O | 100.0 |
| Medicago sativa | T | O | 27.7 |
| Melissa officinalis | T | O | 100.0 |
| Menyanthes trifoliata | T | O | 44.9 |
| Menyanthes trifoliata | T | R | 50.0 |
| Miscanthus sinensis | T | R | 23.5 |
| Miscanthus sinensis | T | O | 24.6 |
| Nepeta cataria | T | O | 78.9 |
| Ocimum Basilicum | T | R | 35.7 |
| Ocimum Basilicum | T | O | 100.0 |
| Oenothera biennis | T | R | 100.0 |
| Origanum vulgare | T | O | 94.7 |
| Origanum vulgare | T | R | 100.0 |
| Oxalis Deppei | T | O | 21.1 |
| oxyria digyna | T | O | 24.6 |
| Panax quinquefolius | T | O | 39.4 |
| Panicum miliaceum | T | R | 20.8 |
| Pastinaca sativa | T | O | 21.3 |
| Pastinaca sativa | T | R | 25.0 |
| Pastinaca sativa | T | R | 25.0 |
| Pastinaca sativa | T | O | 79.4 |
| Pastinaca sativa | T | O | 100.0 |
| Perilla frutescens | T | O | 96.0 |
| Perilla frutescens | T | R | 100.0 |
| Petasites Japonicus | T | O | 29.0 |
| Petroselinum crispum | T | R | 40.0 |
| Peucedanum oreaselinum | T | S | 55.1 |
| Pfaffia paniculata | T | R | 100.0 |
| Phaseolus mungo | T | O | 70.2 |
| Phaseolus vulgaris | T | O | 71.4 |
| Phaseolus vulgaris | T | O | 100.0 |
| Phaseolus vulgaris | T | R | 100.0 |
| Physalis ixocarpa | T | O | 25.5 |
| Pimpinella anisum | T | R | 100.0 |
| Pimpinella anisum | T | O | 100.0 |
| Pisum sativum | T | O | 37.5 |
| Plantago major | T | O | 100.0 |
| Plectranthus sp. | T | O | 36.0 |
| Plectranthus sp. | T | R | 80.0 |
| Poa pratensis | T | O | 38.3 |
| Populus X petrowskyana | T | O | 25.5 |
| Prunella vulgaris | T | O | 23.3 |
| Prunella vulgaris | T | O | 88.1 |
| Raphanus raphanistrum | T | O | 73.7 |
| Raphanus raphanistrum | T | R | 100.0 |
| Raphanus sativus | T | S | 60.3 |
| Raphanus sativus | T | R | 100.0 |
| Reseda luteola | T | O | 100.0 |
| Rheum officinale | T | O | 36.8 |
| Ribes sativum | T | O | 20.4 |
| Ribes Sylvestre | T | R | 44.3 |
| Ricinus communis | T | R | 100.0 |
| Rosmarinus officinalis | T | R | 60.0 |
| Rosmarinus officinalis | T | O | 100.0 |
| Rubus canadensis | T | R | 32.0 |
| Rubus canadensis | T | O | 34.7 |
| Rubus idaeus | T | O | 93.5 |
| Rubus ideaus | T | R | 100.0 |
| Rubus occidentalis | T | O | 38.6 |
| Rubus occidentalis | T | S | 52.3 |
| Rubus occidentalis | T | R | 100.0 |
| Rumex acetosella | T | O | 26.3 |
| Rumex crispus | T | O | 30.0 |
| Rumex scutatus | T | O | 23.0 |
| Ruta graveolens | T | O | 62.1 |
| Saccharum officinarum | T | O | 27.0 |
| Salvia officinalis | T | O | 92.0 |
| Salvia officinalis | T | O | 93.3 |
| Sambucus canadensis | T | O | 42.9 |
| Sanguisorba officinalis | T | O | 68.6 |
| Santolina chamaecyparissus | T | O | 66.7 |
| Saponaria officinalis | T | O | 36.6 |
| Saponaria officinalis | T | O | 84.7 |
| Satureja montana | T | O | 80.5 |
| Satureja repandra | T | O | 47.1 |
| Senecio vulgaris | T | O | 44.3 |
| Setaria italica | T | O | 27.9 |
| Silybum marianum | T | O | 31.0 |
| Sium sisarum | T | O | 24.8 |
| Sium sisarum | T | R | 25.5 |
| Solanum dulcamara | T | R | 21.4 |
| Solanum melongena | T | R | 25.8 |
| Solanum melongena | T | O | 34.9 |
| Solanum tuberosum | T | O | 38.1 |
| Solidago canadensis | T | O | 100.0 |
| Solidago sp | T | O | 73.8 |
| Sonchus oleraceus | T | O | 100.0 |
| Sorghum durra | T | O | 23.8 |
| Spinacia oleracea | T | R | 29.3 |
| Stachys affinis | T | R | 23.6 |
| Stachys affinis | T | O | 23.9 |
| Stachys affinis | T | O | 50.0 |
| Stachys byzantina | T | O | 41.6 |
| Stellaria graminea | T | O | 62.3 |
| Stipa capillata | T | O | 27.1 |
| Symphytum officinale | T | R | 28.9 |
| Symphytum officinale | T | O | 87.7 |
| Symphytum officinale | T | O | 97.8 |
| Tanacetum cinerariifolium | T | O | 62.7 |
| Tanacetum parthenium | T | O | 94.7 |
| Tanacetum vulgare | T | R | 28.9 |
| Tanacetum vulgare | T | S | 47.7 |
| Tanacetum vulgare | T | O | 75.6 |
| Tanacetum vulgare | T | O | 95.2 |
| Tanacetum vulgare | T | O | 100.0 |
| Taraxacum officinale | T | O | 95.3 |
| Thymus praecox subsp arcticus | T | R | 24.4 |
| Thymus praecox subsp arcticus | T | O | 60.0 |
| Thymus praecox subsp arcticus | T | O | 90.0 |
| Thymus-pseudolanuginosus | T | O | 83.9 |
| Thymus serpyllum | T | O | 100.0 |
| Tiarella cordifolia | T | O | 93.3 |
| Tragopogon porrifolius | T | O | 34.4 |
| Tragopogon porrifolius | T | O | 58.0 |
| Trichosanthes kirilowii | T | R | 25.3 |
| Trifolium pannonicum | T | O | 61.1 |
| Trifolium pratense | T | O | 92.9 |
| Trifolium repens | T | O | 100.0 |
| Triticum aestivum | T | O | 29.5 |
| Triticum durum | T | O | 100.0 |
| Triticum turgidum | T | O | 29.7 |
| Ulmus americana | T | O | 76.9 |
| Ulmus americana | T | O | 81.0 |
| Urtica dioica | T | R | 40.9 |
| Vaccinium angustifolium | T | R | 26.3 |
| Vaccinium angustifolium | T | O | 28.3 |
| Vaccinium angustifolium | T | O | 47.6 |
| Vaccinium angustifolium | T | R | 100.0 |
| Vaccinium corymbosum | T | O | 21.4 |
| Vaccinium macrocarpon | T | R | 80.0 |
| Valeriana officinalis | T | O | 43.6 |
| Vicia sativa | T | S | 43.1 |
| Vitiis sp. | T | O | 26.7 |
| Vitiis sp. | T | R | 93.3 |
| Zea mays | T | R | 21.2 |
| Zea mays | T | R | 100.0 |

TABLE 7
Cath G

| | | | Inhibition |
|---|---|---|---|
| Nom latin | Stress | Extrait | (%) |
| Achillea millefolium | A | V | 40.1 |
| Achillea millefolium | A | O | 29.5 |
| Acorus calamus | A | R | 68.6 |
| Adiantum pedatum | A | R | 29.7 |
| Agastache foeniculum | A | O | 36.8 |
| Agastache foeniculum | A | S | 22.4 |
| Agropyron rupens | A | S | 24.5 |
| Alchemilla mollis | A | W | 100.0 |
| Alchemilla mollis | A | S | 81.1 |
| Alchemilla mollis | A | O | 51.5 |
| Alchemilla mollis | A | S | 78.6 |
| Alchemilla mollis | A | O | 82.9 |
| Alchemilla mollis | A | S | 35.6 |
| Alkanna tinctoria | A | O | 51.6 |
| Alkanna tinctoria | A | R | 100.0 |
| Allium Tuberosum | A | S | 20.6 |
| Althaea officinalis | A | R | 21.6 |
| Althaea officinalis | A | S | 39.6 |
| Ambrosia artemisiifolia linné | A | O | 47.6 |
| Ambrosia artemisiifolia linné | A | R | 38.2 |
| Amelanchier sanguinea (Pursh) DC. | A | W | 29.7 |
| Angelica archangelica | A | S | 68.1 |
| Anthemis tinctoria | A | O | 26.0 |
| Anthemis tinctoria | A | V | 28.4 |
| Anthemis tinctorium | A | O | 46.9 |
| Arachis hypogaea | A | V | 84.5 |
| Aralia nudicaulis | A | S | 61.9 |
| Arctostaphylos uva-ursi | A | O | 25.0 |
| Arctostaphylos uva-ursi | A | R | 100.0 |
| Arctostaphylos uva-ursi | A | S | 38.4 |
| Aronia melanocarpa (Michx.) Ell. | A | O | 24.4 |
| Aronia melanocarpa (Michx.) Ell. | A | R | 27.3 |
| Aronia melanocarpa (Michx.) Ell. | A | W | 47.8 |
| Artemisia dracunculus sativa | A | W | 32.2 |
| Artemisis Ludoviciana | A | O | 88.8 |
| Aster sp ? | A | O | 47.2 |
| Aster sp ? | A | R | 100.0 |
| Beta vulgaris | A | R | 23.9 |
| Brassica napus | A | R | 22.3 |
| Brassica napus | A | S | 22.8 |
| Brassica nigra | A | S | 47.2 |
| Brassica rapa | A | S | 46.0 |
| Capsella bursa-pastoris (linné) médicus | A | R | 43.4 |
| Chaerophyllum bulbosom | A | V | 90.7 |
| Chaerophyllum bulbosom | A | W | 57.4 |
| chenopodium bonus-henricus | A | R | 23.7 |
| Chichorium endivia | A | O | 53.0 |
| Chrysanthemum leucanthemum linné | A | O | 55.5 |
| Cicer arietinum | A | R | 26.2 |
| Cichorium intybus | A | O | 100.0 |
| Cichorium intybus | A | V | 83.6 |
| Cichorium intybus | A | O | 51.0 |
| Crataegus sp ? | A | O | 100.0 |
| Crataegus sp ? | A | R | 81.6 |
| Cymbopogan citratus | A | S | 33.9 |
| Datisca cannabina | A | S | 20.2 |
| Daucus carota | A | O | 62.0 |
| Daucus carota | A | W | 99.4 |
| Dirca palustris | A | R | 24.9 |
| Dirca palustris | A | S | 47.0 |
| Dryopteris filix-mas | A | O | 24.1 |
| Dryopteris filix-mas | A | R | 95.7 |
| Echinacea purpurea | A | V | 80.7 |
| Echinacea purpurea | A | W | 100.0 |
| Filipendula rubra | A | O | 20.2 |
| Filipendula rubra | A | S | 77.6 |
| Foeniculum vulgare | A | R | 23.3 |
| Fragaria x ananassa | A | O | 32.3 |
| Fragaria x ananassa | A | W | 100.0 |
| Fragaria x ananassa | A | S | 100.0 |
| Fragaria x ananassa | A | S | 100.0 |
| Frangoria x ananassa | A | W | 100.0 |
| Frangoria x ananassa | A | V | 100.0 |
| Galinsoga ciliata (Rofiresque) Blake | A | R | 21.2 |
| Gaultheria hispidula (L.) Muhl. | A | R | 85.3 |
| Gaultheria hispidula (L.) Muhl. | A | R | 100.0 |
| Gaultheria procumbens | A | W | 56.1 |
| Glycine Max | A | S | 36.0 |
| Glycine max | A | S | 38.7 |
| Glycyrrhiza glabra | A | W | 46.2 |
| Glycyrrhiza glabra | A | S | 35.5 |
| Glycyrrhiza glabra | A | R | 100.0 |
| Hamamelis virginiana | A | R | 100.0 |
| Helianthus tuberosus | A | W | 22.6 |
| Helichrysum angustifolium | A | V | 82.6 |
| Heliotropium arborescens | A | O | 57.3 |
| Heliotropium arborescens | A | R | 57.2 |
| Hordeum vulgare | A | O | 34.3 |
| Hypericum henryi | A | O | 30.4 |
| Hypericum perforatum | A | R | 100.0 |
| Inula helenium | A | S | 64.0 |
| Isatis tinctoria | A | O | 94.0 |
| Laurus nobilis | A | S | 49.9 |
| Lavendula latifolia | A | W | 100.0 |
| Lavendula latifolia | A | V | 48.7 |
| Leonorus cardiaca | A | R | 100.0 |
| Levisecum officinale | A | V | 46.8 |
| Lolium multiflorum | A | O | 34.1 |
| Melissa officinalis | A | O | 54.1 |
| Melissa officinalis | A | W | 100.0 |
| Melissa officinalis | A | V | 80.7 |
| Melissa officinalis | A | O | 100.0 |
| Mentha pulegium | A | O | 29.1 |
| Mentha spicata | A | V | 47.0 |
| Nepeta cataria | A | V | 57.6 |
| Ocrothera biennis | A | S | 33.1 |
| Oenothera biennis linné | A | O | 47.4 |
| Oenothera biennis linné | A | R | 100.0 |
| Origanum majorana | A | S | 34.6 |
| Origanum vulgare | A | V | 65.9 |
| Origanum vulgare | A | W | 48.2 |
| Origanum vulgare | A | V | 70.0 |
| Origanum vulgare | A | W | 62.9 |
| Origanum vulgare | A | O | 68.4 |
| Origanum vulgare | A | V | 81.9 |
| Origanum vulgare | A | W | 61.3 |
| Origanum vulgare | A | S | 21.7 |
| Oxyria digyna | A | V | 40.1 |
| Perilla frutescens | A | V | 65.0 |
| Perilla frutescens | A | W | 51.9 |
| Peucedanum cervaria | A | R | 28.3 |
| Peucedanum cervaria | A | R | 45.1 |
| Phaseolus Vulgaris | A | S | 38.4 |
| Phaseolus Vulgaris | A | S | 26.3 |
| Phytolacca americana | A | S | 27.8 |
| Plantago coronopus | A | O | 22.7 |
| Polygonum aviculare linné | A | R | 76.0 |
| Poterium sanguisorba | A | O | 20.1 |
| Poterium sanguisorba | A | R | 93.1 |
| Poterium sanguisorba | A | V | 47.7 |
| Poterium sanguisorba | A | S | 36.1 |
| Pteridium aquilinum | A | O | 25.7 |
| Pteridium aquilinum | A | R | 100.0 |
| Ribes nidigrolaria | A | W | 51.8 |
| Ribes Nigrum | A | W | 100.0 |
| Ribes nigrum | A | S | 33.6 |
| Ribes nigrum L. | A | W | 58.8 |
| Ribes nigrum L. | A | O | 21.5 |
| Ribes Salivum | A | R | 21.4 |
| Ricinus communis | A | R | 100.0 |
| Rosa rugosa thunb. | A | W | 20.1 |
| Rosa rugosa thunb. | A | W | 100.0 |
| Rosa rugosa thunb. | A | R | 100.0 |
| Rosmarinus officinalis | A | O | 100.0 |
| Rosmarinus officinalis | A | R | 64.0 |
| Rosmarinus officinalis | A | W | 55.6 |
| Rosmarinus officinalis | A | V | 76.7 |
| Rubus allegheniensis | A | S | 32.1 |
| Rubus canadensis | A | W | 94.5 |
| Rubus canadensis | A | S | 64.2 |
| Rubus idaeus | A | S | 86.0 |
| Rubus idaeus | A | O | 29.5 |
| Rubus idaeus | A | W | 38.7 |
| Rubus idaeus | A | S | 41.0 |
| Rubus idaeus | A | W | 100.0 |
| Rubus idaeus L. | A | V | 30.2 |
| Rubus idaeus L. | A | W | 29.4 |
| Rubus idaeus L. | A | S | 100.0 |
| Rubus ideaus | A | R | 100.0 |
| Rubus ideaus | A | S | 67.1 |
| Rubus occidentalis | A | S | 100.0 |
| Rumex crispus linné | A | R | 100.0 |
| Salvia elegens | A | W | 69.7 |
| Salvia officinalis | A | W | 100.0 |
| Salvia officinalis | A | V | 58.0 |
| Salvia officinalis | A | O | 100.0 |
| Salvia officinalis | A | R | 39.9 |
| Salvia officinalis | A | V | 45.7 |
| Salvia officinalis | A | W | 65.4 |
| Salvia sclarea | A | W | 29.1 |
| Santolina | A | W | 65.5 |
| Satureja montana | A | V | 72.2 |
| Satureja montana | A | W | 100.0 |
| Satureja montana | A | O | 90.5 |
| Satureja montana | A | V | 28.9 |
| Scuttellaria lateriflora | A | S | 23.7 |
| Sonchus oleraceus L. | A | O | 25.9 |
| Sorghum dochra bicolor | A | O | 25.6 |
| Sorghum durra (Stapif) | A | O | 46.9 |
| Symphytum officinale | A | O | 99.4 |
| Symphytum officinale | A | O | 97.8 |
| Tanacetum cinerarifolium | A | W | 28.2 |
| Tanacetum parthenium | A | W | 34.8 |
| Tanacetum vulgare | A | W | 80.0 |
| Tanacetum vulgare | A | V | 53.8 |
| Tanacetum vulgare | A | O | 35.9 |
| Tanacetum vulgare | A | R | 68.8 |
| Tanacetum vulgare “Goldsticks” | A | V | 51.9 |
| Taraxacum officinale | A | W | 28.5 |
| Taraxacum officinale | A | V | 82.3 |
| Thymus praecox subsp arctitus | A | O | 43.4 |
| Thymus pseudolanuginosus | A | V | 29.7 |
| Thymus serpyllum | A | O | 100.0 |
| Thymus serpyllum | A | W | 73.6 |
| Thymus serpyllum | A | V | 74.9 |
| Thymus vulgaris | A | O | 35.6 |
| Thymus vulgaris | A | R | 66.5 |
| Thymus vulgaris “Argenteus” | A | V | 73.9 |
| Triticum furgidum?? | A | O | 21.6 |
| Vaccinum augustifolium | A | S | 26.1 |
| Vaccinum Corymbosum | A | W | 95.7 |
| Vaccinum macrocarpon | A | W | 46.1 |
| Valerianella locusta | A | S | 96.0 |
| Veronica officinalis | A | S | 26.4 |
| Viburnum trilobum Marsh. | A | W | 25.0 |
| Vicia sativa | A | O | 28.2 |
| Vicia villosa | A | O | 34.5 |
| Vitia sp. | A | W | 26.0 |
| Vitia sp. | A | S | 41.6 |
| Vitia sp. | A | W | 100.0 |
| Vitia sp. | A | S | 30.8 |
| Vitia sp. | A | O | 22.3 |
| Vitia sp. | A | S | 28.5 |
| Zea Mays | A | S | 32.3 |
| Zea Mays | A | S | 34.5 |
| Achillea millefolium | G | W | 30.6 |
| Achillea millefolium | G | V | 71.1 |
| Aconitum napellus | G | R | 100.0 |
| Acorus calamus | G | R | 27.8 |
| Adiantum pedatum | G | R | 100.0 |
| Agastache toeniculum “Snow Pike” | G | V | 46.9 |
| Agastache toeniculum “Snow Pike” | G | W | 71.5 |
| Alchemilla mollis | G | W | 100.0 |
| Alchemilla mollis | G | O | 52.6 |
| Alchemilla mollis | G | S | 80.7 |
| Alchemilla mollis | G | O | 33.4 |
| Alchemilla mollis | G | S | 38.7 |
| althaea officinalis | G | R | 27.5 |
| althaea officinalis | G | S | 36.9 |
| Ambrosia artemisiifolia linné | G | O | 48.4 |
| Ambrosia artemisiifolia linné | G | R | 36.0 |
| Amelanchier sanguinea (Pursh) DC. | G | W | 46.5 |
| Angelica archangelica | G | S | 39.1 |
| Arachis hypogaea | G | V | 81.8 |
| Aralia nudicaulis | G | S | 44.9 |
| Arctium minus (Hill) Bernhardi | G | O | 35.6 |
| Arctostaphylos uva-ursi | G | S | 59.9 |
| Aronia melanocarpa (Michx.) Ell. | G | W | 28.4 |
| Artemisia Ludoviciana | G | O | 66.0 |
| Aster sp ? | G | O | 51.8 |
| Aster sp ? | G | R | 100.0 |
| Beta vulgaris | G | R | 26.5 |
| Brassica napus | G | R | 32.9 |
| Brassica napus | G | S | 33.5 |
| Brassica oleracea | G | S | 100.0 |
| Calamintha nepeta | G | V | 51.5 |
| Calendula officinalis L. | G | O | 26.7 |
| Canna edulis | G | O | 20.6 |
| Chaerophyllum bulbosum | G | O | 37.0 |
| Chaerophyllum bulbosum | G | V | 88.6 |
| Chaerophyllum bulbosum | G | W | 26.5 |
| Chichorium endivia | G | S | 25.2 |
| Chrysanthemum leucanthemum linné | G | O | 44.2 |
| Cicer arietinum | G | R | 26.1 |
| Cichorium endivia | G | O | 23.7 |
| Cichorium intybus | G | O | 100.0 |
| Cichorium intybus | G | V | 79.2 |
| Cichorium intybus | G | O | 82.5 |
| Crataegus sp ? | G | W | 27.9 |
| Cynara scolymus | G | O | 66.3 |
| Dirca palustris | G | R | 28.8 |
| Dirca palustris | G | S | 85.2 |
| Dryopteris filix-mas | G | R | 100.0 |
| Echinacea purpurea | G | V | 84.2 |
| Echinacea purpurea | G | O | 83.2 |
| Erigeron speciosus (Lindl.) D.C. | G | O | 46.1 |
| Fagopyrum esculentum | G | O | 27.5 |
| Filipendula rubra | G | S | 59.6 |
| Galinsoga ciliata (Rofiresque) Blake | G | R | 20.5 |
| Galium odoratum | G | R | 56.8 |
| Gaultheria hispidula (L.) Muhl | G | O | 100.0 |
| Glycine max | G | O | 22.8 |
| Glycyrrhiza glabra | G | S | 28.4 |
| Hamamelis virginiana | G | O | 33.8 |
| Hamamelis virginiana | G | R | 100.0 |
| Helianthus annus | G | R | 26.5 |
| Helianthus strumosus | G | O | 21.2 |
| Helianthus tuberosus L. | G | W | 48.4 |
| Helichrysum angustifolium | G | W | 38.1 |
| Helichrysum angustifolium | G | V | 83.8 |
| Helichrysum thianschanicum Regel | G | O | 61.3 |
| Heliotropium arborescens | G | O | 56.2 |
| Heliotropium arborescens | G | R | 54.9 |
| Humulus lupulus | G | V | 70.5 |
| Humulus lupulus | G | S | 43.0 |
| Hypericum henryi | G | O | 31.0 |
| Hypericum perforatum | G | R | 100.0 |
| Inula helenium | G | W | 85.3 |
| Inula helenium | G | V | 74.7 |
| Inula helenium | G | S | 37.4 |
| Ipomea batatas | G | O | 39.0 |
| Isatis tinctoria | G | O | 100.0 |
| Laportea canadensis | G | O | 26.9 |
| Laurus nobilis | G | W | 51.5 |
| Laurus nobilis | G | S | 100.0 |
| Lavendula angustifolia | G | V | 44.4 |
| Lavendula latifolia | G | V | 44.8 |
| Ledum groenlandicum | G | S | 100.0 |
| Levistecum officinale | G | W | 39.6 |
| Matricaria recutita | G | O | 100.0 |
| Melissa officinalis | G | W | 98.0 |
| Melissa officinalis | G | V | 76.3 |
| Melissa officinalis | G | R | 36.6 |
| Melissa officinalis | G | O | 80.6 |
| Mentha arvensis | G | O | 83.5 |
| Mentha piperita | G | O | 79.0 |
| Mentha piperita vulgaris | G | V | 45.9 |
| Mentha pulegium | G | O | 47.0 |
| Mentha spicata | G | V | 73.9 |
| Mentha spicata | G | O | 81.3 |
| Mentha spicata | G | O | 93.0 |
| Monarda didyma | G | S | 35.8 |
| N | G | R | 100.0 |
| N | G | R | 34.8 |
| Nepeta cataria | G | V | 38.4 |
| Ocimum basilicum | G | W | 20.4 |
| Ocimum basilicum | G | O | 89.9 |
| Ocimum basilicum | G | V | 31.3 |
| Ocimum basilicum | G | W | 82.3 |
| Oenothera biennis linné | G | O | 62.8 |
| Oenothera biennis linné | G | R | 100.0 |
| Oenothera biennis linné | G | R | 100.0 |
| Oenothera biennis Linne | G | S | 100.0 |
| Origanum vulgare | G | V | 67.1 |
| Origanum vulgare | G | V | 65.5 |
| Origanum vulgare | G | W | 58.1 |
| Origanum vulgare | G | V | 70.5 |
| Origanum vulgare | G | W | 34.5 |
| Origanum vulgare | G | V | 60.1 |
| Origanum vulgare | G | O | 100.0 |
| Origanum vulgare | G | S | 28.5 |
| Origanum vulgare | G | O | 83.7 |
| Origanum vulgare | G | S | 22.1 |
| Oxyria digyna | G | V | 57.7 |
| Perilla frutescens | G | V | 75.8 |
| Peucedanum cervaria | G | R | 37.5 |
| Peucedanum cervaria | G | R | 25.3 |
| Plantago major | G | O | 31.7 |
| Plectranthus sp. | G | V | 28.5 |
| Portulaca oleracera linné | G | O | 37.8 |
| Potentilla anserina | G | S | 21.1 |
| Poterium sanguisorba | G | V | 72.1 |
| Poterium sanguisorba | G | S | 65.9 |
| Poterium sanquisorba | G | O | 63.6 |
| Poterium sanquisorba | G | W | 28.7 |
| Prunella vulgaris | G | O | 40.7 |
| Pteridium aquilinum | G | O | 25.7 |
| Pteridium aquilinum | G | R | 100.0 |
| Raphanus Raphanistrum | G | R | 42.7 |
| Ribes nidigrolaria | G | W | 45.9 |
| Ribes nigrum | G | W | 35.9 |
| Ribes Silvestris | G | W | 34.9 |
| Ribes Uva-crispa | G | S | 30.5 |
| Ricinus communis | G | R | 95.0 |
| Ricinus communis | G | S | 48.3 |
| Rosa rugosa thunb. | G | W | 40.3 |
| Rosa rugosa thunb. | G | S | 97.8 |
| Rosmarinus officinalis | G | O | 100.0 |
| Rosmarinus officinalis | G | R | 54.1 |
| Rosmarinus officinalis | G | W | 77.7 |
| Rosmarinus officinalis | G | V | 72.2 |
| Rubus canadensis | G | S | 25.3 |
| Rubus idaeus L. | G | W | 31.1 |
| Rubus ideaus | G | S | 100.0 |
| Rubus ideaus | G | R | 37.6 |
| Rubus ideaus | G | O | 34.8 |
| Rubus occidentalis | G | S | 93.3 |
| Rubus occidentalis | G | O | 22.7 |
| Rubus occidentalis | G | S | 21.6 |
| Rumex crispus linné | G | R | 100.0 |
| Rumex crispus linné | G | R | 100.0 |
| Salvia elegens | G | V | 41.3 |
| Salvia elegens | G | W | 62.9 |
| Salvia officinalis | G | R | 43.3 |
| Salvia officinalis | G | O | 55.1 |
| Salvia officinalis | G | W | 100.0 |
| Salvia officinalis | G | V | 52.5 |
| Salvia officinalis | G | O | 100.0 |
| Salvia officinalis | G | R | 38.8 |
| Salvia officinalis | G | V | 49.5 |
| Salvia officinalis | G | W | 95.3 |
| Salvia officinalis | G | W | 41.3 |
| Salvia sclarea | G | W | 31.1 |
| Sarriette commune | G | O | 59.7 |
| Sarriette vivace | G | O | 72.3 |
| Sarriette vivace | G | S | 26.0 |
| Satureja montana | G | V | 78.5 |
| Satureja montana | G | W | 100.0 |
| Solanum tuberosum | G | O | 35.8 |
| Sonchus oleraceus L. | G | O | 41.0 |
| Sorghum dochna | G | S | 100.0 |
| Sorghum sudanense | G | O | 32.6 |
| Sorghum sudanense | G | W | 39.7 |
| Symphytum officinale | G | V | 79.4 |
| Symphytum officinale | G | O | 74.6 |
| Tanacetum parthenium | G | V | 23.1 |
| Tanacetum parthenium | G | W | 24.3 |
| Tanacetum vulgare | G | W | 20.8 |
| Tanacetum vulgare | G | O | 32.0 |
| Tanacetum vulgare | G | O | 58.5 |
| Tanacetum vulgare “Goldsticks” | G | V | 44.8 |
| Taraxacum officinale | G | V | 58.2 |
| Thymus fragantissumus | G | R | 39.9 |
| Thymus herba-barona | G | W | 26.6 |
| Thymus herba-barona | G | V | 35.7 |
| Thymus praecox subsp arctitus | G | O | 78.0 |
| Thymus serpyllum | G | V | 47.4 |
| Thymus serpyllum | G | O | 100.0 |
| Thymus serpyllum | G | W | 22.6 |
| Thymus serpyllum | G | V | 70.2 |
| Thymus vulgaris | G | O | 40.8 |
| Thymus vulgaris | G | W | 37.3 |
| Thymus vulgaris “Argenteus” | G | V | 87.7 |
| Thymus x citriodorus | G | W | 27.2 |
| Vaccinum angustifolium | G | S | 41.7 |
| Vaccinum macrocarpon | G | W | 63.5 |
| Viburnum trilobum Marsh. | G | R | 67.7 |
| Viburnum trilobum Marsh. | G | W | 23.6 |
| Vicia sativa | G | O | 38.5 |
| Vicia villosa | G | O | 25.2 |
| Vitia sp. | G | S | 24.8 |
| Vitia sp. | G | W | 100.0 |
| Vitia sp. | G | R | 100.0 |
| Vitia sp. | G | S | 20.8 |
| Zea mays | G | O | 53.7 |
| Achillea millefolium | T | W | 41.8 |
| Achillea millefolium | T | V | 31.5 |
| Acorus calamus | T | R | 68.4 |
| Acorus calamus | T | S | 39.2 |
| Adiantum pedatum | T | R | 100.0 |
| Agastache foeniculum | T | O | 78.0 |
| Agastache foeniculum “Snow Pike” | T | W | 34.5 |
| Agastache foeniculum “Snow Pike” | T | V | 54.3 |
| Agrimonia eupatoria | T | W | 100.0 |
| Alchemilla mollis | T | V | 37.1 |
| Alchemilla mollis | T | W | 100.0 |
| Alchemilla mollis | T | S | 98.8 |
| Alchemilla mollis | T | O | 24.3 |
| Alchemilla mollis | T | S | 83.7 |
| Alchemilla mollis | T | O | 80.0 |
| Althaea officinalis | T | S | 34.1 |
| Althaea officinalis | T | S | 34.3 |
| Althaea officinalis | T | S | 30.8 |
| Ambrosia artemisiifolia linné | T | O | 61.6 |
| Ambrosia artemisiifolia linné | T | R | 52.1 |
| Amelanchier sanguinea x A. laevis | T | S | 38.6 |
| angelica archangelica | T | S | 54.8 |
| Anthemis tinctorium | T | O | 67.7 |
| Arachis hypogaea | T | V | 85.1 |
| Aralia nudicaulis | T | S | 74.2 |
| Arctostaphylos uva-ursi | T | R | 98.8 |
| Arctostaphylos uva-ursi | T | S | 82.4 |
| Aronia prunifolia | T | W | 27.3 |
| Artemisia draculus | T | S | 20.2 |
| Artemisia dracunlus | T | S | 37.2 |
| Artemisia Ludoviciana | T | O | 54.8 |
| Aster sp ? | T | O | 43.4 |
| Aster sp ? | T | R | 99.9 |
| Ayperus esculentus | T | W | 46.9 |
| Beta vulgaris | T | R | 81.4 |
| Beta vulgaris | T | O | 30.6 |
| Betula glandulosa | T | W | 58.2 |
| Borago officinalis | T | O | 20.2 |
| Brassica juncea | T | R | 56.6 |
| Brassica napus | T | R | 34.1 |
| Brassica nigra | T | S | 32.3 |
| Brassica rapa | T | R | 21.4 |
| Calamintha nepeta | T | V | 71.4 |
| Calamintha nepeta | T | W | 30.3 |
| Canna edulis | T | O | 31.9 |
| Canneberge | T | R | 66.3 |
| Capsella bursa-pastoris (linné) médicus | T | R | 37.1 |
| Carya cordiformis | T | W | 100.0 |
| Chaerophyllum bulbosum | T | V | 86.0 |
| Chrysanthemum leucanthemum linné | T | O | 45.4 |
| Cichorium intybus | T | V | 74.8 |
| Cichorium intybus | T | W | 23.8 |
| Cichorium intybus | T | O | 38.9 |
| Cimicifuga racemosa | T | W | 65.1 |
| Citrullus colocynthus | T | S | 50.2 |
| Citrus limettoides | T | O | 45.1 |
| Citrus limettoides | T | V | 28.9 |
| Citrus limon | T | O | 25.9 |
| Citrus limon | T | V | 43.3 |
| Coix Lacryma-Jobi | T | O | 22.1 |
| Coriandrum sativum | T | W | 62.0 |
| Crataegus sp ? | T | R | 44.0 |
| Crataegus submollis | T | S | 40.7 |
| Crataegus submollis | T | S | 29.3 |
| Curcuma longa syn. C. domestica | T | O | 22.2 |
| Cynara scolymus | T | R | 42.2 |
| Dioscorea batatas | T | O | 29.1 |
| Dioscorea batatas | T | O | 28.9 |
| Diospiros Kaki | T | V | 57.8 |
| Dirca palustris | T | S | 39.2 |
| Dolichus lablab | T | R | 42.9 |
| Dryopteris filix-mas | T | O | 24.9 |
| Dryopteris filix-mas | T | R | 100.0 |
| Echinacea purpurea | T | V | 78.9 |
| Echinacea purpurea | T | W | 95.8 |
| Echinacea purpurea | T | O | 53.7 |
| Erigeron speciosus (Lindl.) D.C. | T | O | 96.2 |
| Fragaria | T | O | 42.7 |
| Fragaria x ananassa | T | S | 100.0 |
| Fragaria x ananassa | T | S | 100.0 |
| Fruit de la passion | T | O | 30.2 |
| Fucus vesicutosis | T | O | 93.3 |
| Galinsoga ciliata (Rofiresque) Blake. | T | R | 33.0 |
| Galium odoratum | T | R | 27.0 |
| Gaultheria hispidula (L) Muhl | T | W | 100.0 |
| Gaultheria procumbens | T | W | 30.0 |
| Gaultheria procumbens | T | S | 100.0 |
| Glycine max Envy | T | O | 20.1 |
| Glycyrrhiza glabra | T | W | 47.9 |
| Guizotia abyssinica | T | R | 74.1 |
| Guizotia abyssinica | T | S | 22.7 |
| Hamamelis virginiana | T | O | 100.0 |
| Hamamelis virginiana | T | R | 100.0 |
| Helenium hoopesii | T | O | 21.7 |
| Helenium hoopesii | T | S | 24.6 |
| Helianthus annus | T | O | 21.0 |
| Helianthus strumosus | T | O | 85.6 |
| Helianthus tuberosa | T | V | 64.5 |
| Helianthus tuberosa | T | W | 100.0 |
| Helichrysum angustifolium | T | O | 100.0 |
| Helichrysum angustifolium | T | W | 87.0 |
| Helichrysum angustifolium | T | V | 84.4 |
| Helichrysum angustifolium | T | S | 92.3 |
| Helichrysum thianschanicum Regel | T | O | 59.5 |
| Heliotropium arborescens | T | O | 85.1 |
| Hibiscus cannabinus | T | O | 25.0 |
| Humulus lupulus | T | S | 21.4 |
| Humulus lupulus | T | S | 21.5 |
| Humulus lupulus | T | R | 88.4 |
| Humulus lupulus | T | S | 22.5 |
| Hypericum perforatum | T | R | 100.0 |
| Inula helenium | T | V | 97.1 |
| Inula helenium | T | W | 69.0 |
| Inula helenium | T | S | 29.3 |
| Ipomea batalas | T | O | 27.0 |
| Iris versicolor | T | R | 22.9 |
| Juniperus communis | T | R | 100.0 |
| Krameria Triandra | T | O | 52.6 |
| Lathyrus sylvestris | T | R | 32.5 |
| Laurus nobilis | T | S | 100.0 |
| Lavendula angustifolia | T | V | 74.8 |
| Lavendula angustifolia | T | W | 70.2 |
| Lavendula latifolia | T | W | 85.6 |
| Lavendula latifolia | T | V | 63.3 |
| Lavendula latifolia | T | O | 20.2 |
| Ledum groenlandicum | T | R | 100.0 |
| Ledum groenlandicum | T | S | 94.1 |
| Lepidium sativum | T | O | 20.5 |
| Litchi chinensis | T | S | 100.0 |
| Lolium multiflorum | T | O | 22.7 |
| Lonicera ramosissima | T | S | 30.9 |
| Lotus corniculatus | T | R | 60.2 |
| Malus | T | V | 23.1 |
| Malva moschata | T | S | 31.4 |
| Melissa officinalis | T | V | 81.4 |
| Melissa officinalis | T | W | 87.5 |
| Melissa officinalis | T | O | 100.0 |
| Melissa officinalis | T | V | 36.0 |
| Melissa officinalis | T | W | 36.8 |
| Melissa officinalis | T | O | 100.0 |
| Melissa officinalis | T | R | 30.3 |
| mentha arvensis | T | R | 67.2 |
| Mentha piperita | T | S | 20.8 |
| Mentha piperita | T | O | 100.0 |
| Mentha piperita | T | S | 26.9 |
| Mentha piperita | T | O | 97.8 |
| Mentha piperita vulgaris | T | W | 20.2 |
| Mentha piperita vulgaris | T | V | 42.5 |
| Mentha pulegium | T | O | 100.0 |
| Mentha spicata | T | W | 51.6 |
| Mentha spicata | T | V | 81.8 |
| Mentha spicata | T | O | 100.0 |
| Mentha spicata | T | O | 100.0 |
| Mentha spicata | T | S | 23.2 |
| Nepeta cataria | T | V | 62.8 |
| Ocimum Basilicum | T | V | 41.1 |
| Ocimum Basilicum | T | W | 40.0 |
| Ocimum Basilicum | T | O | 28.4 |
| Oenothera biennis linné | T | O | 67.3 |
| Oenothera biennis linné | T | R | 100.0 |
| Onobrychis viciafolia | T | O | 34.0 |
| Origanum marjonara | T | O | 29.5 |
| Origanum vulgare | T | V | 55.5 |
| Origanum vulgare | T | W | 67.7 |
| Origanum vulgare | T | W | 46.4 |
| Origanum vulgare | T | V | 68.6 |
| Origanum vulgare | T | W | 99.9 |
| Origanum vulgare | T | V | 42.0 |
| Origanum Vulgare | T | V | 28.8 |
| Origanum Vulgare | T | W | 46.7 |
| Origanum vulgare | T | O | 100.0 |
| Origanum vulgare | T | W | 51.7 |
| Origanum vulgare | T | S | 30.8 |
| Origanum vulgare | T | O | 25.4 |
| Origanum vulgare | T | S | 38.2 |
| oxyria digyna | T | V | 23.1 |
| Pastinaca sativa | T | O | 33.1 |
| Pastinaca sativa | T | R | 22.2 |
| Perilla frutescens | T | O | 100.0 |
| Perilla frutescens | T | W | 61.7 |
| Perilla frutescens | T | V | 75.6 |
| Petroselinum crispum Nyman ex. A. W. Hill | T | W | 24.8 |
| Peucedanum cervaria | T | R | 53.0 |
| Peucedanum cervaria | T | R | 35.9 |
| Plaffia paniculala | T | O | 85.9 |
| Phaseolus vulgaris | T | O | 35.7 |
| Phytolacca americana | T | S | 28.6 |
| Phytolacca decandra syn. P. americana | T | O | 31.6 |
| Plectranthus sp. | T | V | 66.0 |
| Polygonium chinense | T | S | 33.2 |
| Polygonum aviculare linné | T | R | 100.0 |
| Populus x petrowskyana | T | O | 25.4 |
| Potentilla anserina | T | S | 55.8 |
| Poterium sanguisorba | T | W | 100.0 |
| Poterium sanguisorba | T | V | 82.3 |
| Prunella vulgaris | T | O | 52.6 |
| Psoralea corylifolia | T | O | 21.3 |
| Psoralea corylifolia | T | S | 26.0 |
| Psoralea corylifolia | T | S | 27.4 |
| Pteridium aquilinum | T | R | 100.0 |
| Punica granatum | T | V | 21.3 |
| Punica granatum | T | W | 77.1 |
| Punica granatum | T | S | 43.9 |
| Radix Rehmannia | T | O | 23.9 |
| Raphanus raphanistrum | T | R | 36.5 |
| Raphanus raphanistrum | T | R | 30.5 |
| Rhamnus frangula | T | R | 100.0 |
| Rheum palmatum | T | W | 100.0 |
| Rianus communis | T | R | 100.0 |
| Rianus communis | T | S | 100.0 |
| Rianus communis | T | S | 68.2 |
| Ribes Grossularia L. | T | W | 61.1 |
| Ribes nidigrolaria | T | W | 32.1 |
| Ribes nigrum | T | O | 90.2 |
| Ribes nigrum | T | S | 20.3 |
| Ribes nigrum L. | T | W | 21.1 |
| Ribes nigrum L. | T | W | 51.6 |
| Ribes sativam syme | T | W | 20.9 |
| Ribes uva-crispa | T | S | 41.8 |
| Rosa rugosa | T | S | 100.0 |
| Rosa rugosa thumb. | T | W | 94.1 |
| Rosmarinum officinalis | T | O | 100.0 |
| Rosmarinum officinalis | T | R | 40.0 |
| Rosmarinum officinalis | T | V | 76.9 |
| Rubus canadensis | T | S | 31.3 |
| Rubus canadensis | T | V | 22.8 |
| Rubus canadensis | T | W | 100.0 |
| Rubus idaeus | T | V | 25.0 |
| Rubus idaeus L. | T | S | 100.0 |
| Rubus ideaus | T | S | 46.1 |
| Rubus ideaus | T | R | 32.0 |
| Rubus ideaus | T | O | 28.5 |
| Rubus occidentalis | T | R | 100.0 |
| Rubus occidentalis | T | O | 23.5 |
| Rumes scutatus | T | O | 27.1 |
| Rumex acetosella linné | T | O | 23.0 |
| Rumex crispus linné | T | R | 100.0 |
| Rumex crispus linné | T | R | 100.0 |
| Salvia (elegens) | T | O | 100.0 |
| Salvia elegens | T | W | 63.5 |
| Salvia officinalis | T | O | 34.0 |
| Salvia officinalis | T | R | 41.7 |
| Salvia officinalis | T | V | 64.3 |
| Salvia officinalis | T | W | 100.0 |
| Salvia officinalis | T | R | 38.8 |
| Salvia officinalis | T | O | 73.4 |
| Salvia officinalis | T | W | 95.3 |
| Salvia officinalis | T | V | 56.8 |
| Salvia officinalis | T | W | 25.1 |
| Salvia sclarea | T | W | 28.6 |
| Sambucus canadensis | T | S | 40.1 |
| Sambucus canadensis L. | T | O | 50.2 |
| Sambucus caradensis | T | S | 29.7 |
| Sanguisorba minor | T | V | 32.0 |
| Sanguisorba minor | T | W | 59.5 |
| Sanguisorba minor | T | S | 58.5 |
| Sanguisorba minor | T | S | 68.5 |
| Satureja hortensis | T | O | 66.5 |
| Satureja hortensis | T | S | 20.1 |
| Satureja montana | T | O | 43.3 |
| Satureja montana | T | R | 36.7 |
| Satureja montana | T | W | 100.0 |
| Satureja montana | T | V | 81.1 |
| Satureja montana | T | S | 40.6 |
| Satureja montana | T | V | 54.0 |
| Satureja montana | T | O | 90.1 |
| Satureja repandra | T | R | 35.8 |
| Satureja repandra | T | W | 100.0 |
| Satureja repandra | T | V | 75.0 |
| Solanum Tuberosum | T | O | 30.9 |
| Solidago canadensis | T | R | 91.8 |
| Sonchus oleraceus L. | T | O | 45.9 |
| Sorghum dochna Snowdrew | T | O | 31.5 |
| Sorghum sudanense | T | O | 33.6 |
| Stipa capillata L. | T | O | 33.0 |
| Symphytum officinale | T | O | 94.1 |
| Symphytum officinale | T | O | 42.8 |
| Tanacetum parthenium | T | W | 40.1 |
| Tanacetum parthenium | T | V | 33.6 |
| Tanacetum vulgare | T | V | 36.5 |
| Tanacetum vulgare | T | W | 51.2 |
| Tanacetum vulgare | T | O | 95.6 |
| Tanacetum vulgare | T | O | 38.4 |
| Tanacetum vulgare | T | R | 27.4 |
| Tanacetum vulgare “Goldsticks” | T | V | 37.9 |
| Taraxacum officinale | T | V | 57.8 |
| Thymus fragantissumus | T | R | 34.0 |
| Thymus fragantissumus | T | W | 72.7 |
| Thymus fragantissumus | T | V | 71.0 |
| Thymus praecox subsp arctitus | T | O | 59.2 |
| Thymus pseudolanuginosus | T | O | 85.7 |
| Thymus pseudolanuginosus | T | W | 20.9 |
| Thymus serpyllum | T | O | 94.8 |
| Thymus serpyllum | T | W | 38.4 |
| Thymus vulgaris | T | O | 100.0 |
| Thymus vulgaris “Argenteus” | T | V | 80.4 |
| Thymus X citriodorus | T | O | 100.0 |
| Tiarella cordifolia | T | R | 100.0 |
| Trichosanthes kirilowii | T | O | 100.0 |
| Triticale sp. | T | O | 24.4 |
| Tropaeolum majus | T | O | 20.6 |
| Ulmus americana | T | O | 43.7 |
| Urtica dioica | T | R | 28.9 |
| Vaccinium angustifolium | T | S | 43.2 |
| Vaccinium angustifolium | T | S | 42.4 |
| Vaccinium macrocarpon | T | W | 59.2 |
| Vaccinium macrocarpon | T | S | 27.2 |
| Vaccinium macrocarpon | T | S | 21.6 |
| Vaccinum macrocarpon | T | V | 62.6 |
| Veronica officinalis | T | S | 52.6 |
| Viburnum trilobum Marsh. | T | R | 100.0 |
| Vicia villosa | T | O | 36.6 |
| Vitia sp. | T | W | 58.9 |
| Vitis sp | T | S | 24.7 |
| Vitis sp. | T | S | 22.8 |
| Vitis sp. | T | S | 21.7 |
| Zea mays | T | S | 20.5 |

TABLE 8
Cath L

| Nom latin | Stress | Extrait | Inhibition (%) |
|---|---|---|---|
| Actinidia arguta | A | R | 63.3 |
| Actinidia arguta | A | O | 46.3 |
| Achillea millefolium | A | O | 32.4 |
| Achillea millefolium | A | R | 26.3 |
| Aconitum napellus | A | O | 30.0 |
| Acorus calamus | A | R | 25.9 |
| Adiantum pedatum | A | O | 20.2 |
| Adiantum pedatum | A | R | 22.2 |
| Agropyron repens | A | O | 98.6 |
| Agropyron repens | A | R | 61.8 |
| Alchemilla mollis | A | O | 75.7 |
| Alchemilla mollis | A | R | 36.5 |
| Allium porrum | A | R | 39.7 |
| Allium porrum | A | O | 58.2 |
| Allium cepa | A | O | 51.0 |
| Allium sativum | A | O | 53.8 |
| Allium schoenoprasum | A | O | 74.6 |
| Allium Tuberosum | A | O | 69.5 |
| Aloe vera | A | R | 44.7 |
| Aloe vera | A | O | 55.6 |
| Althaea officinalis | A | O | 95.0 |
| Althaea officinalis | A | R | 33.4 |
| Amaranthus retroflexus | A | R | 74.5 |
| Amaranthus retroflexus | A | O | 98.4 |
| Anethum graveolens | A | R | 37.4 |
| Anethum graveolens | A | O | 58.7 |
| Angelica archangelica | A | O | 79.1 |
| Apium graveolens | A | R | 27.9 |
| Apium graveolens | A | O | 46.5 |
| Aralia nudicaulis | A | O | 89.3 |
| Aralia nudicaulis | A | R | 55.4 |
| Arctium lappa | A | R | 32.8 |
| Arctium minus | A | R | 72.5 |
| Arctium minus | A | O | 61.3 |
| Armoracia rusticana | A | O | 95.8 |
| Aronia melanocarpa | A | R | 39.8 |
| Aronia melanocarpa | A | O | 28.2 |
| Artemisia Absinthium | A | R | 51.7 |
| Artemisia Absinthium | A | O | 63.7 |
| Artemisia dracunculus | A | O | 45.4 |
| Aster sp | A | R | 41.8 |
| Aster sp | A | O | 91.5 |
| Atropa belladonna | A | O | 47.3 |
| Atropa belladonna | A | R | 31.7 |
| Beckmannia eruciformis | A | R | 40.5 |
| Beckmannia eruciformis | A | O | 60.8 |
| Beta vulgaris | A | R | 66.1 |
| Beta vulgaris | A | O | 79.5 |
| Beta vulgaris spp. Maritima | A | O | 63.3 |
| Beta vulgaris spp. Maritima | A | R | 59.1 |
| Borago officinalis | A | O | 40.9 |
| Brassica napus | A | O | 64.6 |
| Brassica napus | A | R | 21.1 |
| Brassica oleracea | A | R | 66.6 |
| Brassica oleracea | A | O | 68.6 |
| Brassica rapa | A | O | 99.0 |
| Brassica rapa | A | R | 99.3 |
| Campanula rapunculus | A | R | 59.0 |
| Campanula rapunculus | A | O | 50.6 |
| Canna edulis | A | O | 23.9 |
| Capsella bursa-pastoris | A | R | 49.0 |
| Capsella bursa-pastoris | A | O | 47.0 |
| Capsicum annuum | A | R | 29.1 |
| Carum carvi | A | O | 60.4 |
| Chaerophyllum bulbosum | A | O | 48.6 |
| Chaerophyllum bulbosum | A | R | 48.2 |
| Chelidonium majus | A | O | 35.5 |
| Chelidonium majus | A | R | 23.1 |
| Chenopodium bonus-henricus | A | O | 65.9 |
| Chenopodium quinoa | A | R | 62.3 |
| Chenopodium quinoa | A | O | 90.0 |
| Cicer arietinum | A | O | 82.4 |
| Cichorium intybus | A | R | 58.0 |
| Cichorium intybus | A | O | 81.7 |
| Coix Lacryma-Jobi | A | R | 32.6 |
| Coix Lacryma-Jobi | A | O | 43.4 |
| Coriandrum sativum | A | R | 26.9 |
| Coriandrum sativum | A | O | 65.0 |
| Cornus canadensis | A | R | 99.7 |
| Cornus canadensis | A | O | 60.6 |
| Crataegus sp | A | R | 25.9 |
| Crataegus sp | A | O | 28.2 |
| Cryptotaenia canadensis | A | O | 73.3 |
| Cryptotaenia canadensis | A | R | 36.1 |
| Cymbopogon citratus | A | O | 32.7 |
| Cyperus esculentus | A | R | 41.3 |
| Cyperus esculentus | A | O | 33.8 |
| Daucus carota | A | R | 63.6 |
| Daucus carota | A | O | 43.4 |
| Dirca palustris | A | O | 61.1 |
| Dirca palustris | A | R | 46.6 |
| Echinacea purpurea | A | O | 54.8 |
| Eleusine coracana | A | O | 36.4 |
| Fagopyrum esculentum | A | R | 37.9 |
| Fagopyrum esculentum | A | O | 43.3 |
| Fagopyrum tataricum | A | R | 28.4 |
| Fagopyrum tataricum | A | O | 32.8 |
| Foeniculum vulgare | A | O | 48.8 |
| Fragaria x ananassa | A | R | 46.3 |
| Fragaria x ananassa | A | O | 78.8 |
| Galinsoga ciliata | A | O | 46.0 |
| Galium odoratum | A | R | 59.8 |
| Galium odoratum | A | O | 79.5 |
| Gaultheria hispidula | A | R | 53.4 |
| Gaultheria hispidula | A | O | 54.3 |
| Glechoma hederacea | A | O | 23.4 |
| Glechoma hederacea | A | R | 26.9 |
| Glycine max | A | R | 20.5 |
| Glycine max | A | O | 73.8 |
| Glycyrrhiza glabra | A | O | 57.7 |
| Glycyrrhiza glabra | A | R | 53.8 |
| Guizotia abyssinica | A | R | 29.6 |
| Guizotia abyssinica | A | O | 78.6 |
| Hamamelis virginiana | A | R | 41.2 |
| Hedeoma pulegioides | A | O | 26.3 |
| Helleborus niger | A | O | 36.9 |
| Helleborus niger | A | R | 35.4 |
| Hordeum hexastichon | A | R | 31.1 |
| Hyssopus officinalis | A | R | 84.8 |
| Hyssopus officinalis | A | O | 85.8 |
| Inula helenium | A | O | 58.4 |
| Inula helenium | A | R | 32.7 |
| Ipomoea Batatas | A | O | 29.6 |
| Lathyrus sativus | A | R | 31.7 |
| Lathyrus sativus | A | O | 71.1 |
| Lathyrus sylvestris | A | R | 65.3 |
| Lathyrus sylvestris | A | O | 66.4 |
| Laurus nobilis | A | R | 43.1 |
| Laurus nobilis | A | O | 46.1 |
| Leonurus cardiaca | A | O | 63.3 |
| Leonurus cardiaca | A | R | 24.5 |
| Levisticum officinale | A | R | 20.9 |
| Levisticum officinale | A | O | 43.8 |
| Lotus corniculatus | A | R | 59.0 |
| Lotus corniculatus | A | O | 87.4 |
| Lycopersicon esculentum | A | R | 28.0 |
| Malva sylvestris | A | O | 23.1 |
| Medicago sativa | A | R | 63.8 |
| Medicago sativa | A | O | 53.6 |
| Melilotus albus | A | O | 93.7 |
| Melilotus albus | A | R | 80.1 |
| Melissa officinalis | A | R | 40.8 |
| Melissa officinalis | A | O | 69.5 |
| Mentha piperita | A | R | 61.0 |
| Mentha piperita | A | O | 73.2 |
| Mentha pulegium | A | O | 69.0 |
| Mentha spicata | A | O | 94.6 |
| Mentha suaveolens | A | O | 55.2 |
| Nepeta cataria | A | R | 45.9 |
| Nepeta cataria | A | O | 66.3 |
| Nicotiana tabacum | A | R | 46.8 |
| Oenothera biennis | A | R | 69.8 |
| Oenothera biennis | A | O | 47.3 |
| Origanum majorana | A | O | 38.5 |
| Origanum vulgare | A | R | 43.3 |
| Origanum vulgare | A | O | 68.2 |
| Panax quinquefolius | A | R | 41.7 |
| Panax quinquefolius | A | O | 83.7 |
| Pastinaca sativa | A | O | 62.8 |
| Pastinaca sativa | A | R | 44.2 |
| Perilla frutescens | A | O | 66.2 |
| Petasites japonicus | A | R | 22.6 |
| Petasites japonicus | A | O | 25.5 |
| Petroselinum crispum | A | O | 79.1 |
| Petroselinum crispum | A | R | 32.3 |
| Phalaris canariensis | A | R | 45.4 |
| Phaseolus vulgaris | A | R | 31.0 |
| Phaseolus Vulgaris | A | O | 61.8 |
| Pimpinella anisum | A | O | 38.1 |
| Plantago major | A | O | 95.1 |
| Plectranthus sp. | A | R | 76.9 |
| Plectranthus sp. | A | O | 58.0 |
| Polygonum aviculare | A | R | 28.0 |
| Polygonum aviculare | A | O | 49.7 |
| Potentilla anserina | A | R | 26.6 |
| Poterium Sanquisorba | A | O | 58.0 |
| Pteridium aquilinum | A | R | 32.9 |
| Raphanus raphanistrum | A | R | 70.7 |
| Raphanus raphanistrum | A | O | 83.2 |
| Raphanus salivus | A | R | 90.9 |
| Raphanus sativus | A | O | 95.4 |
| Rheum rhabarbarum | A | R | 26.0 |
| Rheum rhabarbarum | A | O | 62.9 |
| Ribes nigrum | A | O | 62.9 |
| Ribes Sylvestre | A | R | 34.5 |
| Ribes Sylvestre | A | O | 80.3 |
| Ricinus communis | A | R | 89.9 |
| Ricinus communis | A | O | 81.0 |
| Rosa rugosa | A | R | 32.9 |
| Rosa rugosa | A | O | 35.9 |
| Rosmarinus officinalis | A | O | 78.2 |
| Rubus allegheniensis | A | O | 76.8 |
| Rubus canadensis | A | R | 40.7 |
| Rubus canadensis | A | O | 72.6 |
| Rubus idaeus | A | R | 35.5 |
| Rubus idaeus | A | O | 97.9 |
| Rumex Acetosa | A | O | 32.0 |
| Rumex acetosella | A | R | 73.2 |
| Rumex acetosella | A | O | 56.9 |
| Rumex crispus | A | R | 49.7 |
| Rumex crispus | A | O | 37.5 |
| Rumex Scutatus | A | O | 53.1 |
| Rumex Scutatus | A | R | 25.9 |
| Ruta graveolens | A | O | 56.2 |
| Salix purpurea | A | R | 71.4 |
| Salix purpurea | A | O | 24.7 |
| Salvia elegans | A | O | 67.6 |
| Salvia officinalis | A | O | 70.5 |
| Salvia officinalis | A | R | 56.6 |
| Salvia sclarea | A | O | 70.1 |
| Santolina chamaecyparissus | A | R | 59.5 |
| Santolina chamaecyparissus | A | O | 59.2 |
| Satureja montana | A | O | 71.7 |
| Scorzonera hispanica | A | O | 21.9 |
| Secale cereale | A | R | 33.3 |
| Senecio vulgaris | A | R | 47.5 |
| Senecio vulgaris | A | O | 20.8 |
| Setaria italica | A | R | 48.6 |
| Setaria italica | A | O | 37.1 |
| Sium Sisarum | A | O | 33.8 |
| Sium Sisarum | A | R | 62.5 |
| Solanum tuberosum | A | O | 53.6 |
| Solidago sp | A | R | 54.0 |
| Solidago sp | A | O | 95.1 |
| Sonchus oleraceus | A | R | 59.4 |
| Sonchus oleraceus | A | O | 69.2 |
| Sorghum dochna | A | R | 33.9 |
| Sorghum dochna | A | O | 55.3 |
| Sorghum durra | A | R | 61.3 |
| Sorghum durra | A | O | 83.9 |
| Stachys byzantina | A | R | 61.6 |
| Stachys byzantina | A | O | 73.8 |
| Stellaria graminea | A | R | 40.1 |
| Stellaria graminea | A | O | 55.8 |
| Stellaria media | A | R | 70.9 |
| Stellaria media | A | O | 51.4 |
| Tanacetum cinerariifolium | A | O | 67.7 |
| Tanacetum parthenium | A | R | 50.8 |
| Tanacetum parthenium | A | O | 81.9 |
| Tanacetum vulgare | A | R | 56.2 |
| Tanacetum vulgare | A | O | 51.9 |
| Taraxacum officinale | A | O | 98.7 |
| Taraxacum officinale | A | R | 82.1 |
| Teucrium chamaedrys | A | O | 62.2 |
| Thymus praecox subsp arcticus | A | R | 42.0 |
| Thymus praecox subsp arcticus | A | O | 54.2 |
| Thymus serpyllum | A | O | 93.4 |
| Thymus serpyllum | A | R | 57.5 |
| Thymus vulgaris | A | R | 68.7 |
| Thymus vulgaris | A | O | 55.8 |
| Thymus x citriodorus | A | O | 72.8 |
| Thymus x citriodorus | A | R | 31.9 |
| Tragopogon porrifolius | A | O | 67.2 |
| Tragopogon porrifolius | A | R | 37.0 |
| Tropaeolum malus | A | O | 62.8 |
| Typha latifolia | A | R | 77.5 |
| Typha latifolia | A | O | 70.6 |
| Vaccinium Corymbosum | A | O | 74.7 |
| Vaccinium Corymbosum | A | R | 69.5 |
| Vaccinium macrocarpon | A | R | 71.4 |
| Vaccinum macrocarpon | A | O | 78.9 |
| Verbascum thapsus | A | O | 76.8 |
| Verbascum thapsus | A | R | 62.0 |
| Vicia sativa | A | R | 79.2 |
| Vicia sativa | A | O | 88.7 |
| Vicia villosa | A | O | 74.5 |
| Vicia villosa | A | R | 61.0 |
| Vinca minor | A | O | 46.7 |
| Vinca minor | A | R | 31.9 |
| Vitiis sp. | A | R | 89.5 |
| Vitiis sp. | A | O | 54.6 |
| Zea mays | A | R | 52.0 |
| Zea mays | A | O | 93.8 |
| Achillea millefolium | G | O | 45.8 |
| Achillea millefolium | G | R | 24.6 |
| Aconitum napellus | G | R | 28.7 |
| Acorus calamus | G | R | 37.5 |
| Acorus calamus | G | O | 32.8 |
| Actinidia arguta | G | R | 47.8 |
| Actinidia arguta | G | O | 78.4 |
| Adiantum pedatum | G | O | 45.9 |
| Adiantum pedatum | G | R | 27.0 |
| Agropyron repens | G | O | 83.0 |
| Agropyron repens | G | R | 31.9 |
| Alchemilla mollis | G | O | 71.0 |
| Allium ampeloprasum | G | R | 36.8 |
| Allium ampeloprasum | G | O | 62.2 |
| Allium cepa | G | R | 56.1 |
| Allium cepa | G | O | 64.4 |
| Allium sativum | G | O | 65.2 |
| Allium schoenoporasum | G | O | 78.4 |
| Allium tuberosum | G | O | 46.6 |
| Aloe vera | G | O | 45.7 |
| Althaca officianalis | G | O | 50.0 |
| althaea officinalis | G | R | 42.2 |
| Amaranthus retroflexus | G | R | 41.7 |
| Amaranthus retroflexus | G | O | 90.3 |
| Anethum graveolens | G | R | 31.3 |
| Anethum graveolens | G | O | 60.5 |
| Angelica archangelica | G | O | 64.3 |
| Angelica archangelica | G | R | 63.3 |
| Apium graveolens | G | O | 57.0 |
| Apium graveolens | G | R | 28.4 |
| Aralia nudicaulis | G | O | 71.8 |
| Aralia nudicaulis | G | R | 38.2 |
| Arctium minus | G | R | 42.4 |
| Arctium minus | G | O | 41.5 |
| Armoracia rusticana | G | O | 67.1 |
| Aronia melanocarpa | G | R | 32.0 |
| Aronia melanocarpa | G | O | 70.0 |
| Artemisia absinthium | G | R | 63.1 |
| Artemisia absinthium | G | O | 61.1 |
| Asclepias incarnata | G | R | 58.4 |
| Asclepias incarnata | G | O | 63.3 |
| Asparagus officinalis | G | R | 61.2 |
| Asparagus officinalis | G | O | 86.3 |
| Aster Linne | G | O | 57.5 |
| Aster sp | G | R | 48.7 |
| Aster sp | G | O | 94.5 |
| Atropa belladonna | G | R | 29.2 |
| Beckmannia eruciformis | G | O | 32.9 |
| Beta vulgaris | G | R | 47.9 |
| Beta vulgaris | G | O | 61.9 |
| Borago officinalis | G | O | 51.9 |
| Brassica Napus | G | O | 92.1 |
| Brassica napus | G | R | 30.2 |
| Brassica oleracea | G | R | 79.0 |
| Brassica oleracea | G | O | 85.4 |
| Brassica rapa | G | O | 81.7 |
| Calamagrostis arundiflora | G | R | 59.7 |
| Campanula rapunculus | G | R | 65.4 |
| Campanula rapunculus | G | O | 54.8 |
| Canna edulis | G | O | 30.0 |
| Capsella bursa-pastoris | G | R | 48.1 |
| Capsella bursa-pastoris | G | O | 50.9 |
| Carum carvi | G | O | 62.4 |
| Cerastium tomentosum | G | R | 45.1 |
| Chaerophyllum bulbosum | G | O | 30.0 |
| Chaerophyllum bulbosum | G | R | 54.5 |
| Chelidonium majus | G | O | 43.2 |
| Chelidonium majus | G | R | 30.7 |
| Chichorium endivia | G | O | 64.2 |
| Chichorium endivia subsp endivia | G | R | 48.3 |
| Chichorium endivia subsp endivia | G | O | 67.0 |
| Cichorium intybus | G | O | 78.3 |
| Cichorium intybus | G | R | 87.8 |
| Circium arvense | G | R | 94.1 |
| Circium arvense | G | O | 58.7 |
| Coix Lacryma-Jobi | G | R | 35.7 |
| Coix Lacryma-Jobi | G | O | 31.4 |
| Cornus canadensis | G | R | 61.3 |
| Cornus canadensis | G | O | 80.6 |
| Crataegus submollis | G | R | 21.0 |
| Crataegus submollis | G | O | 44.4 |
| Cymbopogon citratus | G | R | 39.6 |
| Cyperus esculentus | G | R | 62.4 |
| Cyperus esculentus | G | O | 49.6 |
| Daucus carota | G | O | 36.3 |
| Daucus carota | G | R | 44.3 |
| Dirca palustris | G | O | 85.1 |
| Dirca palustris | G | R | 47.1 |
| Echinacea purpurea | G | O | 36.4 |
| Eleusine coracana | G | O | 65.4 |
| Eleusine coracana | G | R | 36.8 |
| Erigeron speciosus | G | R | 39.1 |
| Erysimum perofskianum | G | R | 58.7 |
| Erysimum perofskianum | G | O | 93.1 |
| Fagopyrum esculentum | G | R | 36.4 |
| Fagopyrum esculentum | G | O | 41.0 |
| Fagopyrum tataricum | G | R | 43.3 |
| Fagopyrum tataricum | G | O | 29.1 |
| Galinsoga ciliata | G | R | 49.8 |
| Galinsoga ciliata | G | O | 58.0 |
| Galium odoratum | G | R | 65.1 |
| Galium odoratum | G | O | 94.2 |
| Gaultheria hispidula | G | R | 55.7 |
| Gaultheria hispidula | G | O | 50.6 |
| Gaultheria procumbens | G | R | 53.3 |
| Gaultheria procumbens | G | O | 67.7 |
| Glechoma hederacea | G | O | 70.9 |
| Glechoma hederacea | G | R | 25.3 |
| Glycine max | G | R | 78.6 |
| Glycine max | G | O | 85.9 |
| Glycyrrhiza glabra | G | R | 59.1 |
| Glycyrrhiza glabra | G | O | 60.6 |
| Guizotia abyssinica | G | R | 41.8 |
| Guizotia abyssinica | G | O | 74.3 |
| Hamamelis virginiana | G | R | 44.2 |
| Helianthus strumosus | G | O | 40.6 |
| Helianthus strumosus | G | R | 61.4 |
| Helianthus tuberosus | G | O | 75.1 |
| Helianthus tuberosus | G | R | 30.1 |
| Helichrysum thianschanicum | G | R | 56.3 |
| Helichrysum thianschanicum | G | O | 84.0 |
| Helleborus niger | G | O | 38.8 |
| Helleborus niger | G | R | 25.9 |
| Hordeum hexastichon | G | O | 62.3 |
| Hordeum hexastichon | G | R | 29.4 |
| Hyssopus officinalis | G | R | 64.7 |
| Hyssopus officinalis | G | O | 71.9 |
| Inula helenium | G | O | 29.4 |
| Inula helenium | G | R | 25.7 |
| Ipomoea batatas | G | O | 36.9 |
| Lactuca sativa | G | O | 70.4 |
| Lactuca sativa | G | R | 49.9 |
| Lathyrus sativus | G | O | 62.8 |
| Lathyrus sativus | G | R | 29.0 |
| Lathyrus sylvestris | G | R | 52.1 |
| Lathyrus sylvestris | G | O | 52.5 |
| Laurus nobilis | G | R | 27.1 |
| Laurus nobilis | G | O | 61.0 |
| Lavandula angustifolia | G | R | 51.9 |
| Lavandula angustifolia | G | O | 57.0 |
| Ledum groenlandicum | G | O | 73.4 |
| Ledum groenlandicum | G | R | 52.6 |
| Leonurus cardiaca | G | O | 88.8 |
| Leonurus cardiaca | G | R | 38.5 |
| Levistecum officinale | G | R | 51.2 |
| Levistecum officinale | G | O | 78.3 |
| Lotus corniculatus | G | O | 86.8 |
| Lotus corniculatus | G | R | 50.3 |
| Lupinus polyphyllus | G | R | 78.9 |
| Lupinus polyphyllus | G | O | 66.7 |
| Malus hupehensis | G | R | 52.7 |
| Malus hupehensis | G | O | 64.1 |
| Malva sylvestris | G | R | 26.2 |
| Medicago sativa | G | R | 43.4 |
| Medicago sativa | G | O | 92.5 |
| Melilotus albus | G | R | 75.5 |
| Melilotus albus | G | O | 70.0 |
| Melissa officinalis | G | O | 81.1 |
| Mentha piperita | G | O | 54.4 |
| Mentha pulegium | G | O | 59.4 |
| Mentha spicata | G | R | 38.8 |
| Mentha spicata | G | O | 83.0 |
| Mentha suaveolens | G | O | 56.5 |
| Nepeta cataria | G | O | 56.2 |
| Ocimum basilicum | G | O | 60.3 |
| Oenothera biennis | G | R | 39.2 |
| Oenothera biennis | G | O | 44.3 |
| Origanum majorana | G | O | 44.7 |
| Origanum vulgare | G | O | 58.1 |
| Origanum vulgare | G | R | 22.9 |
| Oryza Sativa | G | R | 71.8 |
| Oryza Sativa | G | O | 39.8 |
| Oxalis Deppei | G | R | 80.1 |
| Oxalis Deppei | G | O | 28.8 |
| Oxyria digyna | G | R | 51.8 |
| Oxyria digyna | G | O | 36.2 |
| Panax quinquefolius | G | R | 72.1 |
| Panax quinquefolius | G | O | 81.6 |
| Panicum miliaceum | G | O | 93.4 |
| Passiflora caerula | G | R | 33.2 |
| Passiflora caerula | G | O | 63.2 |
| Pastinaca sativa | G | O | 54.0 |
| Pennisetum alopecuroides | G | R | 61.0 |
| Petasites japonicus | G | O | 50.0 |
| Petroselinum crispum | G | R | 49.1 |
| Petroselinum crispum | G | O | 52.2 |
| Phalaris canariensis | G | O | 72.1 |
| Phaseolus vulgaris | G | R | 21.8 |
| Pimpinella anisum | G | O | 86.2 |
| Pisum sativum | G | O | 61.6 |
| Pisum sativum | G | R | 57.5 |
| Plantago major | G | O | 91.9 |
| Plectranthus sp. | G | R | 53.0 |
| Plectranthus sp. | G | O | 73.0 |
| Polygonum aviculare | G | R | 32.2 |
| Polygonum aviculare | G | O | 36.4 |
| Portulaca oleracea | G | R | 82.1 |
| Portulaca oleracea | G | O | 63.3 |
| Potentilla anserina | G | R | 26.3 |
| Poterium sanquisorba | G | O | 79.9 |
| Prunella vulgaris | G | R | 68.8 |
| Prunella vulgaris | G | O | 57.4 |
| Raphanus Raphanistrum | G | R | 91.9 |
| Raphanus Raphanistrum | G | O | 55.2 |
| Rhaphanus sativus | G | R | 55.7 |
| Rhaphanus sativus | G | O | 78.4 |
| Rheum rhabarbarum | G | R | 27.1 |
| Rheum rhabarbarum | G | O | 56.8 |
| Ribes nidigrolaria | G | O | 70.7 |
| Ribes nigrum | G | R | 37.9 |
| Ribes nigrum | G | O | 98.9 |
| Ribes Sylvestris | G | R | 25.2 |
| Ribes Sylvestris | G | O | 65.7 |
| Ricinus communis | G | R | 39.3 |
| Ricinus communis | G | O | 84.3 |
| Rosmarinus officinalis | G | O | 68.6 |
| Rubus idaeus | G | O | 26.3 |
| Rumex crispus | G | R | 54.2 |
| Rumex crispus | G | O | 62.0 |
| Rumex scutatus | G | O | 38.1 |
| Ruta graveolens | G | O | 85.0 |
| Salix purpurea | G | R | 74.7 |
| Salix purpurea | G | O | 38.5 |
| Salvia elegans | G | O | 54.8 |
| Salvia officinalis | G | R | 89.7 |
| Salvia officinalis | G | O | 84.9 |
| Salvia sclarea | G | O | 61.8 |
| Sambucus ebulus | G | R | 48.2 |
| Sambucus ebulus | G | O | 98.2 |
| Santolina chamaecyparissus | G | R | 61.3 |
| Santolina chamaecyparissus | G | O | 88.2 |
| Saponaria officinalis | G | R | 52.9 |
| Saponaria officinalis | G | O | 71.8 |
| Satureja hortensis | G | O | 44.9 |
| Satureja montana | G | O | 76.8 |
| Scorzonera hispanica | G | R | 32.9 |
| Scuttellaria lateriflora | G | O | 49.8 |
| Scuttellaria lateriflora | G | R | 39.6 |
| Secale cereale | G | R | 37.0 |
| Senecio vulgaris | G | R | 31.0 |
| Senecio vulgaris | G | O | 47.0 |
| Setaria italica | G | R | 44.9 |
| Setaria italica | G | O | 42.0 |
| Silene vulgaris | G | R | 76.8 |
| Silene vulgaris | G | O | 92.2 |
| Sium sisarum | G | O | 58.9 |
| Sium sisarum | G | R | 66.6 |
| solanum melongena | G | R | 66.8 |
| Solanum tuberosum | G | O | 47.4 |
| Solidago sp | G | R | 53.6 |
| Solidago sp | G | O | 88.3 |
| Sonchus oleraceus | G | R | 62.5 |
| Sonchus oleraceus | G | O | 55.5 |
| Sorghum dochna | G | R | 67.4 |
| Sorghum dochna | G | O | 73.7 |
| sorghum durra | G | R | 24.8 |
| sorghum durra | G | O | 42.3 |
| Sorghum sudanense | G | R | 35.5 |
| Sorghum sudanense | G | O | 66.3 |
| Stachys byzantina | G | R | 75.5 |
| Stachys byzantina | G | O | 66.7 |
| Stellaria graminea | G | R | 36.9 |
| Stellaria graminea | G | O | 40.1 |
| Stellaria media | G | R | 31.2 |
| Stellaria media | G | O | 51.1 |
| Symphytum officinale | G | R | 90.2 |
| Symphytum officinale | G | O | 90.8 |
| Tanacetum cinerariifolium | G | O | 76.1 |
| Tanacetum parthenium | G | R | 70.1 |
| Tanacetum parthenium | G | O | 62.4 |
| Tanacetum vulgare | G | R | 36.2 |
| Tanacetum vulgare | G | O | 72.5 |
| Taraxacum officinale | G | O | 100.0 |
| Taraxacum officinale | G | R | 78.6 |
| Teucrium chamaedrys | G | O | 50.5 |
| Teucrium chamaedrys | G | R | 40.1 |
| Thymus fragantissimus | G | R | 81.4 |
| Thymus fragantissimus | G | O | 58.4 |
| Thymus praecox subsp arcticus | G | R | 49.2 |
| Thymus praecox subsp arcticus | G | O | 62.4 |
| Thymus serpyllum | G | O | 70.4 |
| Thymus serpyllum | G | R | 54.9 |
| Thymus vulgaris | G | R | 55.1 |
| Thymus x citriodorus | G | O | 47.1 |
| Tiarella cordifolia | G | O | 52.8 |
| Tropaeolum majus | G | R | 22.2 |
| Tropaeolum majus | G | O | 59.1 |
| Typha latifolia | G | R | 65.1 |
| Typha latifolia | G | O | 46.9 |
| Vaccinium macrocarpon | G | O | 76.7 |
| Vaccinium corymbosum | G | O | 54.5 |
| Vaccinium corymbosum | G | R | 82.9 |
| Vaccinium angusutifolium | G | R | 27.9 |
| Vaccinium angusutifolium | G | O | 66.8 |
| Vaccinium macrocarpon | G | R | 40.7 |
| Veratrum viride | G | O | 35.4 |
| Verbascum thapsus | G | O | 72.9 |
| Verbascum thapsus | G | R | 60.5 |
| Viburnum trilobum | G | R | 52.6 |
| Vicia sativa | G | R | 36.6 |
| Vicia sativa | G | O | 83.2 |
| Vicia villosa | G | O | 77.3 |
| Vicia villosa | G | R | 46.8 |
| Vinca minor | G | O | 63.0 |
| Vinca minor | G | R | 30.8 |
| Vitis sp. | G | R | 52.7 |
| Vitis sp. | G | O | 99.2 |
| Zea mays | G | R | 45.1 |
| Zea mays | G | O | 55.3 |
| Achillea millefolium | T | O | 46.0 |
| Achillea millefolium | T | R | 32.9 |
| Aconitum napellus | T | O | 35.2 |
| Aconitum napellus | T | R | 31.9 |
| Acorus calamus | T | O | 40.6 |
| Acorus calamus | T | R | 26.9 |
| Actinidia arguta | T | R | 80.0 |
| Actinidia arguta | T | O | 66.3 |
| Adiantum pedatum | T | O | 43.4 |
| Agrimonia eupatoria | T | O | 37.5 |
| Agropyron repens | T | O | 75.0 |
| Agropyron repens | T | R | 50.0 |
| Alchemilla mollis | T | O | 71.6 |
| Alchemilla mollis | T | R | 81.1 |
| Allium ampeloprasum | T | O | 84.4 |
| Allium cepa | T | O | 49.2 |
| Allium cepa | T | R | 30.1 |
| Allium sativum | T | O | 63.8 |
| Allium schoenoprasum | T | O | 79.6 |
| Allium tuberosum | T | O | 55.8 |
| Allium tuberosum | T | R | 29.6 |
| Aloe vera | T | R | 30.3 |
| Aloe vera | T | O | 42.7 |
| Althaea officinalis | T | R | 42.5 |
| Althaea officinalis | T | O | 46.3 |
| Amaranthus candatus | T | R | 37.3 |
| Amaranthus candatus | T | O | 60.0 |
| Amaranthus retroflexus | T | R | 33.2 |
| Amaranthus retroflexus | T | O | 94.3 |
| angelica archangelica | T | O | 37.4 |
| angelica archangelica | T | R | 55.7 |
| Anthriscus cerefolium | T | O | 86.5 |
| Anthriscus cerefolium | T | R | 69.6 |
| Apium graveolens | T | R | 22.0 |
| Aralia nudicaulis | T | O | 77.5 |
| Aralia nudicaulis | T | R | 28.4 |
| Arctium minus | T | R | 54.4 |
| Arctium minus | T | O | 89.5 |
| Armoracia rusticana | T | O | 84.9 |
| Aronia melanocarpa | T | R | 61.9 |
| Aronia melanocarpa | T | O | 84.5 |
| Artemisia absinthium | T | R | 29.0 |
| Artemisia absinthium | T | O | 55.9 |
| Artemisia dracunculus | T | O | 96.7 |
| Artium lappa | T | O | 26.0 |
| Asclepias incarnata | T | R | 58.5 |
| Asclepias incarnata | T | O | 66.8 |
| Aster spp | T | R | 40.5 |
| Aster spp | T | O | 86.7 |
| Atropa belladonna | T | O | 61.4 |
| Atropa belladonna | T | R | 30.4 |
| Avena sativa | T | R | 38.0 |
| Cyperus esculentus | T | O | 47.6 |
| Cyperus esculentus | T | R | 49.5 |
| Beta vulgaris | T | O | 62.2 |
| Borago officinalis | T | O | 39.1 |
| Brassica Napus | T | O | 89.3 |
| Brassica nigra | T | R | 26.9 |
| Brassica oleracea | T | O | 63.9 |
| Brassica oleracea | T | R | 76.2 |
| Brassica oleracea | T | O | 69.9 |
| Bromus inermis | T | R | 79.8 |
| Bromus inermis | T | O | 88.1 |
| Calamagrostis arundiflora m | T | R | 62.8 |
| Calendula officinalis | T | R | 64.6 |
| Canna edulis | T | O | 47.5 |
| Capsella bursa-pastoris | T | R | 48.7 |
| Capsella bursa-pastoris | T | O | 40.9 |
| Carex morrowii | T | R | 45.7 |
| Carex morrowii | T | O | 70.3 |
| Carum carvi | T | R | 22.7 |
| Cerastium tomentosum | T | R | 46.8 |
| Chaerophyllum bulbosum | T | R | 22.9 |
| Chaerophyllum bulbosum | T | O | 40.9 |
| Chelidonium majus | T | O | 60.7 |
| Chelidonium majus | T | R | 24.0 |
| Chenopodium quinoa | T | R | 41.5 |
| Chenopodium quinoa | T | O | 86.7 |
| Cicer arietinum | T | R | 20.4 |
| Cicer arietinum | T | O | 84.2 |
| Cichorium endivia | T | O | 76.3 |
| Cichorium intybus | T | O | 81.7 |
| Cichorium intybus | T | R | 73.3 |
| Circium arvense | T | R | 50.0 |
| Circium arvense | T | O | 74.8 |
| Citrullus colocynthus | T | O | 62.5 |
| Citrullus colocynthis | T | R | 57.3 |
| Coix Lacryma-Jobi | T | R | 33.7 |
| Coriandrum sativum | T | O | 59.2 |
| Coriandrum sativum | T | R | 37.1 |
| Cornus canadensis | T | R | 82.6 |
| Cornus canadensis | T | O | 47.7 |
| Crataegus sp | T | O | 33.9 |
| Crataegus submollis | T | O | 64.3 |
| Cryptotaenia canadensis | T | O | 60.9 |
| Cryptotaenia canadensis | T | R | 41.5 |
| Cymbopogon citratus | T | R | 65.2 |
| Cymbopogon citratus | T | O | 65.6 |
| Daucus carota | T | R | 27.5 |
| Dioscorea batatas | T | O | 42.3 |
| Dirca palustris | T | O | 57.4 |
| Dirca palustris | T | R | 29.5 |
| Echinacea purpurea | T | O | 83.0 |
| Eleusine coracana | T | O | 70.3 |
| Erysimum perofskianum | T | R | 90.4 |
| Erysimum perofskianum | T | O | 92.2 |
| Fagopyrum esculentum | T | R | 61.6 |
| Fagopyrum esculentum | T | O | 39.0 |
| Fagopyrum tataricum | T | R | 36.7 |
| Fagopyrum tataricum | T | O | 25.6 |
| Foeniculum vulgare | T | O | 79.0 |
| Fragaria x ananassa | T | O | 26.0 |
| Frangula alnus | T | O | 27.0 |
| Frangula alnus | T | R | 45.3 |
| Galinsoga ciliata | T | R | 34.6 |
| Galinsoga ciliata | T | O | 60.3 |
| Galium odoratum | T | R | 98.8 |
| Galium odoratum | T | O | 96.1 |
| Gaultheria hispidula | T | O | 33.1 |
| Gaultheria procumbens | T | O | 84.2 |
| Glechoma hederacea | T | O | 70.1 |
| Glechoma hederacea | T | R | 38.5 |
| Glycine max | T | O | 54.8 |
| Glycine max | T | R | 38.0 |
| Glycine max | T | O | 88.7 |
| Glycyrrhiza glabra | T | O | 65.5 |
| Glycyrrhiza glabra | T | R | 40.5 |
| Guizotia abyssinica | T | R | 48.1 |
| Guizotia abyssinica | T | O | 84.1 |
| Hamamelis virginiana | T | R | 35.9 |
| Hedeoma pulegioides | T | R | 24.8 |
| Helianthus strumosus | T | O | 32.9 |
| Helianthus strumosus | T | R | 31.0 |
| Helianthus tuberosus | T | R | 42.8 |
| Helianthus tuberosus | T | O | 72.1 |
| Helichrysum angustifolium | T | R | 69.6 |
| Helichrysum angustifolium | T | O | 84.9 |
| Helichrysum thianschanicum | T | R | 96.2 |
| Helichrysum thianschanicum | T | O | 80.7 |
| Humulus lupulus | T | O | 71.3 |
| Humulus lupulus | T | R | 60.6 |
| Hyoscyamus niger | T | O | 68.0 |
| Hyssopus officinalis | T | R | 73.3 |
| Hyssopus officinalis | T | O | 76.9 |
| Inula helenium | T | O | 93.3 |
| Inula helenium | T | R | 63.5 |
| Ipomoea batalas | T | O | 99.9 |
| Juniperus communis | T | R | 26.9 |
| Kochia scoparia. | T | O | 76.7 |
| Koeleria glauca | T | R | 89.1 |
| Koeleria glauca | T | O | 67.7 |
| Lactuca sativa | T | O | 75.2 |
| Lactuca sativa | T | R | 55.3 |
| Lathyrus Sativus | T | R | 23.3 |
| Lathyrus Sativus | T | O | 70.6 |
| Lathyrus sylvestris | T | R | 77.1 |
| Lathyrus sylvestris | T | O | 53.0 |
| Laurus nobilis | T | R | 61.6 |
| Laurus nobilis | T | O | 92.7 |
| Lavandula angustifolia | T | R | 54.1 |
| Lavandula angustifolia | T | O | 84.4 |
| Lavandula latifolia | T | R | 55.4 |
| Lavandula latifolia | T | O | 82.9 |
| Ledum groenlandicum | T | O | 96.1 |
| Ledum groenlandicum | T | R | 74.0 |
| Lens culinaris subsp culinaris | T | R | 36.4 |
| Lens culinaris subsp culinaris | T | O | 100.0 |
| Levisticum officinale | T | R | 38.8 |
| Levisticum officinale | T | O | 73.4 |
| Lotus corniculatus | T | O | 81.6 |
| Lotus corniculatus | T | R | 52.0 |
| Lupinus polyphyllus | T | R | 53.3 |
| Lupinus polyphyllus | T | O | 64.4 |
| Luzula sylvatica | T | R | 62.6 |
| Malus | T | O | 70.9 |
| Malus hupehensis | T | R | 77.6 |
| Malus hupehensis | T | O | 72.4 |
| Medicago sativa | T | R | 41.0 |
| Medicago sativa | T | O | 94.1 |
| Melilotus officinalis | T | R | 44.0 |
| Melilotus officinalis | T | O | 90.8 |
| Mentha piperita | T | O | 20.6 |
| Menyanthes trifoliata | T | R | 20.8 |
| Miscanthus sinensis | T | R | 89.0 |
| Miscanthus sinensis | T | O | 73.7 |
| Nepeta cataria | T | R | 25.3 |
| Ocimum Basilicum | T | O | 65.7 |
| Oenothera biennis | T | R | 40.2 |
| Oenothera biennis | T | O | 49.2 |
| Onobrychis viciiafolia | T | R | 53.2 |
| Onobrychis viciiafolia | T | O | 49.2 |
| Origanum vulgare | T | R | 50.6 |
| Origanum vulgare | T | O | 45.1 |
| Oryza sativa | T | R | 40.3 |
| Oryza sativa | T | O | 28.6 |
| Oxalis Deppei | T | R | 35.2 |
| Oxalis Deppei | T | O | 42.1 |
| oxyria digyna | T | R | 42.8 |
| oxyria digyna | T | O | 52.3 |
| Panax quinquefolius | T | O | 78.8 |
| Panicum miliaceum | T | R | 52.6 |
| Passiflora caerulea | T | O | 77.5 |
| Pastinaca sativa | T | R | 52.0 |
| Pastinaca sativa | T | O | 31.8 |
| Pennisetum alopecuroides | T | O | 73.4 |
| Perilla frutescens | T | R | 68.0 |
| Perilla frutescens | T | O | 74.4 |
| Pertoselinum crispum | T | R | 65.2 |
| Petasites Japonicus | T | R | 31.3 |
| Petasites Japonicus | T | O | 24.6 |
| Pertoselinum crispum | T | O | 45.2 |
| Phalaris canariensis | T | R | 33.6 |
| Phalaris canariensis | T | O | 86.5 |
| Phaseolus vulgaris | T | O | 57.0 |
| Physalis pruinosa | T | O | 58.2 |
| Pimpinella anisum | T | O | 95.9 |
| Pimpinella anisum | T | R | 91.7 |
| Pisum sativum | T | R | 30.5 |
| Pisum sativum | T | O | 69.3 |
| Plantago major | T | O | 93.8 |
| Plantago major | T | R | 20.2 |
| Plectranthus sp. | T | R | 44.4 |
| Plectranthus sp. | T | O | 50.8 |
| Polygonum aviculare | T | R | 47.9 |
| Polygonum aviculare | T | O | 72.7 |
| Potentilla anserina | T | R | 21.8 |
| Prunella vulgaris | T | R | 84.3 |
| Prunella vulgaris | T | O | 56.7 |
| Pteridium aquilinum | T | R | 32.6 |
| Raphanus raphanistrum | T | R | 68.6 |
| Raphanus raphanistrum | T | O | 77.0 |
| Raphanus sativus | T | R | 41.0 |
| Raphanus sativus | T | O | 63.1 |
| Ribes Sylvestre | T | O | 87.9 |
| Ribes Siylvestre | T | R | 40.2 |
| Ribes Siylvestre | T | O | 45.2 |
| Ricinus communis | T | R | 22.4 |
| Ricinus communis | T | O | 72.0 |
| Ribes nigrum | T | R | 50.5 |
| Ribes nigrum | T | O | 70.1 |
| Rosmarinus officinalis | T | O | 69.6 |
| Rubus canadensis | T | R | 37.2 |
| Rubus canadensis | T | O | 57.9 |
| Rubus idaeus | T | R | 64.9 |
| Rubus idaeus | T | O | 94.9 |
| Rumes scutatus | T | O | 74.9 |
| Rumes scutatus | T | R | 20.7 |
| Rumex acetosella | T | R | 40.1 |
| Rumex acetosella | T | O | 42.0 |
| Rumex crispus | T | R | 40.7 |
| Rumex crispus | T | O | 51.2 |
| Ruta graveolens | T | O | 91.2 |
| Salix purpurea | T | R | 55.5 |
| Salix purpurea | T | O | 51.2 |
| Salvia officinalis | T | R | 64.7 |
| Salvia officinalis | T | O | 66.6 |
| Sambucus canadensis | T | O | 92.5 |
| Sambucus canadensis | T | R | 64.0 |
| Sanguisorba minor | T | O | 68.4 |
| Santolina chamaecyparissus | T | R | 84.4 |
| Santolina chamaecyparissus | T | O | 33.9 |
| Saponaria officinalis | T | R | 59.3 |
| Saponaria officinalis | T | O | 80.4 |
| Satureja hortensis | T | O | 26.5 |
| Satureja hortensis | T | R | 23.0 |
| Satureja montana | T | R | 57.2 |
| Satureja montana | T | O | 43.5 |
| Satureja repandra | T | R | 47.1 |
| Satureja repandra | T | O | 66.3 |
| Scuttellaria lateriflora | T | O | 20.3 |
| Scuttellaria lateriflora | T | R | 33.8 |
| Secale cereale | T | R | 28.5 |
| Senecio vulgaris | T | R | 34.0 |
| Setaria italica | T | R | 40.7 |
| Silene vulgaris | T | R | 66.3 |
| Silene vulgaris | T | O | 99.7 |
| Sium sisarum | T | O | 90.7 |
| Sium sisarum | T | R | 39.6 |
| Solidago sp | T | R | 44.3 |
| Solidago sp | T | O | 73.6 |
| Sonchus oleraceus | T | R | 53.7 |
| Sonchus oleraceus | T | O | 36.9 |
| Sorghum caffrorum | T | R | 96.4 |
| Sorghum caffrorum | T | O | 80.1 |
| Sorghum dochna | T | R | 95.3 |
| Sorghum dochna | T | O | 70.3 |
| Sorghum dochna | T | R | 98.5 |
| Sorghum dochna | T | O | 85.3 |
| Sorghum durra | T | R | 86.5 |
| Sorghum durra | T | O | 81.7 |
| Sorghum sudanense | T | R | 34.7 |
| Stachys affinis | T | O | 75.7 |
| Stachys affinis | T | R | 33.5 |
| Stachys byzantina | T | R | 60.8 |
| Stachys byzantina | T | O | 77.5 |
| Stellaria graminea | T | R | 37.5 |
| Stellaria graminea | T | O | 54.7 |
| Stellaria media | T | R | 26.0 |
| Stellaria media | T | O | 49.0 |
| Stipa capillata | T | R | 43.4 |
| Symphytum officinale | T | R | 55.1 |
| Symphytum officinale | T | O | 64.0 |
| Tanacetum cinerariifolium | T | O | 65.5 |
| Tanacetum parthenium | T | R | 45.2 |
| Tanacetum parthenium | T | O | 54.7 |
| Tanacetum vulgare | T | R | 59.8 |
| Tanacetum vulgare | T | O | 86.0 |
| Taraxacum officinale | T | O | 100.0 |
| Taraxacum officinale | T | R | 91.3 |
| Teucrium chamaedrys | T | O | 60.8 |
| Teucrium chamaedrys L. | T | R | 69.2 |
| Thymus fragantissimus | T | R | 97.8 |
| Thymus fragantissimus | T | O | 81.7 |
| Thymus praecox subsp arcticus | T | R | 36.1 |
| Thymus praecox subsp arcticus | T | O | 31.8 |
| Thymus pseudolanuginosus | T | R | 33.9 |
| Thymus pseudolanuginosus | T | O | 43.7 |
| Thymus serpyllum | T | R | 39.2 |
| Thymus serpyllum | T | O | 68.6 |
| Thymus x citriodorus | T | O | 70.9 |
| Thymus x citriodorus | T | R | 46.1 |
| Tiarella cordifolia | T | O | 72.0 |
| Tragopogon porrifolius | T | O | 40.9 |
| Tragopogon porrifolius | T | R | 20.5 |
| Triticosecala spp. | T | O | 38.2 |
| Triticum aestivum | T | R | 31.4 |
| Triticum aestivum | T | O | 33.8 |
| Tropaeolum majus | T | R | 29.2 |
| Tropaeolum majus | T | O | 20.9 |
| Typha latifolia | T | R | 67.0 |
| Typha latifolia | T | O | 56.0 |
| Urtica dioica | T | R | 77.8 |
| Urtica dioica | T | O | 75.6 |
| Vaccinium angustifolium | T | O | 58.6 |
| Vaccinium macrocarpon | T | R | 20.1 |
| Vaccinium macrocarpon | T | O | 41.7 |
| Veratrum viride | T | O | 57.1 |
| Veratrum viride | T | R | 26.6 |
| Verbascum thapsus | T | O | 72.8 |
| Verbascum thapsus | T | R | 56.0 |
| Vibumum trilobum | T | R | 49.5 |
| Vibumum trilobum | T | O | 56.8 |
| Vicia sativa | T | O | 73.9 |
| Vicia villosa | T | R | 79.2 |
| Vicia villosa | T | O | 70.9 |
| Vinca minor | T | O | 21.5 |
| Vitis sp. | T | R | 79.7 |
| Vitis sp. | T | O | 97.4 |
| Zea mays | T | R | 83.5 |
| Zea mays | T | O | 58.2 |

TABLE 9
Cath K

| Nom latin | Stress | Extrait | Inhibition |
|---|---|---|---|
| Achillea millefolium | A | O | 27.6 |
| Aconitum napellus | A | O | 74.0 |
| Acorus calamus | A | O | 74.8 |
| Actinidia arguta | A | R | 28.1 |
| Actinidia arguta | A | O | 96.6 |
| Agropyron repens | A | O | 98.0 |
| Alchemilla mollis | A | O | 61.3 |
| Alchemilla mollis | A | R | 95.8 |
| Allium cepa | A | O | 80.6 |
| Allium porrum | A | R | 30.9 |
| Allium porrum | A | O | 87.5 |
| Allium sativum | A | O | 71.2 |
| Allium schoenoprasum | A | O | 78.2 |
| Allium Tuberosum | A | O | 99.6 |
| Aloe vera | A | R | 60.0 |
| Aloe vera | A | O | 78.4 |
| Althaea officinalis | A | O | 98.1 |
| Amaranthus retroflexus | A | R | 37.4 |
| Amaranthus retroflexus | A | O | 43.4 |
| Anethum graveolens | A | O | 33.7 |
| Angelica archangelica | A | R | 36.0 |
| Angelica archangelica | A | O | 85.2 |
| Apium graveolens | A | R | 46.7 |
| Apium graveolens | A | O | 88.8 |
| Aralia nudicaulis | A | R | 79.0 |
| Aralia nudicaulis | A | O | 98.5 |
| Arctium minus | A | R | 24.6 |
| Arctium minus | A | O | 67.9 |
| Arctostaphylos uva-ursi | A | R | 75.1 |
| Arctostaphylos uva-ursi | A | O | 89.8 |
| Armoracia rusticana | A | O | 92.3 |
| Aronia melanocarpa | A | O | 60.1 |
| Aronia melanocarpa | A | R | 61.6 |
| Aronia melanocarpa | A | O | 82.3 |
| Artemisia Absinthium | A | R | 88.6 |
| Artemisia dracunculus | A | O | 55.6 |
| Aster sp | A | R | 50.7 |
| Atropa belladonna | A | O | 89.4 |
| Beckmannia eruciformis | A | R | 86.0 |
| Beckmannia eruciformis | A | O | 96.2 |
| Beta vulgaris | A | R | 69.3 |
| Beta vulgaris | A | O | 87.6 |
| Beta vulgaris ssp. Maritima | A | R | 53.7 |
| Beta vulgaris ssp. Maritima | A | O | 84.2 |
| Borago officinalis | A | O | 38.6 |
| Brassica napus | A | R | 43.5 |
| Brassica napus | A | O | 84.4 |
| Brassica oleracea | A | O | 60.6 |
| Brassica rapa | A | R | 62.1 |
| Brassica rapa | A | O | 98.9 |
| Campanula rapunculus | A | O | 77.0 |
| Canna edulis | A | R | 32.0 |
| Capsella bursa-pastoris | A | R | 71.4 |
| Capsella bursa-pastoris | A | O | 72.8 |
| Capsicum annuum | A | R | 39.0 |
| Chaerophyllum bulbosum | A | O | 86.6 |
| Chelidonium majus | A | O | 90.3 |
| Chenopodium bonus-henricus | A | O | 38.8 |
| Chenopodium quinoa | A | R | 42.3 |
| Chenopodium quinoa | A | O | 84.3 |
| Cicer arietinum | A | O | 91.1 |
| Cichorium intybus | A | R | 21.0 |
| Cichorium intybus | A | O | 94.8 |
| Coix Lacryma-Jobi | A | O | 35.2 |
| Coriandrum sativum | A | R | 63.6 |
| Coriandrum sativum | A | O | 84.4 |
| Cornus canadensis | A | O | 58.6 |
| Cornus canadensis | A | R | 99.4 |
| Crataegus sp | A | R | 22.7 |
| Crataegus submollis | A | O | 45.4 |
| Cryptotaenia canadensis | A | R | 26.3 |
| Cryptotaenia canadensis | A | O | 29.1 |
| Cymbopogon citratus | A | O | 45.2 |
| Cyperus esculentus | A | O | 75.0 |
| Daucus carota | A | O | 92.9 |
| Dirca palustris | A | O | 84.7 |
| Dirca palustris | A | R | 94.2 |
| Dryopteris filix-mas | A | O | 85.7 |
| Echinacea purpurea | A | O | 89.8 |
| Eleusine coracana | A | R | 50.6 |
| Eleusine coracana | A | O | 58.7 |
| Fagopyrum esculentum | A | O | 68.0 |
| Fagopyrum tataricum | A | O | 20.3 |
| Fagopyrum tataricum | A | R | 33.0 |
| Foeniculum vulgare | A | O | 40.3 |
| Fragaria x ananassa | A | R | 44.8 |
| Fragaria x ananassa | A | O | 92.3 |
| Galinsoga ciliata | A | O | 55.3 |
| Galium odoratum | A | O | 88.4 |
| Gaultheria hispidula | A | R | 61.6 |
| Gaultheria hispidula | A | O | 87.1 |
| Glechoma hederacea | A | O | 96.2 |
| Glycine max | A | R | 41.6 |
| Glycine max | A | O | 100.0 |
| Glycyrrhiza glabra | A | R | 50.8 |
| Glycyrrhiza glabra | A | O | 90.2 |
| Guizotia abyssinica | A | R | 23.1 |
| Guizotia abyssinica | A | O | 94.8 |
| Hamamelis virginiana | A | R | 91.8 |
| Hedeoma pulegioides | A | O | 93.3 |
| Helleborus niger | A | O | 82.9 |
| Hordeum hexastichon | A | R | 26.9 |
| Hyssopus officinalis | A | R | 40.2 |
| Inula helenium | A | O | 86.0 |
| Ipomoea Batatas | A | R | 25.6 |
| Lathyrus sativus | A | R | 26.9 |
| Lathyrus sativus | A | O | 84.9 |
| Lathyrus sylvestris | A | R | 22.4 |
| Lathyrus sylvestris | A | O | 93.4 |
| Laurus nobilis | A | O | 64.2 |
| Laurus nobilis | A | R | 64.6 |
| Leonurus cardiaca | A | O | 90.0 |
| Levisticum officinale | A | R | 49.4 |
| Levisticum officinale | A | O | 53.3 |
| Lotus corniculatus | A | R | 67.4 |
| Lotus corniculatus | A | O | 98.8 |
| Lycopersicon esculentum | A | R | 30.1 |
| Malva sylvestris | A | O | 82.3 |
| Medicago sativa | A | R | 44.0 |
| Medicago sativa | A | O | 94.4 |
| Melilotus albus | A | R | 80.7 |
| Melilotus albus | A | O | 98.9 |
| Melissa officinalis | A | O | 89.4 |
| Melissa officinalis | A | R | 93.6 |
| Mentha piperita | A | O | 60.1 |
| Mentha piperita | A | R | 60.8 |
| Mentha pulegium | A | O | 55.4 |
| Mentha spicata | A | O | 97.0 |
| Mentha suaveolens | A | O | 46.8 |
| Nepeta cataria | A | R | 32.6 |
| Nepeta cataria | A | O | 67.2 |
| Nicotiana tabacum | A | R | 34.1 |
| Oenothera biennis | A | R | 48.5 |
| Oenothera biennis | A | O | 83.4 |
| Origanum majorana | A | O | 63.2 |
| Origanum vulgare | A | R | 62.2 |
| Origanum vulgare | A | O | 90.0 |
| Panax quinquefolius | A | O | 32.3 |
| Panax quinquefolius | A | R | 75.9 |
| Panicum miliaceum | A | R | 25.6 |
| Panicum miliaceum | A | O | 45.1 |
| Pastinaca sativa | A | O | 100.0 |
| Petasites japonicus | A | O | 82.7 |
| Petroselinum crispum | A | R | 50.2 |
| Petroselinum crispum | A | O | 85.7 |
| Petroselinum crispum | A | O | 92.2 |
| Phalaris canariensis | A | R | 89.5 |
| Phaseolus vulgaris | A | R | 22.1 |
| Phaseolus Vulgaris | A | O | 90.3 |
| Pimpinella anisum | A | O | 72.4 |
| Plantago major | A | R | 22.2 |
| Plantago major | A | O | 99.8 |
| Plectranthus sp. | A | R | 73.5 |
| Potentilla anserina | A | O | 92.9 |
| Pteridium aquilinum | A | O | 81.9 |
| Raphanus raphanistrum | A | O | 70.2 |
| Raphanus sativus | A | R | 28.4 |
| Raphanus sativus | A | O | 99.0 |
| Rheum rhabarbarum | A | R | 21.4 |
| Rheum rhabarbarum | A | O | 95.6 |
| Ribes nigrum | A | R | 59.3 |
| Ribes nigrum | A | O | 81.8 |
| Ribes Sylvestre | A | O | 98.6 |
| Ricinus communis | A | R | 78.5 |
| Ricinus communis | A | O | 90.2 |
| Rosa rugosa | A | R | 36.1 |
| Rubus allegheniensis | A | O | 59.3 |
| Rubus canadensis | A | O | 94.4 |
| Rubus idaeus | A | R | 58.4 |
| Rubus idaeus | A | O | 97.4 |
| Rumex Acetosa | A | O | 83.9 |
| Rumex acetosella | A | R | 46.7 |
| Rumex acetosella | A | O | 90.9 |
| Rumex crispus | A | R | 32.9 |
| Rumex crispus | A | O | 91.8 |
| Rumex Scutatus | A | O | 94.9 |
| Ruta graveolens | A | O | 92.5 |
| Salix purpurea | A | O | 44.8 |
| Salix purpurea | A | R | 68.1 |
| Salvia elegans | A | O | 64.2 |
| Salvia officinalis | A | O | 67.8 |
| Salvia officinalis | A | R | 85.4 |
| Salvia sclarea | A | O | 61.0 |
| Santolina chamaecyparissus | A | R | 54.1 |
| Santolina chamaecyparissus | A | O | 63.1 |
| Satureja montana | A | O | 75.6 |
| Scorzonera hispanica | A | O | 62.7 |
| Scutellaria lateriflora | A | O | 82.7 |
| Senecio vulgaris L. | A | R | 80.9 |
| Setaria italica | A | R | 30.0 |
| Setaria italica | A | O | 66.2 |
| Sium Sisarum | A | R | 30.0 |
| Sium Sisarum | A | O | 93.3 |
| Solanum tuberosum | A | R | 30.1 |
| Solanum tuberosum | A | O | 79.8 |
| Solidago sp | A | R | 43.7 |
| Solidago sp | A | O | 72.1 |
| Sonchus oleraceus | A | R | 21.6 |
| Sonchus oleraceus | A | O | 92.4 |
| Sorghum dochna | A | O | 60.9 |
| Sorghum durra | A | O | 89.3 |
| Stachys affinis | A | R | 29.3 |
| Stachys byzantina | A | R | 28.3 |
| Stellaria graminea | A | R | 49.9 |
| Stellaria graminea | A | O | 87.6 |
| Stellaria media | A | R | 25.7 |
| Stellaria media | A | O | 26.0 |
| Tanacetum parthenium | A | R | 64.6 |
| Tanacetum vulgare | A | R | 36.0 |
| Tanacetum vulgare | A | O | 85.7 |
| Taraxacum officinale | A | R | 36.9 |
| Taraxacum officinale | A | O | 100.0 |
| Teucrium chamaedrys | A | O | 92.5 |
| Thymus praecox subsp arcticus | A | O | 50.1 |
| Thymus serpyllum | A | R | 27.3 |
| Thymus serpyllum | A | O | 88.9 |
| Thymus vulgaris | A | R | 60.9 |
| Thymus vulgaris | A | O | 74.3 |
| Thymus x citriodorus | A | O | 80.9 |
| Tragopogon porrifolius | A | R | 43.2 |
| Tragopogon porrifolius | A | O | 81.9 |
| Tropaeolum majus | A | R | 42.6 |
| Tropaeolum majus | A | O | 82.6 |
| Typha latifolia | A | O | 49.5 |
| Typha latifolia | A | R | 65.4 |
| Vaccinium Corymbosum | A | O | 94.5 |
| Vaccinium macrocarpon | A | O | 94.1 |
| Veratrum viride | A | O | 78.4 |
| Verbascum thapsus | A | O | 96.4 |
| Vicia sativa | A | O | 98.7 |
| Vicia villosa | A | R | 29.0 |
| Vicia villosa | A | O | 97.6 |
| Vinca minor | A | O | 74.6 |
| Vitis sp. | A | R | 82.1 |
| Vitis sp. | A | O | 99.5 |
| Zea mays | A | R | 24.4 |
| Zea mays | A | O | 99.2 |
| Achillea millefolium | G | O | 42.8 |
| Aconitum napellus | G | O | 37.1 |
| Acorus calamus | G | O | 89.0 |
| Actinidia arguta | G | R | 35.5 |
| Actinidia arguta | G | O | 45.4 |
| Adiantum pedatum | G | O | 25.0 |
| Agropyron repens | G | O | 98.2 |
| Alchemilla mollis | G | O | 65.5 |
| Alchemilla mollis | G | R | 88.9 |
| Allium ampeloprasum | G | R | 39.0 |
| Allium ampeloprasum | G | O | 53.8 |
| Allium cepa | G | R | 35.6 |
| Allium cepa | G | O | 75.1 |
| Allium sativum | G | O | 82.4 |
| Allium schoenoporasum | G | O | 88.7 |
| Allium tuberosum | G | O | 80.3 |
| Aloe vera | G | R | 28.8 |
| althaea officinalis | G | O | 94.5 |
| Amaranthus retroflexus | G | R | 35.3 |
| Amaranthus retroflexus | G | O | 73.8 |
| Anethum graveolens | G | O | 52.0 |
| Angelica archangelica | G | R | 39.0 |
| Angelica archangelica | G | O | 80.6 |
| Apium graveolens | G | R | 37.7 |
| Apium graveolens | G | O | 83.9 |
| Aralia nudicaulis | G | O | 86.7 |
| Aralia nudicaulis | G | R | 89.5 |
| Arctium minus | G | R | 27.1 |
| Arctium minus | G | O | 93.4 |
| Arctostaphylos uva-ursi | G | R | 73.3 |
| Armoracia rusticana | G | O | 53.8 |
| Aronia melanocarpa | G | R | 73.2 |
| Aronia melanocarpa | G | O | 81.2 |
| Artemisia absinthium | G | R | 92.0 |
| Artemisia dracunculus | G | R | 36.0 |
| Artemisia dracunculus | G | O | 72.7 |
| Asclepias incarnata | G | R | 67.4 |
| Asclepias incarnata | G | O | 87.0 |
| Asparagus officinalis | G | O | 98.2 |
| Aster | G | O | 37.4 |
| Aster sp | G | R | 37.3 |
| Aster sp | G | O | 81.3 |
| Beckmannia eruciformis | G | O | 90.0 |
| Beta vulgaris | G | O | 29.0 |
| Beta vulgaris | G | R | 71.5 |
| Borago officinalis | G | O | 36.4 |
| Brassica napus | G | R | 26.6 |
| Brassica napus | G | O | 98.8 |
| Brassica oleracea | G | O | 97.8 |
| Brassica rapa | G | R | 25.3 |
| Brassica rapa | G | O | 67.8 |
| Calamagrostis arundiflora | G | R | 23.2 |
| Campanula rapunculus | G | O | 80.2 |
| Canna edulis | G | R | 31.6 |
| Canna edulis | G | O | 44.2 |
| Capsella bursa-pastoris | G | R | 63.0 |
| Capsella bursa-pastoris | G | O | 69.5 |
| Carum carvi | G | O | 32.3 |
| Chaerophyllum bulbosum | G | R | 30.7 |
| Chaerophyllum bulbosum | G | O | 38.0 |
| Chelidonium majus | G | O | 91.3 |
| Cicer arietinum | G | R | 44.7 |
| Cicer arietinum | G | O | 92.7 |
| Cichorium endivia subsp. Endivia | G | O | 94.9 |
| Cichorium intybus | G | R | 25.8 |
| Cichorium intybus | G | O | 95.8 |
| Circium arvense | G | O | 73.0 |
| Circium arvense | G | R | 96.5 |
| Coix Lacryma-Jobi | G | O | 57.4 |
| Cornus canadensis | G | O | 62.5 |
| Cornus canadensis | G | R | 68.0 |
| Crataegus submollis | G | O | 58.3 |
| Crataegus submollis | G | R | 73.2 |
| Cymbopogon citratus | G | R | 65.5 |
| Cymbopogon citratus | G | O | 70.9 |
| Cyperus esculentus | G | O | 85.0 |
| Daucus carota | G | R | 23.3 |
| Daucus carota | G | O | 57.3 |
| Dirca palustris | G | R | 67.1 |
| Dirca palustris | G | O | 97.2 |
| Dryopteris filix-mas | G | O | 52.2 |
| Echinacea purpurea | G | O | 74.4 |
| Eleusine coracana | G | R | 38.7 |
| Eleusine coracana | G | O | 76.8 |
| Erigeron speciosus | G | R | 26.8 |
| Erysimum perofskianum | G | R | 59.8 |
| Erysimum perofskianum | G | O | 100.2 |
| Fagopyrum esculentum | G | R | 37.6 |
| Fagopyrum tartaricum | G | O | 27.3 |
| Fagopyrum tartaricum | G | R | 30.7 |
| Galinsoga ciliata | G | O | 30.9 |
| Galinsoga ciliata | G | R | 51.3 |
| Galium odoratum | G | O | 96.9 |
| Gaultheria hispidula | G | R | 70.9 |
| Gaultheria hispidula | G | O | 82.2 |
| Gaultheria procumbens | G | O | 69.6 |
| Glechoma hederacea | G | O | 94.0 |
| Glycine max | G | R | 76.1 |
| Glycine max | G | O | 100.0 |
| Glycyrrhiza glabra | G | R | 33.3 |
| Glycyrrhiza glabra | G | O | 94.5 |
| Guizotia abyssinica | G | R | 41.5 |
| Guizotia abyssinica | G | O | 95.4 |
| Hamamelis virginiana | G | O | 79.7 |
| Hamamelis virginiana | G | R | 90.8 |
| Helianthus strumosus | G | R | 31.7 |
| Helianthus strumosus | G | O | 39.4 |
| Helianthus tuberosus | G | R | 31.5 |
| Helianthus tuberosus | G | O | 70.6 |
| Helichrysum thianschanicum | G | R | 40.4 |
| Helichrysum thianschanicum | G | O | 69.2 |
| Helleborus niger | G | R | 43.8 |
| Helleborus niger | G | O | 90.6 |
| Hordeum hexastichon | G | R | 22.6 |
| Hordeum hexastichon | G | O | 86.0 |
| Hyssopus officinalis | G | R | 25.8 |
| Inula helenium | G | O | 82.2 |
| Lactuca sativa | G | R | 28.5 |
| Lactuca sativa | G | O | 95.5 |
| Lathyrus sylvestris | G | R | 22.1 |
| Lathyrus sylvestris | G | O | 79.5 |
| Laurus nobilis | G | R | 49.6 |
| Laurus nobilis | G | O | 72.3 |
| Lavandula angustifolia | G | O | 57.6 |
| Lavandula angustifolia | G | R | 65.2 |
| Ledum groenlandicum | G | R | 35.1 |
| Ledum groenlandicum | G | O | 97.9 |
| Leonurus cardiaca | G | O | 99.9 |
| Levisticum officinale | G | R | 75.1 |
| Levisticum officinale | G | O | 92.5 |
| Lotus corniculatus | G | R | 25.7 |
| Lotus corniculatus | G | O | 98.5 |
| Lupinus polyphyllus | G | O | 94.5 |
| Lupinus polyphyllus | G | R | 99.9 |
| Lycopersicon esculentum | G | R | 70.0 |
| Lycopersicon esculentum | G | O | 90.2 |
| Malus hupehensis | G | R | 44.8 |
| Malus hupehensis | G | O | 82.9 |
| Medicago sativa | G | R | 26.2 |
| Medicago sativa | G | O | 99.2 |
| Melilotus alba | G | R | 96.9 |
| Melilotus alba | G | O | 99.0 |
| Melissa officinalis | G | O | 33.2 |
| Melissa officinalis | G | R | 90.6 |
| Mentha piperita | G | O | 41.8 |
| Mentha pulegium | G | O | 38.7 |
| Mentha spicata | G | R | 32.7 |
| Mentha spicata | G | O | 80.1 |
| Mentha suaveolens | G | O | 55.7 |
| Nepeta cataria | G | R | 93.1 |
| Ocimum basilicum | G | O | 75.6 |
| Oenothera biennis | G | R | 42.9 |
| Oenothera biennis | G | O | 86.1 |
| Origanum majorana | G | O | 65.8 |
| Origanum vulgare | G | O | 89.6 |
| Origanum vulgare | G | R | 92.3 |
| Oryza Sativa | G | O | 95.6 |
| Oxalis Deppei | G | O | 86.8 |
| Oxalis Deppei | G | R | 87.8 |
| Oxyria digyna | G | R | 20.8 |
| Oxyria digyna | G | O | 89.3 |
| Panax quinquefolius | G | R | 52.7 |
| Panicum miliaceum | G | R | 31.5 |
| Panicum miliaceum | G | O | 94.4 |
| Passiflora caerulae | G | R | 21.1 |
| Passiflora caerulae | G | O | 60.6 |
| Pastinaca sativa | G | O | 72.8 |
| Pennisetum alopecuroides | G | R | 30.6 |
| Petasites japonicus | G | O | 81.6 |
| Petroselinum crispum | G | R | 62.9 |
| Petroselinum crispum | G | O | 76.3 |
| Phalaris canariensis | G | O | 22.0 |
| Phalaris canariensis | G | R | 36.7 |
| Phaseolus vulgaris | G | R | 65.5 |
| Phaseolus vulgaris | G | O | 88.2 |
| Pimpinella anisum | G | O | 46.2 |
| Pisum sativum | G | O | 52.5 |
| Plantago major | G | R | 29.0 |
| Plantago major | G | O | 96.3 |
| Plectranthus sp. | G | R | 54.5 |
| Polygonum aviculare | G | O | 29.6 |
| Portulaca oleracera | G | R | 50.9 |
| Potentilla anserina | G | O | 92.5 |
| Poterium sanquisorba | G | O | 74.2 |
| Prunella vulgaris | G | O | 77.1 |
| Prunella vulgaris | G | R | 91.8 |
| Pteridium aquilinum | G | O | 87.5 |
| Rhaphanus sativus | G | R | 24.0 |
| Rhaphanus sativus | G | O | 85.0 |
| Rheum rhabarbarum | G | R | 22.9 |
| Rheum rhabarbarum | G | O | 85.5 |
| Ribes nidigrolaria | G | O | 59.7 |
| Ribes nigrum | G | O | 80.4 |
| Ribes nigrum | G | R | 81.5 |
| Ribes Sylvestre | G | O | 91.7 |
| Ricinus communis | G | R | 27.0 |
| Ricinus communis | G | O | 98.3 |
| Rosmarinus officinalis | G | O | 27.5 |
| Rubus idaeus | G | R | 38.7 |
| Rubus idaeus | G | O | 51.2 |
| Rumex crispus | G | R | 37.1 |
| Rumex crispus | G | O | 95.0 |
| Rumex scutatus | G | O | 88.5 |
| Ruta graveolens | G | R | 46.4 |
| Ruta graveolens | G | O | 84.6 |
| Salix purpurea | G | O | 32.4 |
| Salix purpurea | G | R | 95.3 |
| Salvia elegans | G | O | 57.0 |
| Salvia officinalis | G | O | 65.8 |
| Salvia officinalis | G | R | 94.9 |
| Salvia sclarea | G | O | 58.5 |
| Sambucus ebulus | G | R | 32.1 |
| Sambucus ebulus | G | O | 67.7 |
| Santolina chamaecyparissus | G | R | 49.3 |
| Saponaria officinalis | G | R | 22.3 |
| Saponaria officinalis | G | O | 88.5 |
| Satureja hortensis | G | O | 73.3 |
| Satureja montana | G | O | 74.8 |
| Scorzonera hispanica | G | R | 43.1 |
| Scorzonera hispanica | G | O | 52.1 |
| Scutellaria lateriflora | G | O | 92.0 |
| Secale cereale | G | R | 23.7 |
| Senecio vulgaris | G | R | 29.1 |
| Setaria italica | G | R | 21.9 |
| Setaria italica | G | O | 83.2 |
| Silene vulgaris | G | R | 24.1 |
| Sium sisarum | G | R | 37.9 |
| Sium sisarum | G | O | 100.0 |
| solanum melongena | G | R | 22.7 |
| Solanum tuberosum | G | R | 50.2 |
| Solanum tuberosum | G | O | 73.3 |
| Solidago sp | G | R | 32.9 |
| Solidago sp | G | O | 87.3 |
| Sonchus oleraceus | G | R | 37.8 |
| Sonchus oleraceus | G | O | 48.1 |
| Sorghum dochna | G | R | 43.1 |
| Sorghum dochna | G | O | 91.3 |
| sorghum durra | G | R | 56.4 |
| sorghum durra | G | O | 63.2 |
| Sorghum sudanense | G | R | 56.1 |
| Sorghum sudanense | G | O | 89.7 |
| Stachys Affinis | G | R | 27.9 |
| Stachys byzantina | G | R | 42.8 |
| Stachys byzantina | G | O | 72.1 |
| Stellaria graminea | G | R | 39.7 |
| Stellaria media | G | R | 27.9 |
| Stellaria media | G | O | 50.0 |
| Symphytum officinale | G | O | 43.5 |
| Symphytum officinale | G | R | 74.2 |
| Tanacetum cinerariifolium | G | O | 72.2 |
| Tanacetum parthenium | G | R | 67.9 |
| Tanacetum vulgare | G | R | 49.5 |
| Tanacetum vulgare | G | O | 97.8 |
| Taraxacum officinale | G | R | 45.4 |
| taraxacum officinale | G | O | 100.0 |
| Teucrium chamaedrys | G | R | 61.7 |
| Teucrium chamaedrys | G | O | 89.8 |
| Thymus fragantissimus | G | O | 64.0 |
| Thymus fragantissimus | G | R | 85.4 |
| Thymus praecox subsp arcticus | G | R | 28.3 |
| Thymus praecox subsp arcticus | G | O | 39.1 |
| Thymus serpyllum | G | R | 28.4 |
| Thymus serpyllum | G | O | 90.3 |
| Thymus vulgaris | G | R | 69.0 |
| Thymus vulgaris | G | O | 70.6 |
| Thymus x citriodorus | G | O | 70.7 |
| Tiarella cordifolia | G | O | 88.4 |
| Tropaelum majus | G | O | 76.8 |
| Typha latifolia | G | O | 76.4 |
| Typha latifolia | G | R | 82.9 |
| Vaccinium corymbosum | G | R | 72.1 |
| Vaccinium corymbosum | G | O | 95.4 |
| Vaccinium macrocarpon | G | O | 95.3 |
| Veratrum viride | G | O | 80.8 |
| Verbascum thapsus | G | R | 27.3 |
| Verbascum thapsus | G | O | 91.3 |
| Viburnum trilobum | G | O | 68.5 |
| Viburnum trilobum | G | R | 72.6 |
| Vicia sativa | G | R | 32.2 |
| Vicia sativa | G | O | 96.8 |
| Vicia villosa | G | R | 29.7 |
| Vicia villosa | G | O | 98.7 |
| Vinca minor | G | O | 35.8 |
| Vitis sp. | G | R | 77.5 |
| Vitis sp. | G | O | 99.8 |
| Zea mays | G | O | 54.2 |
| Zea mays | G | R | 56.0 |
| Achillea millefolium | T | O | 89.0 |
| Aconitum napellus | T | O | 63.6 |
| Acorus calamus | T | O | 94.2 |
| Actinidia arguta | T | R | 52.4 |
| Actinidia arguta | T | O | 84.8 |
| Adiantum pedatum | T | O | 92.2 |
| Agrimonia eupatoria | T | O | 39.2 |
| Agropyron rupens | T | O | 97.3 |
| Alchemilla mollis | T | O | 85.2 |
| Alchemilla mollis | T | R | 96.8 |
| Allium ampeloprasum | T | R | 33.5 |
| Allium ampeloprasum | T | O | 94.1 |
| Allium cepa | T | R | 54.4 |
| Allium cepa | T | O | 100.0 |
| Allium sativum | T | O | 76.5 |
| Allium schoenoprasum | T | O | 87.0 |
| Allium tuberosum | T | R | 53.6 |
| Allium tuberosum | T | O | 98.7 |
| Aloe vera | T | R | 43.7 |
| Aloe vera | T | O | 79.9 |
| Althaea officinalis | T | O | 95.8 |
| Amaranthus caudathus | T | R | 20.7 |
| Amaranthus caudathus | T | O | 69.3 |
| Amaranthus retroflexus | T | R | 32.4 |
| angelica archangelica | T | R | 44.2 |
| angelica archangelica | T | O | 55.7 |
| Anthriscus cerefolium | T | O | 96.1 |
| Apium graveolens | T | R | 30.3 |
| Aralia nudicaulis | T | R | 68.2 |
| Aralia nudicaulis | T | O | 97.8 |
| Arctium minus | T | O | 92.9 |
| Arctostaphylos uva-ursi | T | O | 72.0 |
| Arctostaphylos uva-ursi | T | R | 79.8 |
| Armoracia rusticana | T | O | 88.0 |
| Aronia melanocarpa | T | R | 74.9 |
| Aronia melanocarpa | T | O | 80.0 |
| Artemisia absinthium | T | O | 41.7 |
| Artemisia absinthium | T | R | 96.1 |
| Artemisia dracunculus | T | O | 96.2 |
| Artium lappa | T | O | 21.1 |
| Asclepias incarnata | T | O | 81.5 |
| Asclepias incarnata | T | R | 86.7 |
| Aster | T | O | 34.1 |
| Aster sp | T | R | 46.8 |
| Aster sp | T | O | 49.7 |
| Atropa belladonna | T | O | 71.7 |
| Avena sativa | T | R | 40.4 |
| Beta vulgaris | T | O | 30.6 |
| Beta vulgaris | T | R | 41.7 |
| Borago officinalis | T | R | 59.2 |
| Borago officinalis | T | O | 76.5 |
| Brassica napus | T | R | 35.8 |
| Brassica Napus | T | O | 91.9 |
| Brassica nigra | T | R | 24.3 |
| Brassica oleracea | T | O | 83.8 |
| Bromus inermis | T | O | 69.6 |
| Bromus inermis | T | R | 91.2 |
| Calendula officinalis | T | R | 34.5 |
| Canna edulis | T | R | 20.5 |
| Canna edulis | T | O | 73.5 |
| Capsella bursa-pastoris | T | R | 32.1 |
| Capsella bursa-pastoris | T | O | 75.1 |
| Carex morrowii | T | R | 44.0 |
| Carex morrowii | T | O | 94.3 |
| Carum carvi | T | R | 20.5 |
| Cerastium tomentosum | T | R | 36.8 |
| Chaerophyllum bulbosum | T | R | 23.0 |
| Chaerophyllum bulbosum | T | O | 80.2 |
| Chelidonium majus | T | O | 94.3 |
| Chenopodium quinoa | T | O | 48.2 |
| Chenopodium quinoa | T | R | 48.3 |
| Cicer arietinum | T | R | 25.6 |
| Cicer arietinum | T | O | 81.7 |
| Cichorium endivia subsp endivia | T | R | 20.8 |
| Cichorium endivia subsp endivia | T | O | 95.5 |
| Cichorium intybus | T | R | 20.4 |
| Cichorium intybus | T | O | 96.0 |
| Circium arvense | T | R | 58.3 |
| Circium arvense | T | O | 79.6 |
| Citrullus colocynthis | T | R | 41.2 |
| Citrullus colocynthis | T | O | 84.9 |
| Coriandrum sativum | T | O | 38.4 |
| Coriandrum sativum | T | R | 48.8 |
| Cornus canadensis | T | O | 32.1 |
| Cornus canadensis | T | R | 80.2 |
| Crataegus sp | T | R | 22.9 |
| Crataegus submollis | T | O | 81.5 |
| Cryptotaenia canadensis | T | R | 20.9 |
| Cymbopogon citratus | T | R | 40.5 |
| Cymbopogon citratus | T | O | 77.0 |
| Cyperus esculentus | T | R | 20.9 |
| Cyperus esculentus | T | O | 72.0 |
| Dirca palustris | T | R | 67.1 |
| Dirca palustris | T | O | 82.2 |
| Dryopteris filix-mas | T | O | 23.9 |
| Echinacea purpurea | T | O | 92.2 |
| Eleusine coracana | T | R | 30.0 |
| Erysimum perofskianum | T | R | 81.7 |
| Erysimum perofskianum | T | O | 98.8 |
| Fagopyrum esculentum | T | O | 35.5 |
| Fagopyrum tararicum | T | O | 40.0 |
| Fagopyrum tataricum | T | R | 30.1 |
| Foeniculum vulgare | T | O | 21.0 |
| Fpomoea batatas | T | O | 98.6 |
| Fragaria x ananassa | T | O | 44.3 |
| Galinsoga ciliata | T | R | 49.4 |
| Galinsoga ciliata | T | O | 56.9 |
| Galium odoratum | T | R | 59.4 |
| Galium odoratum | T | O | 95.3 |
| Gaultheria hispidula | T | R | 37.9 |
| Gaultheria hispidula | T | O | 78.5 |
| Gaultheria procumbens | T | O | 85.7 |
| Glechoma hederacea | T | O | 95.9 |
| Glycine max | T | O | 96.8 |
| Glycine max | T | R | 32.8 |
| Glycine max | T | O | 100.0 |
| Glycyrrhiza glabra | T | R | 70.2 |
| Glycyrrhiza glabra | T | O | 90.3 |
| Guizotia abyssinica | T | R | 34.4 |
| Guizotia abyssinica | T | O | 97.9 |
| Hamamelis virginiana | T | R | 72.1 |
| Hamamelis virginiana | T | O | 77.1 |
| Hedeoma pulegioides | T | O | 34.7 |
| Helianthus strumosus | T | R | 20.6 |
| Helianthus strumosus | T | O | 57.2 |
| Helianthus tuberosa | T | O | 61.0 |
| Helianthus tuberosus | T | R | 46.9 |
| Helichrysum angustifolium | T | O | 23.5 |
| Helichrysum angustifolium | T | R | 94.5 |
| Helichrysum thianschanicum | T | R | 98.1 |
| Helleborus niger | T | O | 26.2 |
| Humulus lupulus | T | R | 38.0 |
| Humulus lupulus | T | O | 93.8 |
| Hyoscyamus niger | T | O | 41.5 |
| Hyssopus officinalis | T | R | 44.6 |
| Inula helenium | T | O | 97.6 |
| Juniperus communis | T | R | 80.0 |
| Koeleria glauca | T | O | 94.7 |
| Koeleria glauca | T | R | 99.4 |
| Lactuca sativa | T | O | 94.0 |
| Lathyrus Sativus | T | R | 24.0 |
| Lathyrus Sativus | T | O | 33.0 |
| Lathyrus sylvestris | T | O | 43.1 |
| Laurus nobilis | T | R | 51.7 |
| Laurus nobilis | T | O | 87.2 |
| Lavandula latifolia | T | R | 75.5 |
| Lavendula angustifolia | T | R | 81.9 |
| Ledum groenlandicum | T | R | 45.9 |
| Ledum groenlandicum | T | O | 99.5 |
| Lens culinaris subsp. Culinaris | T | R | 28.0 |
| Lens culinaris subsp. Culinaris | T | O | 97.6 |
| Levisticum officinale | T | R | 51.4 |
| Levisticum officinale | T | O | 87.8 |
| Lotus corniculatus | T | R | 53.7 |
| Lotus corniculatus | T | O | 97.4 |
| Lupinus polyphyllus | T | O | 95.8 |
| Lupinus polyphyllus | T | R | 99.3 |
| Luzula sylvatica | T | R | 29.5 |
| Malus hupehensis | T | R | 58.7 |
| Malus hupehensis | T | O | 62.5 |
| Malus spp. | T | O | 25.7 |
| Malva sylvestris | T | O | 73.5 |
| Medicago sativa | T | R | 46.2 |
| Medicago sativa | T | O | 94.9 |
| Melilotus officinalis | T | O | 99.4 |
| Melissa officinalis | T | R | 91.0 |
| Mentha piperita | T | O | 86.8 |
| Menyanthes trifoliata | T | O | 64.3 |
| Miscanthus sinensis Andress | T | R | 36.1 |
| Miscanthus sinensis Andress | T | O | 66.6 |
| Nepeta cataria | T | O | 23.6 |
| Ocimum Basilicum | T | O | 81.3 |
| Oenothera biennis | T | R | 35.7 |
| Oenothera biennis | T | O | 75.6 |
| Onobrychis viciifolia | T | R | 44.5 |
| Onobrychis viciifolia | T | O | 90.7 |
| Origanum vulgare | T | R | 76.5 |
| Origanum vulgare | T | O | 82.9 |
| Oryza sativa | T | O | 51.4 |
| Oxalis Deppei | T | R | 48.4 |
| Oxalis Deppei | T | O | 73.4 |
| oxyria digyna | T | R | 23.6 |
| oxyria digyna | T | O | 92.5 |
| Panax quinquefolius | T | O | 24.8 |
| Panax quinquefolius | T | R | 36.6 |
| Panicum miliaceum | T | R | 26.9 |
| Passiflora caerulea | T | R | 55.3 |
| Passiflora caerulea | T | O | 77.6 |
| Pastinaca sativa | T | O | 49.2 |
| Pastinaca sativa | T | O | 82.9 |
| Pennisetum alopecuroides | T | O | 74.9 |
| Perilla frutescens | T | R | 83.5 |
| Petasites Japonicus | T | R | 22.9 |
| Petasites Japonicus | T | O | 79.5 |
| Petroselinum crispum | T | O | 61.1 |
| Petroselinum crispum | T | O | 83.7 |
| Petroselinum crispum | T | R | 99.0 |
| Phalaris canariensis | T | R | 29.5 |
| Phalaris canariensis | T | O | 67.2 |
| Phaseolus vulgaris | T | O | 93.1 |
| Physalis pruinosa | T | O | 64.2 |
| Pimpinella anisum | T | R | 59.0 |
| Pimpinella anisum | T | O | 88.5 |
| Pisum sativum | T | O | 75.4 |
| Plantago major | T | O | 99.6 |
| Plectranthus sp. | T | R | 49.4 |
| Podophyllum peltatum | T | O | 87.3 |
| Polygonum aviculare | T | R | 32.8 |
| Polygonum aviculare | T | O | 53.9 |
| Potentilla anserina | T | O | 94.9 |
| Prunella vulgaris | T | O | 76.4 |
| Prunella vulgaris | T | R | 94.7 |
| Pteridium aquilinum | T | O | 90.1 |
| Raphanus raphanistrum | T | R | 39.5 |
| Raphanus raphanistrum | T | O | 91.0 |
| Raphanus sativus | T | O | 79.1 |
| Ribes nigrum | T | R | 89.6 |
| Ribes nigrum | T | O | 95.4 |
| Ribes Sylvestre | T | R | 20.1 |
| Ribes Sylvestre | T | O | 97.4 |
| Ricinus communis | T | R | 26.5 |
| Ricinus communis | T | O | 92.4 |
| Rosa rugosa | T | O | 41.6 |
| Rubus canadensis | T | O | 96.4 |
| Rubus idaeus | T | R | 44.8 |
| Rubus idaeus | T | O | 88.7 |
| Rumes scutatus | T | O | 88.7 |
| Rumex acetosella | T | R | 40.9 |
| Rumex acetosella | T | O | 90.9 |
| Rumex crispus | T | R | 33.4 |
| Rumex crispus | T | O | 89.3 |
| Ruta graveolens | T | O | 68.5 |
| Salix purpurea | T | R | 37.1 |
| Salix purpurea | T | O | 46.1 |
| Salvia officinalis | T | O | 67.7 |
| Salvia officinalis | T | R | 91.1 |
| Sambucus canadensis | T | R | 35.7 |
| Sambucus canadensis | T | O | 99.0 |
| Sanguisorba minor | T | O | 90.6 |
| Santolina | T | O | 62.7 |
| Santolina | T | R | 73.4 |
| Saponaria officinalis | T | O | 93.2 |
| Satureja hortensis | T | R | 43.1 |
| Satureja hortensis | T | O | 87.9 |
| Satureja montana | T | R | 55.1 |
| Satureja montana | T | O | 79.2 |
| Satureja repandra | T | R | 49.7 |
| Satureja repandra | T | O | 73.3 |
| Scorzorera hipanica | T | O | 63.3 |
| Scuttellaria lateriflora | T | O | 29.3 |
| Setaria italica | T | R | 20.8 |
| Silene vulgaris | T | O | 96.8 |
| Sium sisarum | T | R | 27.4 |
| Sium sisarum | T | O | 88.8 |
| Solanum melongens | T | R | 21.9 |
| Solidago sp | T | R | 45.9 |
| Solidago sp | T | O | 74.0 |
| Sonchus oleraceus | T | R | 22.7 |
| Sonchus oleraceus | T | O | 38.1 |
| Sorghum caffrorum | T | O | 57.0 |
| Sorghum caffrorum | T | R | 74.0 |
| Sorghum dochna | T | O | 44.3 |
| Sorghum dochna | T | O | 65.8 |
| Sorghum dochna | T | R | 70.7 |
| Sorghum dochna | T | R | 89.0 |
| Sorghum durra | T | R | 39.6 |
| Sorghum durra | T | O | 76.5 |
| Sorghum sudanense | T | O | 40.5 |
| Stachys affinis | T | R | 67.2 |
| Stachys affinis | T | O | 86.6 |
| Stachys byzantina | T | R | 85.7 |
| Stellaria graminea | T | O | 43.3 |
| Stellaria graminea linne | T | R | 39.2 |
| Stellaria media | T | R | 21.1 |
| Stipa capillata | T | R | 24.2 |
| Symphytum officinale | T | R | 64.4 |
| Tanacetum parthenium | T | R | 62.2 |
| Tanacetum vulgare | T | R | 42.5 |
| Tanacetum vulgare | T | O | 97.5 |
| Taraxacum officinale | T | R | 47.5 |
| Taraxacum officinale | T | O | 100.0 |
| Teucrium chamaedrys | T | R | 40.0 |
| Thymus fragantissimus | T | O | 93.7 |
| Thymus fragantissimus | T | R | 97.3 |
| Thymus praecox subsp arcticus | T | O | 46.0 |
| Thymus pseudolanuginosus | T | R | 74.3 |
| Thymus serpyllum | T | O | 88.6 |
| Thymus x citriodorus | T | R | 66.4 |
| Thymus x citriodorus | T | O | 97.8 |
| Tiarella cordifolia | T | O | 94.9 |
| Tragopogon porrifolius | T | R | 45.0 |
| Tragopogon porrifolius | T | O | 72.0 |
| Triticosecale spp | T | R | 27.8 |
| Triticosecale spp | T | O | 87.8 |
| Triticum aestivum | T | R | 26.6 |
| Triticum aestivum | T | O | 42.6 |
| Tropaeolum majus | T | R | 21.4 |
| Tropaeolum majus | T | O | 81.5 |
| Typha latifolia | T | O | 44.8 |
| Typha latifolia | T | R | 72.5 |
| Urtica dioica | T | R | 35.2 |
| Urtica dioica | T | O | 62.9 |
| Vaccinium angustifolium | T | R | 27.4 |
| Vaccinium macrocarpon | T | R | 78.0 |
| Vaccinium macrocarpon | T | O | 87.8 |
| Veratrum viride | T | O | 90.2 |
| Verbascum thapsus | T | O | 84.3 |
| Viburnum trilobum | T | R | 45.2 |
| Viburnum trilobum | T | O | 70.0 |
| Vicia sativa | T | O | 99.0 |
| Vicia villosa | T | R | 44.2 |
| Vicia villosa | T | O | 98.3 |
| Vinca minor | T | O | 21.5 |
| Vitis sp. | T | O | 99.9 |
| Zea mays | T | R | 31.7 |
| Zea mays | T | O | 90.2 |

TABLE 10
HLE

| Nom latin | Stress | Extrait | Inhibition |
|---|---|---|---|
| Achillea millefolium | A | O | 21.9 |
| Achillea millefolium | A | S | 24.5 |
| Aconitum napellus | A | O | 25.8 |
| Adiantum pedatum | A | R | 27.6 |
| Agrimonia eupatoria | A | V | 26.0 |
| Agropyron cristatum | A | R | 21.0 |
| Agropyron repens | A | S | 23.4 |
| Agropyron repens | A | R | 28.2 |
| Agropyron repens | A | S | 39.8 |
| Agrostis Stofonifera | A | O | 38.9 |
| Alchemilla mollis | A | V | 27.9 |
| Alchemilla mollis | A | O | 66.0 |
| Alchemilla mollis | A | R | 100.0 |
| Alchemilla mollis | A | S | 23.5 |
| Alkanna tinctoria | A | S | 26.2 |
| Allium Tuberosum | A | S | 57.9 |
| Aloe vera | A | O | 20.5 |
| Ambrosia artemisiifolia | A | O | 29.1 |
| Amelanchier sanguinea | A | W | 96.5 |
| Amelanchier sanguinea | A | V | 52.4 |
| Anethum graveolens | A | O | 32.1 |
| Anethum graveolens | A | W | 22.8 |
| Angelica archangelica | A | S | 39.2 |
| Anthemis nobilis | A | O | 37.6 |
| Anthemis nobilis | A | S | 26.4 |
| Anthemis tinctoria | A | O | 31.9 |
| Anthemis tinctoria | A | S | 38.4 |
| Apium graveolens | A | S | 49.2 |
| Arctium minus | A | O | 46.4 |
| Arclostaphylos uva-ursi | A | R | 100.0 |
| Aronia melanocarpa | A | O | 21.9 |
| Aronia melanocarpa | A | W | 78.4 |
| Aronia melanocarpa | A | V | 100.0 |
| Aronia melanocarpa | A | R | 29.0 |
| Aronia melanocarpa | A | O | 33.6 |
| Artemisia dracunculus | A | W | 89.2 |
| Aster sp | A | R | 26.2 |
| Beta vulgaris | A | R | 100.0 |
| Beta vulgaris ssp. Maritima | A | R | 92.2 |
| Borago officinalis | A | S | 22.6 |
| Brassica napus | A | S | 68.3 |
| Brassica napus | A | R | 29.5 |
| Brassica nigra | A | S | 32.6 |
| Brassica oleracea | A | O | 22.9 |
| Brassica oleracea | A | V | 20.8 |
| Brassica oleracea | A | R | 22.2 |
| Brassica rapa | A | S | 23.2 |
| Brassica rapa | A | R | 26.9 |
| Bromus inermis | A | O | 34.1 |
| Bromus inermis | A | R | 21.9 |
| Calamintha nepeta | A | O | 35.4 |
| Canna edulis | A | O | 56.4 |
| Canna edulis | A | R | 21.4 |
| Carum carvi | A | O | 24.2 |
| Chaerophyllum bulbosum | A | O | 25.5 |
| Chenopodium bonus-henricus | A | R | 24.0 |
| Chenopodium bonus-henricus | A | S | 85.8 |
| Chenopodium quinoa | A | S | 50.4 |
| Chrysanthemum coronarium | A | O | 26.0 |
| Cicer arietinum | A | S | 23.3 |
| Cichorium intybus | A | S | 32.1 |
| Citrullus lanatus | A | R | 26.3 |
| Coix Lacryma-Jobi | A | S | 66.1 |
| Cosmos sulphureus | A | O | 38.8 |
| Cosmos sulphureus | A | S | 20.7 |
| Crataegus sp | A | O | 84.1 |
| Crataegus sp | A | R | 23.6 |
| Crataegus sp | A | S | 21.7 |
| Crataegus submollis | A | S | 34.0 |
| Cryptotaenia canadensis | A | V | 22.1 |
| Cucumis anguria | A | O | 26.2 |
| Cucumis Anguria | A | R | 53.4 |
| Cucumis melo | A | S | 53.6 |
| Cucumis sativus | A | R | 53.3 |
| Curcuma zedoaria | A | O | 24.3 |
| Cymbopogon citratus | A | S | 91.2 |
| Datisca cannabina | A | S | 55.7 |
| Daucus carota | A | R | 100.0 |
| Daucus carota | A | V | 24.7 |
| Daucus carota | A | O | 37.9 |
| Digitalis purpurea | A | S | 34.0 |
| Dirca palustris | A | R | 20.3 |
| Dirca palustris | A | S | 27.9 |
| Dolichos Lablab | A | R | 21.5 |
| Dryopteris filix-mas | A | R | 58.8 |
| Dryopteris filix-mas | A | S | 22.0 |
| Echinacea purpurea | A | O | 38.2 |
| Echinacea purpurea | A | S | 28.1 |
| Eleusine coracana | A | S | 20.7 |
| Erigeron canadensis | A | O | 29.6 |
| Fagopyrum esculentum | A | S | 29.3 |
| Fagopyrum tataricum | A | S | 24.4 |
| Foeniculum vulgare | A | O | 25.1 |
| Fragaria x ananassa | A | O | 22.3 |
| Fragaria x ananassa | A | W | 100.0 |
| Fragaria x ananassa | A | V | 21.4 |
| Fragaria x ananassa | A | S | 29.4 |
| Fragaria x ananassa | A | V | 21.6 |
| Galinsoga ciliata | A | R | 61.6 |
| Galium odoratum | A | R | 21.0 |
| Gaultheria hispidula | A | O | 33.7 |
| Gentiana lutea | A | R | 52.1 |
| Glechoma hederacea | A | O | 21.8 |
| Glycine Max | A | S | 81.3 |
| Glycyrrhiza glabra | A | W | 100.0 |
| Glycyrrhiza glabra | A | S | 63.3 |
| Guizotia abyssinica | A | R | 36.9 |
| Hamamelis virginiana | A | R | 100.0 |
| Helianthus Tuberosus | A | S | 32.1 |
| Heliotropium arborescens | A | R | 22.8 |
| Heliotropium arborescens | A | S | 24.9 |
| Helleborus niger | A | S | 25.6 |
| Hordeum vulgare | A | O | 58.1 |
| Hypericum perforatum | A | S | 24.8 |
| Hyssopus officinalis | A | O | 21.1 |
| Hyssopus officinalis | A | S | 93.6 |
| Lactuca serriola | A | S | 34.3 |
| Laurus nobilis | A | W | 100.0 |
| Lavandula latifolia | A | W | 57.1 |
| Lavandula latifolia | A | O | 43.7 |
| Lavandula latifolia | A | S | 42.2 |
| Leonurus cardiaca | A | R | 100.0 |
| Lepidium sativum | A | O | 100.0 |
| Lolium multiflorum | A | O | 31.0 |
| Lolium perenne | A | O | 20.8 |
| Lolium perenne | A | R | 21.7 |
| Lolium perenne | A | S | 22.1 |
| Ludoviciana | A | O | 33.4 |
| Ludoviciana | A | S | 20.7 |
| Malva sylvestris | A | S | 22.9 |
| Matricaria recutita | A | O | 28.5 |
| Melaleuca alternifolia | A | O | 21.9 |
| Melissa officinalis | A | S | 23.4 |
| Mentha piperita | A | O | 31.6 |
| Mentha piperita | A | W | 33.2 |
| Mentha pulegium | A | O | 42.2 |
| Mentha pulegium | A | V | 21.5 |
| Mentha pulegium | A | S | 33.8 |
| Mentha spicata | A | O | 24.3 |
| Oenothera biennis | A | O | 25.2 |
| Oenothera biennis | A | R | 78.8 |
| Origanum majorana | A | V | 37.4 |
| Oxyria digyna | A | V | 28.2 |
| Panicum miliaceum | A | O | 33.3 |
| Peucedanum cervaria | A | R | 23.4 |
| Phalaris arundinacea | A | R | 22.4 |
| Phalaris canariensis | A | O | 27.8 |
| Phaseolus coccineus | A | S | 28.3 |
| Phaseolus mungo | A | R | 37.8 |
| Phaseolus vulgaris | A | O | 24.3 |
| Phaseolus Vulgaris | A | S | 74.3 |
| Phleum pratense | A | R | 27.8 |
| Physalis ixocarpa | A | O | 21.5 |
| Physalis Ixocarpa | A | S | 26.5 |
| Physalis Pruinosa | A | S | 60.2 |
| Phytolacca americana | A | S | 100.0 |
| Plantago coronopus | A | O | 21.1 |
| Plantago coronopus | A | S | 25.7 |
| Plantago major | A | O | 26.0 |
| Plectranthus sp. | A | O | 23.1 |
| Poa pratensis | A | O | 21.7 |
| Polygonum aviculare | A | R | 79.7 |
| Portulaca olevcae | A | O | 34.5 |
| Poterium sanguisorba | A | R | 25.8 |
| Poterium sanguisorba | A | O | 34.6 |
| Poterium sanguisorba | A | W | 31.0 |
| Pteridium aquilinum | A | R | 54.4 |
| Raphanus sativus | A | S | 66.4 |
| Raphanus sativus | A | R | 81.8 |
| Rheum officinale | A | S | 37.9 |
| Ribes nigrum | A | W | 100.0 |
| Ribes nigrum | A | S | 47.6 |
| Ribes nigrum | A | V | 27.5 |
| Ribes rubrum | A | R | 35.4 |
| Ribes Sylvestre | A | W | 100.0 |
| Rosa rugosa | A | W | 95.1 |
| Rosa rugosa | A | R | 24.6 |
| Rosmarinus officinalis | A | R | 58.4 |
| Rubus idaeus | A | W | 27.6 |
| Rubus idaeus | A | S | 33.0 |
| Rubus idaeus | A | R | 27.9 |
| Rubus idaeus | A | O | 37.4 |
| Rumex Acetosa | A | S | 45.2 |
| Rumex crispus | A | O | 26.1 |
| Rumex crispus | A | R | 100.0 |
| Rumex Scutatus | A | V | 43.8 |
| Ruta graveolens | A | O | 28.7 |
| Saccharum officinarum | A | O | 29.6 |
| Saccharum officinarum | A | R | 23.8 |
| Salvia elegans | A | O | 100.0 |
| Salvia officinalis | A | O | 95.7 |
| Salvia officinalis | A | W | 77.9 |
| Salvia officinalis | A | R | 83.7 |
| Salvia officinalis | A | S | 20.5 |
| Salvia sclarea | A | O | 100.0 |
| Salvia sclarea | A | V | 28.6 |
| Santolina chamaecyparissus | A | O | 27.1 |
| Satureja montana | A | W | 23.2 |
| Satureja montana | A | S | 27.7 |
| Scorzonera hispanica | A | R | 60.1 |
| Scutellaria lateriflora | A | S | 45.9 |
| Senecio vulgaris | A | R | 34.0 |
| Sonchus oleraceus | A | O | 29.1 |
| Sorghum dochna | A | O | 21.1 |
| Sorghum dochna | A | V | 24.4 |
| Sorghum durra | A | O | 23.4 |
| Sorghum durra | A | V | 23.6 |
| Spinacia oleracea | A | S | 26.8 |
| Stellaria graminea | A | O | 24.8 |
| Symphytum officinale | A | O | 91.6 |
| Tanacetum cinerariifolium | A | R | 28.3 |
| Tanacetum vulgare | A | O | 46.3 |
| Tanacetum vulgare | A | S | 33.7 |
| Taraxacum officinale | A | W | 26.4 |
| Taraxacum officinale | A | V | 24.0 |
| Taraxacum officinale | A | O | 21.0 |
| Teucrium chamaedrys | A | O | 37.0 |
| Thymus fragantissimus | A | W | 20.2 |
| Thymus herba-barona | A | W | 20.8 |
| Thymus vulgaris | A | R | 77.9 |
| Thymus vulgaris | A | W | 23.6 |
| Thymus x citriodorus | A | W | 21.3 |
| Thymus x citriodorus | A | S | 21.1 |
| Trichosanthes kirilowii | A | O | 23.2 |
| Trigonella foenum graecum | A | S | 32.0 |
| Triticum durum | A | S | 22.0 |
| Triticum turgidum | A | O | 60.0 |
| Triticum spelta | A | S | 47.6 |
| Urtica dioica | A | O | 33.3 |
| Vaccinium augustifolium | A | W | 42.6 |
| Vaccinium Corymbosum | A | W | 22.4 |
| Vaccinium Corymbosum | A | S | 21.6 |
| Vaccinium macrocarpon | A | W | 22.5 |
| Vaccinium macrocarpon | A | S | 54.8 |
| Valerianella locusta | A | O | 49.2 |
| Veronica officinalis | A | O | 43.7 |
| Viburnum trilobum Marsh. | A | W | 75.4 |
| Vitis | A | S | 33.8 |
| Vitis | A | W | 100.0 |
| Vitis | A | O | 21.0 |
| Zea Mays | A | S | 95.2 |
| Achillea millefolium | G | O | 28.8 |
| Achillea millefolium | G | S | 27.3 |
| Aconitum napellus | G | O | 23.1 |
| Aconitum napellus | G | R | 97.7 |
| Acorus calamus | G | S | 20.0 |
| Adiantum pedatum | G | R | 100.0 |
| Agastache foeniculum | G | W | 25.3 |
| Ageratum conyzoides | G | O | 28.5 |
| Agropyron cristatum | G | R | 37.3 |
| Agropyron repens | G | R | 31.4 |
| Alchemilla mollis | G | W | 20.6 |
| Alchemilla mollis | G | O | 56.1 |
| Alchemilla mollis | G | R | 28.1 |
| Alchemilla mollis | G | S | 25.3 |
| Allium cepa | G | O | 20.2 |
| Allium sativum | G | O | 100.0 |
| Allium tuberosum | G | O | 100.0 |
| Althaea officinalis | G | S | 30.8 |
| Amaranthus caudatus | G | S | 22.3 |
| Amelanchier sanguinea | G | W | 88.3 |
| Anethum graveolens | G | O | 26.2 |
| Angelica archangelica | G | S | 43.2 |
| Anthemis nobilis | G | S | 21.7 |
| Arctostaphylos uva-ursi | G | O | 33.1 |
| Arctostaphylos uva-ursi | G | R | 100.0 |
| Arctostaphylos uva-ursi | G | S | 23.4 |
| Armoracia rusticana | G | O | 22.5 |
| Aronia melanocarpa | G | W | 79.0 |
| Aronia melanocarpa | G | V | 100.0 |
| Aronia melanocarpa | G | S | 22.7 |
| Aronia melanocarpa | G | O | 29.6 |
| Artemisia absinthium | G | O | 31.5 |
| Artemisia absinthium | G | V | 24.2 |
| Aster | G | S | 29.2 |
| Beckmannia eruciformis | G | O | 22.7 |
| Beta vulgaris | G | R | 100.0 |
| Betula glandulosa | G | S | 26.7 |
| Borago officinalis | G | O | 25.7 |
| Brassica Napus | G | S | 50.4 |
| Brassica napus | G | R | 48.2 |
| Brassica nigra | G | S | 23.9 |
| Brassica oleracea | G | R | 28.1 |
| Brassica oleracea | G | S | 22.5 |
| Brassica rapa | G | R | 56.4 |
| Calamintha nepeta | G | V | 24.8 |
| Calamintha nepeta | G | O | 38.8 |
| Canna edulis | G | O | 66.3 |
| Capsella bursa-pastoris | G | R | 25.8 |
| Carthamus tinctorius | G | R | 22.2 |
| Chelidonium majus | G | O | 31.6 |
| Chenopodium album | G | S | 21.3 |
| Cichorium endivia subsp. Endivia | G | S | 21.4 |
| Cicer arietinum | G | S | 50.7 |
| Cichorium endivia subsp. Endivia | G | O | 48.5 |
| Cichorium endivia subsp. Endivia | G | S | 27.9 |
| Coix Lacryma-Jobi | G | O | 24.5 |
| Cornus canadensis | G | S | 36.1 |
| Crataegus sp | G | W | 57.8 |
| Cucurbita Pepo | G | R | 23.1 |
| Curcuma zedoaria | G | O | 24.0 |
| Datura metel | G | O | 21.0 |
| Daucus carota | G | O | 32.3 |
| Daucus carrota | G | R | 90.9 |
| Dipsacus sativus | G | O | 32.7 |
| Dirca palustris | G | S | 33.5 |
| Dolichos Lablab | G | R | 32.1 |
| Dryopteris filix-mas | G | R | 80.9 |
| Echinacea purpurea | G | S | 63.0 |
| Elymus junceus | G | R | 25.9 |
| Erigeron canadensis | G | O | 43.0 |
| Erigeron speciosus | G | O | 22.8 |
| Erigeron speciosus | G | S | 24.2 |
| Erysimum perofskianum | G | O | 20.8 |
| Fagopyrum esculentum | G | S | 32.9 |
| Fagopyrum tataricum | G | S | 41.2 |
| Foeniculum vulgare | G | V | 25.7 |
| Foeniculum vulgare | G | S | 42.5 |
| Foeniculum Vulgare | G | O | 24.1 |
| Galinsoga ciliata | G | S | 25.0 |
| Galium odoratum | G | R | 89.4 |
| Gaultheria hispidula | G | O | 35.1 |
| Gaultheria hispidula | G | R | 67.2 |
| Gaultheria procumbens | G | S | 74.7 |
| Glycine max | G | R | 24.6 |
| Glycyrrhiza glabra | G | W | 56.8 |
| Glycyrrhiza glabra | G | V | 30.0 |
| Glycyrrhiza glabra | G | R | 92.4 |
| Glycyrrhiza glabra | G | S | 28.6 |
| Hamamelis virginiana | G | R | 100.0 |
| Hamamelis virginiana | G | S | 29.3 |
| Hedeoma pulegioides | G | O | 60.0 |
| Helenium hoopesii | G | O | 37.3 |
| Helenium hoopesii | G | S | 34.7 |
| Helianthus tuberosus | G | V | 21.4 |
| Helichrysum thianschanicum | G | O | 43.0 |
| Helichrysum thianschanicum | G | R | 39.2 |
| Heliotropium arborescens | G | R | 22.8 |
| Heliotropium arborescens | G | S | 39.5 |
| Helleborus niger | G | S | 34.2 |
| Hordeum vulgare subsp. Vulgare | G | O | 33.4 |
| Hypericum henryi | G | S | 23.7 |
| Hypericum perforatum | G | S | 23.8 |
| Hyssopus officinalis | G | W | 45.1 |
| Hyssopus officinalis | G | S | 24.2 |
| Inula helenium | G | W | 96.2 |
| Ipomola batatas | G | V | 21.9 |
| Lactuca sativa | G | W | 35.1 |
| Laportea canadensis | G | O | 25.1 |
| Laportea canadensis | G | S | 26.5 |
| Laserpitium latifolium | G | S | 22.1 |
| Lathyrus sativus | G | O | 29.9 |
| Lathyrus sativus | G | W | 27.8 |
| Lathyrus sativus | G | S | 28.1 |
| Laurus nobilis | G | W | 100.0 |
| Lavandula angustifolia | G | O | 65.7 |
| Ledum groenlandicum | G | O | 100.0 |
| Leonorus cardiaca | G | R | 61.3 |
| Lepidium sativum | G | O | 100.0 |
| Levisticum officinale | G | W | 91.4 |
| Lolium perenne | G | O | 37.3 |
| Lotus tetragonolobus | G | S | 21.8 |
| Lupinus polyphylius | G | O | 42.3 |
| Malus hupehensis | G | S | 25.9 |
| Medicago sativa | G | S | 32.1 |
| Melaleuca alternifolia | G | O | 40.0 |
| Melissa officinalis | G | S | 23.1 |
| Mentha arvensis | G | S | 65.5 |
| Mentha piperita | G | O | 24.2 |
| Mentha piperita | G | S | 23.7 |
| Mentha piperita | G | V | 34.2 |
| Mentha pulegium | G | O | 63.3 |
| Mentha pulegium | G | V | 30.2 |
| Mentha spicata | G | S | 45.9 |
| Monarda didyma | G | S | 47.7 |
| Nepeta cataria | G | R | 100.0 |
| Nicotiana tabacum | G | O | 75.8 |
| Ocimum basilicum | G | O | 40.1 |
| Ocimum basilicum | G | S | 27.9 |
| Oenothera biennis | G | O | 26.3 |
| Oenothera biennis | G | R | 100.0 |
| Oenothera biennis | G | O | 49.6 |
| Oenothera biennis | G | S | 54.0 |
| Origanum vulgare | G | W | 100.0 |
| Origanum vulgare | G | O | 26.7 |
| Origanum vulgare | G | S | 21.3 |
| Oryza Sativa | G | S | 34.5 |
| Oxalis Deppei Lodd. | G | O | 27.4 |
| Panicum miliaceum | G | O | 25.3 |
| Pastinaca sativa | G | R | 95.0 |
| Petroselinum crispum | G | R | 44.5 |
| Petroselinum crispum | G | S | 26.5 |
| Peucedanum cervaria | G | R | 25.1 |
| Phaseolus coccineus | G | R | 30.9 |
| Phaseolus coccineus | G | O | 27.5 |
| Phaseolus mungo | G | R | 24.3 |
| Phlox paniculata | G | S | 37.9 |
| Physalis pruinosa | G | S | 26.5 |
| Phytolacca americana | G | S | 100.0 |
| Pimpinella anisum | G | S | 23.7 |
| Plantago coronopus | G | O | 25.1 |
| Plantago major | G | O | 25.0 |
| Plantago major | G | R | 20.5 |
| Plantago major | G | S | 23.6 |
| Poa compressa | G | O | 28.5 |
| Poa pratensis | G | O | 37.5 |
| Polygonum aviculare | G | R | 25.4 |
| Polygonum pensylvanicum | G | O | 21.3 |
| Portulaca oleracea | G | O | 28.0 |
| Poterium sanguisorba | G | O | 25.6 |
| Poterium sanguisorba | G | V | 21.9 |
| Prunella vulgaris | G | O | 23.4 |
| Pteridium aquilinum | G | R | 43.1 |
| Reseda odorata | G | O | 46.5 |
| Rhaphanus sativus | G | S | 32.6 |
| Rheum x cultorum | G | S | 20.9 |
| Ribes nidigrolaria | G | W | 29.8 |
| Ribes nidigrolaria | G | V | 53.7 |
| Ribes nigrum | G | V | 20.3 |
| Ribes Silvestre | G | W | 91.6 |
| Ricinus communis | G | S | 46.0 |
| Rosmarinus officinalis | G | R | 60.4 |
| Rubus idaeus | G | W | 28.2 |
| Rubus occidentalis | G | R | 93.6 |
| Rubus occidentalis | G | O | 40.0 |
| Rumex acetosella | G | V | 24.3 |
| Rumex crispus | G | R | 100.0 |
| Rumex patientia | G | O | 32.0 |
| Rumex scutatus | G | V | 28.6 |
| Ruta graveolens | G | S | 23.4 |
| Saccharum officinarum | G | O | 30.2 |
| Salix purpurea | G | S | 24.8 |
| Salvia elegans | G | O | 100.0 |
| Salvia officinalis | G | W | 52.4 |
| Salvia officinalis | G | R | 100.0 |
| Salvia officinalis | G | O | 100.0 |
| Salvia sclarea | G | O | 100.0 |
| Salvia sclarea | G | V | 23.0 |
| Salvia sclarea | G | W | 31.1 |
| Sambucus ebulus | G | O | 52.1 |
| Sambucus ebulus | G | R | 48.6 |
| Sanguisorba officinalis | G | R | 100.0 |
| Santolina chamaecyparissus | G | O | 100.0 |
| Serratula tinctoria | G | S | 56.8 |
| Satureja montana | G | O | 34.1 |
| Scolymus hispanicus | G | R | 37.9 |
| Scutellaria lateriflora | G | S | 54.7 |
| Senecio vulgaris | G | R | 35.3 |
| Solidago sp | G | S | 22.6 |
| Sonchus oleraceus | G | O | 23.7 |
| Sorghum caffrorum | G | V | 27.1 |
| Sorghum dochna | G | S | 40.7 |
| Sorghum dochna | G | O | 21.4 |
| Sorghum sudanense | G | V | 23.3 |
| Sorghum sudanense | G | W | 92.9 |
| Stellaria graminea | G | O | 25.4 |
| Stellaria media | G | O | 30.4 |
| Stellaria media | G | R | 22.0 |
| Tanacetum vulgare | G | O | 57.3 |
| Tanacetum vulgare | G | S | 38.4 |
| Tanacetum vulgare | G | O | 38.2 |
| Tanacetum vulgare | G | W | 26.3 |
| Taraxacum officinale | G | V | 20.0 |
| taraxacum officinale | G | O | 28.0 |
| Thymus fragantissimus | G | R | 79.9 |
| Thymus fragantissimus | G | O | 26.2 |
| Thymus herba-barona | G | W | 20.2 |
| Thymus serpyllum | G | V | 22.2 |
| Triticosecale spp. | G | S | 29.7 |
| Triticum durum | G | S | 37.8 |
| Triticum spelta | G | O | 31.0 |
| Triticum spelta | G | S | 37.9 |
| Typha latifolia | G | S | 27.5 |
| Urtica diolca | G | O | 60.3 |
| Vaccinium corymbosum | G | S | 33.2 |
| Vaccinium angustifolium | G | S | 43.7 |
| Vaccinium macrocarpon | G | W | 57.8 |
| Vaccinium macrocarpon | G | S | 59.9 |
| Valerianella locusta | G | O | 32.1 |
| Veratrum viride | G | O | 22.1 |
| Verbascum thapsus | G | S | 33.8 |
| Viburnum trilobum | G | V | 21.3 |
| Viburnum trilobum | G | W | 73.0 |
| Vicia faba | G | S | 21.2 |
| Vigna unguiculata | G | R | 20.1 |
| Vitis | G | V | 26.0 |
| Vitis | G | W | 66.1 |
| Vitis | G | O | 41.7 |
| Vitis | G | S | 30.7 |
| Xanthium sibiricum | G | O | 22.1 |
| Zea mays | G | S | 20.3 |
| Abies lasiocarpa | T | S | 22.4 |
| Achillea millefolium | T | S | 21.1 |
| Aconitum napellus | T | O | 100.0 |
| Acorus calamus | T | S | 21.0 |
| Agaricus bisporatus | T | S | 25.8 |
| Ageratum conyzoides | T | O | 20.1 |
| Agrimonia eupatoria | T | W | 59.6 |
| Agropyron cristatum | T | R | 53.4 |
| Agropyron repens | T | S | 22.6 |
| Agrostis alba | T | O | 25.3 |
| Alchemilla mollis | T | W | 88.7 |
| Alchemilla mollis | T | O | 42.6 |
| Alchemilla mollis | T | R | 70.4 |
| Alchemilla mollis | T | S | 31.2 |
| Allium ascalonicum | T | S | 42.9 |
| Allium sativum | T | O | 100.0 |
| Allium tuberosum | T | O | 100.0 |
| Alpinia officinarum | T | O | 21.9 |
| Alpinia officinarum | T | S | 100.0 |
| Amaranthus candatus | T | S | 36.0 |
| Amaranthus gangeticus | T | S | 66.8 |
| Ananas comosus | T | O | 20.3 |
| Ananas comosus | T | W | 23.8 |
| Anethum graveolens | T | O | 35.8 |
| angelica archangelica | T | R | 53.5 |
| Anthemis nobilis | T | O | 45.3 |
| Anthemis tinctorium | T | S | 47.5 |
| Anthriscus cerefolium | T | O | 20.5 |
| Arctium minus | T | O | 54.1 |
| Arctostaphylos uva-ursi | T | O | 28.1 |
| Arctostaphylos uva-ursi | T | R | 100.0 |
| Aronia melanocarpa | T | V | 100.0 |
| Aronia melanocarpa | T | W | 42.7 |
| Aronia prunifolia | T | W | 39.0 |
| Artemisia absinthium | T | O | 25.6 |
| Artemisia dracunulus | T | O | 31.3 |
| Artemisia dracunulus | T | S | 22.3 |
| Aster | T | S | 20.9 |
| Avena sativa | T | S | 100.0 |
| Averrhoa carambola | T | O | 25.8 |
| Beta vulgaris | T | R | 100.0 |
| Beta vulgaris | T | O | 59.3 |
| Beta vulgaris | T | S | 41.4 |
| Betula glandulosa | T | S | 61.8 |
| Boesenbergia rotunda | T | O | 36.9 |
| Boesenbergia rotunda | T | S | 42.5 |
| Boletus edulis | T | S | 43.1 |
| Borago officinalis | T | S | 36.3 |
| Brassica hirta | T | S | 30.2 |
| Brassica juncea | T | R | 41.4 |
| Brassica Napus | T | S | 29.9 |
| Brassica napus | T | R | 22.9 |
| Brassica oleracea | T | R | 25.6 |
| Brassica oleracea | T | V | 27.0 |
| Brassica oleracea | T | R | 26.5 |
| Brassica rapa | T | R | 24.8 |
| Bromus inermis | T | O | 27.8 |
| Canna edulis | T | O | 40.3 |
| Capsicum annuum | T | S | 22.6 |
| Carex morrowii | T | O | 26.0 |
| Carex morrowii | T | R | 49.8 |
| Carya cordiformis | T | S | 28.8 |
| Carya cordiformis | T | O | 21.0 |
| Carya cordiformis | T | W | 88.7 |
| Clematis armandii | T | O | 20.1 |
| Chaerophyllum bulbosum | T | O | 22.8 |
| Chaerophyllum bulbosum | T | S | 24.3 |
| Agaricus bisporatus | T | S | 25.4 |
| Chelidonium majus | T | O | 39.0 |
| Chenopodium bonus-henricus | T | S | 44.3 |
| chrysanthemum coronarium | T | O | 33.4 |
| chrysanthemum coronarium | T | S | 23.9 |
| Cichorium endivia subs. Endivia | T | O | 44.3 |
| Cichorium endivia subs. Endivia | T | S | 20.5 |
| Circium arvense | T | R | 49.7 |
| Citrullus colocynthis | T | R | 37.0 |
| Citrullus colocynthis | T | S | 35.5 |
| Citrus limettoides | T | O | 47.1 |
| Citrus limon | T | S | 26.2 |
| Citrus limon | T | O | 73.9 |
| Citrus reticulata | T | V | 32.7 |
| Citrus reticulata | T | S | 29.4 |
| Citrus sinensis | T | V | 25.2 |
| Coix Lacryma-Jobi | T | O | 32.7 |
| Coix Lacryma-Jobi | T | S | 31.4 |
| Corchorus olitorius | T | O | 24.4 |
| Cornus canadensis | T | S | 41.3 |
| Crataegus sp | T | S | 34.0 |
| Crataegus submollis | T | S | 39.6 |
| Curcuma longa | T | O | 55.3 |
| Curcuma zedoaria | T | O | 24.4 |
| Cydonia oblonga | T | V | 35.2 |
| Cynara scolymus | T | O | 41.2 |
| Cynara scolymus | T | R | 36.8 |
| Dactilis Glomerata | T | O | 31.9 |
| Datura metel | T | O | 36.9 |
| Datura metel | T | S | 21.4 |
| Datura stramonium | T | S | 25.9 |
| Daucus carota | T | R | 92.3 |
| Daucus carota | T | O | 31.0 |
| Dipsacus sativus | T | O | 100.0 |
| Dirca palustris | T | S | 31.4 |
| Dolichos lablab | T | O | 23.1 |
| Dryopteris filix-mas | T | R | 68.2 |
| Echinacea purpurea | T | S | 38.2 |
| Eleusine coracana | T | O | 22.1 |
| Elymus junceus | T | R | 37.9 |
| Erigeron speciosus | T | O | 35.0 |
| Erysimum perofskianum | T | O | 22.6 |
| Erysimum perofskianum | T | S | 23.2 |
| Fagopyrum esculentum | T | S | 24.7 |
| Foeniculum vulgare | T | O | 31.4 |
| Foeniculum vulgare | T | V | 69.1 |
| Foeniculum vulgare | T | S | 38.5 |
| Fragaria x ananassa | T | O | 50.4 |
| Fragaria x ananassa | T | V | 30.2 |
| Fragaria x ananassa | T | S | 28.4 |
| Frangula alnus | T | R | 65.3 |
| Frangula alnus | T | S | 40.7 |
| Fucus vesiculosus | T | O | 42.7 |
| Galinsoga ciliata | T | R | 49.3 |
| Gaultheria hispidula | T | W | 36.9 |
| Gentiana macrophylla | T | S | 26.1 |
| Ginkgo biloba | T | V | 27.1 |
| Glycyrrhiza glabra | T | W | 58.1 |
| Glycyrrhiza glabra | T | S | 50.4 |
| Glycyrrhiza glabra | T | R | 25.1 |
| Gossypium herbaceum | T | O | 22.7 |
| Gossypium herbaceum | T | S | 27.3 |
| Guizotia abyssinica | T | S | 38.5 |
| Hamamelis virginiana | T | O | 37.1 |
| Hamamelis virginiana | T | R | 100.0 |
| Hedeoma pulegioides | T | O | 28.5 |
| Hedeoma pulegioides | T | S | 28.2 |
| Helenium hoopesii | T | O | 31.7 |
| Helenium hoopesii | T | S | 56.0 |
| Helianthus tuberosus | T | V | 23.7 |
| Helichrysum thianschanicum | T | O | 38.4 |
| Helichrysum thianschanicum | T | R | 27.0 |
| Helleborus niger | T | S | 32.1 |
| Hibiscus cannabinus | T | O | 39.9 |
| Hibiscus cannabinus | T | S | 21.1 |
| Humulus lupulus | T | S | 54.8 |
| Humulus lupulus | T | R | 50.5 |
| Hydrastis canadensis | T | O | 20.9 |
| Hypericum henryi | T | O | 32.5 |
| Hypericum perforatum | T | S | 27.9 |
| Hypericum sp | T | W | 55.9 |
| Hypomyces lactifluorum | T | S | 42.7 |
| Iberis amara | T | S | 100.0 |
| Inula helenium | T | S | 30.1 |
| Ipomola batatas | T | V | 27.4 |
| Ipomola batatas | T | S | 44.9 |
| Juniperus communis | T | S | 57.8 |
| Laportea canadensis | T | S | 63.5 |
| Laurus nobilis | T | W | 73.6 |
| Laurus nobilis | T | S | 21.2 |
| Lavandula angustifolia | T | O | 22.7 |
| Lavandula angustifolia | T | S | 25.1 |
| Lavandula latifolia | T | O | 100.0 |
| Lavandula latifolia | T | S | 28.5 |
| Ledum groenlandicum | T | O | 54.3 |
| Lentinus edodes | T | S | 25.7 |
| Leonurus cardiaca | T | R | 24.3 |
| Lepidium sativum | T | O | 100.0 |
| Levisticum officinale | T | R | 41.2 |
| Litchi chinensis | T | S | 100.0 |
| Lolium multiflorum | T | O | 24.0 |
| Lolium perenne | T | O | 27.8 |
| Lonicera ramosissima | T | S | 20.9 |
| Lupinus polyphyllus | T | O | 35.1 |
| Lupinus polyphyllus | T | S | 20.5 |
| Luzula sylvatica | T | R | 22.6 |
| Majorana hortensis | T | V | 20.1 |
| Malus spp. | T | V | 37.8 |
| Malus spp. | T | S | 45.1 |
| Malus hupehensis | T | S | 24.4 |
| Melaleuca alternifolia | T | O | 26.7 |
| Melissa officinalis | T | S | 20.7 |
| mentha arvensis | T | R | 34.0 |
| Mentha piperita | T | S | 60.1 |
| Mentha pulegium | T | V | 24.5 |
| Mentha pulegium | T | W | 24.8 |
| Mentha spicata | T | O | 24.4 |
| Mentha suaveolens | T | S | 28.9 |
| Monarda didyma | T | O | 54.7 |
| Musa paradisiaca | T | O | 21.4 |
| Musa paradisiaca | T | W | 32.8 |
| nasturtium officinale | T | O | 100.0 |
| Nepeta cataria | T | O | 60.1 |
| Nepeta cataria | T | S | 23.4 |
| Nigella sativa | T | S | 23.2 |
| Ocimum Basilicum | T | V | 30.7 |
| Ocimum Basilicum | T | W | 30.9 |
| Ocimum Basilicum | T | O | 39.1 |
| Oenothera biennis | T | S | 29.6 |
| Oenothera biennis | T | O | 24.2 |
| Oenothera biennis | T | R | 58.6 |
| Onobrychis viciifolia | T | O | 42.6 |
| Origanum vulgare | T | S | 53.8 |
| Oryza sativa | T | S | 33.3 |
| Oxalis Deppei | T | O | 30.8 |
| Panicum miliaceum | T | S | 21.2 |
| Passiflora spp. | T | O | 30.2 |
| Passiflora spp. | T | V | 59.4 |
| Passiflora spp. | T | S | 24.4 |
| Pastinaca sativa | T | S | 53.9 |
| Pastinaca sativa | T | R | 20.8 |
| Pastinaca sativa | T | O | 26.9 |
| Petroselinum crispum | T | R | 58.2 |
| Phaseolus coccineus | T | S | 27.1 |
| Phaseolus vulgaris | T | W | 37.9 |
| Phaseolus vulgaris | T | O | 22.2 |
| Phaseolus vulgaris | T | S | 23.2 |
| Phlox paniculata | T | S | 21.3 |
| Physalis pruinosa | T | S | 35.2 |
| Phytolacca americana | T | S | 100.0 |
| Plantago coronopus | T | O | 21.2 |
| Plantago coronopus | T | S | 48.2 |
| Pleurotus spp. | T | S | 31.6 |
| Poa pratensis | T | O | 50.7 |
| Podophyllum peltatum | T | S | 27.9 |
| Polygonum chinense | T | S | 25.0 |
| Polygonum aviculare | T | O | 26.0 |
| Polygonum aviculare | T | R | 100.0 |
| Polygonum pensylvanicum | T | O | 42.3 |
| Polygonum persicaria | T | O | 28.8 |
| Populus incrassata | T | S | 100.0 |
| Populus Tremula | T | S | 48.5 |
| Populus x petrowskyana | T | S | 44.1 |
| Populus x petrowskyana | T | O | 100.0 |
| Populus x petrowskyana | T | W | 72.0 |
| Portulaca oleracera | T | O | 33.7 |
| Poterium sanguisorba | T | W | 100.0 |
| Prunus spp. | T | S | 39.6 |
| Prunus persica | T | O | 21.4 |
| Prunus persica | T | V | 26.6 |
| Psidium guajava | T | V | 37.7 |
| Psidium spp. | T | S | 28.3 |
| Psoralea corylifolia | T | S | 51.5 |
| Pteridium aquilinum | T | R | 76.2 |
| Pteridium aquilinum | T | S | 27.9 |
| Punica granatum | T | W | 66.4 |
| Rehmannia glutinosa | T | O | 83.0 |
| Raphanus sativus | T | R | 36.5 |
| Raphanus sativus | T | S | 22.4 |
| Reseda luteola | T | S | 23.6 |
| Reseda odorata | T | O | 20.3 |
| Rheum officinale | T | O | 100.0 |
| Rheum officinale | T | S | 33.3 |
| Rheum x cultorum | T | S | 34.0 |
| Ricinus communis | T | S | 27.5 |
| Ribes Grossularia | T | W | 24.8 |
| Ribes nidigrolaria | T | W | 24.4 |
| Ribes nigrum | T | S | 50.1 |
| Ribes nigrum | T | V | 23.8 |
| Ribes nigrum | T | W | 64.1 |
| Ribes Sylvestre | T | W | 32.4 |
| Rosa rugosa | T | W | 100.0 |
| Rosmarinus officinalis | T | R | 75.8 |
| Rosmarinus officinalis | T | W | 46.6 |
| Rubus idaeus | T | O | 27.6 |
| Rubus idaeus | T | S | 24.3 |
| Rubus idaeus | T | O | 35.5 |
| Rubus occidentalis | T | R | 93.2 |
| Rubus occidentalis | T | O | 42.1 |
| Rubus occidentalis | T | S | 20.5 |
| Rumex acetosella | T | V | 44.9 |
| Rumex crispus | T | O | 31.3 |
| Rumex crispus | T | R | 100.0 |
| Rumex crispus | T | S | 20.8 |
| Ruta graveolens | T | O | 24.1 |
| Serenoa repens | T | S | 28.5 |
| Salvia officinalis | T | R | 66.5 |
| Salvia officinalis | T | O | 54.0 |
| Salvia officinalis | T | W | 47.2 |
| Sambucus canadensis | T | S | 23.2 |
| Sambucus canadensis | T | O | 35.0 |
| Sambucus canadensis | T | R | 32.6 |
| Sambucus canadensis | T | W | 54.0 |
| Sanguisorba minor | T | W | 50.0 |
| Santolina chamaecyparissus | T | O | 75.8 |
| Santolina chamaecyparissus | T | R | 33.3 |
| Satureja montana | T | O | 100.0 |
| Satureja montana | T | R | 66.8 |
| Satureja repandra | T | R | 87.4 |
| Schizonepeta tenuifolia | T | O | 29.1 |
| Schizonepeta tenuifolia | T | S | 21.1 |
| Scorzorera hispanica | T | R | 42.3 |
| Scorzorera hispanica | T | S | 20.8 |
| Scutellaria lateriflora | T | S | 36.6 |
| Serratula tinctoria | T | S | 36.3 |
| Sium sisarum | T | O | 22.1 |
| Solanum melongena | T | O | 22.4 |
| Solidago sp | T | S | 22.6 |
| Sonchus oleraceus | T | R | 41.8 |
| Sorghum caffrorum | T | O | 23.0 |
| Sorghum dochna | T | O | 30.3 |
| Sorghum dochna | T | O | 53.5 |
| Sorghum durra | T | V | 21.6 |
| Sorghum sudanense | T | V | 23.7 |
| Stachys byzantina | T | O | 25.3 |
| Stellaria graminea | T | O | 27.6 |
| Stellaria graminea | T | S | 36.7 |
| Stellaria media | T | O | 22.6 |
| Stipa capillata | T | O | 36.7 |
| Symphytum officinale | T | O | 20.6 |
| Symphytum officinale | T | V | 25.0 |
| Tanacetum cinerariifolium | T | R | 24.9 |
| Tanacetum vulgare | T | O | 46.4 |
| Tanacetum vulgare | T | S | 32.0 |
| Taraxacum officinale | T | O | 63.1 |
| Thlaspi arvense | T | O | 32.5 |
| Thymus fragantissimus | T | R | 36.7 |
| Thymus fragantissimus | T | O | 100.0 |
| Thymus praecox subsp arcticus | T | O | 38.7 |
| Thymus pseudolanuginosus | T | R | 21.5 |
| Thymus vulgaris | T | W | 20.0 |
| Triticosecale spp. | T | O | 26.0 |
| Triticum aestivum | T | O | 20.9 |
| Triticum turgidum | T | O | 49.4 |
| Triticum spelta | T | O | 35.0 |
| Tropaeofum majus | T | S | 23.5 |
| Tsuga diversifolia | T | S | 34.3 |
| Tsuga mertensiana | T | S | 32.8 |
| Typha latifolia | T | S | 36.1 |
| Urtica dioica | T | O | 32.8 |
| Vaccinium angustifolium | T | S | 33.7 |
| Vaccinium macrocarpon | T | V | 24.1 |
| Vaccinium macrocarpon | T | W | 30.3 |
| Vaccinium macrocarpon | T | S | 70.9 |
| Vaccinium macrocarpon | T | O | 57.2 |
| Valeriana officinalis | T | O | 26.0 |
| Valerianella locusta | T | O | 53.7 |
| Verbascum thapsus | T | O | 22.8 |
| Verbascum thapsus | T | S | 25.2 |
| Veronica officinalis | T | O | 29.9 |
| Vitis | T | S | 39.1 |
| Vitis | T | O | 40.0 |
| Vitis | T | W | 23.5 |
| Vitis | T | S | 26.4 |
| Weigela coraeensis | T | S | 20.1 |
| Weigela hortensis | T | S | 25.3 |
| Xanthium sibiricum | T | O | 28.4 |
| Zea mays | T | S | 38.4 |
| Achillea plarmica | A | O | 54.3 |
| Achillea plarmica | G | O | 64.3 |
| Geranium pratense | T | R | 93.4 |
| Geranium pratense | A | R | 98.5 |
| Geranium pratense | G | R | 97.4 |
| Thalictrum aquilegiifolium | T | O | 53.6 |
| Thalictrum aquilegiifolium | G | O | 60.4 |
| Veronica spicata | T | O | 55.9 |
| Veronica spicata | A | O | 59.2 |
| Veronica spicata | G | O | 56.2 |
| Helenium spp. | T | O | 55.7 |
| Salvia sylvestris | T | O | 77.4 |
| Salvia sylvestris | A | O | 66.9 |
| Salvia sylvestris | G | O | 55.0 |
| Salvia regeliana | T | O | 62.6 |
| Crambe cordifolia | G | R | 56.3 |
| Crambe cordifolia | G | O | 56.7 |
| Rudbeckia maxima | G | O | 68.4 |
| Trollius x cultorum | T | R | 97.6 |
| Trollius x cultorum | A | R | 93.2 |
| Trollius x cultorum | G | R | 100.1 |
| Amsonia tabernaemontana | A | R | 53.2 |
| Oenothera fruticosa spp. | T | R | 109.8 |
| Oenothera fruticosa spp. | T | O | 61.3 |
| Oenothera fruticosa spp. | A | R | 97.5 |
| Oenothera fruticosa spp. | G | R | 105.9 |
| Veronica austriaca ssp teucrium | T | O | 68.6 |
| Veronica austriaca ssp teucrium | G | O | 58.1 |
| Coreopsis verticillata | T | R | 55.6 |
| Coreopsis verticillata | G | O | 70.4 |
| Potentilla fruticosa | T | R | 104.8 |
| Potentilla fruticosa | A | R | 99.4 |
| Lysimachia clethroides | G | O | 67.8 |
| Magnolia x loebneri | T | R | 61.4 |
| Iberis sempervirens | T | O | 62.4 |
| Iberis sempervirens | G | O | 63.8 |
| Filipendula vulgaris | T | R | 98.3 |
| Filipendula vulgaris | A | R | 94.5 |
| Filipendula vulgaris | G | R | 96.3 |
| Geranium sanguineum | T | R | 89.4 |
| Geranium sanguineum | T | O | 63.3 |
| Geranium sanguineum | A | R | 82.6 |
| Geranium sanguineum | A | O | 53.2 |
| Garanium sanguineum | G | R | 88.8 |
| Garanium sanguineum | G | O | 57.7 |
| Philadelphus coronarius | A | O | 55.5 |
| paeonia suffruticosa | T | R | 58.9 |
| paeonia suffruticosa | T | O | 52.1 |
| Paeonia suffruticosa | A | R | 73.8 |
| Paeonia suffruticosa | A | O | 52.2 |
| Paeonia suffruticosa | G | R | 58.7 |
| Paeonia suffruticosa | G | O | 50.4 |
| Dahlia spp. | T | R | 77.4 |
| Begonia convolvulacea | T | O | 69.8 |
| Begonia convolvulacea | A | O | 67.5 |
| Begonia convolvulacea | G | O | 72.6 |
| Begonia eminii | T | O | 72.8 |
| Begonia eminii | A | O | 77.2 |
| Begonia eminii | G | O | 75.4 |
| Begonia glabra | T | O | 82.3 |
| Begonia mannii | A | O | 82.5 |
| Begonia mannii | G | O | 72.8 |
| Begonia polygonoides | T | O | 79.0 |
| Begonia polygonoides | A | O | 74.8 |
| Begonia polygonoides | G | O | 73.2 |
| Fushia spp. | T | R | 76.6 |
| Fushia spp. | A | R | 70.7 |
| Fushia spp. | G | R | 76.9 |
| Butomus umbellatus | A | O | 58.8 |
| Onoclea sensibilis | G | O | 54.7 |
| Onoclea sensibilis | G | R | 50.1 |
| Pinus cembra | A | R | 83.2 |
| Pinus cembra | G | R | 76.3 |
| Cornus sericea | T | R | 104.0 |
| Cornus sericea | A | O | 53.4 |
| Cornus sericea | A | R | 91.8 |
| Cornus sericea | G | O | 51.0 |
| Cornus sericea | G | R | 98.5 |
| Hydrangea quercifolia | T | R | 58.1 |
| Solidago caesia | T | R | 60.7 |
| Solidago caesia | A | R | 60.5 |
| Cornus alba | T | R | 98.9 |
| Cornus alba | A | R | 106.7 |
| Cornus alba | G | R | 85.3 |
| Carpinus caroliniana | T | R | 95.4 |
| Carpinus caroliniana | A | R | 86.2 |
| Carpinus caroliniana | G | R | 94.5 |
| Astilbe chinensis | T | R | 54.3 |
| Astilbe chinensis | G | R | 50.3 |
| Symphoricarpos albus | G | R | 52.0 |
| Euphorbia amygdaloides | T | R | 103.8 |
| Euphorbia amygdaloides | A | R | 75.2 |
| Euphorbia amygdaloides | G | R | 71.3 |
| Viburnum plicatum | A | R | 61.0 |
| Rubus arcticus | T | R | 89.3 |
| Rubus arcticus | A | R | 85.5 |
| Rubus Phoenicolasius | G | R | 93.2 |
| ribes americanum | T | R | 70.4 |
| Passiflora spp. | T | O | 62.4 |
| Rubus occidentalis | T | R | 70.9 |
| Nicotiana tabacum | G | O | 60.9 |
| Beta vulgaris | T | O | 71.3 |
| Oenothera biennis | A | R | 80.3 |
| Alchemilla mollis | T | R | 96.0 |
| Alchemilla mollis | A | R | 87.2 |
| Symphytum officinale | A | O | 80.2 |
| Fragaria x ananassa | A | R | 97.9 |
| Fragaria x ananassa | G | R | 93.8 |
| Vaccinium corymbosum | G | R | 58.6 |
| Vaccinium augustifolium | A | R | 71.8 |
| Vaccinium augustifolium | G | R | 53.6 |
| Vitis | A | R | 62.5 |
| Vitis | G | R | 79.4 |
| Petasites japonicus | A | R | 56.5 |
| Petasites japonicus | G | R | 53.0 |
| Nicotiana rustica | G | O | 61.1 |
| Pysalis ixocarpa | A | R | 53.8 |
| Pteridium aquilinum | T | O | 69.2 |
| Pteridium aquilinum | A | R | 66.2 |
| Pteridium aquilinum | G | R | 56.3 |
| Pteridium aquilinum | G | O | 56.2 |
| Matteuccia pensylvanica | T | R | 67.2 |
| Matteuccia pensylvanica | A | R | 59.0 |
| Ocimum tenuiflorum | T | O | 54.8 |
| Carthamus tinctorius | A | R | 50.9 |
| Carthamus tinctorius | G | R | 69.0 |
| Ligustrum vulgare | T | O | 87.0 |
| Ligustrum vulgare | A | O | 76.2 |
| Ligustrum vulgare | G | O | 85.7 |
| Malva verticillata | T | R | 80.1 |
| Malva verticillata | A | R | 82.9 |
| Malva verticillata | G | R | 82.4 |
| Hamamelis virginiana | T | R | 56.1 |
| Arctostaphylos uva-ursi | T | R | 74.8 |
| Arctostaphylos uva-ursi | G | R | 86.0 |
| Vicia faba | T | O | 84.6 |
| Sempervivum tectorum | T | O | 57.3 |
| Sempervivum tectorum | A | O | 74.8 |
| Sempervivum tectorum | G | O | 52.3 |
| Ajuga reptans | T | O | 55.3 |
| Ajuga reptans | A | O | 52.3 |
| Ajuga reptans | G | O | 72.1 |
| Phlox paniculata | T | O | 66.2 |
| Ligularia dentata | A | O | 52.1 |
| Ligularia dentata | G | R | 50.8 |
| Ligularia dentata | G | O | 52.6 |
| Achillea ptarmica | T | O | 50.9 |
| Potentilla fruticosa | G | R | 98.6 |
| Vernonia gigantea | A | R | 50.4 |
| Vernonia gigantea | A | O | 62.3 |
| Vernonia gigantea | G | R | 51.2 |
| Vernonia gigantea | G | O | 50.7 |
| Penstemon digitalis | T | R | 64.5 |
| Penstemon digitalis | A | R | 63.5 |
| Penstemon digitalis | A | O | 57.3 |
| Penstemon digitalis | G | R | 63.4 |
| Penstemon digitalis | G | O | 67.8 |
| Malus spp. | T | R | 56.1 |
| Malus spp. | T | O | 56.7 |
| Malus spp. | A | R | 50.8 |
| Malus spp. | G | R | 51.2 |
| Hosta sieboldiana | G | O | 50.9 |
| Hamamelis mollis | T | R | 99.1 |
| Hamamelis mollis | A | R | 94.1 |
| Hamamelis mollis | G | R | 89.4 |
| Chaenomeles x superba | T | R | 56.2 |
| Chaenomeles x superba | A | R | 71.9 |
| Chaenomeles x superba | G | R | 66.6 |
| Chaenomeles x superba | G | O | 52.0 |
| Centaurea dealbata | T | R | 50.9 |
| Centaurea dealbata | A | R | 74.1 |
| Paeonia spp. | T | R | 79.8 |
| Paeonia spp. | T | O | 58.6 |
| Paeonia spp. | A | R | 79.6 |
| Paeonia spp. | A | O | 58.5 |
| Paeonia spp. | G | R | 82.0 |
| Paeonia spp. | G | O | 60.0 |
| Lysimachia clethroides | T | R | 83.3 |
| Lysimachia clethroides | T | O | 64.3 |
| Lysimachia clethroides | G | R | 85.8 |
| Viburnum plicatum | G | R | 57.9 |
| Buxus microphylla | T | R | 58.0 |
| Astilboides tabularis | T | R | 104.2 |
| Astilboides tabularis | A | R | 108.1 |
| Astilboides tabularis | G | R | 100.3 |
| Staphylea trifolia | A | R | 63.6 |
| Bergenia x schmidtii | T | R | 100.5 |
| Bergenia x schmidtii | A | R | 113.7 |
| Bergenia x schmidtii | G | R | 99.3 |
| Rodgersia podophylla | T | R | 68.9 |
| Rodgersia podophylla | A | R | 59.4 |
| Rodgersia podophylla | G | R | 56.5 |
| Geranium phaeum | T | R | 92.7 |
| Geranium phaeum | A | R | 84.3 |
| Geranium phaeum | G | R | 101.0 |
| Rubus pubescens | T | R | 71.5 |
| Rubus pubescens | A | R | 76.2 |
| Rubus pubescens | G | R | 82.8 |
| Taxus x media | T | R | 60.1 |
| Taxus x media | A | R | 61.6 |
| Taxus x media | G | R | 52.3 |
| Geranium x cantabrigiense | T | R | 106.1 |
| Geranium x cantabrigiense | A | R | 94.2 |
| Geranium x cantabrigiense | G | R | 95.9 |
| Fuchia magellanica | T | R | 100.2 |
| Fuchia magellanica | A | R | 91.9 |
| Fuchia magellanica | G | R | 102.2 |
| Microbiata decussata | A | R | 51.5 |
| Microbiata decussata | G | R | 51.9 |
| Rhododendron spp. | G | R | 51.2 |
| Stephanandra incisa | T | R | 102.5 |
| Stephanandra incisa | A | R | 104.6 |
| Stephanandra incisa | G | R | 99.1 |
| Corylus maxima | A | R | 50.8 |
| Corylus maxima | G | R | 57.1 |
| Cyperus alternifolius | G | R | 56.2 |
| Soleirolia soleirolii | A | R | 51.2 |
| Soleirolia soleirolii | G | R | 68.0 |
| Strelitzia reginae | T | R | 106.5 |
| Strelitzia reginae | A | R | 94.3 |
| Strelitzia reginae | G | R | 111.7 |
| Hedychium coronarium | T | R | 53.5 |
| Hedychium coronarium | A | R | 86.9 |
| Hedychium coronarium | G | R | 74.6 |
| Strelitzia reginae | T | R | 78.6 |
| Strelitzia reginae | A | R | 78.0 |
| Strelitzia reginae | G | R | 107.3 |
| Symphoricarpos orbiculatus | G | R | 58.7 |
| Rodgersia spp. | A | R | 59.5 |
| Rodgersia spp. | G | R | 59.0 |
| Lamiastrum galeobdolon | T | R | 91.5 |
| Astilbe x arendsii | A | R | 84.5 |
| Clematis alpina | A | R | 54.4 |
| Stewartia pseudocamellia | T | R | 75.5 |
| Stewartia pseudocamellia | A | R | 84.1 |
| Stewartia pseudocamellia | G | R | 81.3 |
| Pinus mugo | T | R | 58.9 |
| Pinus mugo | A | R | 53.7 |
| Pinus mugo | G | R | 61.7 |
| Rubus thibetanus | T | R | 97.6 |
| Rubus thibetanus | A | R | 97.9 |
| Rubus thibetanus | G | R | 95.4 |

TABLE 11
Clostripain

| | | | Inhibition |
|---|---|---|---|
| Nom latin | Stress | Extrait | (%) |
| Achidinia arguta | A | R | 34.1 |
| Anthoxanthum odoratum | A | R | 35.0 |
| Apocynum cannabinum | A | R | 47.6 |
| Arctium minus (Hill) Bernhardi | A | R | 34.5 |
| Beckmannia erucaeformis | A | O | 47.3 |
| Beta vulgaris | A | O | 37.2 |
| Brassica rapa | A | O | 24.6 |
| Buddleja davidii | A | R | 27.6 |
| Bupleurum falcatum | A | O | 34.6 |
| Capsicum annuum | A | S | 36.8 |
| Capsicum annuum | A | R | 24.9 |
| Cotinus coggygria | A | R | 21.0 |
| Kolkwitzia amabilis | A | R | 27.9 |
| Laserpitium latifolium | A | R | 20.4 |
| Lindera benzoin | A | R | 38.6 |
| Lolium perenne | A | S | 34.7 |
| Miscanthus sacchariflorus | A | O | 39.9 |
| Ophiopogon japonicus | A | R | 20.5 |
| Phaseolus mungo | A | S | 30.0 |
| Phaseolus Vulgaris | A | O | 36.4 |
| Phaseolus Vulgaris | A | R | 23.4 |
| Plumbago zeylanica | A | O | 26.5 |
| Portulacea oleracea | A | O | 22.2 |
| Salix purpurea F. Gracilis | A | R | 38.6 |
| Solanum melanocerasum | A | S | 26.0 |
| Stellaria media (linné) Cyrillo | A | O | 31.6 |
| Tanacetum vulgare | A | S | 35.3 |
| Tanacetum vulgare | A | O | 35.4 |
| Trifolium incarnatum | A | S | 22.0 |
| Vaccinum augustifolium | A | O | 34.0 |
| Zea Mays | A | O | 21.9 |
| Aframomum melegueta | G | O | 27.9 |
| Allium sativum | G | O | 35.3 |
| Anthemis nobilis | G | O | 35.8 |
| Anthurium guildingii | G | O | 55.2 |
| Astilbe x arendsii | G | R | 25.6 |
| Beta vulgaris | G | R | 28.0 |
| Campanula rapunculus | G | S | 24.5 |
| Cirsium arvense | G | R | 30.0 |
| Cissus discolor | G | O | 40.8 |
| Coccoloba caracasana | G | R | 24.9 |
| Convallaria majalis | G | R | 28.5 |
| Cucurbita pepo | G | O | 20.9 |
| Cucurbita pepo | G | S | 42.5 |
| Errhenatherum elatius | G | S | 21.6 |
| Filipendula rubra | G | R | 44.3 |
| Galium odoratum | G | O | 31.2 |
| Glycyrrhiza glabra | G | O | 27.6 |
| Hedychium sp. | G | O | 35.6 |
| Houttuynia cordata | G | O | 30.2 |
| Lactuca sativa | G | O | 28.8 |
| Lactuca sativa | G | O | 21.6 |
| Lotus tetragonolobus | G | S | 42.9 |
| Lycopersicon esculentum | G | R | 32.3 |
| Lysimachia clethroides | G | R | 22.7 |
| Magnolia stellata | G | R | 23.6 |
| Microlepia plalyphylla | G | O | 21.0 |
| Miscanthus sacchariflorus | G | R | 25.6 |
| Myrica pensylvanica | G | O | 22.7 |
| N | G | O | 24.4 |
| Nicotiana tabacum | G | R | 22.8 |
| Paeonia | G | R | 31.3 |
| Pastinaca sativa | G | R | 29.2 |
| pastinaca sativa | G | S | 44.7 |
| Phaseolus vulgaris | G | O | 36.7 |
| Pteridium aquilinum | G | O | 22.2 |
| Solidago sp ? | G | S | 40.8 |
| Symphytum officinale | G | S | 22.7 |
| Tanacetum vulgare | G | S | 31.4 |
| Thymus fragantissumus | G | O | 20.1 |
| Urtica dioica | G | O | 32.6 |
| Zea mays | G | O | 22.4 |
| Abies balsamea | T | O | 38.6 |
| Allium ampeloprasum | T | S | 30.3 |
| Allium sativum | T | O | 55.5 |
| Amaranthus gangeticus | T | R | 75.4 |
| Apium graveolens | T | R | 21.7 |
| Aralia cordata | T | S | 48.2 |
| Asclepias tuberosa | T | O | 20.2 |
| Asctinidia chinensis | T | O | 47.7 |
| Baptisia tinctoria | T | O | 50.4 |
| Betula alleghaniensis | T | R | 24.9 |
| Brassica oleracea | T | R | 21.4 |
| Brassica rapa | T | R | 30.5 |
| Caladium sp. | T | O | 39.8 |
| Carica papaya | T | R | 23.8 |
| Chaerophyllum bulbosum | T | R | 24.3 |
| Chrysanthenum coronarium | T | O | 32.7 |
| Clematis chiisanensis | T | R | 21.6 |
| Coccoloba caracasana | T | O | 40.1 |
| Cocos nucifera | T | R | 22.5 |
| Cornus mas | T | R | 34.2 |
| Cucurbita pepo | T | S | 24.9 |
| Cymbopogon citratus | T | O | 20.4 |
| Forsythia x intermedia | T | S | 44.0 |
| Heliotropium arborescens | T | O | 27.1 |
| Lonicera ramosissima | T | O | 34.9 |
| Malus pranifolia | T | R | 23.6 |
| Marrubium vulgare | T | R | 49.3 |
| Miscanthus sinensis Anchess | T | R | 26.9 |
| Nephelium longana ou Euphoria longana | T | O | 42.6 |
| Psoralea corylifolia | T | S | 54.0 |
| Raphanus sativus | T | O | 21.4 |
| Ribes Nigrum | T | R | 40.9 |
| Rubus thibetanus | T | R | 24.2 |
| Rumex acetosella linné | T | O | 35.2 |
| Sechium edule | T | R | 25.6 |
| Stachys macrantha | T | O | 25.9 |
| Tepary | T | R | 34.9 |
| Thymus vulgaris “Argenteus” | T | O | 25.3 |
| Trifolium pratense | T | R | 31.3 |
| Trollius x cultorum | T | R | 26.5 |
| Uvularia perfoliata | T | R | 38.3 |
| Vaccinum macrocarpon | T | O | 39.2 |
| Verbena officinalis | T | R | 46.2 |
| Zea mays | T | R | 32.5 |

TABLE 12
Subtilisin

| Nom latin | Stress | Extrait | Inhibition (%) |
|---|---|---|---|
| Actaea racemosa | A | O | 20.6 |
| Alchemilla motlis | A | S | 23.5 |
| Borago officinalis | A | S | 20.5 |
| Capsicum annuum | A | S | 24.7 |
| Cornus canadensis L. | A | S | 22.6 |
| Genista multibracteata | A | R | 21.3 |
| Glycine max | A | S | 26.0 |
| Lolium perenne | A | S | 75.9 |
| Matricaria recutita | A | S | 23.2 |
| Phaseolus Vulgaris | A | O | 34.7 |
| Prunus Tomentosa | A | R | 20.4 |
| Scuttellaria lateriflora | A | O | 33.5 |
| Solidago canadensis | A | O | 42.0 |
| Spinacia oleracea | A | S | 100.0 |
| Tanacetum vulgare | A | S | 42.4 |
| Tanacetum vulgare | A | O | 26.7 |
| Typha latifolia L. | A | O | 24.9 |
| Zea mays | A | S | 20.9 |
| Zea Mays | A | O | 34.7 |
| Adiantum pedatum | G | S | 22.4 |
| Cichorium endivia | G | O | 26.7 |
| Cucurbita pepo | G | O | 20.8 |
| Echinacea purpurea | G | O | 27.6 |
| Lactuca sativa | G | O | 36.4 |
| pastinaca sativa | G | S | 52.1 |
| Pastinaca sativa | G | S | 20.1 |
| Ribes nigrum | G | O | 41.2 |
| Symphytum officinale | G | O | 30.0 |
| Urtica dioica | G | O | 38.2 |
| Vitis sp. | G | S | 22.3 |
| Alchemilla mollis | T | S | 22.6 |
| Althacea officinalis | T | O | 33.5 |
| Althaea officinalis | T | S | 53.5 |
| Aralia cordata | T | S | 21.0 |
| Asctinidia chinensis | T | O | 38.6 |
| Astilboides tabularis | T | O | 41.0 |
| Averrhoa carambola | T | S | 20.9 |
| Baptisia tinctoria | T | O | 25.5 |
| Beta vulgaris | T | S | 24.2 |
| Convallaria majalis | T | O | 48.2 |
| Datura stramonium | T | O | 27.3 |
| Dioscorea batatas | T | S | 36.4 |
| Eleusine coracana | T | S | 26.2 |
| Fragaria x ananassa | T | O | 39.5 |
| Ginkgo biloba | T | O | 98.8 |
| Heliotropium arborescens | T | O | 35.2 |
| Hibiscus cannabinus | T | S | 25.2 |
| Hypericum perforatum | T | O | 30.3 |
| Ipomea batalas | T | S | 22.1 |
| Lathyrus sylvestris | T | S | 21.8 |
| Lonicera ramosissima | T | O | 29.6 |
| Lonicera ramosissima | T | S | 39.9 |
| Lonicera syringantha | T | R | 31.1 |
| Madia sativa | T | O | 27.5 |
| Monarda | T | O | 28.2 |
| Ocimum Basilicum | T | S | 27.2 |
| Peucedanum oreaselinum | T | S | 29.2 |
| Psoralea corylifolia | T | S | 20.9 |
| Rahmnus frangula | T | O | 26.4 |
| Raphanus sativus | T | S | 25.5 |
| Rheum rhabarbarum | T | S | 21.6 |
| Ribes Nigrum | T | R | 28.9 |
| Rubus occidentalis | T | S | 22.8 |
| Rumes scutatus | T | S | 21.4 |
| Solidago Hybrida | T | O | 34.5 |
| Tanacetum balsamila | T | O | 33.9 |
| Vaccinum macrocarpon | T | O | 81.2 |
| Xanthium sibiricum | T | S | 31.7 |
| Zea mays | T | S | 28.3 |

TABLE 13(con)

| | | | Endothelial Cell Migration | | | | | | | |
|---|---|---|---|---|---|---|---|---|---|---|
| | | | Cellular | | | | | | | |
| | | | Migration Assay | | | | Cord Formation Assay | | | |
| | | | % inhibition | | | | % inhibition | | | |
| Plant | Stress¹ | Part of Plant² | 2.5x | 1.25x | 0.62x | 0.31x | 2.5x | 1.25x | 0.62x | 0.31x |
| Amaranthus candathus | G | L | 100 | 72 | 100 | 81 | 100 | 100 | 100 | 100 |
| Ambrosia artemisiifolia | N | Fl | 99 | 91 | 61 | 57 | 100 | 90 | 4 | 0 |
| Aronia x prunifolia | N | L/St | | 93 | 75 | 93 | 50 | 26 | 20 | 19 |
| Brassica napus | N | L | 51 | 33 | 0 | 0 | 77 | 59 | 43 | 41 |
| Brassica oleracea | N | L | 35 | 15 | 0 | 4 | 50 | 29 | 30 | 20 |
| Brassica oleracea | A | L | 49 | 28 | 27 | 6 | 65 | 32 | 15 | 21 |
| Bromus inermis | A | L | 21 | 14 | 0 | 93 | 90 | 44 | 36 | 17 |
| Chenopodium quinoa | N | L/St/Se | 90 | 85 | 53 | 42 | 100 | 100 | 44 | 26 |
| Citrullus lanatus | A | L | 21 | 17 | 6 | 0 | 88 | 35 | 23 | 14 |
| Cynara cardunculus | G | Fr | 36 | 0 | 36 | 0 | 4 | 0 | 0 | 10 |
| subsp. Cardunculus | | | | | | | | | | |
| Dolichos lablab | G | Fl/Fr | 0 | 0 | 0 | 0 | 60 | 64 | 68 | 83 |
| Foeniculum vulgare | N | L | 69 | 21 | 23 | 11 | 64 | 47 | 62 | 61 |
| Hypomyces | N | Fr | 77 | 67 | 20 | 11 | 85 | 59 | 31 | 5 |
| lactifluorum | | | | | | | | | | |
| Lotus corniculatus | A | L/Fr/St | 9 | 0 | 0 | 0 | 93 | 83 | 77 | 57 |
| Lotus corniculatus | N | Se | 0 | 0 | 0 | 0 | 58 | 11 | 26 | 0 |
| Manihot esculenta | N | Fr | 39 | 0 | 0 | 0 | 33 | 30 | 25 | 26 |
| Matricaria recutita | G | L/Fl/St | 34 | 31 | 4 | 0 | 74 | 6 | 1 | 20 |
| Melilotus albus | G | L/St | | | | 0 | 70 | 15 | 0 | 0 |
| Phaseolus vulgaris | A | L | 51 | 17 | 4 | 7 | 54 | 29 | 10 | 18 |
| Phaseolus vulgaris | G | L | 33 | 13 | 25 | 18 | 82 | 56 | 51 | 41 |
| Pisum sativum | N | L/St | 16 | 24 | 4 | 0 | 38 | 16 | 13 | 0 |
| Raphanus raphanistrum | G | L | 46 | 24 | 10 | 0 | 88 | 46 | 23 | 23 |
| Ribes sylvestre | N | L | 96 | 87 | 56 | 26 | 59 | 49 | 69 | 56 |
| Rumex crispus | A | R | 96 | 83 | 0 | 18 | 96 | 46 | 17 | 13 |
| Rumex crispus | G | R | 36 | 0 | 36 | 0 | 80 | 100 | 86 | 36 |
| Rumex scutatus | N | L | 70 | 6 | 0 | 0 | 100 | 20 | 0 | 0 |
| Tanacetum | G | L | 100 | 99 | 56 | 0 | 100 | 100 | 42 | 18 |
| cinerariifolium | | | | | | | | | | |
| Tropaeolum majus | G | L | 7 | 0 | 0 | 0 | 65 | 29 | 18 | 4 |
| Tsuga canadensis | N | L/Fr/St | | 80 | 82 | 64 | 68 | 41 | 31 | 31 |
| Tsuga diversifolia | N | L/St | 57 | 8 | 0 | 0 | 99 | 43 | 18 | 27 |
| Vaccinium | N | Fr | 59 | 15 | 6 | 0 | 62 | 7 | 11 | 24 |
| angustifolium | | | | | | | | | | |
| Zea mays | N | L | 11 | 0 | 0 | 11 | 66 | 24 | 14 | 6 |
| Zingiber officinale | N | Fr | 0 | 0 | 0 | 0 | 59 | 38 | 27 | 30 |

¹N: no stress; A: stress A; G: stress G.
²EP: Entire plant; Fl: Flower; Fr: Fruit; L: Leaf; R: Root; Se: Seed; St: Stem

TABLE 14(con)

| | | | Migration of Cancer Cells | | | |
|---|---|---|---|---|---|---|
| | | Part of | % inhibition | | | |
| 254Plant | Stress¹ | plant² | 2.5x | 1.25x | 0.62x | 0.31x |
| Allium tuberosum | G | Fr/Fl | 68 | 0 | 0 | 0 |
| Allium tuberosum | A | Fr/Fl | 73 | 76 | 80 | 36 |
| Althacea officinalis | N | L/St | 66 | 0 | 0 | 0 |
| Amaranthus candathus | G | L | 100 | 100 | 100 | 98 |
| Ambrosia | N | Fl | 92 | 76 | 0 | 0 |
| artemisiifolia | | | | | | |
| Angelica sinensis | N | EP | 100 | 75 | 32 | 53 |
| Aronia x prunifolia | N | L/St | 95 | 94 | 95 | 97 |
| Asarum europaeum | G | L | 67 | 49 | 0 | 73 |
| Begonia Hannii | A | L/Fl/Fr/St | 100 | 100 | 14 | 0 |
| Begonia polygonoides | A | L/Fl/St | 100 | 0 | 0 | 0 |
| Brassica oleracea | N | L | 78 | 45 | 49 | 57 |
| Bromus inermis | A | L | 91 | 91 | 93 | 90 |
| Chenopodium quinoa | N | L/St/Se | 100 | 99 | 58 | 31 |
| Conyza canadensis | G | EP | 65 | 8 | 0 | 0 |
| Cynara cardunculus | G | Fr | 99 | 39 | 33 | 48 |
| subsp. Cardunculus | | | | | | |
| Daucus carota | G | L | 0 | 30 | 0 | 38 |
| Dolichos lablab | G | Fl/Fr | 81 | 86 | 92 | 77 |
| Foeniculum vulgare | N | L | 6 | 6 | 5 | 0 |
| Hypomyces | N | Fr | 66 | 72 | 0 | 0 |
| lactifluorum | | | | | | |
| Iberis sempervirens | A | L/St | 100 | 42 | 4 | 0 |
| Iberis sempervirens | G | L/St | 100 | 100 | 98 | 91 |
| Lotus corniculatus | A | L/Fr/St | 88 | 51 | 35 | 21 |
| Lotus corniculatus | N | Se | 47 | 71 | 80 | 55 |
| Lunaria annua | N | Fr | 100 | 100 | 68 | 9 |
| Melilotus albus | G | L/St | 54 | 0 | 0 | 0 |
| Phaseolus vulgaris | G | L | 43 | 2 | 0 | 0 |
| Physostegia virginiana | G | L/St | 78 | 0 | 0 | 0 |
| Pisum sativum | N | L/St | 27 | 23 | 12 | 9 |
| Rheum rhabarbarum | A | L | 90 | 90 | 87 | 87 |
| Ribes sylvestre | N | L | 91 | 87 | 17 | 0 |
| Rubus occidentalis | N | Fr | 84 | 82 | 89 | 90 |
| Rumex crispus | A | R | 96 | 89 | 8 | 0 |
| Rumex crispus | G | R | 99 | 86 | 0 | 0 |
| Rumex scutatus | N | L | 100 | 88 | 0 | 0 |
| Salvia officinalis | N | L/St | 59 | 0 | 0 | 0 |
| Salvia officinalis | A | L/St | | 98 | 89 | 39 |
| Solidago canadensis | G | Fl | 100 | 100 | 93 | 93 |
| Solidago sp. | A | L/Fl/St | 100 | 83 | 0 | 0 |
| Solidago x hybrida | N | L/St | 100 | 96 | 70 | 7 |
| Solidago x hybrida | A | L/St | 100 | 90 | 0 | 0 |
| Solidago x hybrida | N | Fl | 100 | 51 | 13 | 0 |
| Solidago x hybrida | A | Fl | 100 | 99 | 91 | 89 |
| Tanacetum | G | L | 100 | 100 | 99 | 65 |
| cinerariifolium | | | | | | |
| Taraxacum officinale | N | L | 100 | 71 | 47 | 0 |
| Tsuga canadensis | N | L/Fr/St | 65 | 64 | 63 | 0 |
| Tsuga diversifolia | N | L/St | 100 | 63 | 38 | 90 |
| Zea mays | N | L | 36 | 35 | 25 | 24 |
| Zingiber officinale | N | Fr | 90 | 56 | 13 | 0 |

¹N: no stress; A: stress A; G: stress G.
²EP: Entire plant; Fl: Flower; Fr: Fruit; L: Leaf; R: Root; Se: Seed; St: Stem

I claim:

1. A formulation comprising a physiologically acceptable diluent, excipient or carrier, and one or more plant extracts that inhibit the activity of at least one extracellular protease selected from the group of: matrix metalloprotease-1 (MMP-1), matrix metalloprotease-2 (MMP-2), matrix metalloprotease-3 (MMP-3), matrix metalloprotease-9 (MMP-9), and cathepsin B, each plant extract having at least one of the following properties:
(i) inhibits migration of endothelial cells,
(ii) inhibits migration of neoplastic cells,
and each of said one or more plant extracts derived from Allium tuberosum, Althacea officinalis, Amaranthus candathus, Ambrosia artemesiifolia, Angelica sinensis, Aronia×prunifolia, Asarum europaeum, Begonia hannii, Begonia polygonoides, Brassica napus, Brassica oleracea, Bromus inermis, Chenopodium quinoa, Citrullus lanatus, Conyza canadensis, Cynara cardunculus subsp. Cardunculus, Daucus carota, Dolichos lablab, Foeniculum vulgare, Hypomyces lactifluorum, Iberis sempervirens, Lotus corniculatus, Lunaria annua, Manihot esculenta, Matricaria recutita, Melilotus albus, Phaseolus vulgaris, Physostegia virginiana, Pisum sativum, Raphanus raphanistrum, Rheum rhabarbarum, Ribes sylvestre, Rubus occidentalis, Rumex crispus, Rumex scutatus, Salvia officinalis, Solidago canadensis, Solidago spp., Solidago×hybrida, Tanacetum cinerariifolium, Taraxacum officinale, Tropaeolum majus, Tsuga canadensis, Tsuga diversifolia, Vaccinium angustifolium, Zea mays or Zingiber officinale.

2. The formulation according to claim 1, wherein said formulation inhibits angiogenesis.

3. The formulation according to claim 1, wherein said formulation inhibits prevents metastasis of cancer cells.

4. The formulation according to claim 1, wherein at least one of said one or more plant extracts is derived from a plant that has been subjected to one or more stress prior to harvest.

5. The formulation according to claim 4, wherein said one or more stress is a chemical stress.

6. The formulation according to claim 5, wherein said chemical stress comprises treatment with gamma linolenic acid or arachidonic acid.

7. The formulation according to claim 1, wherein each of said one or more plant extracts is selected from:
an extract derived from fruits, flowers, or a combination thereof of Allium tuberosum,
an extract derived from leaves, stems, or a combination thereof of Althacea officinalis,
an extract derived from leaves of Amaranthus candathus,
an extract derived from flowers of Ambrosia artemesiifolia,
an extract derived from entire Angelica sinensis plants,
an extract derived from leaves, stems, or a combination thereof of Aronia×prunifolia,
an extract derived from leaves of Asarum europaeum,
an extract derived from leaves, flowers, fruits, stems, or a combination thereof of Begonia hannii,
an extract derived from leaves, flowers, stems, or a combination thereof of Begonia polygonoides,
an extract derived from leaves of Brassica napus,
an extract derived from leaves of Brassica oleracea,
an extract derived from leaves of Bromus inermis,
an extract derived from leaves, stems, seeds, or a combination thereof of Chenopodium quinoa,
an extract derived from leaves of Citrullus lanatus,
an extract derived from entire Conyza canadensis plants,
an extract derived from fruits of Cynara cardunculus subsp. Cardunculus,
an extract derived from leaves of Daucus carota,
an extract derived from flowers, fruits, or a combination thereof of Dolichos lablab,
an extract derived from leaves of Foeniculum vulgare,
an extract derived from fruits of Hypomyces lactifluorum,
an extract derived from leaves, stems, or a combination thereof of Iberis sempervirens,
an extract derived from leaves, fruits, stems, seeds, or a combination thereof of Lotus corniculatus,
an extract derived from fruits of Lunaria annua,
an extract derived from fruits of Manihot esculenta,
an extract derived from leaves, flowers, stems, or a combination thereof of Matricaria recutita,
an extract derived from leaves, stems, or a combination thereof of Melilotus albus,
an extract derived from leaves of Phaseolus vulgaris,
an extract derived from leaves, stems, or a combination thereof of Physostegia virginiana,
an extract derived from leaves, stems, or a combination thereof of Pisum sativum,
an extract derived from leaves of Raphanus raphanistrum,
an extract derived from leaves of Rheum rhabarbarum,
an extract derived from leaves of Ribes sylvestre,
an extract derived from fruits of Rubus occidentalis,
an extract derived from roots of Rumex crispus,
an extract derived from leaves of Rumex scutatus,
an extract derived from leaves, stems, or a combination thereof of Salvia officinalis,
an extract derived from flowers of Solidago canadensis,
an extract derived from leaves, flowers, stems, or a combination thereof of Solidago spp.,
an extract derived from leaves, flowers, stems, or a combination thereof of Solidago×hybrida,
an extract derived from leaves of Tanacetum cinerariifolium,
an extract derived from leaves of Taraxacum officinale,
an extract derived from leaves of Tropaeolum majus,
an extract derived from leaves, fruits, stems, or a combination thereof of Tsuga canadensis,
an extract derived from leaves, stems, or a combination thereof of Tsuga diversifolia,
an extract derived from fruits of Vaccinium angustifolium,
an extract derived from leaves of Zea mays, and
an extract derived from fruits of Zingiber officinale.

8. The formulation according to claim 1, wherein each of said one or more plant extracts is prepared by extraction of plant material with an alcoholic solvent, an aqueous solvent, or a combination thereof.

9. The formulation according to claim 1, wherein said formulation is formulated for oral administration.

10. The formulation according to claim 1, wherein said formulation is formulated for topical administration.

11. The formulation according to claim 9, wherein said formulation is a nutraceutical formulation.

12. The formulation according to claim 1, wherein said formulation is a pharmaceutical composition and said physiologically acceptable diluent, excipient or carrier is a pharmaceutically acceptable diluent, excipient or carrier.

13. A method of inhibiting abnormal cell migration comprising contacting neoplastic or endothelial cells with an effective amount of the formulation according to claim 1.

14. The method according to claim 13, wherein at least one of said one or more plant extracts is derived from a plant that has been subjected to one or more stress prior to harvest.

15. The method according to claim 14, wherein said one or more stress is a chemical stress.

16. The method according to claim 15, wherein said chemical stress comprises treatment with gamma linolenic acid or arachidonic acid.

17. The method according to claim 13, wherein each of said one or more plant extracts is selected from:
an extract derived from fruits, flowers, or a combination thereof of Allium tuberosum,
an extract derived from leaves, stems, or a combination thereof of Althacea officinalis,
an extract derived from leaves of Amaranthus candathus,
an extract derived from flowers of Ambrosia artemesiifolia,
an extract derived from entire Angelica sinensis plants,
an extract derived from leaves, stems, or a combination thereof of Aronia×prunifolia,
an extract derived from leaves of Asarum europaeum,
an extract derived from leaves, flowers, fruits, stems, or a combination thereof of Begonia hannii,
an extract derived from leaves, flowers, stems, or a combination thereof of Begonia polygonoides,
an extract derived from leaves of Brassica napus,
an extract derived from leaves of Brassica oleracea,
an extract derived from leaves of Bromus inermis,
an extract derived from leaves, stems, seeds, or a combination thereof of Chenopodium quinoa,
an extract derived from leaves of Citrullus lanatus,
an extract derived from entire Conyza canadensis plants,
an extract derived from fruits of Cynara cardunculus subsp. Cardunculus,
an extract derived from leaves of Daucus carota,
an extract derived from flowers, fruits, or a combination thereof of Dolichos lablab,
an extract derived from leaves of Foeniculum vulgare,
an extract derived from fruits of Hypomyces lactifluorum,
an extract derived from leaves, stems, or a combination thereof of Iberis sempervirens,
an extract derived from leaves, fruits, stems, seeds or a combination thereof of Lotus corniculatus,
an extract derived from fruits of Lunaria annua,
an extract derived from fruits of Manihot esculenta,
an extract derived from leaves, flowers, stems, or a combination thereof of Matricaria recutita,
an extract derived from leaves, stems, or a combination thereof of Melilotus albus,
an extract derived from leaves of Phaseolus vulgaris,
an extract derived from leaves, stems, or a combination thereof of Physostegia virginiana,
an extract derived from leaves, stems, or a combination thereof of Pisum sativum,
an extract derived from leaves of Raphanus raphanistrum,
an extract derived from leaves of Rheum rhabarbarum,
an extract derived from leaves of Ribes sylvestre,
an extract derived from fruits of Rubus occidentalis,
an extract derived from roots of Rumex crispus,
an extract derived from leaves of Rumex scutatus,
an extract derived from leaves, stems, or a combination thereof of Salvia officinalis,
an extract derived from flowers of Solidago canadensis,
an extract derived from leaves, flowers, stems, or a combination thereof of Solidago spp.,
an extract derived from leaves, flowers, stems, or a combination thereof of Solidago×hybrida,
an extract derived from leaves of Tanacetum cinerariifolium,
an extract derived from leaves of Taraxacum officinale,
an extract derived from leaves of Tropaeolum majus,
an extract derived from leaves, fruits, stems, or a combination thereof of Tsuga canadensis,
an extract derived from leaves, stems, or a combination thereof of Tsuga diversifolia,
an extract derived from fruits of Vaccinium angustifolium,
an extract derived from leaves of Zea mays, and
an extract derived from fruits of Zingiber officinale.

18. The method according to claim 13, wherein each of said one or more plant extracts is prepared by extraction of plant material with an alcoholic solvent, an aqueous solvent, or a combination thereof.

19. The method according to claim 13, wherein said method is an in vivo method.

20. A method of inhibiting angiogenesis in a subject comprising administering to said subject an effective amount of the formulation according to claim 1, wherein said formulation inhibits migration of endothelial cells.

21. The method according to claim 20, wherein at least one of said one or more plant extracts is derived from a plant that have been subjected to one or more stress prior to harvest.

22. The method according to claim 21, wherein said one or more stress is a chemical stress.

23. The method according to claim 22, wherein said chemical stress comprises treatment with gamma linolenic acid or arachidonic acid.

24. The method according to claim 20, wherein each of said one or more plant extracts is selected from:
an extract derived from fruits, flowers, or a combination thereof of Allium tuberosum,
an extract derived from leaves, stems, or a combination thereof of Althacea officinalis,
an extract derived from leaves of Amaranthus candathus,
an extract derived from flowers of Ambrosia artemesiifolia,
an extract derived from entire Angelica sinensis plants,
an extract derived from leaves, stems, or a combination thereof of Aronia×prunifolia,
an extract derived from leaves of Asarum europaeum,
an extract derived from leaves, flowers, fruits, stems, or a combination thereof of Begonia hannii,
an extract derived from leaves, flowers, stems, or a combination thereof of Begonia polygonoides,
an extract derived from leaves of Brassica napus,
an extract derived from leaves of Brassica oleracea,
an extract derived from leaves of Bromus inermis,
an extract derived from leaves, stems, seeds, or a combination thereof of Chenopodium quinoa,
an extract derived from leaves of Citrullus lanatus,
an extract derived from entire Conyza canadensis plants,
an extract derived from fruits of Cynara cardunculus subsp. Cardunculus,
an extract derived from leaves of Daucus carota,
an extract derived from flowers, fruits, or a combination thereof of Dolichos lablab,
an extract derived from leaves of Foeniculum vulgare,
an extract derived from fruits of Hypomyces lactifluorum,
an extract derived from leaves, stems, or a combination thereof of Iberis sempervirens,
an extract derived from leaves, fruits, stems, seeds, or a combination thereof of Lotus corniculatus,
an extract derived from fruits of Lunaria annua,
an extract derived from fruits of Manihot esculenta,
an extract derived from leaves, flowers, stems, or a combination thereof of Matricaria recutita,
an extract derived from leaves, stems, or a combination thereof of Melilotus albus,
an extract derived from leaves of Phaseolus vulgaris,
an extract derived from leaves, stems, or a combination thereof of Physostegia virginiana,
an extract derived from leaves, stems, or a combination thereof of Pisum sativum,
an extract derived from leaves of Raphanus raphanistrum,
an extract derived from leaves of Rheum rhabarbarum,
an extract derived from leaves of Ribes sylvestre,
an extract derived from fruits of Rubus occidentalis,
an extract derived from roots of Rumex crispus,
an extract derived from leaves of Rumex scutatus,
an extract derived from leaves, stems, or a combination thereof of Salvia officinalis,
an extract derived from flowers of Solidago canadensis,
an extract derived from leaves, flowers, stems, or a combination thereof of Solidago spp.,
an extract derived from leaves, flowers, stems, or a combination thereof of Solidago×hybrida,
an extract derived from leaves of Tanacetum cinerariifolium,
an extract derived from leaves of Taraxacum officinale,
an extract derived from leaves of Tropaeolum majus,
an extract derived from leaves, fruits, stems, or a combination thereof of Tsuga canadensis,
an extract derived from leaves, stems, or a combination thereof of Tsuga diversifolia,
an extract derived from fruits of Vaccinium angustifolium,
an extract derived from leaves of Zea mays, and
an extract derived from fruits of Zingiber officinale.

25. The method according to claim 20, wherein each of said one or more plant extracts is prepared by extraction of plant material with an alcoholic solvent, an aqueous solvent, or a combination thereof.

26. The method according to claim 20, wherein each of said one or more plant extracts is derived from Amaranthus candathus, Ambrosia artemesiifolia, Aronia xprunifolia, Brassica napus, Brassica oleracea, Bromus inermis, Chenopodium quinoa, Citrullus lanatus, Dolichos lablab, Foeniculum vulgare, Hypomyces lactifluorum, Lotus corniculatus, Manihot esculenta, Matricaria recutita, Melilotus albus, Phaseolus vulgaris, Pisum sativum, Raphanus raphanistrum, Ribes sylvestre, Rumex crispus, Rumex scutatus, Tanacetum cinerariifolium, Tropaeolum majus, Tsuga canadensis, Tsuga diversifolia, Vaccinium angustifolium, Zea mays or Zingiber officinale.

27. The method according to claim 26, wherein each of said one or more plant extracts is selected from:
an extract derived from leaves of Amaranthus candathus,
an extract derived from flowers of Ambrosia artemesiifolia,
an extract derived from leaves, stems, or a combination thereof of Aronia×prunifolia,
an extract derived from leaves of Brassica napus,
an extract derived from leaves of Brassica oleracea,
an extract derived from leaves of Bromus inermis,
an extract derived from leaves, stems, seeds, or a combination thereof of Chenopodium quinoa,
an extract derived from leaves of Citrullus lanatus,
an extract derived from flowers, fruits, or a combination thereof of Dolichos lablab,
an extract derived from leaves of Foeniculum vulgare,
an extract derived from fruits of Hypomyces lactifluorum,
an extract derived from leaves, fruits, stems, seeds, or a combination thereof of Lotus corniculatus,
an extract derived from fruits of Manihot esculenta,
an extract derived from leaves, flowers, stems, or a combination thereof of Matricaria recutita,
an extract derived from leaves, stems, or a combination thereof of Melilotus albus,
an extract derived from leaves of Phaseolus vulgaris,
an extract derived from leaves, stems, or a combination thereof of Pisum sativum,
an extract derived from leaves of Raphanus raphanistrum,
an extract derived from leaves of Ribes sylvestre,
an extract derived from roots of Rumex crispus,
an extract derived from leaves of Rumex scutatus,
an extract derived from leaves of Tanacetum cinerariifolium,
an extract derived from leaves of Tropaeolum majus,
an extract derived from leaves, fruits, stems, or a combination thereof of Tsuga canadensis,
an extract derived from leaves, stems, or a combination thereof of Tsuga diversifolia,
an extract derived from fruits of Vaccinium angustifolium,
an extract derived from leaves of Zea mays, and
an extract derived from fruits of Zingiber officinale.

28. The method according to claim 20, wherein said formulation is administered orally to said subject.

29. The method according to claim 20, wherein said formulation is administered topically to said subject.

30. A method of inhibiting metastasis of cancer cells in a subject comprising administering to said subject an effective amount of the formulation according to claim 1, wherein said formulation inhibits migration of neoplastic cells.

31. The method according to claim 30, wherein at least one of said one or more plant extracts is derived from a plant that has been subjected to one or more stress prior to harvest.

32. The method according to claim 31, wherein said one or more stress is a chemical stress.

33. The method according to claim 32, wherein said chemical stress comprises treatment with gamma linolenic acid or arachidonic acid.

34. The method according to claim 30, wherein each of said one or more plant extracts is selected from:
an extract derived from fruits, flowers, or a combination thereof of Allium tuberosum,
an extract derived from leaves, stems, or a combination thereof of Althacea officinalis,
an extract derived from leaves of Amaranthus candathus,
an extract derived from flowers of Ambrosia artemesiifolia,
an extract derived from entire Angelica sinensis plants,
an extract derived from leaves, stems, or a combination thereof of Aronia×prunifolia,
an extract derived from leaves of Asarum europaeum,
an extract derived from leaves, flowers, fruits, stems, or a combination thereof of Begonia hannii,
an extract derived from leaves, flowers, stems, or a combination thereof of Begonia polygonoides,
an extract derived from leaves of Brassica napus,
an extract derived from leaves of Brassica oleracea,
an extract derived from leaves of Bromus inermis,
an extract derived from leaves, stems, seeds, or a combination thereof of Chenopodium quinoa,
an extract derived from leaves of Citrullus lanatus,
an extract derived from entire Conyza canadensis plants,
an extract derived from fruits of Cynara cardunculus subsp. Cardunculus,
an extract derived from leaves of Daucus carota,
an extract derived from flowers, fruits, or a combination thereof of Dolichos lablab,
an extract derived from leaves of Foeniculum vulgare,
an extract derived from fruits of Hypomyces lactifluorum,
an extract derived from leaves, stems, or a combination thereof of Iberis sempervirens,
an extract derived from leaves, fruits, stems, seeds, or a combination thereof of Lotus corniculatus,
an extract derived from fruits of Lunaria annua,
an extract derived from fruits of Manihot esculenta,
an extract derived from leaves, flowers, stems, or a combination thereof of Matricaria recutita,
an extract derived from leaves, stems, or a combination thereof of Melilotus albus,
an extract derived from leaves of Phaseolus vulgaris,
an extract derived from leaves, stems, or a combination thereof of Physostegia virginiana,
an extract derived from leaves, stems, or a combination thereof of Pisum sativum,
an extract derived from leaves of Raphanus raphanistrum,
an extract derived from leaves of Rheum rhabarbarum,
an extract derived from leaves of Ribes sylvestre,
an extract derived from fruits of Rubus occidentalis,
an extract derived from roots of Rumex crispus,
an extract derived from leaves of Rumex scutatus,
an extract derived from leaves, stems, or a combination thereof of Salvia officinalis,
an extract derived from flowers of Solidago canadensis,
an extract derived from leaves, flowers, stems, or a combination thereof of Solidago spp.,
an extract derived from leaves, flowers, stems, or a combination thereof of Solidago×hybrida,
an extract derived from leaves of Tanacetum cinerariifolium,
an extract derived from leaves of Taraxacum officinale,
an extract derived from leaves of Tropaeolum majus,
an extract derived from leaves, fruits, stems, or a combination thereof of Tsuga canadensis,
an extract derived from leaves, stems, or a combination thereof of Tsuga diversifolia,
an extract derived from fruits of Vaccinium angustifolium,
an extract derived from leaves of Zea mays, and
an extract derived from fruits of Zingiber officinale.

35. The method according to claim 30, wherein each of said one or more plant extracts is prepared by extraction of plant material with an alcoholic solvent, an aqueous solvent, or a combination thereof.

36. The method according to claim 30, wherein each of said one or more plant extracts is derived from Allium tuberosum, Althacea officinalis, Amaranthus candathus, Ambrosia artemesiifolia, Angelica sinensis, Aronia×prunifolia, Asarum europaeum, Begonia hannii, Begonia polygonoides, Brassica napa, Brassica oleracea, Bromus inermis, Chenopodium quinoa, Citrullus lanatus, Conyza canadensis, Cynara cardunculus, Dolichos lablab, Hypomyces lactifluorum, Iberis sempervirens, Lotus corniculatus, Lunaria annua, Manihot esculenta, Matricaria recutita, Melilotus albus, Phaseolus vulgaris, Physostegia virginiana, Pisum sativum, Raphanus raphanistrum, Rheum rabarbarum, Ribes sylvestre, Rubus occidentalis, Rumex crispus, Rumex scutatus, Salvia officinalis, Solidago canadensis, Solidago spp., Solidago×hybrida, Tanacetum cinerariifolium, Taraxacum officinale, Tropaeolum majus, Tsuga canadensis, Tsuga diversifolia, Vaccinium angustifolium, Zea mays or Zingiber officinale.

37. The method according to claim 36, wherein each of said one or more plant extracts is selected from:
an extract derived from fruits, flowers, or a combination thereof of Allium tuberosum,
an extract derived from leaves, stems, or a combination thereof of Althacea officinalis,
an extract derived from leaves of Amaranthus candathus,
an extract derived from flowers of Ambrosia artemesiifolia,
an extract derived from entire Angelica sinensis plants,
an extract derived from leaves, stems, or a combination thereof of Aronia×prunifolia,
an extract derived from leaves of Asarum europaeum,
an extract derived from leaves, flowers, fruits, stems, or a combination thereof of Begonia hannii,
an extract derived from leaves, flowers, stems, or a combination thereof of Begonia polygonoides,
an extract derived from leaves of Brassica napus,
an extract derived from leaves of Brassica oleracea,
an extract derived from leaves of Bromus inermis,
an extract derived from leaves, stems, seeds, or a combination thereof of Chenopodium quinoa,
an extract derived from leaves of Citrullus lanatus,
an extract derived from entire Conyza canadensis plants,
an extract derived from fruits of Cynara cardunculus subsp. Cardunculus,
an extract derived from flowers, fruits, or a combination thereof of Dolichos lablab,
an extract derived from fruits of Hypomyces lactifluorum,
an extract derived from leaves, stems, or a combination thereof of Iberis sempervirens,
an extract derived from leaves, fruits, stems, seeds, or a combination thereof of Lotus corniculatus,
an extract derived from fruits of Lunaria annua,
an extract derived from fruits of Manihot esculenta,
an extract derived from leaves, flowers, stems, or a combination thereof of Matricaria recutita,
an extract derived from leaves, stems, or a combination thereof of Melilotus albus,
an extract derived from leaves of Phaseolus vulgaris,
an extract derived from leaves, stems, or a combination thereof of Physostegia virginiana,
an extract derived from leaves, stems, or a combination thereof of Pisum sativum,
an extract derived from leaves of Raphanus raphanistrum,
an extract derived from leaves of Rheum rhabarbarum,
an extract derived from leaves of Ribes sylvestre,
an extract derived from fruits of Rubus occidentalis,
an extract derived from a root of Rumex crispus,
an extract derived from leaves of Rumex scutatus,
an extract derived from leaves, stems, or a combination thereof of Salvia officinalis,
an extract derived from flowers of Solidago canadensis,
an extract derived from leaves, flowers, stems, or a combination thereof of Solidago spp.,
an extract derived from leaves, flowers, stems, or a combination thereof of Solidago×hybrida,
an extract derived from leaves of Tanacetum cinerariifolium,
an extract derived from leaves of Taraxacum officinale,
an extract derived from leaves of Tropaeolum majus,
an extract derived from leaves, fruits, stems, or a combination thereof of Tsuga canadensis,
an extract derived from leaves, stems, or a combination thereof of Tsuga diversifolia,
an extract derived from fruits of Vaccinium angustifolium,
an extract derived from leaves of Zea mays, and
an extract derived from fruits of Zingiber officinale.

38. The method according to claim 30, wherein said formulation is administered orally to said subject.

39. The method according to claim 30, wherein said formulation is administered topically to said subject.
